(19) United States
(12) Patent Application Publication
(10) Pub. No.: US 2024/0317706 A1
(43) Pub. Date: Sep. 26, 2024

(54) COMPOUNDS THAT MEDIATE PROTEIN DEGRADATION AND METHODS OF USE THEREOF

(71) Applicant: Monte Rosa Therapeutics, Inc., Boston, MA (US)

(72) Inventors: Bernhard Fasching, Basel (CH); Thomas Ryckmans, Basel (CH); Andreas Ritzén, Basel (CH); Vladimiras Oleinikovas, Basel (CH)

(21) Appl. No.: 18/640,987

(22) Filed: Apr. 19, 2024

Related U.S. Application Data
(63) Continuation of application No. PCT/US2022/047437, filed on Oct. 21, 2022.
(60) Provisional application No. 63/270,933, filed on Oct. 22, 2021.

(51) Int. Cl. C07D 401/04; A61K 31/454; A61K 31/4545; C07D 401/14; C07D 405/14; C07D 409/14; C07D 413/14; C07D 493/08

(52) U.S. Cl. CPC C07D 401/04; A61K 31/454; A61K 31/4545; C07D 401/14; C07D 405/14; C07D 409/14; C07D 413/14; C07D 493/08

(57) ABSTRACT

Described herein, in part, are compounds that mediate the degradation of cyclin-dependent kinase 2 (CDK2), and are therefore useful in the treatment of various disorders, such as cancer.

### CROSS-REFERENCE TO RELATED APPLICATIONS

This application is a continuation of International Application Number PCT/US2022/047437, filed on Oct. 21, 2022, which claims the benefit of, and priority to, U.S. Provisional Application No. 63/270,933, filed Oct. 22, 2021, the contents of which are incorporated herein by reference.

### BACKGROUND

The ubiquitin proteasome system can be manipulated with different small molecules to trigger targeted degradation of specific proteins of interest. Promoting the targeted degradation of pathogenic proteins using small molecule degraders is emerging as a new modality in the treatment of diseases. One such modality relies on redirecting the activity of E3 ligases such as cereblon (a phenomenon known as E3 reprogramming) using low molecular weight compounds, which have been termed molecular glues to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein.

There exists a need for therapeutics that effectively mediate the degradation of certain proteins for the treatment of diseases.

### SUMMARY

Described herein, in part, are compounds contemplated as modulators of cereblon to mediate the degradation of a protein, and are therefore are useful in the treatment of disorders, such as cancer. For example, it has been found that compounds of the present disclosure mediate the targeted degradation of the protein cyclin-dependent kinase 2 (CDK2).

In one aspect, described herein is a compound of Formula (I):

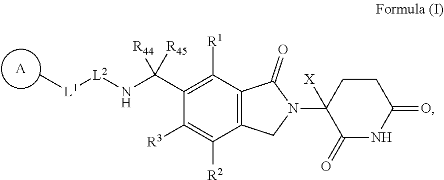

or a pharmaceutically acceptable salt thereof, wherein: X is H or deuterium; L¹ is selected from the group consisting of:

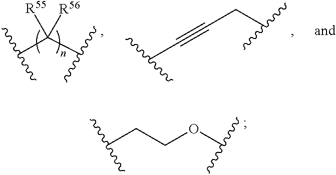

L² is selected from the group consisting of:

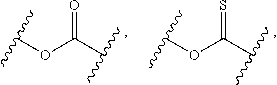 [SMILES: *OC(*)=O ; *OC(*)=S]

or a 5-6 membered heteroaryl; each of R¹, R², R³ is independently selected from the group consisting of hydrogen, halogen, hydroxy, cyano, nitrogen, oxo, —N(R^{Za})(R^{Zb}), C₁₋₆ alkoxy, and C₁₋₆ alkyl, wherein C₁₋₆ alkyl is optionally substituted with one or more halogens; each of R⁴⁴ and R⁴⁵ is independently H or C₁₋₆ alkyl; or R⁴⁴ and R⁴⁵, together with the carbon to which they are attached, form a C₃₋₁₂ cycloalkyl ring; each of R⁵⁵ and R⁵⁶ is independently selected from the group consisting of H, C₁₋₆ alkyl, aryl, and C₃₋₁₂ cycloalkyl, wherein C₁₋₆ alkyl is optionally substituted with one or more substituents selected from the group consisting of C₁₋₆ alkoxy, aryl, and C₃₋₁₂ cycloalkyl; ring A is C₃₋₁₂ cycloalkyl or 3 to 10 membered heterocyclyl, wherein each of C₃₋₁₂ cycloalkyl and 3 to 10 membered heterocyclyl is optionally substituted with one or more occurrences of R⁴; each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; R^{Z} is H or C₁₋₆ alkyl; each of R^{Za} and R^{Zb} independently H or and C₁₋₆ alkyl; and n is 0, 1, 2, 3, or 4.

In an aspect, provided herein is a compound in Table 1 as described herein, or a pharmaceutically acceptable salt thereof.

In an aspect, described herein is a pharmaceutical composition comprising a compound described herein, or pharmaceutically acceptable salt thereof, and a pharmaceutically acceptable excipient.

In an aspect, described herein is a method of degrading CDK2 in a subject suffering from cancer, comprising administering to the subject an effective amount of a compound described herein, or pharmaceutically acceptable salt thereof, or a pharmaceutical composition described herein.

In an aspect, described herein is a method of treating cancer in a subject in need thereof, comprising administering to the subject a therapeutically effective amount of a compound described herein, or pharmaceutically acceptable salt thereof, or a pharmaceutical composition described herein.

In an aspect, described herein is a method of treating a solid tumor in a subject in need thereof, comprising administering to the subject a therapeutically effective amount of a compound described herein, or pharmaceutically acceptable salt thereof, or a pharmaceutical composition described herein.

In an aspect, described herein is a method of treating a liquid tumor in a subject in need thereof, comprising administering to the subject a therapeutically effective amount of a compound described herein, or pharmaceutically acceptable salt thereof, or a pharmaceutical composition described herein.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Compounds

In one aspect, described herein is a compound of Formula (I):

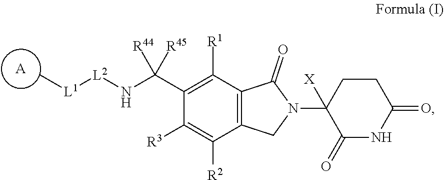

or a pharmaceutically acceptable salt thereof, wherein: X is H or deuterium; L¹ is selected from the group consisting of:

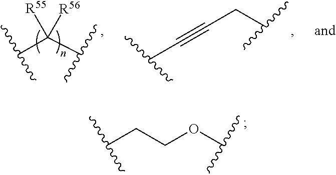

L² is selected from the group consisting of:

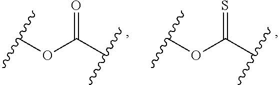 [SMILES: *OC(*)=O ; *OC(*)=S]

or a 5-6 membered heteroaryl; each of R¹, R², R³ is independently selected from the group consisting of hydrogen, halogen, hydroxy, cyano, nitrogen, oxo, —N(R^{Za})(R^{Zb}), C₁₋₆ alkoxy, and C₁₋₆ alkyl, wherein C₁₋₆ alkyl is optionally substituted with one or more halogens; each of R⁴⁴ and R⁴⁵ is independently H or C₁₋₆ alkyl; or R⁴⁴ and R⁴⁵, together with the carbon to which they are attached, form a C₃₋₁₂ cycloalkyl ring; each of R⁵⁵ and R⁵⁶ is independently selected from the group consisting of H, C₁₋₆ alkyl, aryl, and C₃₋₁₂ cycloalkyl, wherein C₁₋₆ alkyl is optionally substituted with one or more substituents selected from the group consisting of C₁₋₆ alkoxy, aryl, and C₃₋₁₂ cycloalkyl; ring A is C₃₋₁₂ cycloalkyl or 3 to 10 membered heterocyclyl, wherein each of C₃₋₁₂ cycloalkyl and 3 to 10 membered heterocyclyl is optionally substituted with one or more occurrences of R⁴; each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; R^{Z} is H or C₁₋₆ alkyl; each of R^{Za} and R^{Zb} independently H or and C₁₋₆ alkyl; and n is 0, 1, 2, 3, or 4.

In one aspect, described herein is a compound of Formula (I):

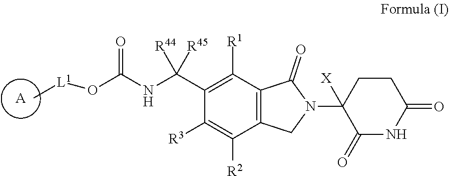

or a pharmaceutically acceptable salt thereof, wherein: X is H or deuterium; L¹ is:

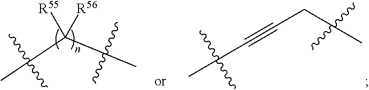

each of R¹, R², R³ is independently H, halogen, or C₁₋₆ alkyl; each of R⁴⁴ and R⁴⁵ is independently H or C₁₋₆ alkyl; each of R⁵⁵ and R⁵⁶ is independently H or C₁₋₆ alkyl, wherein C₁₋₆ alkyl is optionally substituted with one or more occurrences of C₁₋₆ alkoxy; ring A is C₃₋₁₂ cycloalkyl or 3 to 10 membered heterocyclyl, wherein each of C₃₋₁₂ cycloalkyl and 3 to 10 membered heterocyclyl is optionally substituted with one or more occurrences of R⁴; each occurrence of R⁴ is independently halogen, cyano, hydroxyl, oxo, —S(O)₂R^{A}, —NR^{Z}C(O)OR^{B}, —C(O)R^{C} C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; R^{Z} is H or C₁₋₆ alkyl; and n is 0, 1, 2, or 3.

In some embodiments, L² is

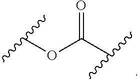 [SMILES: *OC(*)=O]

In some embodiments, L² is

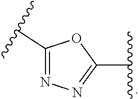 [SMILES: *c1nnc(*)o1]

In some embodiments, the compound is a compound of Formula (I-A):

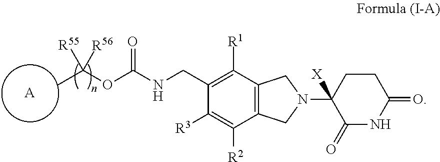

In some embodiments, the compound is a compound of Formula (I-B):

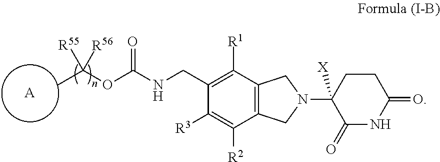

In some embodiments, the compound is a compound of Formula (I-C):

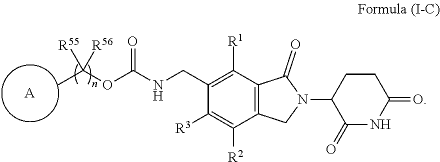

In some embodiments, the compound is a compound of Formula (I-Ca) or Formula (I-Cb):

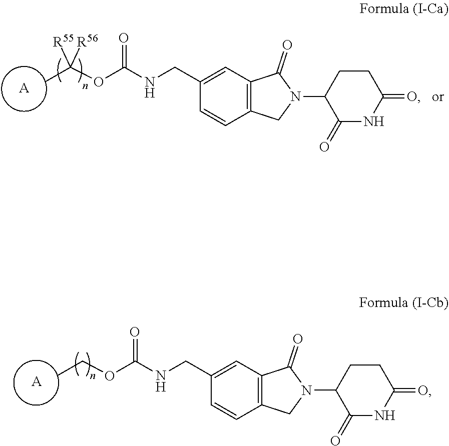

wherein

In some embodiments, the compound is a compound of Formula (I-D):

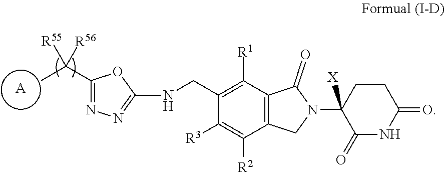

In some embodiments, the compound is a compound of Formula (I-E):

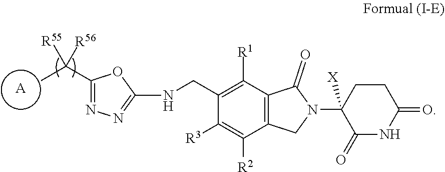

In some embodiments, the compound is a compound of Formula (I-F):

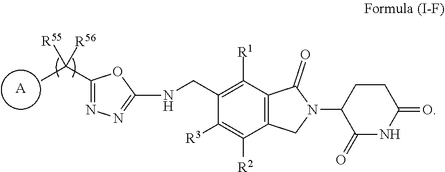

In some embodiments, the compound is a compound of Formula (I-Fa):

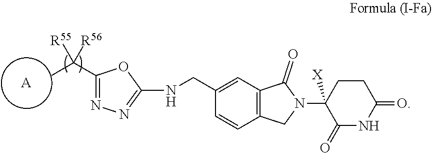

In some embodiments, the compound is a compound of Formula (I-G):

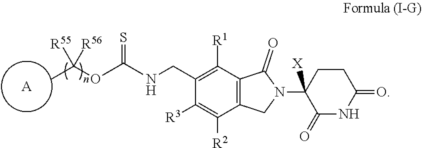

In some embodiments, the compound is a compound of Formula (I-H):

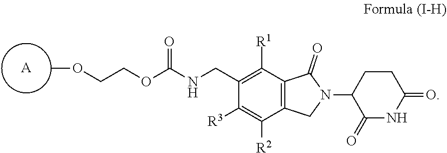

In some embodiments, the compound is a compound of Formula (I-I):

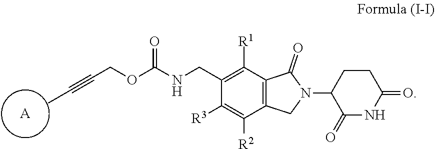

In some embodiments, the compound is a compound of Formula (I-J):

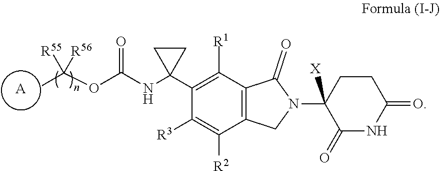

In some embodiments, X is H. In some embodiments, X is deuterium.

In some embodiments, R¹ is H. In some embodiments, R¹ is —OH. In some embodiments, R¹ is —NH₂. In some embodiments, R² is H. In some embodiments, R² is Cl, Br or F. In some embodiments, R² is —CF₃. In some embodiments, R³ is H.

In some embodiments, R¹, R², and R³ are H. In some embodiments, R¹ is —OH, R² is H, and R³ is H. In some embodiments, R¹ is H, R² is Br, and R³ is H. In some embodiments, R¹ is H, R² is —CF₃, and R³ is H. In some embodiments, R¹ is H, R² is Cl, and R³ is H. In some embodiments, R¹ is —OH, R² is Br, and R³ is H. In some embodiments, R¹ is —NH₂, R² is H, and R³ is H.

In some embodiments, R⁴⁴ and R⁴⁵ are H. In some embodiments, R⁴⁴ and R⁴⁵ are methyl. In some embodiments, R⁴⁴ is H or methyl. In some embodiments, R⁴⁵ is H or methyl. In some embodiments, R⁴⁴ and R⁴⁵ are taken together to form a cyclopropyl.

In some embodiments, R⁵⁶ H or methyl. In some embodiments, R⁵⁵ and R⁵⁶ are H. In some embodiments, R⁵⁵ and R⁵⁶ are methyl. In some embodiments, R⁵⁵ is H or methyl. In some embodiments, R⁵⁶ is H or methyl. In some embodiments, R⁵⁵ or R⁵⁶ is a cyclopropyl. In some embodiments, R⁵⁵ or R⁵⁶ is a phenyl.

In some embodiments, n is 4. In some embodiments, n is 3. In some embodiments, n is 2. In some embodiments, n is 1. In some embodiments, n is 0.

In some embodiments, ring A is C₃₋₁₂ cycloalkyl optionally substituted with one or more occurrences of R⁴. In some embodiments, ring A is 3 to 10 membered heterocyclyl optionally substituted with one or more occurrences of R⁴. In some embodiments, ring A is a spirocyclic C₃₋₁₂ cycloalkyl optionally substituted with one or more occurrences of R⁴. In some embodiments, ring A is a spirocyclic 3 to 10 membered heterocyclyl optionally substituted with one or more occurrences of R⁴.

In some embodiments, ring A is selected from the group consisting of:

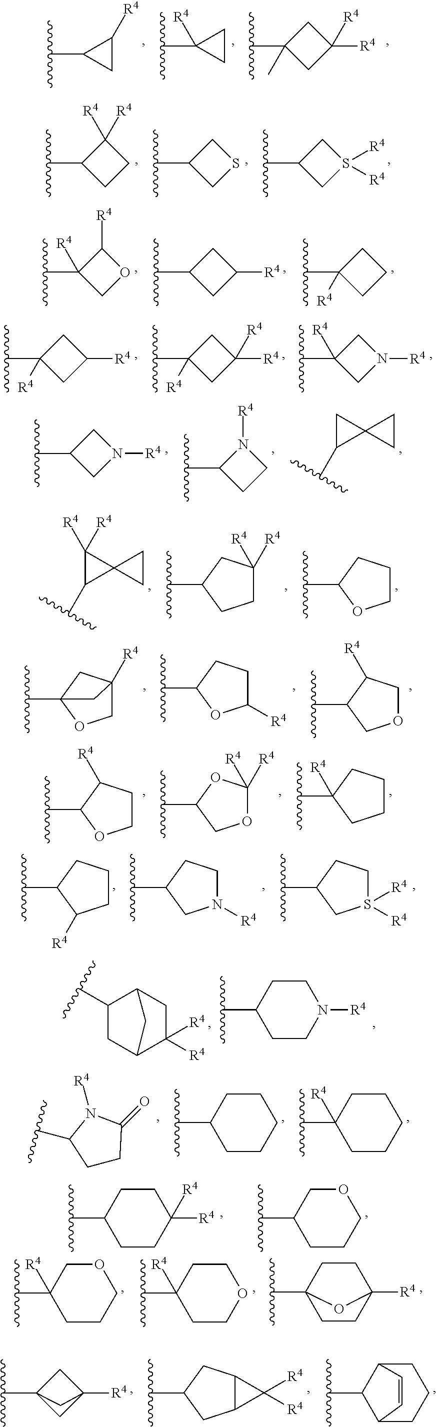

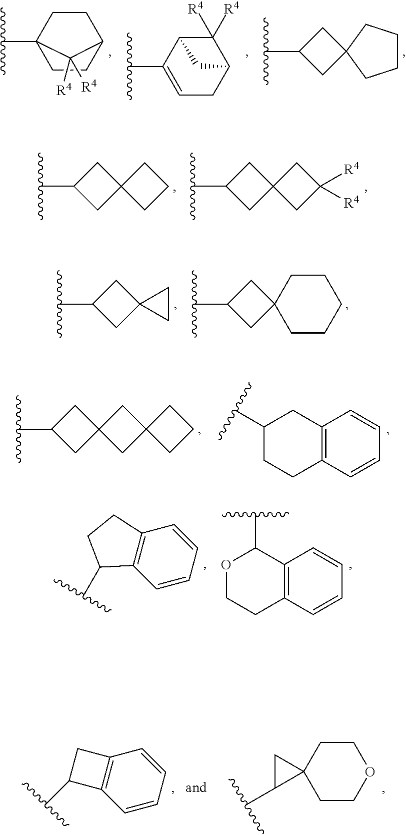

wherein each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₄ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; and R^{Z} is H or C₁₋₆ alkyl.

In some embodiments, ring A selected from the group consisting of:

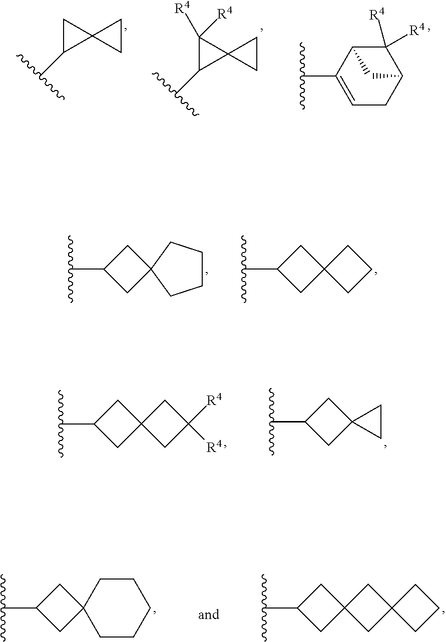

wherein each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; and R^{Z} is H or C₁₋₆ alkyl.

In some embodiments, the compound is a compound described in Table 1 below. Table 1 also includes the compound number of each compound in accordance with the contents of the present specification.

TABLE 1
Exemplary Compounds

| Compound | |
|---|---|
| No. | Structure |
| 1 | 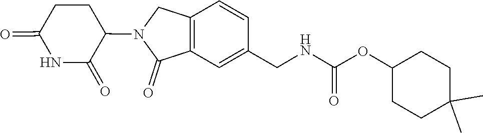 [SMILES: CC1(C)CCC(OC(=O)NCc2ccc3c(c2)C(=O)N(C2CCC(=O)NC2=O)C3)CC1] |
| 2 | 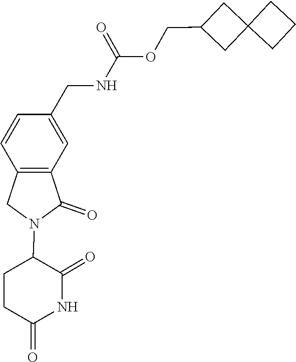 [SMILES: O=C1CCC(N2Cc3ccc(CNC(=O)OCC4CC5(CCC5)C4)cc3C2=O)C(=O)N1] |
| 3 | 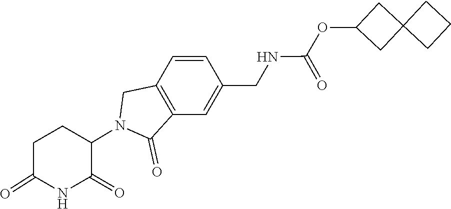 [SMILES: O=C1CCC(N2Cc3ccc(CNC(=O)OC4CC5(CCC5)C4)cc3C2=O)C(=O)N1] |
| 4 | 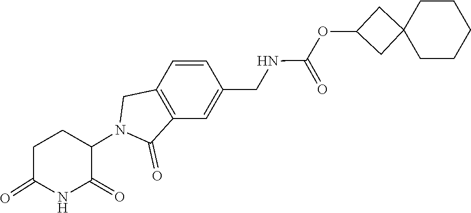 [SMILES: O=C1CCC(N2Cc3ccc(CNC(=O)OC4CC5(CCCCC5)C4)cc3C2=O)C(=O)N1] |
| 5 | 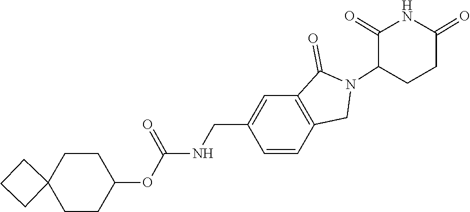 [SMILES: O=C1CCC(N2Cc3ccc(CNC(=O)OC4CCC5(CCC5)CC4)cc3C2=O)C(=O)N1] |
| 6 | 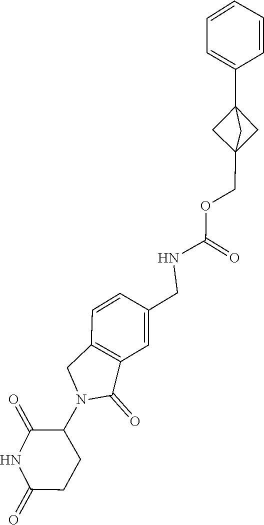 [SMILES: O=C1CCC(N2Cc3ccc(CNC(=O)OCC45CC(c6ccccc6)(C4)C5)cc3C2=O)C(=O)N1] |
| 7 | 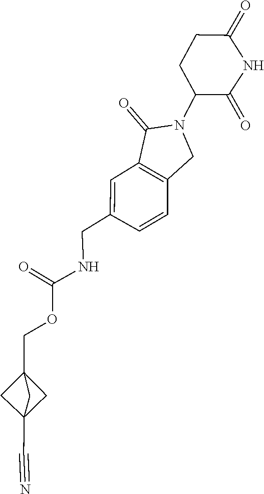 [SMILES: N#CC12CC(COC(=O)NCc3ccc4c(c3)C(=O)N(C3CCC(=O)NC3=O)C4)(C1)C2] |
| 8 | 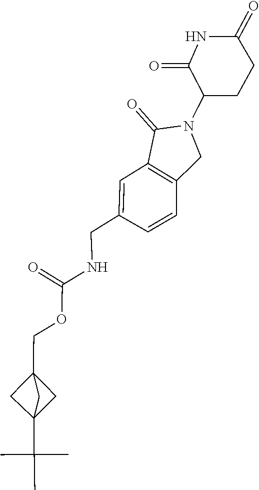 [SMILES: CC(C)(C)C12CC(COC(=O)NCc3ccc4c(c3)C(=O)N(C3CCC(=O)NC3=O)C4)(C1)C2] |
| 9 | 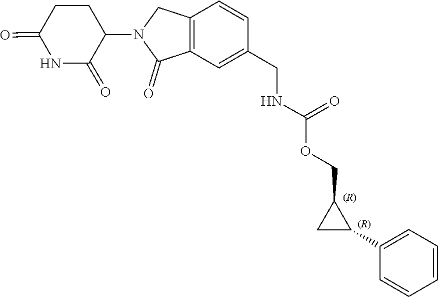 |
| 10 | 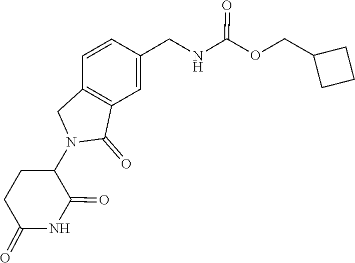 [SMILES: O=C1CCC(N2Cc3ccc(CNC(=O)OCC4CCC4)cc3C2=O)C(=O)N1] |
| 11 | 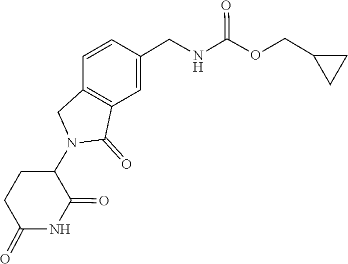 [SMILES: O=C1CCC(N2Cc3ccc(CNC(=O)OCC4CC4)cc3C2=O)C(=O)N1] |
| 12 | 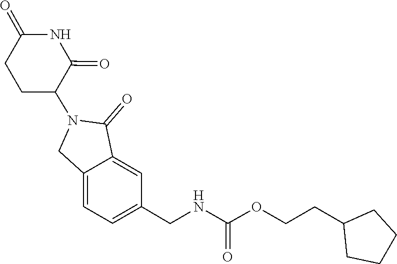 [SMILES: O=C1CCC(N2Cc3ccc(CNC(=O)OCCC4CCCC4)cc3C2=O)C(=O)N1] |
| 13 | 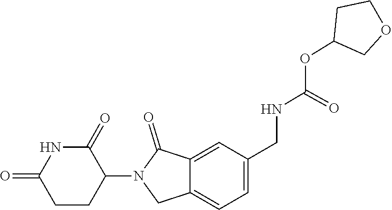 [SMILES: O=C1CCC(N2Cc3ccc(CNC(=O)OC4CCOC4)cc3C2=O)C(=O)N1] |
| 14 | 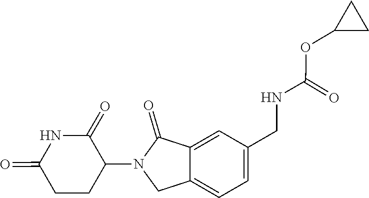 [SMILES: O=C1CCC(N2Cc3ccc(CNC(=O)OC4CC4)cc3C2=O)C(=O)N1] |
| 15 | 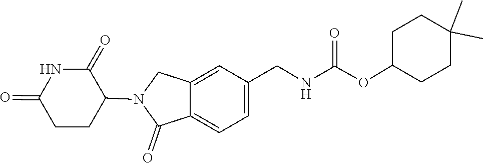 [SMILES: CC1(C)CCC(OC(=O)NCc2ccc3c(c2)CN(C2CCC(=O)NC2=O)C3=O)CC1] |
| 16 | 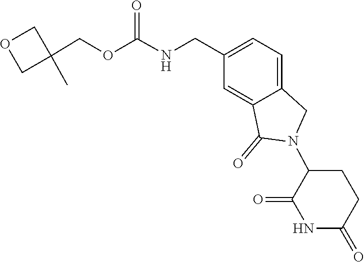 [SMILES: CC1(COC(=O)NCc2ccc3c(c2)C(=O)N(C2CCC(=O)NC2=O)C3)COC1] |
| 17 | 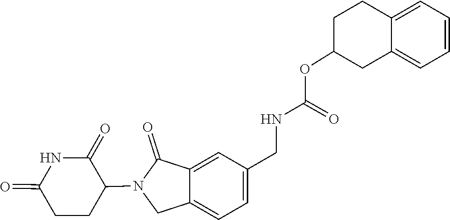 [SMILES: O=C1CCC(N2Cc3ccc(CNC(=O)OC4CCc5ccccc5C4)cc3C2=O)C(=O)N1] |
| 18 | 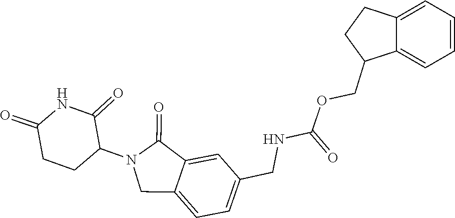 [SMILES: O=C1CCC(N2Cc3ccc(CNC(=O)OCC4CCc5ccccc54)cc3C2=O)C(=O)N1] |
| 19 | 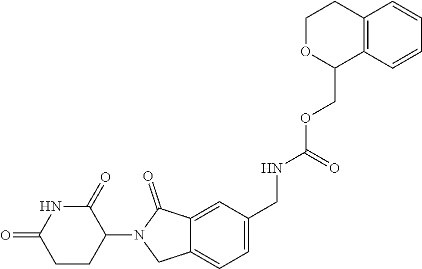 [SMILES: O=C1CCC(N2Cc3ccc(CNC(=O)OCC4OCCc5ccccc54)cc3C2=O)C(=O)N1] |
| 20 | 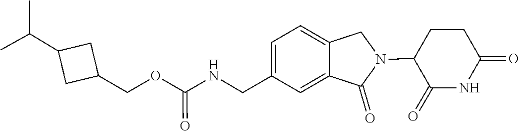 [SMILES: CC(C)C1CC(COC(=O)NCc2ccc3c(c2)C(=O)N(C2CCC(=O)NC2=O)C3)C1] |
| 21 | 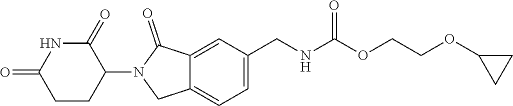 [SMILES: O=C1CCC(N2Cc3ccc(CNC(=O)OCCOC4CC4)cc3C2=O)C(=O)N1] |
| 22 | 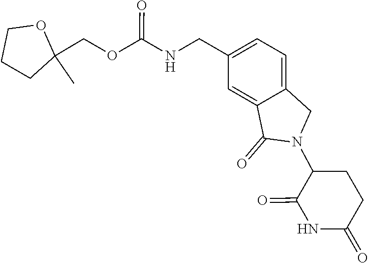 [SMILES: CC1(COC(=O)NCc2ccc3c(c2)C(=O)N(C2CCC(=O)NC2=O)C3)CCCO1] |
| 23 | 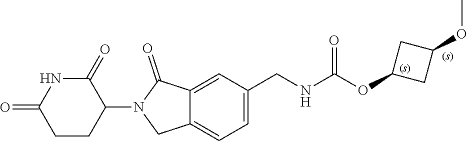 [SMILES: CO[C@H]1C[C@@H](OC(=O)NCc2ccc3c(c2)C(=O)N(C2CCC(=O)NC2=O)C3)C1] |
| 24 | 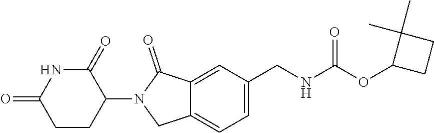 [SMILES: CC1(C)CCC1OC(=O)NCc1ccc2c(c1)C(=O)N(C1CCC(=O)NC1=O)C2] |
| 25 | 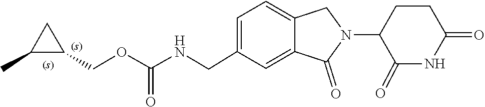 |
| 26 | 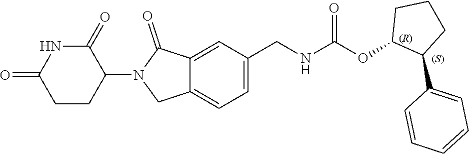 |
| 27 | 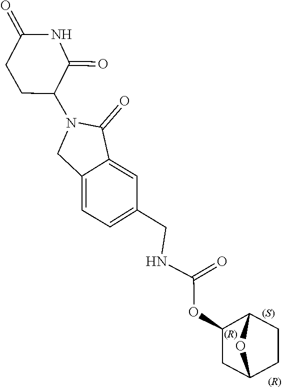 |
| 28 | 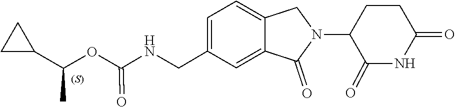 |
| 29 | 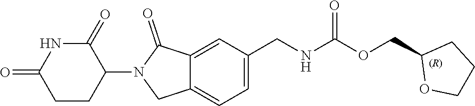 |
| 30 | 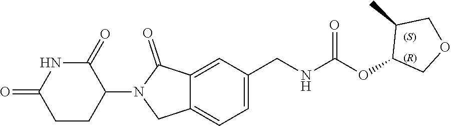 |
| 31 | 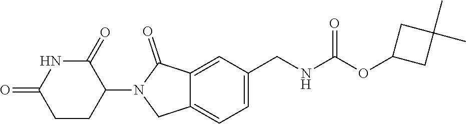 [SMILES: CC1(C)CC(OC(=O)NCc2ccc3c(c2)C(=O)N(C2CCC(=O)NC2=O)C3)C1] |
| 32 | 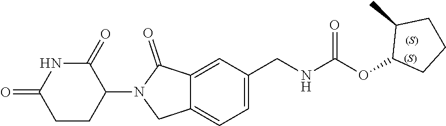 |
| 33 | 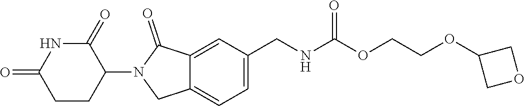 [SMILES: O=C1CCC(N2Cc3ccc(CNC(=O)OCCOC4COC4)cc3C2=O)C(=O)N1] |
| 34 | 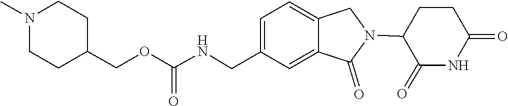 [SMILES: CN1CCC(COC(=O)NCc2ccc3c(c2)C(=O)N(C2CCC(=O)NC2=O)C3)CC1] |
| 35 | 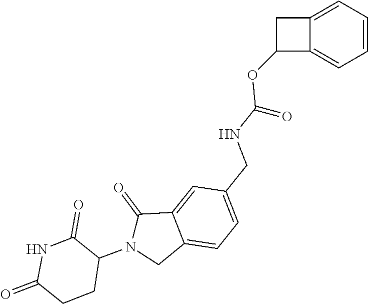 [SMILES: O=C1CCC(N2Cc3ccc(CNC(=O)OC4Cc5ccccc54)cc3C2=O)C(=O)N1] |
| 36 | 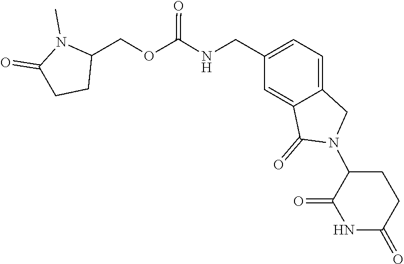 [SMILES: CN1C(=O)CCC1COC(=O)NCc1ccc2c(c1)C(=O)N(C1CCC(=O)NC1=O)C2] |
| 37 | 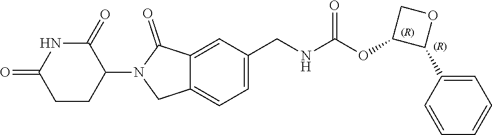 |
| 38 | 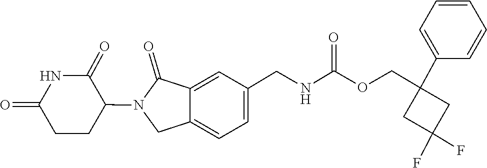 [SMILES: O=C1CCC(N2Cc3ccc(CNC(=O)OCC4(c5ccccc5)CC(F)(F)C4)cc3C2=O)C(=O)N1] |
| 39 | 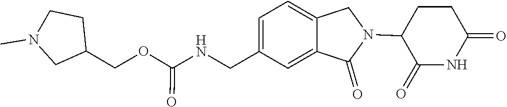 [SMILES: CN1CCC(COC(=O)NCc2ccc3c(c2)C(=O)N(C2CCC(=O)NC2=O)C3)C1] |
| 40 | 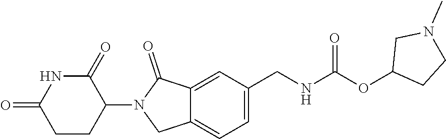 [SMILES: CN1CCC(OC(=O)NCc2ccc3c(c2)C(=O)N(C2CCC(=O)NC2=O)C3)C1] |
| 41 | 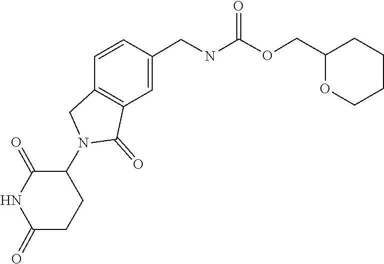 |
| 42 | 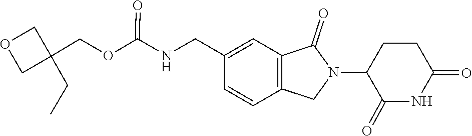 [SMILES: CCC1(COC(=O)NCc2ccc3c(c2)C(=O)N(C2CCC(=O)NC2=O)C3)COC1] |
| 43 | 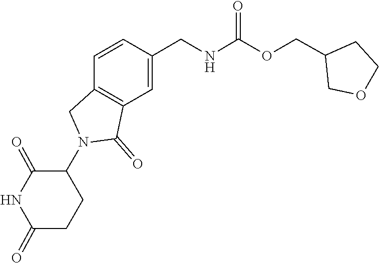 [SMILES: O=C1CCC(N2Cc3ccc(CNC(=O)OCC4CCOC4)cc3C2=O)C(=O)N1] |
| 44 | 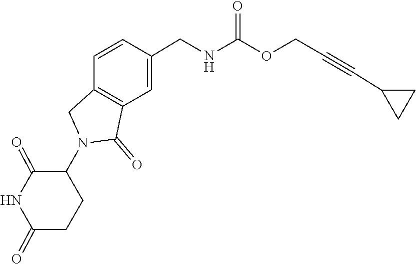 [SMILES: O=C1CCC(N2Cc3ccc(CNC(=O)OCC#CC4CC4)cc3C2=O)C(=O)N1] |
| 45 | 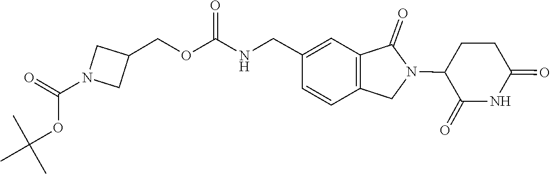 [SMILES: CC(C)(C)OC(=O)N1CC(COC(=O)NCc2ccc3c(c2)C(=O)N(C2CCC(=O)NC2=O)C3)C1] |
| 46 | 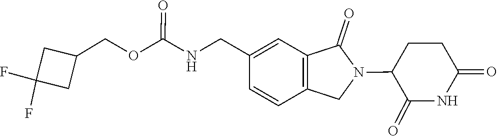 [SMILES: O=C1CCC(N2Cc3ccc(CNC(=O)OCC4CC(F)(F)C4)cc3C2=O)C(=O)N1] |
| 47 | 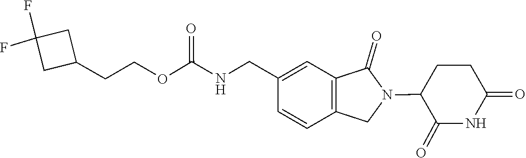 [SMILES: O=C1CCC(N2Cc3ccc(CNC(=O)OCCC4CC(F)(F)C4)cc3C2=O)C(=O)N1] |
| 48 | 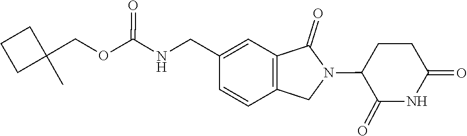 [SMILES: CC1(COC(=O)NCc2ccc3c(c2)C(=O)N(C2CCC(=O)NC2=O)C3)CCC1] |
| 49 | 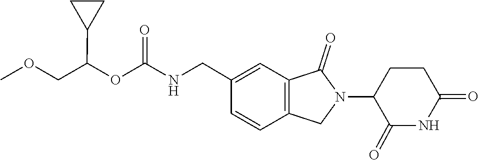 [SMILES: COCC(OC(=O)NCc1ccc2c(c1)C(=O)N(C1CCC(=O)NC1=O)C2)C1CC1] |
| 50 | 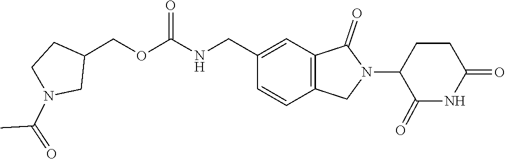 [SMILES: CC(=O)N1CCC(COC(=O)NCc2ccc3c(c2)C(=O)N(C2CCC(=O)NC2=O)C3)C1] |
| 51 | 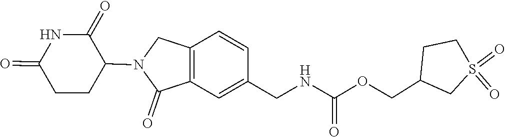 [SMILES: O=C1CCC(N2Cc3ccc(CNC(=O)OCC4CCS(=O)(=O)C4)cc3C2=O)C(=O)N1] |
| 52 | 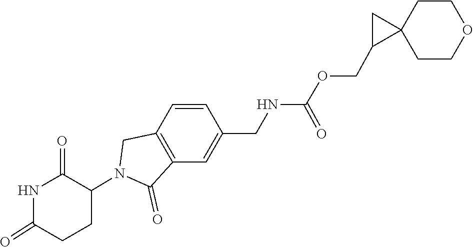 [SMILES: O=C1CCC(N2Cc3ccc(CNC(=O)OCC4CC45CCOCC5)cc3C2=O)C(=O)N1] |
| 53 | 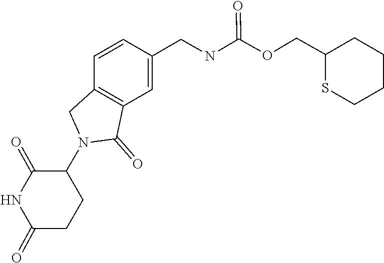 |
| 54 | 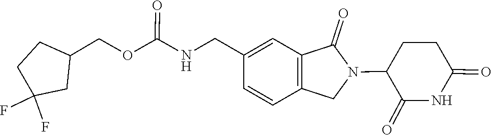 [SMILES: O=C1CCC(N2Cc3ccc(CNC(=O)OCC4CCC(F)(F)C4)cc3C2=O)C(=O)N1] |
| 55 | 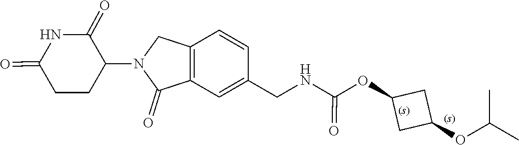 |
| 56 | 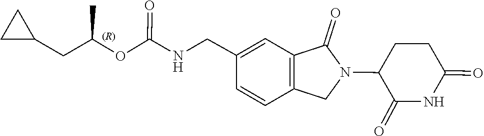 |
| 57 | 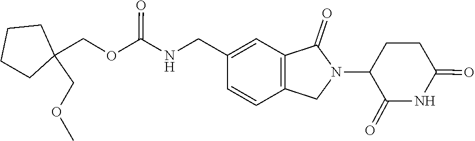 [SMILES: COCC1(COC(=O)NCc2ccc3c(c2)C(=O)N(C2CCC(=O)NC2=O)C3)CCCC1] |
| 58 | 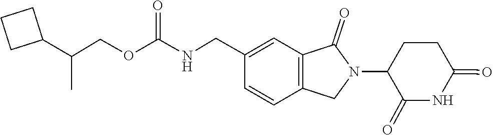 [SMILES: CC(COC(=O)NCc1ccc2c(c1)C(=O)N(C1CCC(=O)NC1=O)C2)C1CCC1] |
| 59 | 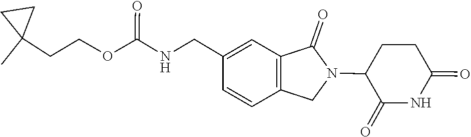 [SMILES: CC1(CCOC(=O)NCc2ccc3c(c2)C(=O)N(C2CCC(=O)NC2=O)C3)CC1] |
| 60 | 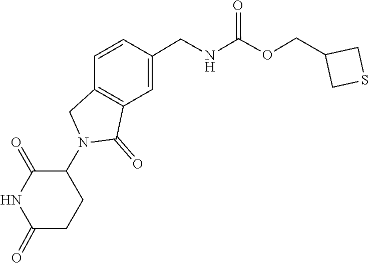 [SMILES: O=C1CCC(N2Cc3ccc(CNC(=O)OCC4CSC4)cc3C2=O)C(=O)N1] |
| 61 | 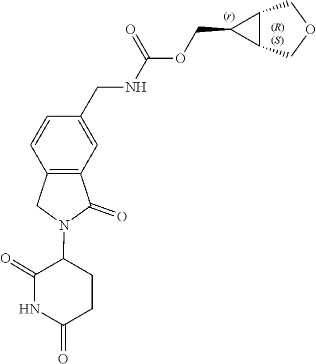 |
| 62 | 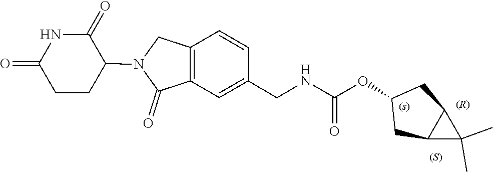 |
| 63 | 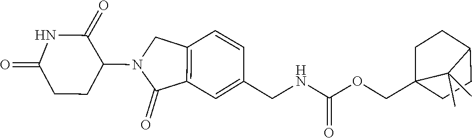 [SMILES: CC1(C)C2CCC1(COC(=O)NCc1ccc3c(c1)C(=O)N(C1CCC(=O)NC1=O)C3)CC2] |
| 64 | 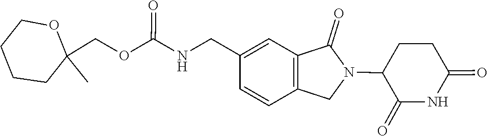 [SMILES: CC1(COC(=O)NCc2ccc3c(c2)C(=O)N(C2CCC(=O)NC2=O)C3)CCCCO1] |
| 65 | 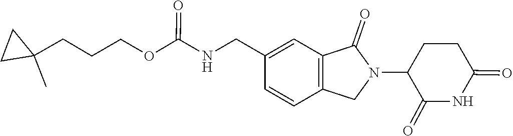 [SMILES: CC1(CCCOC(=O)NCc2ccc3c(c2)C(=O)N(C2CCC(=O)NC2=O)C3)CC1] |
| 66 | 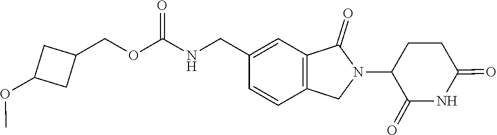 [SMILES: COC1CC(COC(=O)NCc2ccc3c(c2)C(=O)N(C2CCC(=O)NC2=O)C3)C1] |
| 67 | 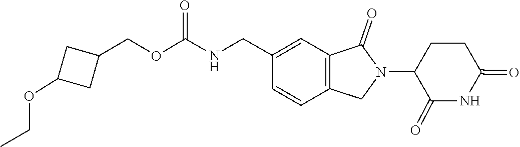 [SMILES: CCOC1CC(COC(=O)NCc2ccc3c(c2)C(=O)N(C2CCC(=O)NC2=O)C3)C1] |
| 68 | 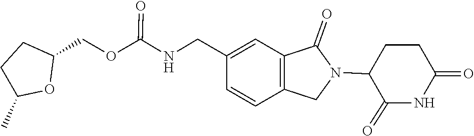 [SMILES: C[C@@H]1CC[C@H](COC(=O)NCc2ccc3c(c2)C(=O)N(C2CCC(=O)NC2=O)C3)O1] |
| 69 | 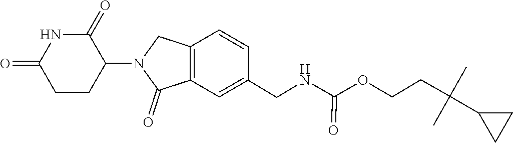 [SMILES: CC(C)(CCOC(=O)NCc1ccc2c(c1)C(=O)N(C1CCC(=O)NC1=O)C2)C1CC1] |
| 70 | 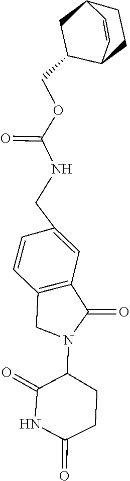 [SMILES: O=C1CCC(N2Cc3ccc(CNC(=O)OC[C@@H]4C[C@@H]5C=C[C@H]4CC5)cc3C2=O)C(=O)N1] |
| 71 | 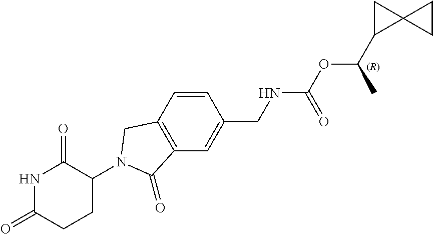 |
| 72 | 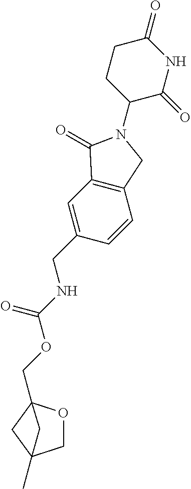 [SMILES: CC12COC(COC(=O)NCc3ccc4c(c3)C(=O)N(C3CCC(=O)NC3=O)C4)(C1)C2] |
| 73 | 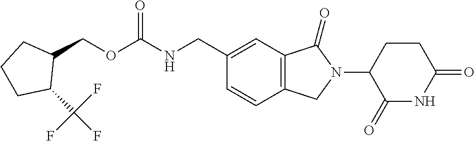 |
| 74 | 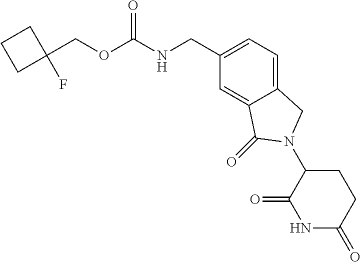 [SMILES: O=C1CCC(N2Cc3ccc(CNC(=O)OCC4(F)CCC4)cc3C2=O)C(=O)N1] |
| 75 | 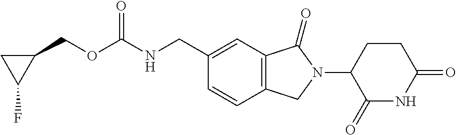 [SMILES: O=C1CCC(N2Cc3ccc(CNC(=O)OC[C@@H]4C[C@H]4F)cc3C2=O)C(=O)N1] |
| 76 | 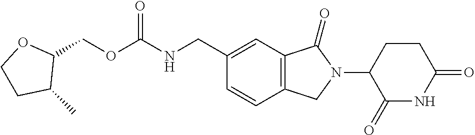 |
| 77 | 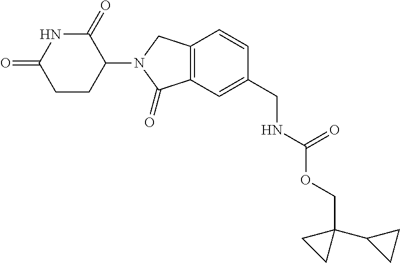 [SMILES: O=C1CCC(N2Cc3ccc(CNC(=O)OCC4(C5CC5)CC4)cc3C2=O)C(=O)N1] |
| 78 | 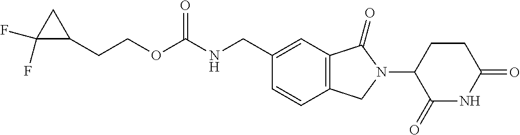 [SMILES: O=C1CCC(N2Cc3ccc(CNC(=O)OCCC4CC4(F)F)cc3C2=O)C(=O)N1] |
| 79 | 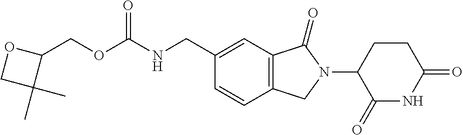 [SMILES: CC1(C)COC1COC(=O)NCc1ccc2c(c1)C(=O)N(C1CCC(=O)NC1=O)C2] |
| 80 | 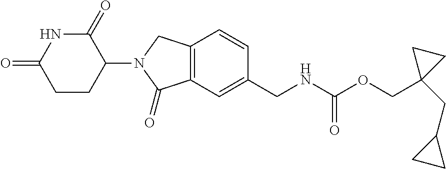 [SMILES: O=C1CCC(N2Cc3ccc(CNC(=O)OCC4(CC5CC5)CC4)cc3C2=O)C(=O)N1] |
| 81 | 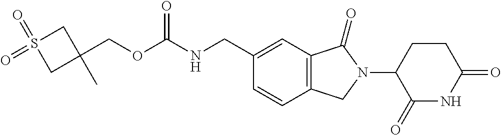 [SMILES: CC1(COC(=O)NCc2ccc3c(c2)C(=O)N(C2CCC(=O)NC2=O)C3)CS(=O)(=O)C1] |
| 82 | 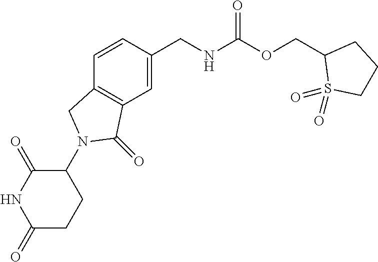 [SMILES: O=C1CCC(N2Cc3ccc(CNC(=O)OCC4CCCS4(=O)=O)cc3C2=O)C(=O)N1] |
| 83 | 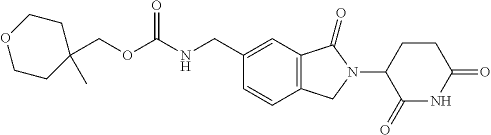 [SMILES: CC1(COC(=O)NCc2ccc3c(c2)C(=O)N(C2CCC(=O)NC2=O)C3)CCOCC1] |
| 84 | 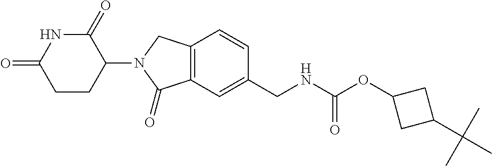 [SMILES: CC(C)(C)C1CC(OC(=O)NCc2ccc3c(c2)C(=O)N(C2CCC(=O)NC2=O)C3)C1] |
| 85 | 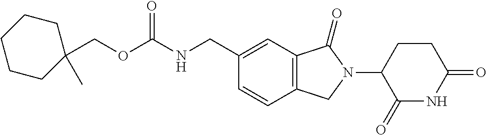 [SMILES: CC1(COC(=O)NCc2ccc3c(c2)C(=O)N(C2CCC(=O)NC2=O)C3)CCCCC1] |
| 86 | 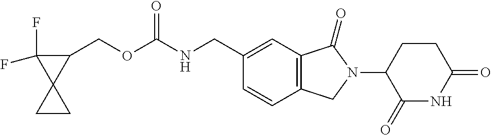 [SMILES: O=C1CCC(N2Cc3ccc(CNC(=O)OCC4C(F)(F)C45CC5)cc3C2=O)C(=O)N1] |
| 87 | 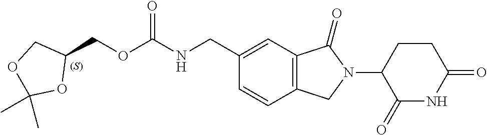 |
| 88 | 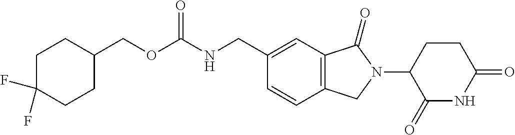 [SMILES: O=C1CCC(N2Cc3ccc(CNC(=O)OCC4CCC(F)(F)CC4)cc3C2=O)C(=O)N1] |
| 89 | 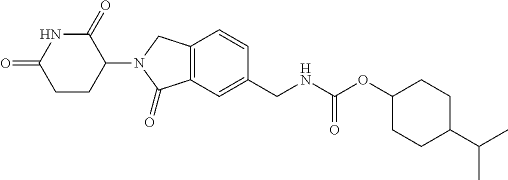 [SMILES: CC(C)C1CCC(OC(=O)NCc2ccc3c(c2)C(=O)N(C2CCC(=O)NC2=O)C3)CC1] |
| 90 | 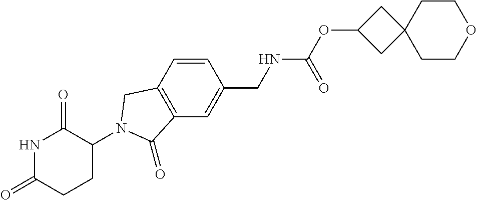 [SMILES: O=C1CCC(N2Cc3ccc(CNC(=O)OC4CC5(CCOCC5)C4)cc3C2=O)C(=O)N1] |
| 91 | 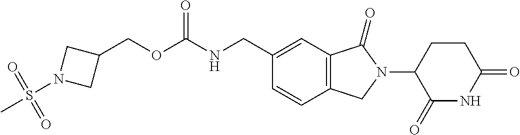 [SMILES: CS(=O)(=O)N1CC(COC(=O)NCc2ccc3c(c2)C(=O)N(C2CCC(=O)NC2=O)C3)C1] |
| 92 | 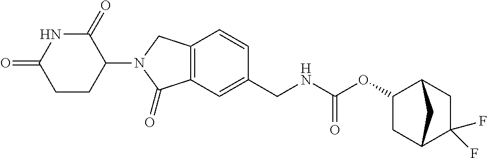 |
| 93 | 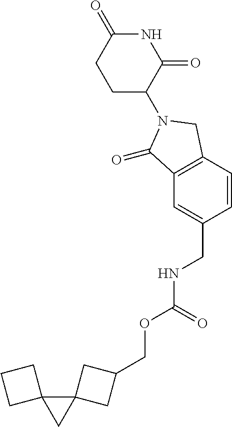 [SMILES: O=C1CCC(N2Cc3ccc(CNC(=O)OCC4CC5(C4)CC54CCC4)cc3C2=O)C(=O)N1] |
| 94 | 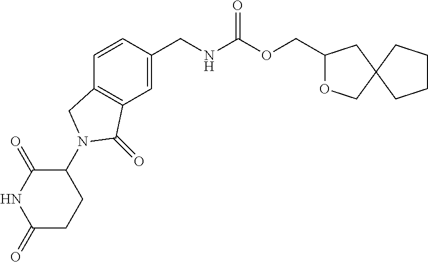 [SMILES: O=C1CCC(N2Cc3ccc(CNC(=O)OCC4CC5(CCCC5)CO4)cc3C2=O)C(=O)N1] |
| 95 | 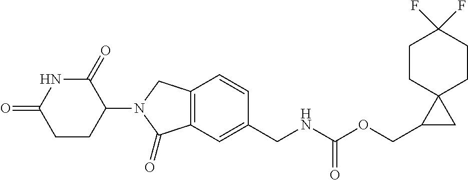 [SMILES: O=C1CCC(N2Cc3ccc(CNC(=O)OCC4CC45CCC(F)(F)CC5)cc3C2=O)C(=O)N1] |
| 96 | 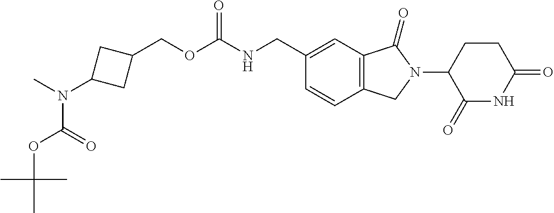 [SMILES: CN(C(=O)OC(C)(C)C)C1CC(COC(=O)NCc2ccc3c(c2)C(=O)N(C2CCC(=O)NC2=O)C3)C1] |
| 97 | 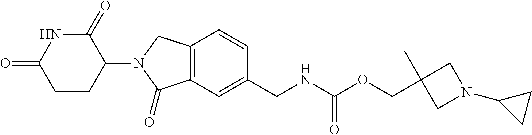 [SMILES: CC1(COC(=O)NCc2ccc3c(c2)C(=O)N(C2CCC(=O)NC2=O)C3)CN(C2CC2)C1] |
| 98 | 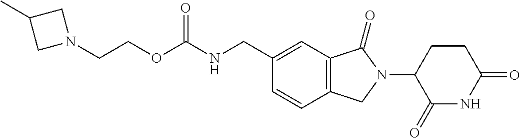 [SMILES: CC1CN(CCOC(=O)NCc2ccc3c(c2)C(=O)N(C2CCC(=O)NC2=O)C3)C1] |
| 99 | 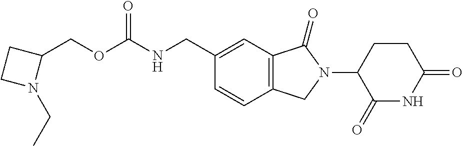 [SMILES: CCN1CCC1COC(=O)NCc1ccc2c(c1)C(=O)N(C1CCC(=O)NC1=O)C2] |
| 100 | 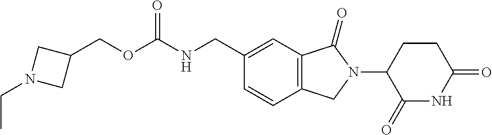 [SMILES: CCN1CC(COC(=O)NCc2ccc3c(c2)C(=O)N(C2CCC(=O)NC2=O)C3)C1] |
| 101 | 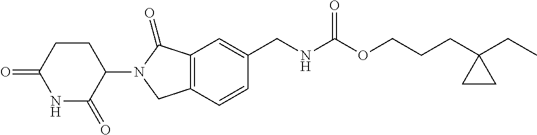 [SMILES: CCC1(CCCOC(=O)NCc2ccc3c(c2)C(=O)N(C2CCC(=O)NC2=O)C3)CC1] |
| 102 | 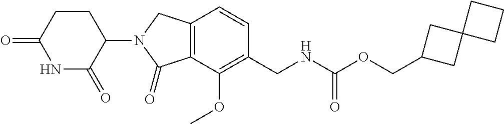 [SMILES: COc1c(CNC(=O)OCC2CC3(CCC3)C2)ccc2c1C(=O)N(C1CCC(=O)NC1=O)C2] |
| 103 | 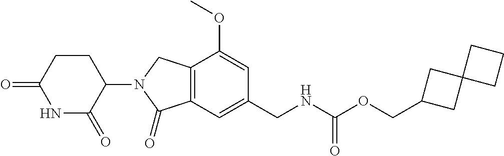 [SMILES: COc1cc(CNC(=O)OCC2CC3(CCC3)C2)cc2c1CN(C1CCC(=O)NC1=O)C2=O] |
| 104 | 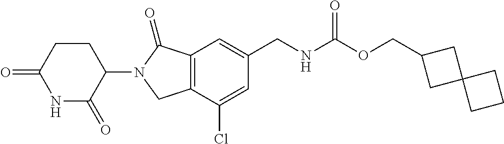 [SMILES: O=C1CCC(N2Cc3c(Cl)cc(CNC(=O)OCC4CC5(CCC5)C4)cc3C2=O)C(=O)N1] |
| 105 | 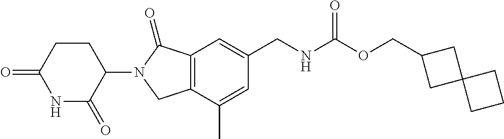 [SMILES: Cc1cc(CNC(=O)OCC2CC3(CCC3)C2)cc2c1CN(C1CCC(=O)NC1=O)C2=O] |
| 106 | 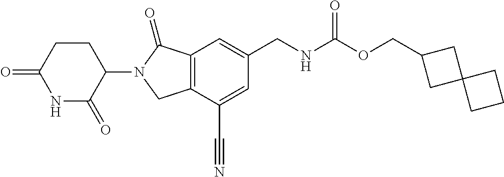 [SMILES: N#Cc1cc(CNC(=O)OCC2CC3(CCC3)C2)cc2c1CN(C1CCC(=O)NC1=O)C2=O] |
| 107 | 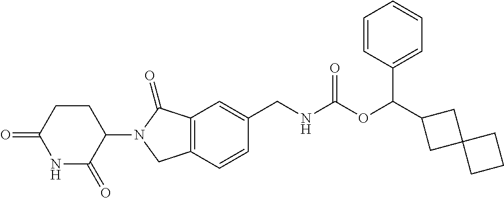 [SMILES: O=C1CCC(N2Cc3ccc(CNC(=O)OC(c4ccccc4)C4CC5(CCC5)C4)cc3C2=O)C(=O)N1] |
| 108 | 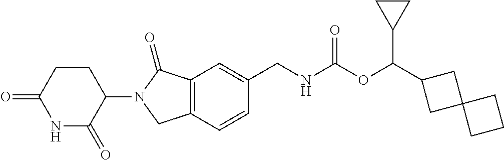 [SMILES: O=C1CCC(N2Cc3ccc(CNC(=O)OC(C4CC4)C4CC5(CCC5)C4)cc3C2=O)C(=O)N1] |
| 109 | 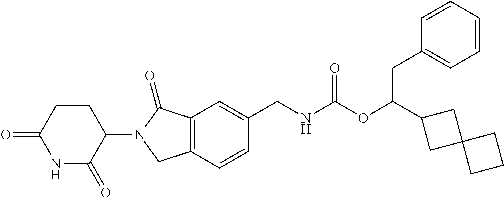 [SMILES: O=C1CCC(N2Cc3ccc(CNC(=O)OC(Cc4ccccc4)C4CC5(CCC5)C4)cc3C2=O)C(=O)N1] |
| 110 | 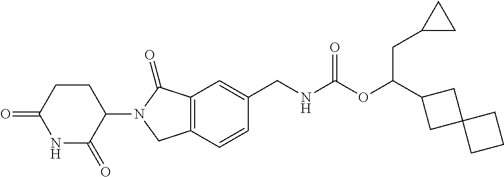 [SMILES: O=C1CCC(N2Cc3ccc(CNC(=O)OC(CC4CC4)C4CC5(CCC5)C4)cc3C2=O)C(=O)N1] |
| 111 | 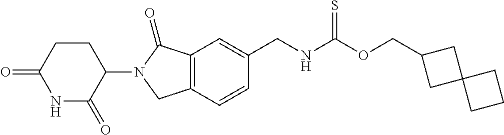 [SMILES: O=C1CCC(N2Cc3ccc(CNC(=S)OCC4CC5(CCC5)C4)cc3C2=O)C(=O)N1] |
| 112 | 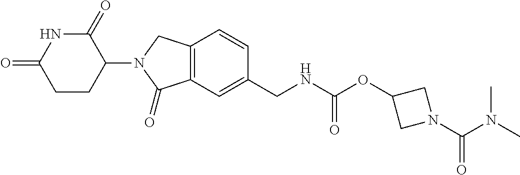 [SMILES: CN(C)C(=O)N1CC(OC(=O)NCc2ccc3c(c2)C(=O)N(C2CCC(=O)NC2=O)C3)C1] |
| 113 | 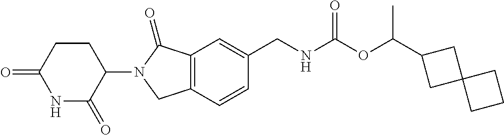 [SMILES: CC(OC(=O)NCc1ccc2c(c1)C(=O)N(C1CCC(=O)NC1=O)C2)C1CC2(CCC2)C1] |
| 114 | 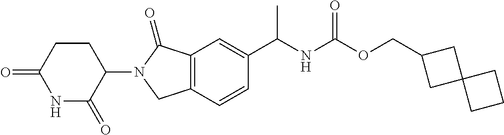 [SMILES: CC(NC(=O)OCC1CC2(CCC2)C1)c1ccc2c(c1)C(=O)N(C1CCC(=O)NC1=O)C2] |
| 115 | 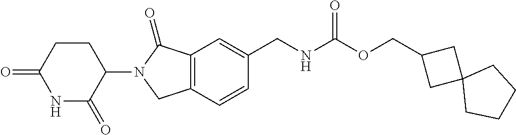 [SMILES: O=C1CCC(N2Cc3ccc(CNC(=O)OCC4CC5(CCCC5)C4)cc3C2=O)C(=O)N1] |
| 116 | 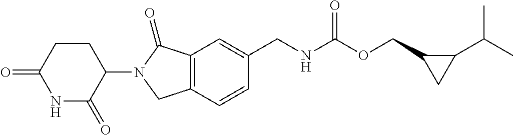 |
| 117 | 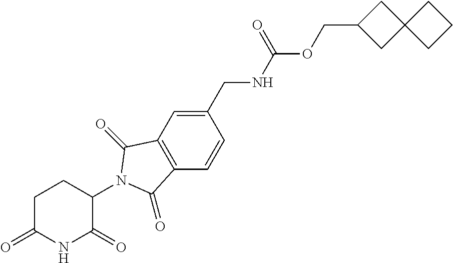 [SMILES: O=C1CCC(N2C(=O)c3ccc(CNC(=O)OCC4CC5(CCC5)C4)cc3C2=O)C(=O)N1] |
| 118 | 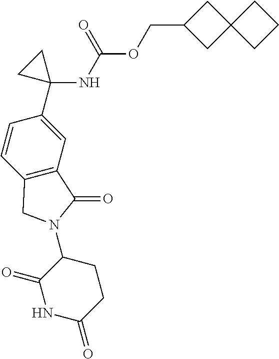 [SMILES: O=C1CCC(N2Cc3ccc(C4(NC(=O)OCC5CC6(CCC6)C5)CC4)cc3C2=O)C(=O)N1] |
| 119 | 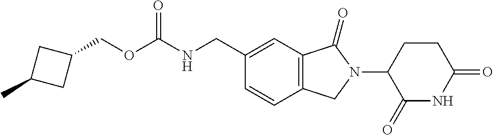 [SMILES: C[C@H]1C[C@H](COC(=O)NCc2ccc3c(c2)C(=O)N(C2CCC(=O)NC2=O)C3)C1] |
| 120 | 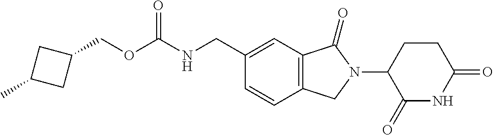 [SMILES: C[C@H]1C[C@@H](COC(=O)NCc2ccc3c(c2)C(=O)N(C2CCC(=O)NC2=O)C3)C1] |
| 121 | 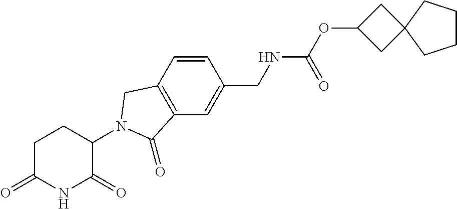 [SMILES: O=C1CCC(N2Cc3ccc(CNC(=O)OC4CC5(CCCC5)C4)cc3C2=O)C(=O)N1] |
| 122 | 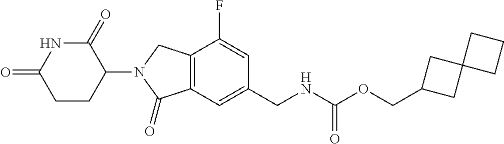 [SMILES: O=C1CCC(N2Cc3c(F)cc(CNC(=O)OCC4CC5(CCC5)C4)cc3C2=O)C(=O)N1] |
| 123 | 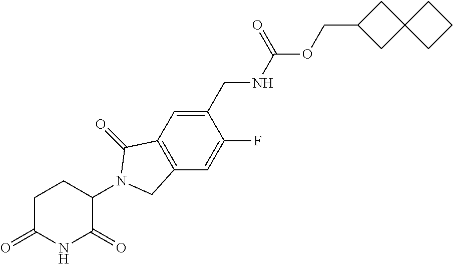 [SMILES: O=C1CCC(N2Cc3cc(F)c(CNC(=O)OCC4CC5(CCC5)C4)cc3C2=O)C(=O)N1] |
| 124 | 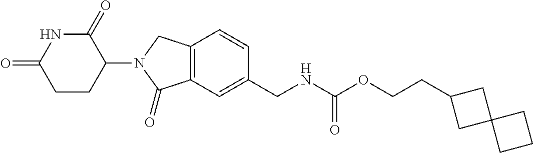 [SMILES: O=C1CCC(N2Cc3ccc(CNC(=O)OCCC4CC5(CCC5)C4)cc3C2=O)C(=O)N1] |
| 125 | 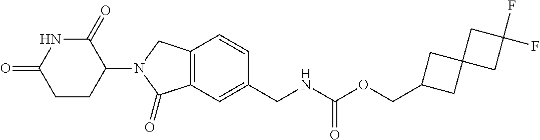 [SMILES: O=C1CCC(N2Cc3ccc(CNC(=O)OCC4CC5(C4)CC(F)(F)C5)cc3C2=O)C(=O)N1] |
| 126 | 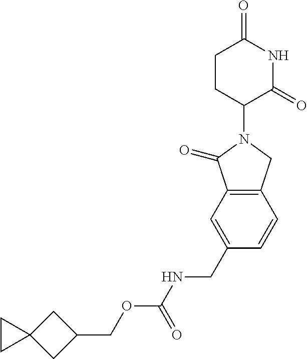 [SMILES: O=C1CCC(N2Cc3ccc(CNC(=O)OCC4CC5(CC5)C4)cc3C2=O)C(=O)N1] |
| 127 | 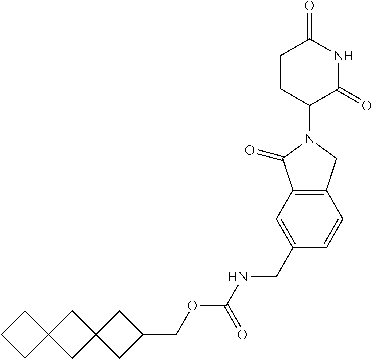 [SMILES: O=C1CCC(N2Cc3ccc(CNC(=O)OCC4CC5(C4)CC4(CCC4)C5)cc3C2=O)C(=O)N1] |
| 128 | 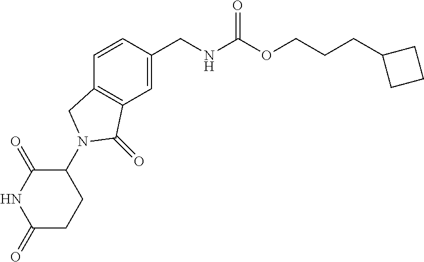 [SMILES: O=C1CCC(N2Cc3ccc(CNC(=O)OCCCC4CCC4)cc3C2=O)C(=O)N1] |
| 129 | 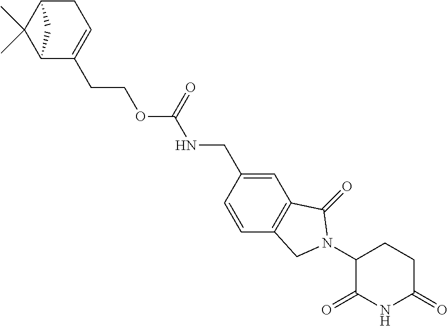 [SMILES: CC1(C)[C@H]2CC=C(CCOC(=O)NCc3ccc4c(c3)C(=O)N(C3CCC(=O)NC3=O)C4)[C@@H]1C2] |
| 130 | 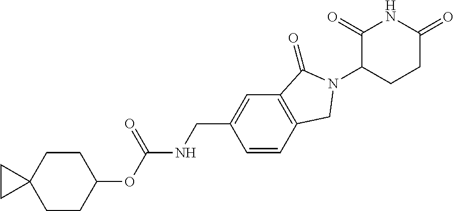 [SMILES: O=C1CCC(N2Cc3ccc(CNC(=O)OC4CCC5(CC4)CC5)cc3C2=O)C(=O)N1] |
| 131 | 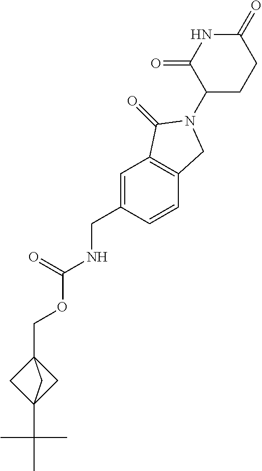 [SMILES: CC(C)(C)C12CC(COC(=O)NCc3ccc4c(c3)C(=O)N(C3CCC(=O)NC3=O)C4)(C1)C2] |
| 132 | 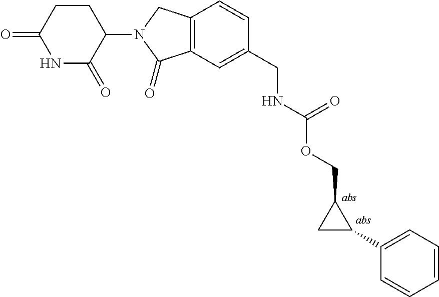 |
| 133 | 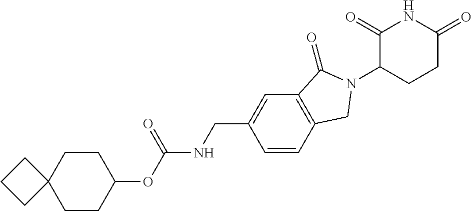 [SMILES: O=C1CCC(N2Cc3ccc(CNC(=O)OC4CCC5(CCC5)CC4)cc3C2=O)C(=O)N1] |
| 134 | 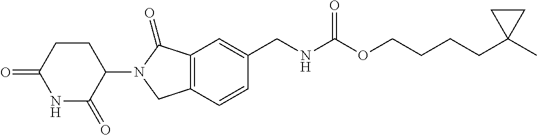 [SMILES: CC1(CCCCOC(=O)NCc2ccc3c(c2)C(=O)N(C2CCC(=O)NC2=O)C3)CC1] |
| 135 | 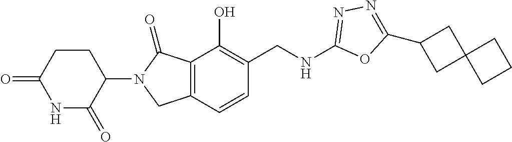 [SMILES: O=C1CCC(N2Cc3ccc(CNc4nnc(C5CC6(CCC6)C5)o4)c(O)c3C2=O)C(=O)N1] |
| 136 | 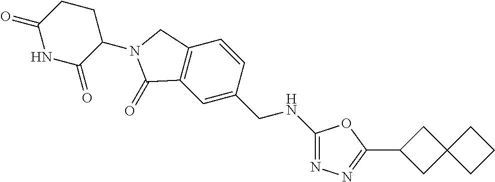 [SMILES: O=C1CCC(N2Cc3ccc(CNc4nnc(C5CC6(CCC6)C5)o4)cc3C2=O)C(=O)N1] |
| 137 | 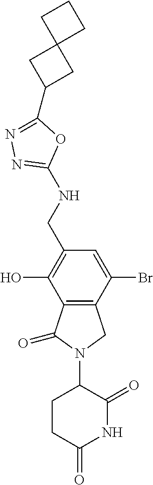 [SMILES: O=C1CCC(N2Cc3c(Br)cc(CNc4nnc(C5CC6(CCC6)C5)o4)c(O)c3C2=O)C(=O)N1] |
| 138 | 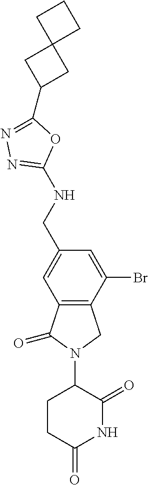 [SMILES: O=C1CCC(N2Cc3c(Br)cc(CNc4nnc(C5CC6(CCC6)C5)o4)cc3C2=O)C(=O)N1] |
| 139 | 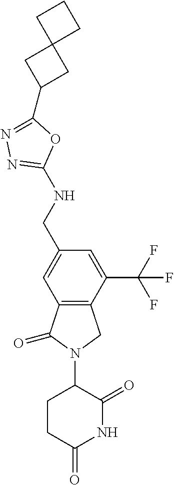 [SMILES: O=C1CCC(N2Cc3c(cc(CNc4nnc(C5CC6(CCC6)C5)o4)cc3C(F)(F)F)C2=O)C(=O)N1] |
| 140 | 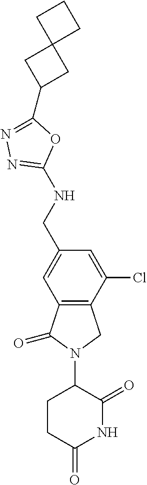 [SMILES: O=C1CCC(N2Cc3c(Cl)cc(CNc4nnc(C5CC6(CCC6)C5)o4)cc3C2=O)C(=O)N1] |
| 141 | 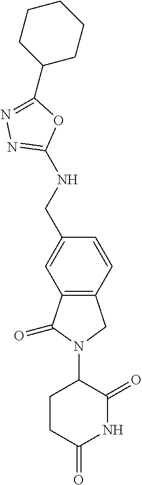 [SMILES: O=C1CCC(N2Cc3ccc(CNc4nnc(C5CCCCC5)o4)cc3C2=O)C(=O)N1] |
| 142 | 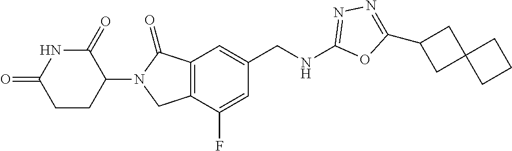 [SMILES: O=C1CCC(N2Cc3c(F)cc(CNc4nnc(C5CC6(CCC6)C5)o4)cc3C2=O)C(=O)N1] |
| 143 | 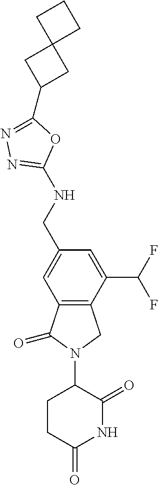 [SMILES: O=C1CCC(N2Cc3c(cc(CNc4nnc(C5CC6(CCC6)C5)o4)cc3C(F)F)C2=O)C(=O)N1] |
| 144 | 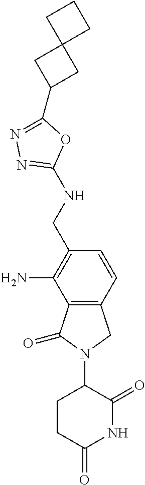 [SMILES: Nc1c(CNc2nnc(C3CC4(CCC4)C3)o2)ccc2c1C(=O)N(C1CCC(=O)NC1=O)C2] |
| 145 | 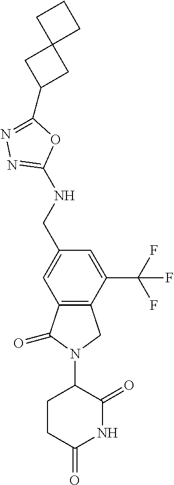 [SMILES: O=C1CCC(N2Cc3c(cc(CNc4nnc(C5CC6(CCC6)C5)o4)cc3C(F)(F)F)C2=O)C(=O)N1] |

### Pharmaceutical Compositions

In another embodiment, the present disclosure provides a pharmaceutical composition comprising a compound described herein, or pharmaceutically acceptable salt thereof, and a pharmaceutically acceptable excipient. In certain embodiments, the pharmaceutical composition comprises an effective amount of the compound. In certain embodiments, the pharmaceutical composition comprises a therapeutically effective amount of the compound.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration.

Compositions for oral administration can take the form of bulk liquid solutions or suspensions, or bulk powders. In some embodiments, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component with the remainder being various vehicles or excipients and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable excipients known in the art. As before, the active compound in such compositions is typically a minor component with the remainder being the injectable excipient and the like.

Transdermal compositions are typically formulated as a topical ointment or cream containing the active ingredient(s). When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or Formulation. All such known transdermal formulations and ingredients are included within the scope of the disclosure provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

### Methods of Treatment and Uses

Furthermore, the compounds and pharmaceutical compositions described herein are contemplated as useful in the treatment or prevention of disorders in subjects in need thereof. Compounds described herein, in one embodiment, are used to degrade CDK2 for the treatment of prevention of a disorder.

Cyclin dependent kinases, or CDKs, are a family of closely related kinases that regulate progression through the cell cycle. CDK activity is further modulated by levels of specific cyclins, for example, cyclin E1 activates cyclin dependent kinase 2, or CDK2. Tumors with CDK2 are activated by (i) the amplification of Cyclin E1 or E2 or the loss of the AMBRA1 gene and (ii) the loss of retinoblastoma. Elimination of CDK2 is contemplated to treat such disorders in patients in need thereof.

Accordingly, in one embodiment of the disclosure, a compound, or pharmaceutically acceptable salt thereof, or pharmaceutical composition described herein is administered to a subject to degrade CDK2 in the subject.

In one aspect of the disclosure, described herein is a method of treating or preventing a disorder in a subject in need thereof, comprising administering to the subject an effective amount of a compound, or pharmaceutically acceptable salt thereof, or pharmaceutical composition described herein.

In another aspect, described herein is a method of degrading CDK2 in a subject suffering from a disorder, comprising administering to the subject a therapeutically effective amount of a compound described herein, or pharmaceutically acceptable salt thereof, or pharmaceutical composition described herein. In some embodiments, the compound binds to cereblon and a CDK2 protein to induce ubiquitination and subsequent proteasomal degradation of the CDK2.

Exemplary disorders that can be treated or prevented by the methods of the present disclosure include but are not limited to, cancer of the bladder, bone, brain, breast, cervix, chest, colon, endometrium, esophagus, eye, head, kidney, liver, lymph nodes, lung, upper aerodigestive tract (including nasal cavity and paranasal sinuses, nasopharynx or cavum, oral cavity, oropharynx, larynx, hypopharynx and salivary glands, neck, ovaries, pancreas, prostate, rectum, skin, stomach, testis, throat, or uterus. Other exemplary disorders include, but are not limited to, amyloidosis, neuroblastoma, meningioma, hemangiopericytoma, multiple brain metastases, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, e.g., neuroendocrine prostate cancer such as castration-resistant neuroendocrine prostate cancer (NEPC) and lung neuroendocrine tumors (Lu-NETs), rectal adenocarcinoma, colorectal cancer, including stage 3 and stage 4 colorectal cancer, unresectable colorectal carcinoma, metastatic hepatocellular carcinoma, Kaposi's sarcoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma; and blood bourne (liquid) or hematological cancers, including but not limited to leukemias, lymphomas, and myelomas, such as diffuse large B-cell lymphoma (DLBCL), B-cell immunoblastic lymphoma, small non-cleaved cell lymphoma, human lymphotropic virus-type 1 (HTLV-1) leukemia/lymphoma, adult T-cell lymphoma, peripheral T-cell lymphoma (PTCL), cutaneous T-cell lymphoma (CTCL), mantle cell lymphoma (MCL), Hodgkin's lymphoma (HL), non-Hodgkin's lymphoma (NHL), AIDS-related lymphoma, follicular lymphoma, small lymphocytic lymphoma, T-cell/histiocyte rich large B-cell lymphoma, transformed lymphoma, primary mediastinal (thymic) large B-cell lymphoma, splenic marginal zone lymphoma, Richter's transformation, nodal marginal zone lymphoma, ALK-positive large B-cell lymphoma, indolent lymphoma (for example, DLBCL, follicular lymphoma, or marginal zone lymphoma), acute myelogenous leukemia (AML), acute lymphocytic leukemia (ALL), adult T-cell leukemia, chronic lymphocytic leukemia (CLL), small lymphocytic lymphoma (SLL), hairy cell leukemia, myelodysplasia, myeloproliferative disorders, chronic myelogenous leukemia (CML), acute monocytic leukemia (AMoL), myelodysplastic syndrome (MDS), human lymphotropic virus-type 1 (HTLV-1) leukemia, mastocytosis, B-cell acute lymphoblastic leukemia, Non-Hodgkin's Lymphoma, Hodgkin's Lymphoma, and multiple myeloma (MM).

In some embodiments, the disorder is breast cancer or ovarian cancer. In some embodiments, the breast cancer is estrogen receptor positive breast cancer or triple negative breast cancer. In some embodiments, the disorder is selected from the group consisting of hormone-receptor positive breast cancer, ovarian cancer, uterine cancer, lung cancer, triple negative breast cancer, and gastric cancer.

In another aspect of the disclosure, described herein is a method of treating cancer (e.g., a cancer described herein) in a subject in need thereof, comprising administering to the subject a therapeutically effective amount of a compound described herein, or pharmaceutically acceptable salt thereof, or pharmaceutical composition described herein.

In another aspect, described herein is a method of degrading CDK2 in a subject suffering from cancer (e.g., a cancer described herein), comprising administering to the subject a therapeutically effective amount of a compound described herein, or pharmaceutically acceptable salt thereof, or pharmaceutical composition described herein.

In another aspect, described herein is a method of treating a solid tumor (e.g., a solid tumor described herein) in a subject in need thereof, comprising administering to the subject a therapeutically effective amount of a compound described herein, or pharmaceutically acceptable salt thereof, or pharmaceutical composition described herein.

In another aspect, described herein is a method of treating a liquid tumor (e.g., a liquid tumor described herein) in a subject in need thereof, comprising administering to the subject a therapeutically effective amount of a compound described herein, or pharmaceutically acceptable salt thereof, or pharmaceutical composition described herein. In some embodiments, the liquid tumor is that of a haematological cancer (e.g., a haematological cancer described herein).

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” as used herein refers to a radical of a straight-chain or branched saturated hydrocarbon group. In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆alkyl”). Examples of C₁₋₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkenyl” as used herein refers to a radical of a straight-chain or branched hydrocarbon group having, one or more carbon-carbon double bonds. In some embodiments, an alkenyl group has 2 to 10 carbon atoms (“C₂₋₁₀ alkenyl”). In some embodiments, an alkenyl group has 2 to 9 carbon atoms (“C₂₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

The term “alkynyl” as used herein refers to a radical of a straight-chain or branched hydrocarbon group having one or more carbon-carbon triple bonds (e.g., 1, 2, 3, or 4 carbon-carbon triple bonds). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The term “cycloalkyl” as used herein refers to a radical of a saturated or partially unsaturated cyclic hydrocarbon group having from 3 to 12 ring carbon atoms (“C₃₋₁₂ cycloalkyl”) and zero heteroatoms in the ring system. In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 3 to 6 ring carbon atoms (“C₃₋₆ cycloalkyl”). In some embodiments, a cycloalkyl group has 5 to 10 ring carbon atoms (“C₅₋₁₀ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ cycloalkyl groups include, without limitation, the aforementioned C₃₋₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ cycloalkyl groups include, without limitation, the aforementioned C₃₋₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) or tricyclic system (“tricyclic cycloalkyl”). “Cycloalkyl” also includes ring systems wherein the cycloalkyl ring, as defined above, is fused with one or more aryl or heteroaryl groups wherein the point of attachment is on the cycloalkyl ring or the one or more aryl or heteroaryl groups, and in such instances, the number of carbons continue to designate the number of carbons in the cycloalkyl ring system.

The term “heterocyclyl” as used herein refers to a radical of a saturated or partially unsaturated 3 to 10-membered ring system having ring carbon atoms and 1 to 4 ring heteroatoms, wherein each heteroatom is independently selected from nitrogen, oxygen, sulfur, boron, phosphorus, and silicon (“3 to 10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring or the one or more aryl or heteroaryl groups, and in such instances, the number of ring members continue to designate the number of ring members in the heterocyclyl ring system.

In some embodiments, a heterocyclyl group is a 5 to 10 membered saturated or partially unsaturated ring system having ring carbon atoms and 1 to 4 ring heteroatoms, wherein each heteroatom is independently selected from nitrogen, oxygen, sulfur, boron, phosphorus, and silicon (“5 to 10 membered heterocyclyl”). In some embodiments, a heterocyclyl group is a 5 to 8 membered saturated or partially unsaturated ring system having ring carbon atoms and 1 to 4 ring heteroatoms, wherein each heteroatom is independently selected from nitrogen, oxygen, and sulfur (“5 to 8 membered heterocyclyl”). In some embodiments, a heterocyclyl group is a 5 to 6 membered saturated or partially unsaturated ring system having ring carbon atoms and 1 to 4 ring heteroatoms, wherein each heteroatom is independently selected from nitrogen, oxygen, and sulfur (“5 to 6 membered heterocyclyl”). In some embodiments, the 5 to 6 membered heterocyclyl has 1 to 3 ring heteroatoms selected from nitrogen, oxygen, and sulfur. In some embodiments, the 5 to 6 membered heterocyclyl has 1 to 2 ring heteroatoms selected from nitrogen, oxygen, and sulfur. In some embodiments, the 5 to 6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thioranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

The term “aryl” as used herein refers to a radical of a monocyclic or polycyclic (e.g., bicyclic or tricyclic) 4n+2 aromatic ring system (e.g., having 6, 10, or 14 π electrons shared in a cyclic array) having 6 to 14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

The term “heteroaryl” as used herein refers to a radical of a 5 to 10 membered monocyclic or bicyclic 4n+2 aromatic ring system (e.g., having 6 or 10 π electrons shared in a cyclic array) having ring carbon atoms and 1 to 4 ring heteroatoms provided in the aromatic ring system, wherein each heteroatom is independently selected from nitrogen, oxygen and sulfur (“5 to 10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5 to 10 membered aromatic ring system having ring carbon atoms and 1 to 4 ring heteroatoms provided in the aromatic ring system, wherein each heteroatom is independently selected from nitrogen, oxygen, and sulfur (“5 to 10 membered heteroaryl”). In some embodiments, a heteroaryl group is a 5 to 8 membered aromatic ring system having ring carbon atoms and 1 to 4 ring heteroatoms provided in the aromatic ring system, wherein each heteroatom is independently selected from nitrogen, oxygen, and sulfur (“5 to 8 membered heteroaryl”). In some embodiments, a heteroaryl group is a monocyclic 5 to 6 membered aromatic ring system having ring carbon atoms and 1 to 4 ring heteroatoms provided in the aromatic ring system, wherein each heteroatom is independently selected from nitrogen, oxygen, and sulfur (“5 to 6 membered heteroaryl”). In some embodiments, the 5 to 6 membered heteroaryl has 1 to 3 ring heteroatoms selected from nitrogen, oxygen, and sulfur. In some embodiments, the 5 to 6 membered heteroaryl has 1 to 2 ring heteroatoms selected from nitrogen, oxygen, and sulfur. In some embodiments, the 5 to 6 membered heteroaryl has 1 ring heteroatom selected from nitrogen, oxygen, and sulfur. In some embodiments, a heteroaryl group is a monocyclic 5 membered aromatic ring system having ring carbon atoms and 1 to 4 ring heteroatoms provided in the aromatic ring system, wherein each heteroatom is independently selected from nitrogen, oxygen, and sulfur (“5-membered heteroaryl”). In some embodiments, a heteroaryl group is a monocyclic 6 membered aromatic ring system having ring carbon atoms and 1 to 4 ring heteroatoms provided in the aromatic ring system, wherein each heteroatom is independently selected from nitrogen, oxygen, and sulfur (“6-membered heteroaryl”).

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

The term “alkoxy” as used herein refers to the group —OR¹⁰⁰ where R¹⁰⁰ is alkyl, alkenyl, alkynyl, cycloalkyl, heterocyclyl, aryl, or heteroaryl. Exemplary alkoxy groups include methoxy, ethoxy, n-propoxy, isopropoxy, n-butoxy, tert-butoxy, sec-butoxy, n-pentoxy, n-hexoxy, and 1,2-dimethylbutoxy. Other exemplary alkoxy groups are lower alkoxy, i.e. with between 1 and 6 carbon atoms. In other examples, alkoxy groups have between 1 and 4 carbon atoms.

The term “cyano” as used herein refers to the radical —CN.

The term “halogen” as used herein refers to F, Cl, Br, or I.

The term “oxo” as used herein refers to ═O.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition. In an alternative embodiment, the present disclosure contemplates administration of the compounds described herein as a prophylactic before a subject begins to suffer from the specified disease, disorder or condition.

In general, the “effective amount” of a compound as used herein refers to an amount sufficient to elicit the desired biological response. As will be appreciated by those of ordinary skill in this art, the effective amount of a compound of the present disclosure may vary depending on such factors as the desired biological endpoint, the pharmacokinetics of the compound, the disease being treated, the mode of administration, and the age, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Isomers, e.g., stereoisomers, can be isolated from mixtures by methods known to those skilled in the art, including chiral high pressure liquid chromatography (HPLC) and the formation of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

The compounds provided herein can be administered as the sole active agent, or they can be administered in combination with other active agents. In some embodiments, the present invention provides a combination of a compound of the present invention and another pharmacologically active agent. Administration in combination can proceed by any technique apparent to those of skill in the art including, for example, separate, sequential, concurrent, and alternating administration.

The present disclosure, in an alternative embodiment, also embraces isotopically labeled compounds which are identical to those recited herein, except that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

### EXAMPLES

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Abbreviations: CDI: 1,1′-Carbonyldiimidazole; CRBN: cereblon; DMF: N,N-dimethylformamide; eq: equivalents; DIEA or DIPEA: N,N-diisopropylethylamine; DMSO: dimethyl sulfoxide; DIPEA: EI: electron ionization; ESI: electrospray ionization; h: hours; HPLC: high-performance liquid chromatography; LCMS: liquid chromatography mass spectrometry; MS: mass spectrometry; MTBE: tert-butyl methyl ether NMR: nuclear magnetic resonance.

### Example 1. Synthesis of Compound 1

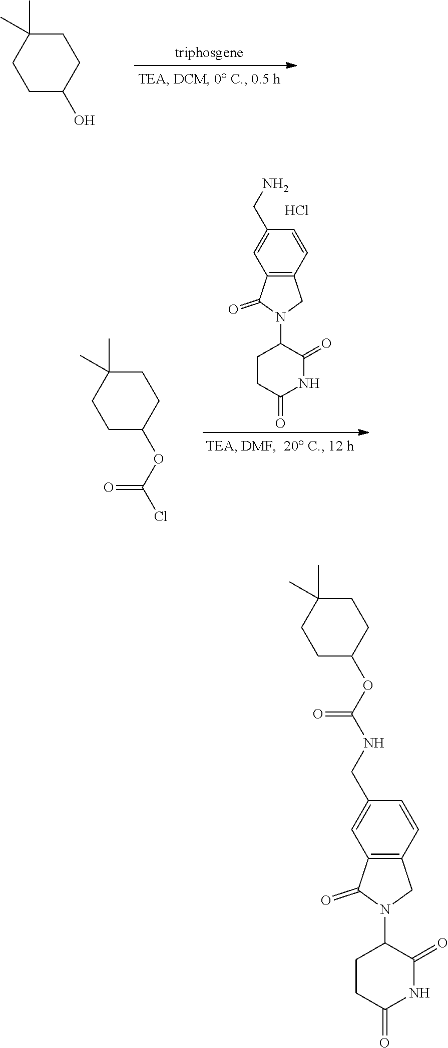

### Step 1. Compound 4,4-dimethylcyclohexyl carbonochloridate

To a solution of 4,4-dimethylcyclohexanol (100 mg, 780 umol, 1.00 eq) and bis(trichloromethyl) carbonate (116 mg, 390 umol, 0.500 eq) in dichloromethane (1.00 mL) was added dropwise triethylamine (78.9 mg, 780 umol, 109 uL, 1.00 eq) at 0° C. The reaction mixture was stirred at 0° C. for 0.5 h. The reaction mixture was concentrated under reduced pressure to afford 4,4-dimethylcyclohexyl carbonochloridate (148 mg, 776 umol, 99% yield) as a yellow solid.

### Step 2. Compound 4,4-dimethylcyclohexyl ((2-(2,6-dioxopiperidin-3-yl)-3-oxoisoindolin-5-yl)methyl)carbamate

To a solution of 4,4-dimethylcyclohexyl carbonochloridate (148 mg, 776 umol, 2.00 eq) and triethylamine (236 mg, 2.33 mmol, 324 uL, 6.00 eq) in dimethylformamide (1.50 mL) was added 3-(6-(aminomethyl)-1-oxoisoindolin-2-yl)piperidine-2,6-dione (120 mg, 388 umol, 1.00 eq, hydrochloride). The reaction mixture was stirred at 20° C. for 12 h. The reaction mixture was filtered. The filtrate was purified by Prep-HPLC (column: Unisil 3-100 C18 Ultra 150*50 mm*3 um; mobile phase: [water(formic acid)-acetonitrile]; B %: 30%-60%, 15 min) and Prep-TLC (petroleum ether/ethyl acetate=1/5) to afford 4,4-dimethylcyclohexyl((2-(2,6-dioxopiperidin-3-yl)-3-oxoisoindolin-5-yl)methyl)carbamate (37.35 mg, 86.50 umol, 22% yield, 99% purity) as a white solid.

1H NMR (400 MHz, DMSO-d6) δ=10.98 (br s, 1H), 7.70 (br t, J=6.1 Hz, 1H), 7.60 (s, 1H), 7.56-7.36 (m, 2H), 5.10 (dd, J=5.1, 13.3 Hz, 1H), 4.54-4.46 (m, 1H), 4.46-4.28 (m, 2H), 4.28-4.22 (m, 2H), 2.97-2.84 (m, 1H), 2.63-2.56 (m, 1H), 2.39 (br dd, J=4.5, 13.0 Hz, 1H), 2.06-1.92 (m, 1H), 1.68 (br d, J=4.3 Hz, 2H), 1.53-1.43 (m, 2H), 1.39 (br dd, J=4.2, 13.3 Hz, 2H), 1.25-1.16 (m, 2H), 0.89 (br d, J=5.4 Hz, 6H).

1H NMR (400 MHz, DMSO-d₆, T=80° C.) δ=10.70 (br s, 1H), 7.63 (s, 1H), 7.56-7.49 (m, 2H), 7.36 (br s, 1H), 5.06 (dd, J=5.2, 13.1 Hz, 1H), 4.53 (tt, J=4.0, 8.4 Hz, 1H), 4.47-4.31 (m, 2H), 4.28 (d, J=6.1 Hz, 2H), 2.96-2.83 (m, 1H), 2.69-2.59 (m, 1H), 2.40 (dq, J=4.5, 13.1 Hz, 1H), 2.09-2.00 (m, 1H), 1.77-1.63 (m, 2H), 1.56-1.46 (m, 2H), 1.39 (br d, J=12.0 Hz, 2H), 1.27-1.17 (m, 2H), 0.90 (s, 6H).

### Example 2. Synthesis of Compound 2

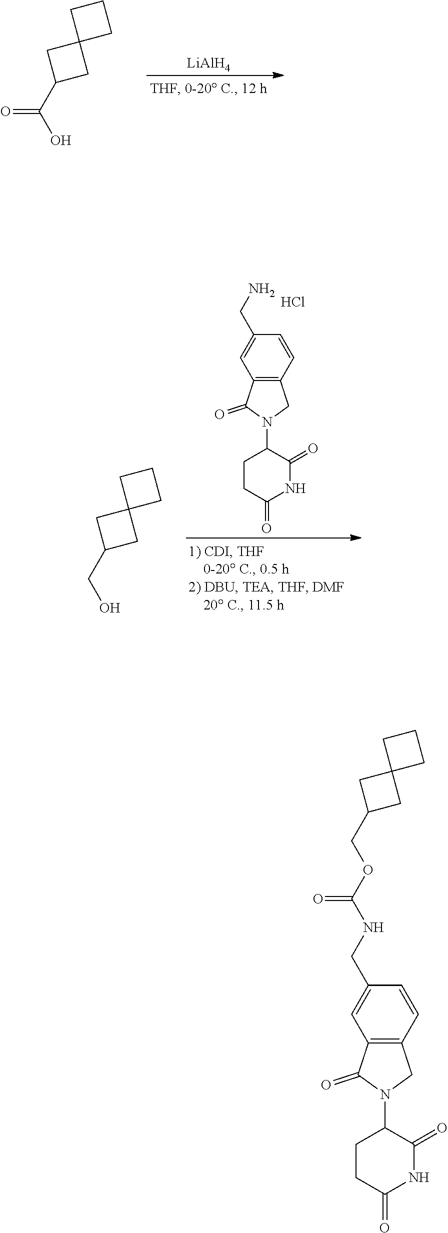 [SMILES: O=C(O)C1CC2(CCC2)C1 ; "LiAlH4" ; OCC1CC2(CCC2)C1 ; NCc1ccc2c(c1)C(=O)N(C1CCC(=O)NC1=O)C2 ; Cl ; O=C1CCC(N2Cc3ccc(CNC(=O)OCC4CC5(CCC5)C4)cc3C2=O)C(=O)N1]

### Step 1. Compound 2-spiro[3.3]heptan-2-ylmethanol

To a solution of spiro[3.3]heptane-2-carboxylic acid (200 mg, 1.43 mmol, 1.00 eq) in tetrahydrofuran (2.00 mL) was added lithium aluminum hydride (81.2 mg, 2.14 mmol, 1.50 eq) at 0° C. The reaction mixture was stirred at 20° C. for 12 h. The reaction mixture was quenched with sodium sulfate decahydrate (1.00 g), then filtered and concentrated under reduced pressure to afford spiro[3.3]heptan-2-ylmethanol (100 mg, 792 umol, 56% yield) as colorless oil.

1H NMR (400 MHz, CDCl3) δ=3.55 (br d, J=6.6 Hz, 2H), 2.33 (td, J=7.5, 15.0 Hz, 1H), 2.13-2.03 (m, 2H), 2.00 (t, J=7.2 Hz, 2H), 1.92-1.86 (m, 2H), 1.83-1.75 (m, 2H), 1.73-1.63 (m, 2H).

### Step 2. Compound spiro[3.3]heptan-2-ylmethyl ((2-(2,6-dioxopiperidin-3-yl)-3-oxoisoindolin-5-yl)methyl)carbamate

To a solution of spiro[3.3]heptan-2-ylmethanol (50.0 mg, 396 umol, 1.00 eq) in tetrahydrofuran (0.500 mL) was added 1,1′-carbonyldiimidazole (128 mg, 792 umol, 2.00 eq) at 0° C. The reaction mixture was stirred at 20° C. for 0.5 h. Then the reaction mixture was added into a solution of 3-(6-(aminomethyl)-1-oxoisoindolin-2-yl)piperidine-2,6-dione (123 mg, 396 umol, 1.00 eq, hydrochloride) and 2,3,4,6,7,8,9,10-octahydropyrimido[1,2-a]azepine (60.3 mg, 396 umol, 59.7 uL, 1.00 eq), triethylamine (40.1 mg, 396 umol, 55.2 uL, 1.00 eq) in dimethyl formamide (0.500 mL) and tetrahydrofuran (0.500 mL). The reaction mixture was stirred at 20° C. for 11.5 h. The reaction mixture was concentrated under reduced pressure to give a residue. The residue was purified by Prep-HPLC (column: Phenomenex Synergi C18 150*25 mm*10 um; mobile phase: [water(formic acid)-acetonitrile]; B %: 35%-65%, 10 min) and lyophilized to afford spiro[3.3]heptan-2-ylmethyl ((2-(2,6-dioxopiperidin-3-yl)-3-oxoisoindolin-5-yl)methyl)carbamate (18.26 mg, 42.49 umol, 11% yield, 99% purity) as a white solid. ¹H NMR (400 MHz, DMSO-d₆) δ=11.10 (s, 1H), 7.88 (br t, J=5.7 Hz, 1H), 7.72 (s, 1H), 7.68-7.59 (m, 2H), 5.23 (dd, J=5.1, 13.3 Hz, 1H), 4.63-4.50 (m, 1H), 4.47-4.31 (m, 3H), 4.03 (d, J=6.7 Hz, 2H), 3.11-2.95 (m, 1H), 2.76-2.67 (m, 1H), 2.58-2.45 (m, 2H), 2.21-2.03 (m, 5H), 2.01-1.94 (m, 2H), 1.90-1.70 (m, 4H).

### Example 3. Synthesis of Compound 3

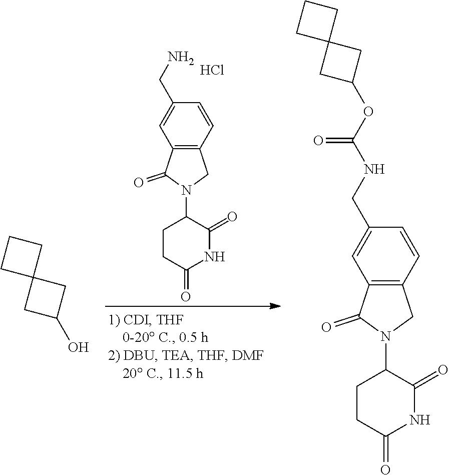 [SMILES: OC1CC2(CCC2)C1 ; NCc1ccc2c(c1)C(=O)N(C1CCC(=O)NC1=O)C2 ; Cl ; O=C1CCC(N2Cc3ccc(CNC(=O)OC4CC5(CCC5)C4)cc3C2=O)C(=O)N1]

### Step 1. Compound spiro[3.3]heptan-2-yl ((2-(2,6-dioxopiperidin-3-yl)-3-oxoisoindolin-5-yl)methyl)carbamate

To a solution of spiro[3.3]heptan-2-ol (20.0 mg, 178.3 umol, 1.00 eq) in tetrahydrofuran (0.500 mL) was added di(1H-imidazol-1-yl)methanone (57.8 mg, 357 umol, 2.00 eq) at 0° C. The reaction mixture was stirred at 20° C. for 0.5 h. Then the reaction mixture was added into a solution of 3-(6-(aminomethyl)-1-oxoisoindolin-2-yl)piperidine-2,6-dione (55.2 mg, 178 umol, 1.00 eq, hydrochloride), 2,3,4,6,7,8,9,10-octahydropyrimido[1,2-a]azepine (27.1 mg, 178 umol, 26.9 uL, 1.00 eq) and triethylamine (18.0 mg, 178 umol, 24.8 uL, 1.00 eq) in tetrahydrofuran (0.500 mL) and dimethyl formamide (0.500 mL). The reaction mixture was stirred at 20° C. for 11.5 h. The reaction mixture was concentrated under reduced pressure to give a residue. The residue was purified by Prep-HPLC(column: Phenomenex Synergi C18 150*25 mm*10 um; mobile phase: [water(formic acid)-acetonitrile]; B %: 34%-54%, 10 min) and lyophilized to afford spiro[3.3]heptan-2-yl ((2-(2,6-dioxopiperidin-3-yl)-3-oxoisoindolin-5-yl)methyl)carbamate (19.46 mg, 46.82 umol, 26% yield, 99% purity) as a white solid.

¹H NMR (400 MHz, DMSO-d₆) δ=11.0 (br s, 1H), 7.76 (br t, J=6.2 Hz, 1H), 7.59 (s, 1H), 7.57-7.53 (m, 1H), 7.52-7.46 (m, 1H), 5.11 (dd, J=5.0, 13.2 Hz, 1H), 4.72 (t, J=7.3 Hz, 1H), 4.52-4.36 (m, 1H), 4.35-4.27 (m, 1H), 4.25 (br d, J=6.1 Hz, 2H), 2.99-2.85 (m, 1H), 2.65-2.56 (m, 1H), 2.42-2.34 (m, 3H), 2.05-1.85 (m, 7H), 1.84-1.71 (m, 2H).

### Example 4. Synthesis of Compound 4

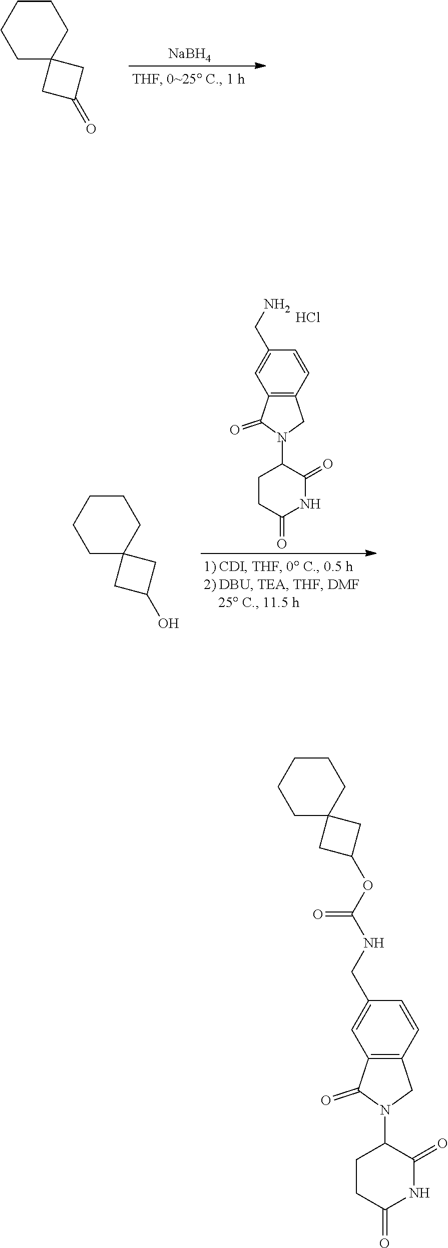 [SMILES: O=C1CC2(CCCCC2)C1 ; "NaBH4" ; OC1CC2(CCCCC2)C1 ; NCc1ccc2c(c1)C(=O)N(C1CCC(=O)NC1=O)C2 ; Cl ; O=C1CCC(N2Cc3ccc(CNC(=O)OC4CC5(CCCCC5)C4)cc3C2=O)C(=O)N1]

### Step 1. Compound spiro[3.5]nonan-2-ol

To a solution of spiro[3.5]nonan-2-one (50.0 mg, 361 umol, 1.00 eq) in tetrahydrofuran (1.00 mL) was added sodium borohydride (20.5 mg, 542 umol, 1.50 eq) at 0° C. Then the mixture was stirred at 25° C. for 1 h. The mixture was quenched with saturated ammonium chloride (5 mL), extracted with ethyl acetate (3×20 mL), washed with brine (3×20 mL), and dried over anhydrous sodium sulfate, filtered and concentrated under reduced pressure to afford spiro[3.5]nonan-2-ol (20.0 mg, 142 umol, 39% yield) as colorless oil.

¹H NMR (400 MHz, CDCl₃) δ=4.27 (m, J=7.2 Hz, 1H), 2.26-2.19 (m, 2H), 1.63-1.54 (m, 4H), 1.47-1.42 (m, 3H), 1.40-1.31 (m, 5H).

### Step 2. Compound spiro[3.5]nonan-2-yl ((2-(2,6-dioxopiperidin-3-yl)-3-oxoisoindolin-5-yl)methyl)carbamate

To a solution of spiro[3.5]nonan-2-ol (10.0 mg, 71.3 umol, 1.00 eq) in tetrahydrofuran (0.300 mL) were added di(1H-imidazol-1-yl)methanone (23.1 mg, 142 umol, 2.00 eq) at 0° C. The mixture was stirred at 25° C. for 0.5 h. Then the mixture was added into a solution of 3-(6-(aminomethyl)-1-oxoisoindolin-2-yl)piperidine-2,6-dione (22.0 mg, 71.3 umol, 1.00 eq, hydrochloride), 2,3,4,6,7,8,9,10-octahydropyrimido[1,2-a]azepine (10.8 mg, 71.3 umol, 10.7 uL, 1.00 eq) and triethylamine (7.22 mg, 71.3 umol, 9.93 uL, 1.00 eq) in tetrahydrofuran (0.300 mL) and dimethylformamide (0.300 mL). Then the mixture was stirred at 25° C. for 11.5 h. The mixture was concentrated under reduced pressure to give a residue. The residue was purified by Prep-HPLC (column: Phenomenex luna C18 150*25 mm*10 um; mobile phase: [water(formic acid)-acetonitrile]; B %: 35%-65%, 10 min) and lyophilized to afford spiro[3.5]nonan-2-yl ((2-(2,6-dioxopiperidin-3-yl)-3-oxoisoindolin-5-yl)methyl)carbamate (2.26 mg, 5.09 umol, 7% yield, 99% purity) as a white solid.

¹H NMR (400 MHz, DMSO-d₆) δ=10.89 (br, 1H), 7.75 (br t, J=6.1 Hz, 1H), 7.59 (s, 1H), 7.56-7.53 (m, 1H), 7.51-7.48 (m, 1H), 5.11 (dd, J=5.1, 13.3 Hz, 1H), 4.83 (m, J=7.1 Hz, 1H), 4.47-4.39 (m, 1H), 4.33-4.27 (m, 1H), 4.25 (d, J=6.1 Hz, 2H), 2.96-2.86 (m, 1H), 2.64-2.56 (m, 1H), 2.39 (br dd, J=4.5, 13.1 Hz, 1H), 2.20-2.13 (m, 2H), 2.04-1.95 (m, 1H), 1.68-1.59 (m, 2H), 1.44-1.22 (m, 10H).

### Example 5. Synthesis of Compound 5

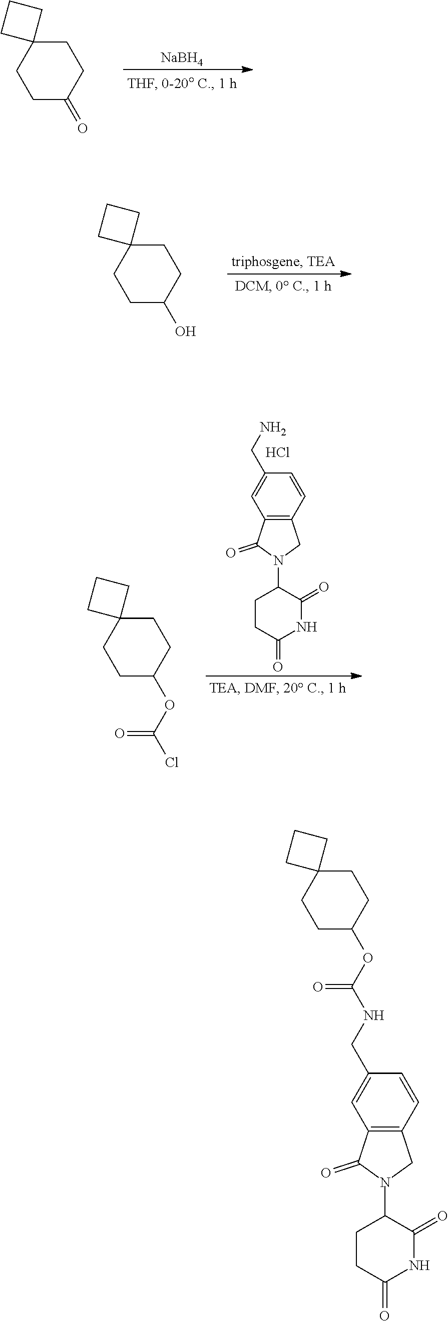

### Step 1. Compound spiro[3.5]nonan-7-ol

To a solution of spiro[3.5]nonan-7-one (100 mg, 724 umol, 1.00 eq) in tetrahydrofuran (2.00 mL) was added sodium borohydride (54.8 mg, 1.45 mmol, 2.00 eq) at 0° C. The reaction mixture was stirred at 20° C. for 1 h. The reaction mixture was quenched with saturated ammonium chloride (7 mL) and then extracted with ethyl acetate (3×10 mL). The organic layers were washed with brine (10 mL), dried over anhydrous sodium sulfate, filtered and concentrated under reduced pressure to afford spiro[3.5]nonan-7-ol (96.0 mg, 685 umol, 95% yield) as colorless oil.

1H NMR (400 MHz, CDCl₃) δ=3.60 (br s, 1H), 1.88-1.81 (m, 2H), 1.77-1.65 (m, 8H), 1.32 (br dd, J=4.8, 9.8 Hz, 4H), 0.93-0.78 (m, 1H).

### Step 2. Compound spiro[3.5]nonan-7-yl carbonochloridate

To a solution of spiro[3.5]nonan-7-ol (80.0 mg, 571 umol, 1.00 eq) and bis(trichloromethyl) carbonate (84.7 mg, 285 umol, 0.500 eq) in dichloromethane (1.00 mL) was added dropwise triethylamine (57.7 mg, 571 umol, 79.4 uL, 1.00 eq) at 0° C. The reaction mixture was stirred at 0° C. for 1 h. The reaction mixture was concentrated under reduced pressure to afford spiro[3.5]nonan-7-yl carbonochloridate (110 mg, crude) as a white solid.

### Step 3. Compound spiro[3.5]nonan-7-yl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (80.0 mg, 258 umol, 1.00 eq, hydrochloride) in dimethylformamide (1.00 mL) were added triethylamine (78.4 mg, 775 umol, 108 uL, 3.00 eq) and spiro[3.5]nonan-7-yl carbonochloridate (105 mg, 517 umol, 2.00 eq). The reaction mixture was stirred at 20° C. for 1 h. The reaction mixture was filtered. The filtrate was purified by Prep-HPLC(column: Phenomenex luna C18 150*25 mm*10 um; mobile phase: [water (formic acid)-acetonitrile]; B %: 35%-65%, 9 min) and lyophilized to afford spiro[3.5]nonan-7-yl ((2-(2,6-dioxopiperidin-3-yl)-3-oxoisoindolin-5-yl)methyl)carbamate (6.28 mg, 14.00 umol, 5% yield, 98% purity) as a yellow solid.

1H NMR (400 MHz, DMSO-d6) δ=10.97 (br s, 1H), 7.68 (br t, J=6.3 Hz, 1H), 7.60 (s, 1H), 7.57-7.52 (m, 1H), 7.52-7.47 (m, 1H), 5.10 (dd, J=4.9, 13.2 Hz, 1H), 4.45 (s, 1H), 4.41 (s, 1H), 4.33-4.22 (m, 3H), 2.96-2.86 (m, 1H), 2.58 (br s, 1H), 2.40 (br dd, J=4.3, 13.4 Hz, 1H), 2.03-1.97 (m, 1H), 1.83-1.77 (m, 2H), 1.73-1.64 (m, 8H), 1.38-1.30 (m, 4H).

### Example 6. Synthesis of Compound 6

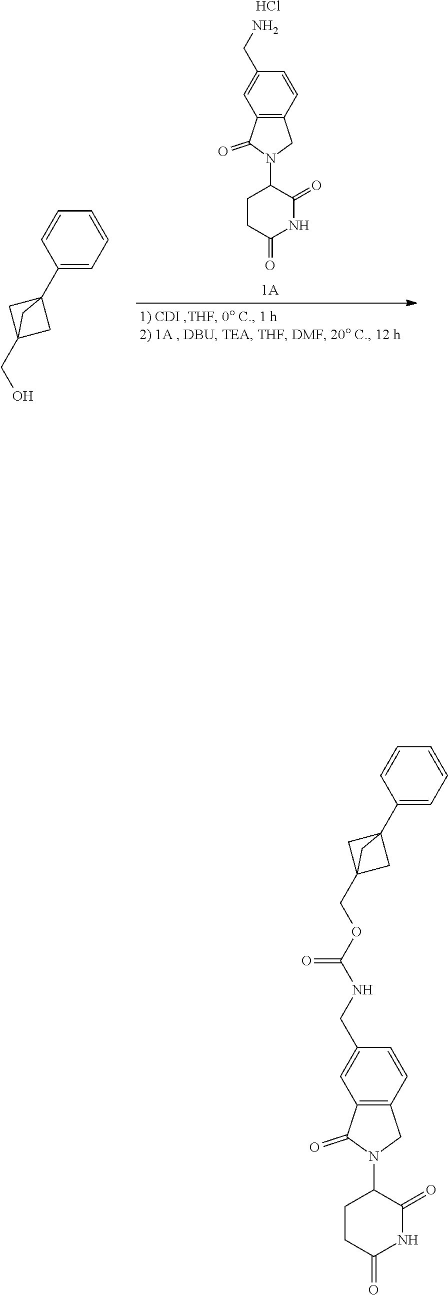 [SMILES: Cl ; OCC12CC(c3ccccc3)(C1)C2 ; NCc1ccc2c(c1)C(=O)N(C1CCC(=O)NC1=O)C2 ; O=C1CCC(N2Cc3ccc(CNC(=O)OCC45CC(c6ccccc6)(C4)C5)cc3C2=O)C(=O)N1]

### Step 1. Compound (3-phenylbicyclo[1.1.1]pentan-1-yl)methyl ((2-(2,6-dioxopiperidin-3-yl)-3-oxoisoindolin-5-yl)methyl)carbamate

To a solution of (3-phenylbicyclo[1.1.1]pentan-1-yl)methanol (10.0 mg, 57.4 umol, 1.00 eq) in tetrahydrofuran (0.200 mL) was added di(1H-imidazol-1-yl)methanone (18.6 mg, 115 umol, 2.00 eq) at 0° C., the mixture was stirred at 0° C. for 1 h. The resulting solution was added to a mixture of 3-(6-(aminomethyl)-1-oxoisoindolin-2-yl)piperidine-2,6-dione (17.8 mg, 57.4 umol, 1.00 eq, hydrochloride), 2,3,4,6,7,8,9,10-octahydropyrimido[1,2-a]azepine (8.74 mg, 57.4 umol, 8.65 uL, 1.00 eq) and triethylamine (5.81 mg, 57.4 umol, 7.99 uL, 1.00 eq) in tetrahydrofuran (0.200 mL) and dimethylformamide (0.200 mL). The reaction mixture was stirred at 20° C. for 12 h. The reaction mixture was concentrated under reduced pressure to give a residue. The residue was diluted with dimethylformamide (1 mL) and then filtered. The filtrate was purified by Prep-HPLC(column: Phenomenex luna C18 150*25 mm*10 um; mobile phase: [water(formic acid)-acetonitrile]; B %: 35%-65%, 9 min) and lyophilized to afford (3-phenylbicyclo[1.1.1]pentan-1-yl)methyl ((2-(2,6-dioxopiperidin-3-yl)-3-oxoisoindolin-5-yl)methyl)carbamate (10.41 mg, 21.10 umol, 37% yield, 96% purity) as a yellow solid.

1H NMR (400 MHz, DMSO-d6) δ=10.97 (br s, 1H), 7.84 (br t, J=6.0 Hz, 1H), 7.63 (s, 1H), 7.59-7.49 (m, 2H), 7.34-7.25 (m, 2H), 7.24-7.10 (m, 3H), 5.10 (dd, J=5.1, 13.4 Hz, 1H), 4.47-4.38 (m, 1H), 4.34-4.25 (m, 3H), 4.08 (s, 2H), 2.95-2.85 (m, 1H), 2.60 (br d, J=17.1 Hz, 1H), 2.39 (br dd, J=4.6, 12.9 Hz, 1H), 2.04-1.96 (m, 1H), 1.94 (s, 5H), 1.78 (br s, 1H)

¹H NMR (400 MHz, DMSO-d₆,T=80° C.) δ=10.82-10.57 (m, 1H), 7.66 (s, 1H), 7.54 (s, 3H), 7.32-7.26 (m, 2H), 7.24-7.17 (m, 3H), 5.06 (dd, J=5.2, 13.1 Hz, 1H), 4.47-4.40 (m, 1H), 4.38-4.29 (m, 3H), 4.09 (s, 2H), 2.94-2.84 (m, 1H), 2.65-2.60 (m, 1H), 2.46-2.37 (m, 1H), 2.07-1.99 (m, 1H), 1.93 (s, 6H)

### Example 7. Synthesis of Compound 7

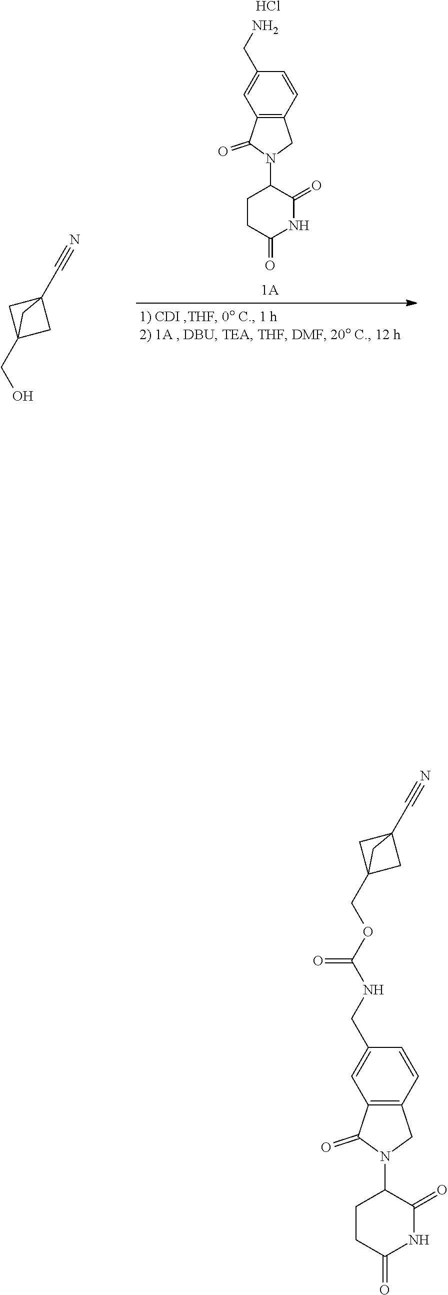 [SMILES: Cl ; N#CC12CC(CO)(C1)C2 ; NCc1ccc2c(c1)C(=O)N(C1CCC(=O)NC1=O)C2 ; N#CC12CC(COC(=O)NCc3ccc4c(c3)C(=O)N(C3CCC(=O)NC3=O)C4)(C1)C2]

### Step 1. Compound (3-cyanobicyclo[1.1.1]pentan-1-yl)methyl ((2-(2,6-dioxopiperidin-3-yl)-3-oxoisoindolin-5-yl)methyl)carbamate

To a solution of 3-(hydroxymethyl)bicyclo[1.1.1]pentane-1-carbonitrile (50.0 mg, 406 umol, 1.00 eq) in tetrahydrofuran (0.500 mL) was added di(1H-imidazol-1-yl)methanone (132 mg, 812 umol, 2.00 eq) at 0° C., the mixture was stirred at 0° C. for 1 h. The resulting solution was added to a mixture of 3-(6-(aminomethyl)-1-oxoisoindolin-2-yl)piperidine-2,6-dione (126 mg, 406 umol, 1.00 eq, hydrochloride), 2,3,4,6,7,8,9,10-octahydropyrimido[1,2-a]azepine (61.8 mg, 406 umol, 61.2 uL, 1.00 eq) and triethylamine (41.1 mg, 406 umol, 56.5 uL, 1.00 eq) in tetrahydrofuran (0.500 mL) and dimethylformamide (0.500 mL). The reaction mixture was stirred at 20° C. for 12 h. The reaction mixture was concentrated under reduced pressure to give a residue. The residue was diluted with dimethylformamide (1 mL) and then filtered. The filtrate was purified by Prep-HPLC(column: Phenomenex luna C18 150*25 mm*10 um; mobile phase: [water(formic acid)-acetonitrile]; B %: 15%-45%, 9 min) and lyophilized to afford (3-cyanobicyclo[1.1.1]pentan-1-yl)methyl ((2-(2,6-dioxopiperidin-3-yl)-3-oxoisoindolin-5-yl)methyl)carbamate (15.76 mg, 36.93 umol, 9% yield, 99% purity) as a yellow solid.

¹H NMR (400 MHz, DMSO-d₆) δ=10.99 (s, 1H), 7.95-7.83 (m, 1H), 7.62-7.48 (m, 3H), 5.16-5.07 (m, 1H), 4.48-4.39 (m, 1H), 4.36-4.24 (m, 3H), 3.99 (s, 2H), 2.96-2.86 (m, 1H), 2.61 (br s, 1H), 2.39 (br dd, J=4.1, 13.2 Hz, 1H), 2.17 (s, 5H), 2.11-2.04 (m, 1H), 2.03-1.97 (m, 1H).

### Example 8. Synthesis of Compound 8

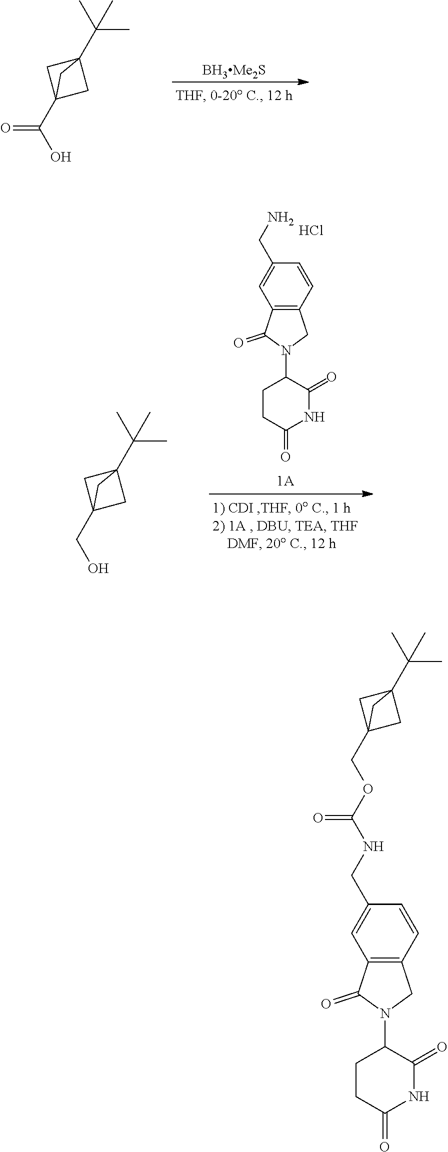 [SMILES: CC(C)(C)C12CC(C(=O)O)(C1)C2 ; "BH3·Me2S" ; CC(C)(C)C12CC(CO)(C1)C2 ; NCc1ccc2c(c1)C(=O)N(C1CCC(=O)NC1=O)C2 ; Cl ; CC(C)(C)C12CC(COC(=O)NCc3ccc4c(c3)C(=O)N(C3CCC(=O)NC3=O)C4)(C1)C2]

### Step 1. Compound (3-(tert-butyl)bicyclo[1.1.1]pentan-1-yl)methanol

To a solution of 3-(tert-butyl)bicyclo[1.1.1]pentane-1-carboxylic acid (100 mg, 594 umol, 1.00 eq) in tetrahydrofuran (1.00 mL) was added borane dimethyl sulfide complex (10.0 M, 119 uL, 2.00 eq) at 0° C. The reaction mixture was stirred at 20° C. for 12 h. The reaction mixture was added to methanol (10.0 mL) to be quenched. Then the mixture was concentrated under reduced pressure to afford (3-(tert-butyl)bicyclo[1.1.1]pentan-1-yl)methanol (77.0 mg, 499 umol, 84% yield) as colorless oil.

1H NMR (400 MHz, CDCl₃) δ=3.60 (s, 2H), 1.49 (s, 6H), 0.85 (s, 9H).

### Step 2. Compound (3-(tert-butyl)bicyclo[1.1.1]pentan-1-yl)methyl((2-(2,6-dioxopiperidin-3-yl)-3-oxoisoindolin-5-yl)methyl)carbamate

To a solution of (3-(tert-butyl)bicyclo[1.1.1]pentan-1-yl)methanol (35.0 mg, 227 umol, 1.00 eq) in tetrahydrofuran (0.300 mL) was added di(1H-imidazol-1-yl)methanone (73.6 mg, 454 umol, 2.00 eq) at 0° C. The mixture was stirred at 20° C. for 1 h. The resulting solution was added to a mixture of 3-(6-(aminomethyl)-1-oxoisoindolin-2-yl)piperidine-2,6-dione (70.3 mg, 227 umol, 1.00 eq, hydrochloride), 2,3,4,6,7,8,9,10-octahydropyrimido[1,2-a]azepine (34.5 mg, 227 umol, 34.2 uL, 1.00 eq) and triethylamine (23.0 mg, 227 umol, 31.6 uL, 1.00 eq) in tetrahydrofuran (0.300 mL) and dimethylformamide (0.300 mL). The reaction mixture was stirred at 20° C. for 12 h. The reaction mixture was concentrated under reduced pressure to give a residue. The residue was dissolved in dimethylformamide (1.5 mL) and then filtered. The filtrate was purified by Prep-HPLC(column: Phenomenex luna C18 150*25 mm*10 um; mobile phase: [water(formic acid)-acetonitrile]; B %: 40%-70%, 9 min) and lyophilized to afford (3-(tert-butyl)bicyclo[1.1.1]pentan-1-yl)methyl((2-(2,6-dioxopiperidin-3-yl)-3-oxoisoindolin-5-yl)methyl)carbamate (21.05 mg, 45.95 umol, 20% yield, 99% purity) as an off-white solid.

1H NMR (400 MHz, DMSO-d6) δ=10.97 (br s, 1H), 7.78 (br t, J=6.1 Hz, 1H), 7.61 (s, 1H), 7.58-7.53 (m, 1H), 7.53-7.47 (m, 1H), 5.11 (dd, J=5.0, 13.4 Hz, 1H), 4.46-4.36 (m, 1H), 4.34-4.21 (m, 3H), 3.98 (s, 2H), 2.98-2.85 (m, 1H), 2.60 (td, J=2.1, 15.3 Hz, 1H), 2.43-2.36 (m, 1H), 2.04-1.93 (m, 1H), 1.44 (s, 5H), 1.27-1.14 (m, 1H), 0.80 (s, 8H), 0.71 (br s, 1H).

1H NMR (400 MHz, DMSO-d₆, T=80° C.) δ=10.68 (br s, 1H), 7.64 (s, 1H), 7.56-7.49 (m, 2H), 7.49-7.33 (m, 1H), 5.06 (dd, J=5.3, 13.1 Hz, 1H), 4.46-4.40 (m, 1H), 4.37-4.27 (m, 3H), 3.99 (s, 2H), 2.89 (ddd, J=5.5, 13.4, 17.5 Hz, 1H), 2.66-2.60 (m, 1H), 2.45-2.35 (m, 1H), 2.03 (dtd, J=2.6, 5.3, 12.8 Hz, 1H), 1.49-1.39 (m, 6H), 0.81 (s, 9H).

### Example 9. Synthesis of compound 9

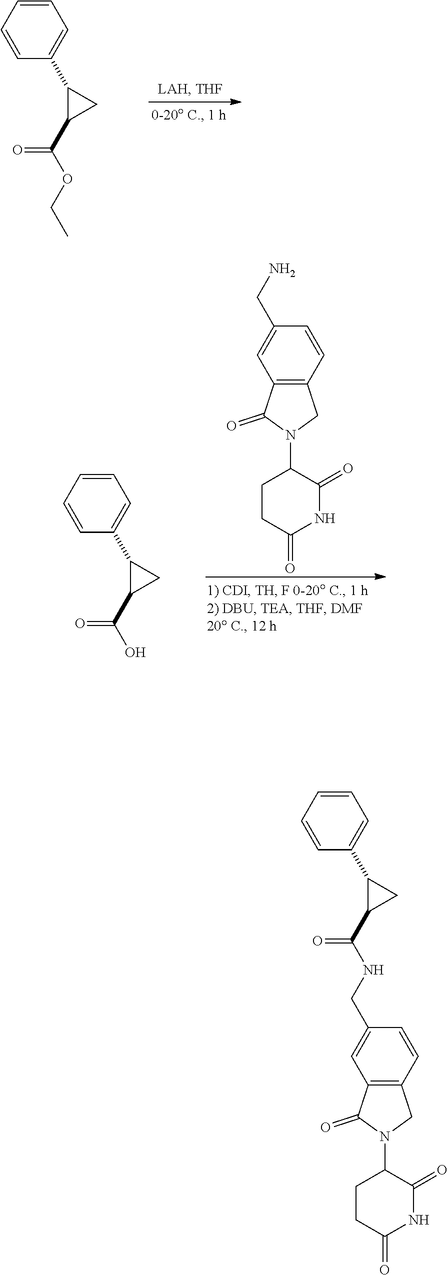

### Step 1. Compound ((1R,2R)-2-phenylcyclopropyl)methanol

To a mixture of (1R,2R)-ethyl 2-phenylcyclopropanecarboxylate (1.00 g, 5.26 mmol, 1.00 eq) in tetrahydrofuran (10.0 mL) was added lithium aluminum hydride (599 mg, 15.8 mmol, 3.00 eq) at 0° C. The mixture was stirred at 20° C. for another 1 h. Two batches of reaction mixture were carried out in parallel and combined. The mixture was quenched with sodium sulfate decahydrate (5.00 g) and filtered. The filtrate was concentrated under reduced pressure to afford ((1R,2R)-2-phenylcyclopropyl)methanol (1.50 g, 10.1 mmol, 96% yield) as colorless oil.

1H NMR (400 MHz, CDCl₃) δ=7.33-7.24 (m, 2H), 7.22-7.16 (m, 1H), 7.13-7.06 (m, 2H), 3.75-3.54 (m, 2H), 1.92-1.76 (m, 1H), 1.55-1.40 (m, 1H), 1.06-0.86 (m, 2H).

### Step 2. ((1R,2R)-2-phenylcyclopropyl)methyl ((2-(2,6-dioxopiperidin-3-yl)-3-oxoisoindolin-5-yl)methyl)carbamate

To a mixture of ((1R,2R)-2-phenylcyclopropyl)methanol (100 mg, 675 umol, 1.00 eq) in tetrahydrofuran (0.500 mL) was added di(1H-imidazol-1-yl)methanone (219 mg, 1.35 mmol, 2.00 eq) at 0° C. The mixture was stirred at 20° C. for 1 h. The reaction mixture was added into a solution of 3-(6-(aminomethyl)-1-oxoisoindolin-2-yl)piperidine-2,6-dione (209 mg, 675 umol, 1.00 eq, hydrochloride), triethylamine (675 umol, 93.9 uL, 1.00 eq) and 2,3,4,6,7,8,9,10-octahydropyrimido[1,2-a]azepine (675 umol, 102 uL, 1.00 eq) in tetrahydrofuran (0.500 mL) and dimethylformamide (0.500 mL). The reaction mixture was stirred at 20° C. for 12 h. The reaction was concentrated under reduced pressure to give a residue. The residue was dissolved in dimethylformamide (1 mL) and then filtered. The filtrate was purified by Prep-HPLC (column: Phenomenex luna C18 150*25 mm*10 um; mobile phase: [water formic acid)-acetonitrile]; B %: 32%-62%, 10 min) and lyophilized to afford ((1R,2R)-2-phenylcyclopropyl)methyl ((2-(2,6-dioxopiperidin-3-yl)-3-oxoisoindolin-5-yl)methyl)carbamate (40.8 mg, 90.27 umol, 13% yield, 99% purity) as a white solid.

1H NMR (400 MHz, DMSO-d₆) δ=10.98 (br s, 1H), 7.84 (br t, J=6.1 Hz, 1H), 7.63 (s, 1H), 7.57-7.43 (m, 2H), 7.29-7.18 (m, 2H), 7.16-7.09 (m, 1H), 7.06 (br d, J=7.4 Hz, 2H), 5.12 (dd, J=5.1, 13.3 Hz, 1H), 4.51-4.40 (m, 1H), 4.37-4.21 (m, 3H), 4.09-3.99 (m, 1H), 3.93 (dd, J=7.5, 11.4 Hz, 1H), 2.98-2.86 (m, 1H), 2.66-2.57 (m, 1H), 2.47-2.38 (m, 1H), 2.06-1.97 (m, 1H), 1.95-1.87 (m, 1H), 1.47-1.35 (m, 1H), 1.05-0.75 (m, 2H).

### Example 10. Synthesis of Compound 10

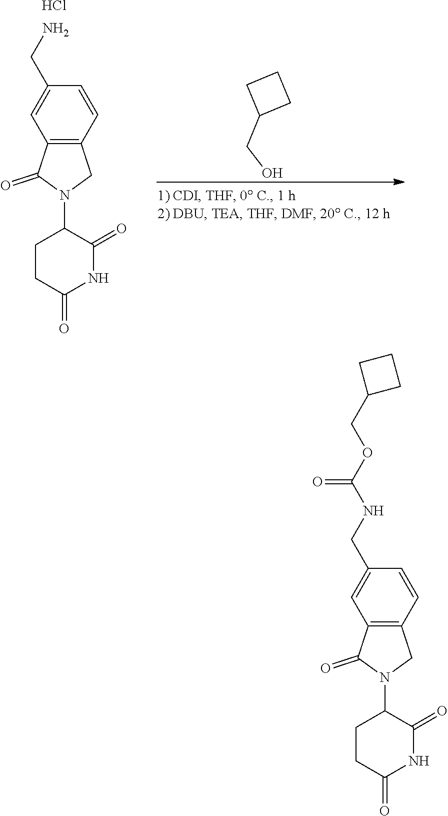 [SMILES: Cl ; NCc1ccc2c(c1)C(=O)N(C1CCC(=O)NC1=O)C2 ; OCC1CCC1 ; O=C1CCC(N2Cc3ccc(CNC(=O)OCC4CCC4)cc3C2=O)C(=O)N1]

### Step 1. Compound cyclobutylmethyl ((2-(2,6-dioxopiperidin-3-yl)-3-oxoisoindolin-5-yl)methyl)carbamate

To solution of cyclobutylmethanol (20.0 mg, 233 umol, 22.0 uL, 1.00 eq) in tetrahydrofuran (0.100 mL) was added di(1H-imidazol-1-yl)methanone (75.3 mg, 465 umol, 2.00 eq) at 0° C. The mixture was stirred at 0° C. for 1 h. The resulting mixture was added to a solution of 3-(6-(aminomethyl)-1-oxoisoindolin-2-yl)piperidine-2,6-dione (72.0 mg, 233 umol, 1.00 eq, hydrochloride), 2,3,4,6,7,8,9,10-octahydropyrimido[1,2-a]azepine (35.4 mg, 233 umol, 35.0 uL, 1.00 eq) and triethylamine (23.5 mg, 233 umol, 32.4 uL, 1.00 eq) in tetrahydrofuran (0.100 mL) and dimethylformamide (0.100 mL). The reaction mixture was stirred at 20° C. for 12 h. The reaction mixture was concentrated under reduced pressure to give a residue. The residue was dissolved in dimethylformamide (0.5 mL) and then filtered. The filtrate was purified by Prep-HPLC(column: Phenomenex luna C18 150*25 mm*10 um; mobile phase: [water(formic acid)-acetonitrile]; B %: 21%-51%, 9 min) and lyophilized to afford cyclobutylmethyl ((2-(2,6-dioxopiperidin-3-yl)-3-oxoisoindolin-5-yl)methyl)carbamate (21.23 mg, 54.53 umol, 24% yield, 99% purity) as an off-white solid.

¹H NMR (400 MHz, DMSO-d₆) δ=10.97 (br s, 1H), 7.77 (br t, J=6.2 Hz, 1H), 7.61 (s, 1H), 7.57-7.53 (m, 1H), 7.52-7.49 (m, 1H), 5.10 (dd, J=5.1, 13.3 Hz, 1H), 4.49-4.37 (m, 1H), 4.34-4.23 (m, 3H), 3.95 (d, J=6.8 Hz, 2H), 2.96-2.85 (m, 1H), 2.63-2.56 (m, 1H), 2.52 (d, J=1.9 Hz, 1H), 2.44-2.37 (m, 1H), 2.04-1.93 (m, 3H), 1.89-1.79 (m, 2H), 1.77-1.66 (m, 2H).

1H NMR (400 MHz, DMSO-d6,T=80° C.) 6=10.84-10.57 (m, 1H), 7.63 (s, 1H), 7.56-7.50 (m, 2H), 7.44 (br dd, J=2.0, 3.8 Hz, 1H), 5.06 (dd, J=5.3, 13.1 Hz, 1H), 4.47-4.41 (m, 1H), 4.37-4.31 (m, 1H), 4.29 (d, J=6.1 Hz, 2H), 3.98 (d, J=6.5 Hz, 2H), 2.89 (ddd, J=5.6, 13.4, 17.5 Hz, 1H), 2.65-2.59 (m, 1H), 2.57-2.53 (m, 1H), 2.41 (dd, J=4.6, 13.1 Hz, 1H), 2.08-1.96 (m, 3H), 1.92-1.80 (m, 2H), 1.79-1.68 (m, 2H).

### Example 11. Synthesis of Compound 11

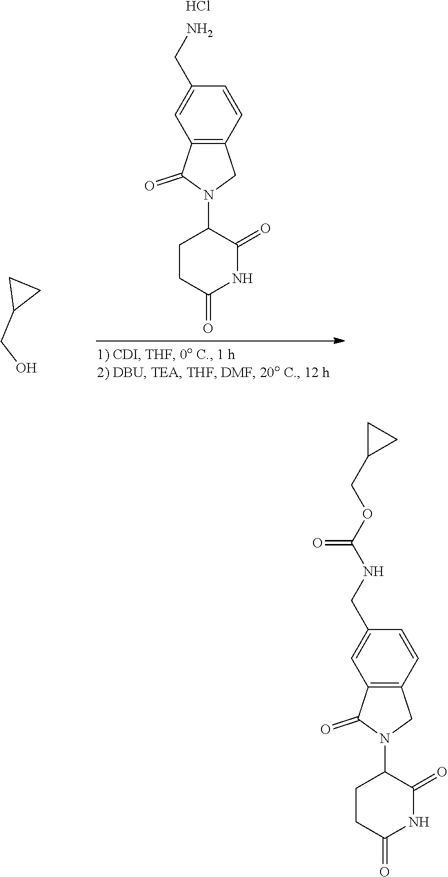 [SMILES: Cl ; OCC1CC1 ; NCc1ccc2c(c1)C(=O)N(C1CCC(=O)NC1=O)C2 ; O=C1CCC(N2Cc3ccc(CNC(=O)OCC4CC4)cc3C2=O)C(=O)N1]

### Step 1. Compound cyclopropylmethyl ((2-(2,6-dioxopiperidin-3-yl)-3-oxoisoindolin-5-yl)methyl)carbamate

To solution of cyclopropylmethanol (10.0 mg, 139 umol, 11.0 uL, 1.00 eq) in tetrahydrofuran (0.100 mL) was added di(1H-imidazol-1-yl)methanone (45.0 mg, 278 umol, 2.00 eq) at 0° C. The mixture was stirred at 0° C. for 1 h. The resulting mixture was added to a solution of 3-(6-(aminomethyl)-1-oxoisoindolin-2-yl)piperidine-2,6-dione (43.0 mg, 139 umol, 1.00 eq, hydrochloride), 2,3,4,6,7,8,9,10-octahydropyrimido[1,2-a]azepine (21.2 mg, 139 umol, 20.9 uL, 1.00 eq) and triethylamine (14.1 mg, 139 umol, 19.3 uL, 1.00 eq) in tetrahydrofuran (0.100 mL) and dimethylformamide (0.100 mL). The reaction mixture was stirred at 20° C. for 12 h. The reaction mixture was concentrated under reduced pressure to give a residue. The residue was dissolved in dimethylformamide (0.5 mL) and then filtered. The filtrate was purified by Prep-HPLC(column: Phenomenex luna C18 150*25 mm*10 um; mobile phase: [water(formic acid)-acetonitrile]; B %: 14%-44%, 9 min) and lyophilized to afford cyclopropylmethyl ((2-(2,6-dioxopiperidin-3-yl)-3-oxoisoindolin-5-yl)methyl)carbamate (12.25 mg, 32.98 umol, 24% yield, 99% purity) as an off-white solid.

1H NMR (400 MHz, DMSO-d6) δ=10.97 (s, 1H), 7.79 (br t, J=6.2 Hz, 1H), 7.61 (s, 1H), 7.53 (q, J=7.8 Hz, 2H), 5.10 (dd, J=5.1, 13.3 Hz, 1H), 4.47-4.40 (m, 1H), 4.34-4.24 (m, 3H), 3.81 (d, J=7.1 Hz, 2H), 2.97-2.85 (m, 1H), 2.63-2.57 (m, 1H), 2.45-2.36 (m, 1H), 2.05-1.96 (m, 1H), 1.11-1.02 (m, 1H), 0.53-0.43 (m, 2H), 0.24 (q, J=4.8 Hz, 2H).

### Example 12. Synthesis of Compound 12

### Synthetic Procedure 1:

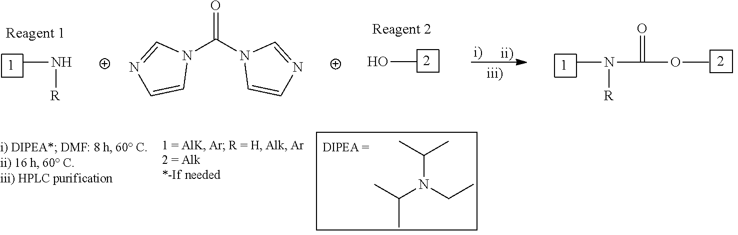

Reagent 2 (appr. 1.3-2 eq.) and 1,1′-Carbonyldiimidazole (CDI) (appr. 1.8-2.5 eq.) were mixed in dry Dimethylformamide*(DMF) (appr. 0.7 ml per 100 mg of product). The reaction mixture was sealed and heated for 8 hours at 60° C. Then the mixture was cooled to the ambient temperature and Reagent 1 (appr. 1 eq.) was added in one portion. The reaction mixture was sealed and heated for 16 hours at 60° C., and cooled to the ambient temperature. The solvent was evaporated under reduced pressure and the residue was dissolved in the DMSO (appr. 1 ml up to 300 mg of product). DMSO solution was filtered, analyzed by LCMS, and transferred for HPLC purification.

(4-cyclopentylphenyl)methyl N-{[2-(2,6-dioxopiperidin-3-yl)-3-oxo-2,3-dihydro-1H-isoindol-5-yl]methyl}carbamate (Compound 12) was obtained by Synthetic procedure 1 with using 43 mg (0.139 mmol) of 3-[6-(aminomethyl)-1-oxo-2,3-dihydro-1H-isoindol-2-yl]piperidine-2,6-dione hydrochloride (Reagent 1), 32 mg (0.182 mmol) of (4-cyclopentylphenyl)methanol (Reagent 2), 42 mg (0.259 mmol) of CDI, and 39.7 mg (0.307 mmol) of DIPEA. Purified by HPLC (gradient: from A-55%: B-45% to A-40%: B-60%; R_{f}=0.71; run time=6.5 min). Yield: 26.3 mg (39.6%). White powder. LCMS purity: 100% (LCMS, R_{f}=0.7, run time=2 min). EI MS m/z: pos. 476.2 (MH⁺).

### Example 13. Syntheses of Compounds 13-40

### Synthetic Procedure 1:

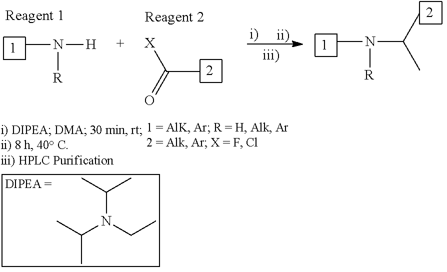

Reagent 1 (appr. 1 eq.), and Reagent 2 (appr. 1.3-2 eq.), and Diisopropylethylamine (DIPEA) (appr. 3.8-5 eq.) were mixed in dry Dimethylacetamide (DMA) (appr. 0.7 ml per 100 mg of product). The reaction mixture was sealed and heated for 8 hours at 40°, then cooled to the ambient temperature, and the solvent was evaporated under reduced pressure. The residue was dissolved in the DMSO (appr. 1 ml up to 300 mg of product) and DMSO solution was filtered, analyzed by LCMS, and transferred for HPLC purification.

### Synthetic Procedure 2:

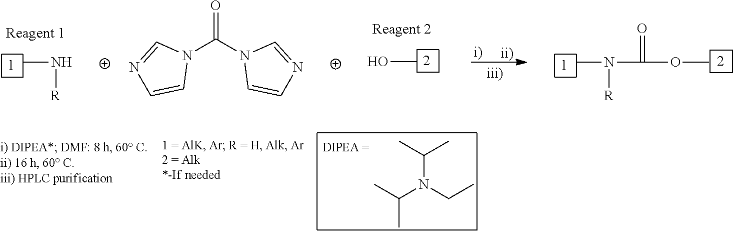

Reagent 2 (appr. 1.3-2 eq.) and 1,1′-Carbonyldiimidazole (CDI) (appr. 1.5-2.5 eq.) were mixed in dry Dimethylacetamide (DMA) (appr. 0.7 ml per 100 mg of product). The reaction mixture was sealed and heated for 8 hours at 60° C. Then the mixture was cooled to the ambient temperature and Reagent 1 (appr. 1 eq.) was added in one portion. The reaction mixture was sealed and heated for 16 hours at 60° C., and cooled to the ambient temperature. The solvent was evaporated under reduced pressure and the residue was dissolved in the DMSO (appr. 1 ml up to 300 mg of product). DMSO solution was filtered, analyzed by LCMS, and transferred for HPLC purification.

oxolan-3-yl N-{[2-(2,6-dioxopiperidin-3-yl)-3-oxo-2,3-dihydro-1H-isoindol-5-yl]methyl}carbamate (Compound 13) was obtained by Synthetic procedure 1 with using 54 mg (0.174 mmol) of 3-[6-(aminomethyl)-1-oxo-2,3-dihydro-1H-isoindol-2-yl]piperidine-2,6-dione hydrochloride (Reagent 1), 42 mg (0.279 mmol) of oxolan-3-yl carbonochloridate (Reagent 2), and 96.1 mg (0.744 mmol) of DIPEA. Purified by HPLC (gradient: from A-95%: B-5% to A-80%: B-20%; Rf=0.54; run time=8.5 min). Yield: 45.2 mg (62.7%). White powder. LCMS purity: 95.1% (LCMS, Rf=0.32, run time=2.5 min). EI MS m/z: pos. 388.2 (MH⁺).

cyclopropyl N-{[2-(2,6-dioxopiperidin-3-yl)-3-oxo-2,3-dihydro-1H-isoindol-5-yl]methyl}carbamate (Compound 14) was obtained by Synthetic procedure 1 with using 39 mg (0.126 mmol) of 3-[6-(aminomethyl)-1-oxo-2,3-dihydro-1H-isoindol-2-yl]piperidine-2,6-dione hydrochloride (Reagent 1), 21 mg (0.174 mmol) of cyclopropyl carbonochloridate (Reagent 2), and 131.6 mg (1.018 mmol) of DIPEA. Purified by HPLC (gradient: from A-80%: B-20% to A-65%: B-35%; Rf=0.32; run time=8.5 min). Yield: 5 mg (10.4%). Beige powder. LCMS purity: 95.2% (LCMS, Rf=0.33, run time=2.5 min). EI MS m/z: pos. 358.2 (MH⁺).

4,4-dimethylcyclohexyl N-{[2-(2,6-dioxopiperidin-3-yl)-1-oxo-2,3-dihydro-1H-isoindol-5-yl]methyl}carbamate (Compound 15) was obtained by Synthetic procedure 2 with using 46 mg (0.149 mmol) of 3-[5-(aminomethyl)-1-oxo-2,3-dihydro-1H-isoindol-2-yl]piperidine-2,6-dione hydrochloride (Reagent 1), 28 mg (0.218 mmol) of 4,4-dimethylcyclohexan-1-ol (Reagent 2), 49 mg (0.302 mmol) of CDI, and 41.4 mg (0.32 mmol) of DIPEA. Purified by HPLC (gradient: from A-70%: B-30% to A-55%: B-45%; R_{f}=0.6; run time=8.5 min). Yield: 8.3 mg (13.3%). Yellow powder. LCMS purity: 100% (LCMS, R_{f}=0.6, run time=2 min). EI MS m/z: pos. 428.4 (MH⁺).

(3-methyloxetan-3-yl)methyl N-{[2-(2,6-dioxopiperidin-3-yl)-3-oxo-2,3-dihydro-1H-isoindol-5-yl]methyl}carbamate (Compound 16) was obtained by Synthetic procedure 2 with using 57 mg (0.184 mmol) of 3-[6-(aminomethyl)-1-oxo-2,3-dihydro-1H-isoindol-2-yl]piperidine-2,6-dione hydrochloride (Reagent 1), 26 mg (0.255 mmol) of (3-methyloxetan-3-yl)methanol (Reagent 2), 57 mg (0.352 mmol) of CDI, and 48.3 mg (0.374 mmol) of DIPEA. Purified by HPLC (gradient: from A-90%: B-10% to A-75%: B-25%; Rf=0.44; run time=8.5 min). Yield: 14 mg (20.6%). White powder. LCMS purity: 100% (LCMS, Rf=0.4, run time=2 min). EI MS m/z: pos. 402.0 (MH⁺).

1,2,3,4-tetrahydronaphthalen-2-yl N-{[2-(2,6-dioxopiperidin-3-yl)-3-oxo-2,3-dihydro-1H-isoindol-5-yl]methyl}carbamate (Compound 17) was obtained by Synthetic procedure 2 with using 54 mg (0.174 mmol) of 3-[6-(aminomethyl)-1-oxo-2,3-dihydro-1H-isoindol-2-yl]piperidine-2,6-dione hydrochloride (Reagent 1), 36 mg (0.243 mmol) of 1,2,3,4-tetrahydronaphthalen-2-ol (Reagent 2), 52 mg (0.321 mmol) of CDI, and 46 mg (0.356 mmol) of DIPEA. Purified by HPLC gradient: from A-70%: B-30% to A-55%: B-45%; Rf=0.55; run time=8.2 min). Yield: 9.2 mg (12.7%). Yellow powder. LCMS purity: 98% (LCMS, Rf=0.58, run time=2 min). EI MS m/z: pos. 448.2 (MH⁺).

(2,3-dihydro-1H-inden-1-yl)methyl N-{[2-(2,6-dioxopiperidin-3-yl)-3-oxo-2,3-dihydro-1H-isoindol-5-yl]methyl}carbamate (Compound 18) was obtained by Synthetic procedure 2 with using 47 mg (0.152 mmol) of 3-[6-(aminomethyl)-1-oxo-2,3-dihydro-1H-isoindol-2-yl]piperidine-2,6-dione hydrochloride (Reagent 1), 30 mg (0.202 mmol) of (2,3-dihydro-1H-inden-1-yl)methanol (Reagent 2), 45 mg (0.278 mmol) of CDI, and 44.3 mg (0.343 mmol) of DIPEA. Purified by HPLC (gradient: from A-70%: B-30% to A-55%: B-45%; Rf=0.56; run time=8.2 min). Yield: 11.5 mg (16.5%). Beige powder. LCMS purity: 96.8% (LCMS, Rf=0.45, run time=2.5 min). EI MS m/z: pos. 448.0 (MH⁺).

(3,4-dihydro-1H-2-benzopyran-1-yl)methyl N-{[2-(2,6-dioxopiperidin-3-yl)-3-oxo-2,3-dihydro-1H-isoindol-5-yl]methyl}carbamate (Compound 19) was obtained by Synthetic procedure 2 with using 51 mg (0.165 mmol) of 3-[6-(aminomethyl)-1-oxo-2,3-dihydro-1H-isoindol-2-yl]piperidine-2,6-dione hydrochloride (Reagent 1), 33 mg (0.201 mmol) of (3,4-dihydro-1H-2-benzopyran-1-yl)methanol (Reagent 2), 47 mg (0.29 mmol) of CDI, and 44 mg (0.34 mmol) of DIPEA. Purified by HPLC (gradient: from A-80%: B-20% to A-65%: B-35%; Rf=0.54; run time=8.2 min). Yield: 4.7 mg (6.6%). Yellow powder. LCMS purity: 95.2% (LCMS, Rf=0.51, run time=2 min). EI MS m/z: pos. 464.2 (MH⁺).

[3-(propan-2-yl)cyclobutyl]methyl N-{[2-(2,6-dioxopiperidin-3-yl)-3-oxo-2,3-dihydro-1H-isoindol-5-yl]methyl}carbamate (Compound 20) was obtained by Synthetic procedure 2 with using 54 mg (0.174 mmol) of 3-[6-(aminomethyl)-1-oxo-2,3-dihydro-1H-isoindol-2-yl]piperidine-2,6-dione hydrochloride (Reagent 1), 27 mg (0.211 mmol) of [3-(propan-2-yl)cyclobutyl]methanol (Reagent 2), 45 mg (0.278 mmol) of CDI, and 46.1 mg (0.357 mmol) of DIPEA. Purified by HPLC (gradient: from A-65%: B-35% to A-50%: B-50%; Rf=0.57; run time=8.5 min). Yield: 7.5 mg (10.8%). Yellow powder.). 1H NMR purity: 95%.

2-cyclopropoxyethyl N-{[2-(2,6-dioxopiperidin-3-yl)-3-oxo-2,3-dihydro-1H-isoindol-5-yl]methyl}carbamate (Compound 21) was obtained by Synthetic procedure 2 with using 56 mg (0.181 mmol) of 3-[6-(aminomethyl)-1-oxo-2,3-dihydro-1H-isoindol-2-yl]piperidine-2,6-dione hydrochloride (Reagent 1), 25 mg (0.245 mmol) of 2-cyclopropoxyethan-1-ol (Reagent 2), 55 mg (0.339 mmol) of CDI, and 53.5 mg (0.414 mmol) of DIPEA. Purified by HPLC (gradient: from A-90%: B-10% to A-75%: B-25%; Rf=0.58; run time=8.5 min). Yield: 11.1 mg (14.7%). Beige powder.). 1H NMR purity: 95%.

(2-methyloxolan-2-yl)methyl N-{[2-(2,6-dioxopiperidin-3-yl)-3-oxo-2,3-dihydro-1H-isoindol-5-yl]methyl}carbamate (Compound 22) was obtained by Synthetic procedure 2 with using 58 mg (0.187 mmol) of 3-[6-(aminomethyl)-1-oxo-2,3-dihydro-1H-isoindol-2-yl]piperidine-2,6-dione hydrochloride (Reagent 1), 28 mg (0.241 mmol) of (2-methyloxolan-2-yl)methanol (Reagent 2), 51 mg (0.315 mmol) of CDI, and 52.7 mg (0.408 mmol) of DIPEA. Purified by HPLC (gradient: from A-90%: B-10% to A-75%: B-25%; Rf=0.54; run time=8.5 min). Yield: 14.2 mg (18.4%). Yellow powder. LCMS purity: 95.4% (LCMS, Rf=0.34, run time=2.5 min). EI MS m/z: pos. 416.0 (MH+).

(1s,3s)-3-methoxycyclobutyl N-{[2-(2,6-dioxopiperidin-3-yl)-3-oxo-2,3-dihydro-1H-isoindol-5-yl]methyl}carbamate (Compound 23) was obtained by Synthetic procedure 2 with using 60 mg (0.194 mmol) of 3-[6-(aminomethyl)-1-oxo-2,3-dihydro-1H-isoindol-2-yl]piperidine-2,6-dione hydrochloride (Reagent 1), 30 mg (0.294 mmol) of (1s,3s)-3-methoxycyclobutan-1-ol (Reagent 2), 64 mg (0.395 mmol) of CDI, and 55.7 mg (0.431 mmol) of DIPEA. Purified by HPLC gradient: from A-90%: B-10% to A-65%: B-35%; Rf=0.6; run time=8.5 min). Yield: 14 mg (17.8%). White powder. LCMS purity: 100% (LCMS, Rf=0.47, run time=2 min). EI MS m/z: pos. 402.2 (MH+).

2,2-dimethylcyclobutyl N-{[2-(2,6-dioxopiperidin-3-yl)-3-oxo-2,3-dihydro-1H-isoindol-5-yl]methyl}carbamate (Compound 24) was obtained by Synthetic procedure 2 with using 55 mg (0.178 mmol) of 3-[6-(aminomethyl)-1-oxo-2,3-dihydro-1H-isoindol-2-yl]piperidine-2,6-dione hydrochloride (Reagent 1), 28 mg (0.28 mmol) of 2,2-dimethylcyclobutan-1-ol (Reagent 2), 63 mg (0.389 mmol) of CDI, and 53 mg (0.41 mmol) of DIPEA. Purified by HPLC (gradient: from A-75%: B-25% to A-60%: B-40%; Rf=0.56; run time=8.5 min). Yield: 6.9 mg (9.3%). White powder. LCMS purity: 98.7% (LCMS, Rf=0.59, run time=2 min). EI MS m/z: pos. 400.0 (MH+).

[(1S,2S)-2-methylcyclopropyl]methyl N-{[2-(2,6-dioxopiperidin-3-yl)-3-oxo-2,3-dihydro-1H-isoindol-5-yl]methyl}carbamate (Compound 25) was obtained by Synthetic procedure 2 with using 80 mg (0.258 mmol) of 3-[6-(aminomethyl)-1-oxo-2,3-dihydro-1H-isoindol-2-yl]piperidine-2,6-dione hydrochloride (Reagent 1), 29 mg (0.337 mmol) of [(1S,2S)-2-methylcyclopropyl]methanol (Reagent 2), 78 mg (0.481 mmol) of CDI, and 73.6 mg (0.569 mmol) of DIPEA. Purified by HPLC (gradient: from A-75%: B-25% to A-60%: B-40%; Rf=0.46; run time=8.5 min). Yield: 13.6 mg (13.6%). Yellow powder. LCMS purity: 97.8% (LCMS, Rf=0.55, run time=2 min). EI MS m/z: pos. 386.0 (MH+).

rac-(1R,2S)-2-phenylcyclopentyl N-{[2-(2,6-dioxopiperidin-3-yl)-3-oxo-2,3-dihydro-1H-isoindol-5-yl]methyl}carbamate; trifluoroacetic acid (Compound 26) was obtained by Synthetic procedure 2 with using 55 mg (0.178 mmol) of 3-[6-(aminomethyl)-1-oxo-2,3-dihydro-1H-isoindol-2-yl]piperidine-2,6-dione hydrochloride (Reagent 1), 42 mg (0.259 mmol) of rac-(1R,2S)-2-phenylcyclopentan-1-ol (Reagent 2), 57 mg (0.352 mmol) of CDI, and 49.1 mg (0.38 mmol) of DIPEA. Purified by HPLC (gradient: from A-70%: B-30% to A-55%: B-45%; Rf=0.69; run time=8.5 min). Yield: 11.1 mg (13.9%). Light brown powder. LCMS purity: 97.4% (LCMS, Rf=0.49, run time=2.5 min). EI MS m/z: pos. 462.2 (MH+).

rac-(1R,2S,4S)-7-oxabicyclo[2.2.1]heptan-2-yl N-{[2-(2,6-dioxopiperidin-3-yl)-3-oxo-2,3-dihydro-1H-isoindol-5-yl]methyl}carbamate (Compound 27) was obtained by Synthetic procedure 2 with using 53 mg (0.171 mmol) of 3-[6-(aminomethyl)-1-oxo-2,3-dihydro-1H-isoindol-2-yl]piperidine-2,6-dione hydrochloride (Reagent 1), 30 mg (0.263 mmol) of rac-(1R,2S,4S)-7-oxabicyclo[2.2.1]heptan-2-ol (Reagent 2), 58 mg (0.358 mmol) of CDI, and 49.8 mg (0.385 mmol) of DIPEA. Purified by HPLC (gradient: from A-85%: B-15% to A-70%: B-30%; Rf=0.55; run time=8.5 min). Yield: 11 mg (15.2%). White powder. LCMS purity: 98.5% (LCMS, Rf=0.46, run time=2 min). EI MS m/z: pos. 414.0 (MH+).

(1S)-1-cyclopropylethyl N-{[2-(2,6-dioxopiperidin-3-yl)-3-oxo-2,3-dihydro-1H-isoindol-5-yl]methyl}carbamate (Compound 28) was obtained by Synthetic procedure 2 with using 72 mg (0.232 mmol) of 3-[6-(aminomethyl)-1-oxo-2,3-dihydro-1H-isoindol-2-yl]piperidine-2,6-dione hydrochloride (Reagent 1), 29 mg (0.337 mmol) of (1S)-1-cyclopropylethan-1-ol (Reagent 2), 73 mg (0.45 mmol) of CDI, and 63.8 mg (0.494 mmol) of DIPEA. Purified by HPLC (gradient: from A-75%: B-25% to A-60%: B-40%; Rf=0.52; run time=8.5 min). Yield: 6.2 mg (7.2%). Yellow powder. LCMS purity: 95.1% (LCMS, Rf=0.66, run time=2.5 min). EI MS m/z: pos. 386.0 (MH+).

[(2R)-oxolan-2-yl]methyl N-{[2-(2,6-dioxopiperidin-3-yl)-3-oxo-2,3-dihydro-1H-isoindol-5-yl]methyl}carbamate (Compound 29) was obtained by Synthetic procedure 2 with using 73 mg (0.236 mmol) of 3-[6-(aminomethyl)-1-oxo-2,3-dihydro-1H-isoindol-2-yl]piperidine-2,6-dione hydrochloride (Reagent 1), 31 mg (0.304 mmol) of [(2R)-oxolan-2-yl]methanol (Reagent 2), 69 mg (0.426 mmol) of CDI, and 66.4 mg (0.514 mmol) of DIPEA. Purified by HPLC (gradient: from A-95%: B-5% to A-80%: B-20%; Rf=0.63; run time=8.5 min). Yield: 16.2 mg (17.3%). Beige powder. LCMS purity: 97.7% (LCMS, Rf=0.49, run time=2 min). EI MS m/z: pos. 402.2 (MH+).

(3R,4S)-4-methyloxolan-3-yl N-{[2-(2,6-dioxopiperidin-3-yl)-3-oxo-2,3-dihydro-1H-isoindol-5-yl]methyl}carbamate (Compound 30) was obtained by Synthetic procedure 2 with using 72 mg (0.232 mmol) of 3-[6-(aminomethyl)-1-oxo-2,3-dihydro-1H-isoindol-2-yl]piperidine-2,6-dione hydrochloride (Reagent 1), 34 mg (0.333 mmol) of (3R,4S)-4-methyloxolan-3-ol (Reagent 2), 71 mg (0.438 mmol) of CDI, and 63.1 mg (0.488 mmol) of DIPEA. Purified by HPLC (gradient: from A-95%: B-5% to A-80%: B-20%; Rf=0.66; run time=8.5 min). Yield: 9.8 mg (11%). White powder.). 1H NMR purity: 95%.

3,3-dimethylcyclobutyl N-{[2-(2,6-dioxopiperidin-3-yl)-3-oxo-2,3-dihydro-1H-isoindol-5-yl]methyl}carbamate (Compound 31) was obtained by Synthetic procedure 2 with using 58 mg (0.187 mmol) of 3-[6-(aminomethyl)-1-oxo-2,3-dihydro-1H-isoindol-2-yl]piperidine-2,6-dione hydrochloride (Reagent 1), 26 mg (0.26 mmol) of 3,3-dimethylcyclobutan-1-ol (Reagent 2), 53 mg (0.327 mmol) of CDI, and 49.2 mg (0.381 mmol) of DIPEA. Purified by HPLC (gradient: from A-80%: B-20% to A-60%: B-40%; Rf=0.64; run time=8.2 min). Yield: 13.9 mg (20.1%). Beige powder. LCMS purity: 100% (LCMS, Rf=0.51, run time=2 min). EI MS m/z: pos. 400.4 (MH+).

(1S,2S)-2-methylcyclopentyl N-{[2-(2,6-dioxopiperidin-3-yl)-3-oxo-2,3-dihydro-1H-isoindol-5-yl]methyl}carbamate (Compound 32) was obtained by Synthetic procedure 2 with using 53 mg (0.171 mmol) of 3-[6-(aminomethyl)-1-oxo-2,3-dihydro-1H-isoindol-2-yl]piperidine-2,6-dione hydrochloride (Reagent 1), 26 mg (0.26 mmol) of (1S,2S)-2-methylcyclopentan-1-ol (Reagent 2), 53 mg (0.327 mmol) of CDI, and 49.2 mg (0.381 mmol) of DIPEA. Purified by HPLC (gradient: from A-75%: B-25% to A-60%: B-40%; Rf=0.54; run time=8.5 min). Yield: 7.7 mg (11.1%). White powder. LCMS purity: 97.5% (LCMS, Rf=0.74, run time=2.5 min). EI MS m/z: pos. 400.0 (MH+).

2-(oxetan-3-yloxy)ethyl N-{[2-(2,6-dioxopiperidin-3-yl)-3-oxo-2,3-dihydro-1H-isoindol-5-yl]methyl}carbamate (Compound 33) was obtained by Synthetic procedure 2 with using 56 mg (0.181 mmol) of 3-[6-(aminomethyl)-1-oxo-2,3-dihydro-1H-isoindol-2-yl]piperidine-2,6-dione hydrochloride (Reagent 1), 26 mg (0.22 mmol) of 2-(oxetan-3-yloxy)ethan-1-ol (Reagent 2), 48 mg (0.296 mmol) of CDI, and 48.1 mg (0.372 mmol) of DIPEA. Purified by HPLC (gradient: from A-90%: B-10% to A-75%: B-25%; Rf=0.56; run time=8.5 min). Yield: 16.9 mg (23.9%). Yellow sticky oil. LCMS purity: 95% (LCMS, Rf=0.39, run time=2 min). EI MS m/z: pos. 418.2 (MH+).

(1-methylpiperidin-4-yl)methyl N-{[2-(2,6-dioxopiperidin-3-yl)-3-oxo-2,3-dihydro-1H-isoindol-5-yl]methyl}carbamate; trifluoroacetic acid (Compound 34) was obtained by Synthetic procedure 2 with using 76 mg (0.245 mmol) of 3-[6-(aminomethyl)-1-oxo-2,3-dihydro-1H-isoindol-2-yl]piperidine-2,6-dione hydrochloride (Reagent 1), 62 mg (0.48 mmol) of (1-methylpiperidin-4-yl)methanol (Reagent 2), 99 mg (0.611 mmol) of CDI, and 68.2 mg (0.528 mmol) of DIPEA. Purified by HPLC (gradient: from A-100%: B-0% to A-90%: B-10%; Rf=0.7; run time=6.5 min). Yield: 14 mg (13.6%). Yellow sticky oil. LCMS purity: 92.5% (LCMS, Rf=0.26, run time=2.5 min). EI MS m/z: pos. 429.2 (MH+).

bicyclo[4.2.0]octa-1,3,5-trien-7-yl N-{[2-(2,6-dioxopiperidin-3-yl)-3-oxo-2,3-dihydro-1H-isoindol-5-yl]methyl}carbamate (Compound 35) was obtained by Synthetic procedure 2 with using 58 mg (0.187 mmol) of 3-[6-(aminomethyl)-1-oxo-2,3-dihydro-1H-isoindol-2-yl]piperidine-2,6-dione hydrochloride (Reagent 1), 33 mg (0.275 mmol) of bicyclo[4.2.0]octa-1,3,5-trien-7-ol (Reagent 2), 62 mg (0.382 mmol) of CDI, and 52.1 mg (0.403 mmol) of DIPEA. Purified by HPLC (gradient: from A-70%: B-30% to A-55%: B-45%; Rf=0.53; run time=8.5 min). Yield: 22.7 mg (29.6%). White powder. LCMS purity: 100% (LCMS, Rf=0.57, run time=2 min). EI MS m/z: pos. 420.2 (MH+).

(1-methyl-5-oxopyrrolidin-2-yl)methyl N-{[2-(2,6-dioxopiperidin-3-yl)-3-oxo-2,3-dihydro-1H-isoindol-5-yl]methyl}carbamate (Compound 36) was obtained by Synthetic procedure 2 with using 61 mg (0.197 mmol) of 3-[6-(aminomethyl)-1-oxo-2,3-dihydro-1H-isoindol-2-yl]piperidine-2,6-dione hydrochloride (Reagent 1), 32 mg (0.248 mmol) of 5-(hydroxymethyl)-1-methylpyrrolidin-2-one (Reagent 2), 60 mg (0.37 mmol) of CDI, and 54.2 mg (0.419 mmol) of DIPEA. Purified by HPLC (gradient: from A-95%: B-5% to A-80%: B-20%; Rf=0.48; run time=8.5 min). Yield: 31.2 mg (38.2%). Colorless sticky oil. LCMS purity: 97.1% (LCMS, Rf=0.41, run time=2 min). EI MS m/z: pos. 429.1 (MH+).

rac-(2R,3R)-2-phenyloxetan-3-yl N-{[2-(2,6-dioxopiperidin-3-yl)-3-oxo-2,3-dihydro-1H-isoindol-5-yl]methyl}carbamate (Compound 37) was obtained by Synthetic procedure 2 with using 45 mg (0.145 mmol) of 3-[6-(aminomethyl)-1-oxo-2,3-dihydro-1H-isoindol-2-yl]piperidine-2,6-dione hydrochloride (Reagent 1), 34 mg (0.226 mmol) of rac-(2R,3R)-2-phenyloxetan-3-ol (Reagent 2), 52 mg (0.321 mmol) of CDI, and 42.9 mg (0.332 mmol) of DIPEA. Purified by HPLC (gradient: from A-95%: B-5% to A-80%: B-20%; Rf=0.85; run time=5 min). Yield: 7.5 mg (11.1%). Yellow powder. LCMS purity: 100% (LCMS, Rf=0.36, run time=2.5 min). EI MS m/z: pos. 450.0 (MH+).

(3,3-difluoro-1-phenylcyclobutyl)methyl N-{[2-(2,6-dioxopiperidin-3-yl)-3-oxo-2,3-dihydro-1H-isoindol-5-yl]methyl}carbamate (Compound 38) was obtained by Synthetic procedure 2 with using 50 mg (0.161 mmol) of 3-[6-(aminomethyl)-1-oxo-2,3-dihydro-1H-isoindol-2-yl]piperidine-2,6-dione hydrochloride (Reagent 1), 40 mg (0.202 mmol) of (3,3-difluoro-1-phenylcyclobutyl)methanol (Reagent 2), 44 mg (0.271 mmol) of CDI, and 44.1 mg (0.341 mmol) of DIPEA. Purified by HPLC (gradient: from A-70%: B-30% to A-55%: B-45%; Rf=0.54; run time=8.2 min). Yield: 14 mg (18.1%). White powder. LCMS purity: 95.5% (LCMS, Rf=0.56, run time=2 min). EI MS m/z: pos. 498.2 (MH+).

(1-methylpyrrolidin-3-yl)methyl N-{[2-(2,6-dioxopiperidin-3-yl)-3-oxo-2,3-dihydro-1H-isoindol-5-yl]methyl}carbamate; trifluoroacetic acid (Compound 39) was obtained by Synthetic procedure 2 with using 80 mg (0.258 mmol) of 3-[6-(aminomethyl)-1-oxo-2,3-dihydro-1H-isoindol-2-yl]piperidine-2,6-dione hydrochloride (Reagent 1), 45 mg (0.391 mmol) of (1-methylpyrrolidin-3-yl)methanol (Reagent 2), 88 mg (0.543 mmol) of CDI, and 74.1 mg (0.573 mmol) of DIPEA. Purified by HPLC (gradient: from A-100%: B-0% to A-90%: B-10%; Rf=0.65; run time=6.5 min). Yield: 13.9 mg (12.9%). Yellow powder. LCMS purity: 97% (LCMS, Rf=0.37, run time=2 min). EI MS m/z: pos. 415.0 (MH+).

1-methylpyrrolidin-3-yl N-{[2-(2,6-dioxopiperidin-3-yl)-3-oxo-2,3-dihydro-1H-isoindol-5-yl]methyl}carbamate; trifluoroacetic acid (Compound 40) was obtained by Synthetic procedure 2 with using 84 mg (0.271 mmol) of 3-[6-(aminomethyl)-1-oxo-2,3-dihydro-1H-isoindol-2-yl]piperidine-2,6-dione hydrochloride (Reagent 1), 54 mg (0.534 mmol) of 1-methylpyrrolidin-3-ol (Reagent 2), 108 mg (0.666 mmol) of CDI, and 75.9 mg (0.587 mmol) of DIPEA. Purified by HPLC (gradient: from A-80%: B-20% to A-55%: B-45%; Rf=0.63; run time=8.5 min). Yield: 17.3 mg (16.2%). Yellow powder. LCMS purity: 100% (LCMS, Rf=0.35, run time=2 min). EI MS m/z: pos. 401.2 (MH+).

### Example 14. Syntheses of Compounds 41-100

### Synthetic Procedure 1

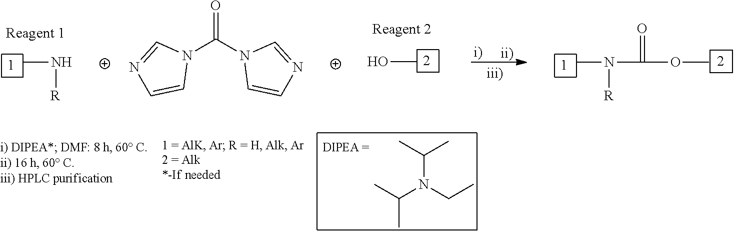

Reagent 2 (appr. 1.2-2 eq.) and 1,1′-Carbonyldiimidazole (CDI) (appr. 0.1 eq.) were mixed in dry Dimethylformamide*(DMF) (appr. 0.7 ml per 100 mg of product). The reaction mixture was sealed and heated for 8 hours at 60° C. Then the mixture was cooled to the ambient temperature and Reagent 1 (appr. 1 eq.) was added in one portion. The reaction mixture was sealed and heated for 16 hours at 60° C., and cooled to the ambient temperature. The solvent was evaporated under reduced pressure and the residue was dissolved in the DMSO (appr. 1 ml up to 300 mg of product). DMSO solution was filtered, analyzed by LCMS, and transferred for HPLC purification.

(oxan-2-yl)methyl N-{[2-(2,6-dioxopiperidin-3-yl)-3-oxo-2,3-dihydro-1H-isoindol-5-yl]methyl}carbamate (Compound 41) was obtained by Synthetic procedure 1 with using 53 mg (0,171 mmol) of 3-[6-(aminomethyl)-1-oxo-2,3-dihydro-1H-isoindol-2-yl]piperidine-2,6-dione hydrochloride (Reagent 1), 39 mg (0,336 mmol) of (oxan-2-yl)methanol (Reagent 2), 10.2 mg (0,063 mmol) of CDI, and 47.7 mg (0,369 mmol) of DIPEA. Purified by HPLC (gradient: from A-90%: B-10% to A-65%: B-35%; Rf=0.55; run time=8.5 min). Yield: 48 mg (68.8%). Yellow powder. LCMS purity: 98.2% (LCMS, R_{f}=0.42, run time=2 min). EI MS m/z: pos. 416.2 (MH⁺).

(3-ethyloxetan-3-yl)methyl N-{[2-(2,6-dioxopiperidin-3-yl)-3-oxo-2,3-dihydro-1H-isoindol-5-yl]methyl}carbamate (Compound 42) was obtained by Synthetic procedure 1 with using 54 mg (0,174 mmol) of 3-[6-(aminomethyl)-1-oxo-2,3-dihydro-1H-isoindol-2-yl]piperidine-2,6-dione hydrochloride (Reagent 1), 41 mg (0,353 mmol) of (3-ethyloxetan-3-yl)methanol (Reagent 2), 10.3 mg (0,064 mmol) of CDI, and 50.2 mg (0,388 mmol) of DIPEA. Purified by HPLC (gradient: from A-90%: B-10% to A-65%: B-35%; Rf=0.55; run time=8.5 min). Yield: 32.2 mg (43.9%). Gray powder. LCMS purity: 100% (LCMS, Rf=0.34, run time=2.5 min). EI MS m/z: pos. 416.0 (MH+).

(oxolan-3-yl)methyl N-{[2-(2,6-dioxopiperidin-3-yl)-3-oxo-2,3-dihydro-1H-isoindol-5-yl]methyl}carbamate (Compound 43) was obtained by Synthetic procedure 1 with using 42 mg (0,136 mmol) of 3-[6-(aminomethyl)-1-oxo-2,3-dihydro-1H-isoindol-2-yl]piperidine-2,6-dione hydrochloride (Reagent 1), 27 mg (0,264 mmol) of (oxolan-3-yl)methanol (Reagent 2), 8.5 mg (0,052 mmol) of CDI, and 37.6 mg (0,291 mmol) of DIPEA. Purified by HPLC (gradient: from A-95%: B-5% to A-80%: B-20%; Rf=0.59; run time=8.5 min). Yield: 27.4 mg (51.6%). Yellow powder. LCMS purity: 95.6% (LCMS, Rf=0.47, run time=2 min). ELI MS m/z: pos. 402.1 (MH+).

3-cyclopropylprop-2-yn-1-yl N-{[2-(2,6-dioxopiperidin-3-yl)-3-oxo-2,3-dihydro-1H-isoindol-5-yl]methyl}carbamate (Compound 44) was obtained by Synthetic procedure 1 with using 53 mg (0,171 mmol) of 3-[6-(aminomethyl)-1-oxo-2,3-dihydro-1H-isoindol-2-yl]piperidine-2,6-dione hydrochloride (Reagent 1), 26 mg (0.27 mmol) of 3-cyclopropylprop-2-yn-1-ol (Reagent 2), 9.4 mg (0,058 mmol) of CDI, and 51.3 mg (0,397 mmol) of DIPEA. Purified by HPLC (gradient: from A-80%: B-20% to A-60%: B-40%; R_{f}=0.55; run time=8.2 min). Yield: 29.9 mg (41.9%). Yellow powder. LCMS purity: 96.7% (LCMS, R_{f}=0.4, run time=2.5 min). EI MS m/z: pos. 396.2 (MH⁺).

tert-butyl 3-{[({[2-(2,6-dioxopiperidin-3-yl)-3-oxo-2,3-dihydro-1H-isoindol-5-yl]methyl}carbamoyl)oxy]methyl}azetidine-1-carboxylate (Compound 45) was obtained by Synthetic procedure 1 with using 46 mg (0,149 mmol) of 3-[6-(aminomethyl)-1-oxo-2,3-dihydro-1H-isoindol-2-yl]piperidine-2,6-dione hydrochloride (Reagent 1), 53 mg (0,283 mmol) of tert-butyl 3-(hydroxymethyl)azetidine-1-carboxylate (Reagent 2), 9.3 mg (0,057 mmol) of CDI, and 40.2 mg (0,311 mmol) of DIPEA. Purified by HPLC (gradient: from A-80%: B-20% to A-60%: B-40%; Rf=0.63; run time=8.2 min). Yield: 31.5 mg (45.7%). White powder. LCMS purity: 100% (LCMS, Rf=0.56, run time=2 min). EI MS m/z: pos. 487.1 (MH+).

(3,3-difluorocyclobutyl)methyl N-{[2-(2,6-dioxopiperidin-3-yl)-3-oxo-2,3-dihydro-1H-isoindol-5-yl]methyl}carbamate (Compound 46) was obtained by Synthetic procedure 1 with using 45 mg (0,145 mmol) of 3-[6-(aminomethyl)-1-oxo-2,3-dihydro-1H-isoindol-2-yl]piperidine-2,6-dione hydrochloride (Reagent 1), 25 mg (0,205 mmol) of (3,3-difluorocyclobutyl)methanol (Reagent 2), 6.3 mg (0,039 mmol) of CDI, and 38.8 mg (0.3 mmol) of DIPEA. Purified by HPLC (gradient: from A-90%: B-10% to A-65%: B-35%; Rf=0.7; run time=8.5 min). Yield: 23.4 mg (40.7%). Beige powder. LCMS purity: 98.7% (LCMS, Rf=0.45, run time=2 min). EI MS m/z: pos. 422.2 (MH+).

2-(3,3-difluorocyclobutyl)ethyl N-{[2-(2,6-dioxopiperidin-3-yl)-3-oxo-2,3-dihydro-1H-isoindol-5-yl]methyl}carbamate (Compound 47) was obtained by Synthetic procedure 1 with using 44 mg (0,142 mmol) of 3-[6-(aminomethyl)-1-oxo-2,3-dihydro-1H-isoindol-2-yl]piperidine-2,6-dione hydrochloride (Reagent 1), 27 mg (0,198 mmol) of 2-(3,3-difluorocyclobutyl)ethan-1-ol (Reagent 2), 6.4 mg (0,039 mmol) of CDI, and 37.6 mg (0,291 mmol) of DIPEA. Purified by HPLC (gradient: from A-85%: B-15% to A-60%: B-40%; Rf=0.68; run time=8.5 min). Yield: 27.4 mg (47.6%). Olive powder. LCMS purity: 100% (LCMS, Rf=0.47, run time=2 min). EI MS m/z: pos. 436.2 (MH+).

(1-methylcyclobutyl)methyl N-{[2-(2,6-dioxopiperidin-3-yl)-3-oxo-2,3-dihydro-1H-isoindol-5-yl]methyl}carbamate (Compound 48) was obtained by Synthetic procedure 1 with using 41 mg (0,132 mmol) of 3-[6-(aminomethyl)-1-oxo-2,3-dihydro-1H-isoindol-2-yl]piperidine-2,6-dione hydrochloride (Reagent 1), 20 mg (0.2 mmol) of (1-methylcyclobutyl)methanol (Reagent 2), 5.8 mg (0,036 mmol) of CDI, and 37.9 mg (0,293 mmol) of DIPEA. Purified by HPLC (gradient: from A-75%: B-25% to A-55%: B-45%; Rf=0.54; run time=8.5 min). Yield: 20.3 mg (38.2%). White powder. LCMS purity: 100% (LCMS, Rf=0.59, run time=2 min). EI MS m/z: pos. 400.2 (MH+).

1-cyclopropyl-2-methoxyethyl N-{[2-(2,6-dioxopiperidin-3-yl)-3-oxo-2,3-dihydro-1H-isoindol-5-yl]methyl}carbamate (Compound 49) was obtained by Synthetic procedure 1 with using 43 mg (0,139 mmol) of 3-[6-(aminomethyl)-1-oxo-2,3-dihydro-1H-isoindol-2-yl]piperidine-2,6-dione hydrochloride (Reagent 1), 25 mg (0,215 mmol) of 1-cyclopropyl-2-methoxyethan-1-ol (Reagent 2), 7.8 mg (0,048 mmol) of CDI, and 40.8 mg (0,316 mmol) of DIPEA. Purified by HPLC (gradient: from A-90%: B-10% to A-65%: B-35%; Rf=0.59; run time=8.5 min). Yield: 16.2 mg (27.2%). Yellow powder. LCMS purity: 100% (LCMS, Rf=0.43, run time=2 min). EI MS m/z: pos. 416.2 (MH+).

(1-acetylpyrrolidin-3-yl)methyl N-{[2-(2,6-dioxopiperidin-3-yl)-3-oxo-2,3-dihydro-1H-isoindol-5-yl]methyl}carbamate (Compound 50) was obtained by Synthetic procedure 1 with using 36 mg (0,116 mmol) of 3-[6-(aminomethyl)-1-oxo-2,3-dihydro-1H-isoindol-2-yl]piperidine-2,6-dione hydrochloride (Reagent 1), 24 mg (0,168 mmol) of 1-[3-(hydroxymethyl)pyrrolidin-1-yl]ethan-1-one (Reagent 2), 5.2 mg (0,032 mmol) of CDI, and 31.8 mg (0,246 mmol) of DIPEA. Purified by HPLC (gradient: from A-95%: B-5% to A-80%: B-20%; Rf=0.56; run time=8.5 min). Yield: 13.4 mg (27.1%). Yellow powder. LCMS purity: 98.3% (LCMS, Rf=0.3, run time=2.5 min). EI MS m/z: pos. 443.0 (MH+).

(1,1-dioxo-1lambda6-thiolan-3-yl)methyl N-{[2-(2,6-dioxopiperidin-3-yl)-3-oxo-2,3-dihydro-1H-isoindol-5-yl]methyl}carbamate (Compound 51) was obtained by Synthetic procedure 1 with using 36 mg (0,116 mmol) of 3-[6-(aminomethyl)-1-oxo-2,3-dihydro-1H-isoindol-2-yl]piperidine-2,6-dione hydrochloride (Reagent 1), 26 mg (0,173 mmol) of 3-(hydroxymethyl)-1lambda6-thiolane-1,1-dione (Reagent 2), 6.9 mg (0,043 mmol) of CDI, and 32.8 mg (0,254 mmol) of DIPEA. Purified by HPLC (gradient: from A-100%: B-0% to A-85%: B-15%; Rf=0.68; run time=8.5 min). Yield: 19.7 mg (38%). Yellow powder. LCMS purity: 100% (LCMS, Rf=0.44, run time=2 min). EI MS m/z: pos. 450.1 (MH+).

{6-oxaspiro[2.5]octan-1-yl}methyl N-{[2-(2,6-dioxopiperidin-3-yl)-3-oxo-2,3-dihydro-1H-isoindol-5-yl]methyl}carbamate (Compound 52) was obtained by Synthetic procedure 1 with using 38 mg (0,123 mmol) of 3-[6-(aminomethyl)-1-oxo-2,3-dihydro-1H-isoindol-2-yl]piperidine-2,6-dione hydrochloride (Reagent 1), 26 mg (0,183 mmol) of {6-oxaspiro[2.5]octan-1-yl}methanol (Reagent 2), 5.7 mg (0,035 mmol) of CDI, and 34.7 mg (0,268 mmol) of DIPEA. Purified by HPLC (gradient: from A-95%: B-5% to A-70%: B-30%; Rf=0.72; run time=8.5 min). Yield: 20.7 mg (38.5%). Yellow powder. LCMS purity: 100% (LCMS, Rf=0.43, run time=2 min). EI MS m/z: pos. 442.2 (MH+).

(thian-2-yl)methyl N-{[2-(2,6-dioxopiperidin-3-yl)-3-oxo-2,3-dihydro-1H-isoindol-5-yl]methyl}carbamate (Compound 53) was obtained by Synthetic procedure 1 with using 40 mg (0,129 mmol) of 3-[6-(aminomethyl)-1-oxo-2,3-dihydro-1H-isoindol-2-yl]piperidine-2,6-dione hydrochloride (Reagent 1), 25 mg (0,189 mmol) of (thian-2-yl)methanol (Reagent 2), 6.9 mg (0,043 mmol) of CDI, and 35.8 mg (0,277 mmol) of DIPEA. Purified by HPLC (gradient: from A-80%: B-20% to A-55%: B-45%; Rf=0.43; run time=8.5 min). Yield: 6.2 mg (11.4%). Beige powder. LCMS purity: 91% (LCMS, Rf=0.55, run time=2 min). EI MS m/z: pos. 115.2, 546.2, 454.0.

(3,3-difluorocyclopentyl)methyl N-{[2-(2,6-dioxopiperidin-3-yl)-3-oxo-2,3-dihydro-1H-isoindol-5-yl]methyl}carbamate (Compound 54) was obtained by Synthetic procedure 1 with using 38 mg (0,123 mmol) of 3-[6-(aminomethyl)-1-oxo-2,3-dihydro-1H-isoindol-2-yl]piperidine-2,6-dione hydrochloride (Reagent 1), 24 mg (0,176 mmol) of (3,3-difluorocyclopentyl)methanol (Reagent 2), 6 mg (0,037 mmol) of CDI, and 33.4 mg (0,258 mmol) of DIPEA. Purified by HPLC (gradient: from A-85%: B-15% to A-60%: B-40%; Rf=0.65; run time=8.5 min). Yield: 20.8 mg (40.6%). Beige powder. LCMS purity: 100% (LCMS, Rf=0.47, run time=2 min). EI MS m/z: pos. 436.2 (MH+).

(1s,3s)-3-(propan-2-yloxy)cyclobutyl N-{[2-(2,6-dioxopiperidin-3-yl)-3-oxo-2,3-dihydro-1H-isoindol-5-yl]methyl}carbamate (Compound 55) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 22 mg (0,169 mmol) of (1s,3s)-3-(propan-2-yloxy)cyclobutan-1-ol (Reagent 2), 5.7 mg (0,035 mmol) of CDI, and 32 mg (0,248 mmol) of DIPEA. Purified by HPLC (gradient: from A-85%: B-15% to A-60%: B-40%; Rf=0.59; run time=8.5 min). Yield: 17.2 mg (35.5%). Beige powder. LCMS purity: 100% (LCMS, Rf=0.55, run time=2 min). EI MS m/z: pos. 430.2 (MH+).

(2R)-1-cyclopropylpropan-2-yl N-{[2-(2,6-dioxopiperidin-3-yl)-3-oxo-2,3-dihydro-1H-isoindol-5-yl]methyl}carbamate (Compound 56) was obtained by Synthetic procedure 1 with using 39 mg (0,126 mmol) of 3-[6-(aminomethyl)-1-oxo-2,3-dihydro-1H-isoindol-2-yl]piperidine-2,6-dione hydrochloride (Reagent 1), 18 mg (0.18 mmol) of (2R)-1-cyclopropylpropan-2-ol (Reagent 2), 6 mg (0,037 mmol) of CDI, and 34.1 mg (0,264 mmol) of DIPEA. Purified by HPLC (gradient: from A-80%: B-20% to A-55%: B-45%; Rf=0.52; run time=8.5 min). Yield: 8.9 mg (18.6%). Beige powder. LCMS purity: 98.7% (LCMS, Rf=0.46, run time=2.5 min). EI MS m/z: pos. 400.2 (MH+).

[1-(methoxymethyl)cyclopentyl]methyl N-{[2-(2,6-dioxopiperidin-3-yl)-3-oxo-2,3-dihydro-1H-isoindol-5-yl]methyl}carbamate (Compound 57) was obtained by Synthetic procedure 1 with using 42 mg (0,136 mmol) of 3-[6-(aminomethyl)-1-oxo-2,3-dihydro-1H-isoindol-2-yl]piperidine-2,6-dione hydrochloride (Reagent 1), 27 mg (0,187 mmol) of [1-(methoxymethyl)cyclopentyl]methanol (Reagent 2), 5.7 mg (0,035 mmol) of CDI, and 35.5 mg (0,275 mmol) of DIPEA. Purified by HPLC (gradient: from A-80%: B-20% to A-55%: B-45%; Rf=0.6; run time=8.3 min). Yield: 16.3 mg (29.4%). Yellow powder. LCMS purity: 97% (LCMS, Rf=0.5, run time=2 min). EI MS m/z: pos. 444.2 (MH+).

2-cyclobutylpropyl N-{[2-(2,6-dioxopiperidin-3-yl)-3-oxo-2,3-dihydro-1H-isoindol-5-yl]methyl}carbamate (Compound 58) was obtained by Synthetic procedure 1 with using 33 mg (0,107 mmol) of 3-[6-(aminomethyl)-1-oxo-2,3-dihydro-1H-isoindol-2-yl]piperidine-2,6-dione hydrochloride (Reagent 1), 19 mg (0,166 mmol) of 2-cyclobutylpropan-1-ol (Reagent 2), 5.8 mg (0,036 mmol) of CDI, and 31.5 mg (0,244 mmol) of DIPEA. Purified by HPLC (gradient: from A-70%: B-30% to A-55%: B-45%; Rf=0.59; run time=8.5 min). Yield: 21.2 mg (46.2%). Yellow powder. LCMS purity: 100% (LCMS, Rf=0.54, run time=2 min). EI MS m/z: pos. 414.2 (MH+).

2-(1-methylcyclopropyl)ethyl N-{[2-(2,6-dioxopiperidin-3-yl)-3-oxo-2,3-dihydro-1H-isoindol-5-yl]methyl}carbamate (Compound 59) was obtained by Synthetic procedure 1 with using 43 mg (0,139 mmol) of 3-[6-(aminomethyl)-1-oxo-2,3-dihydro-1H-isoindol-2-yl]piperidine-2,6-dione hydrochloride (Reagent 1), 20 mg (0.2 mmol) of 2-(1-methylcyclopropyl)ethan-1-ol (Reagent 2), 6.4 mg (0,039 mmol) of CDI, and 37.9 mg (0,293 mmol) of DIPEA. Purified by HPLC (gradient: from A-80%: B-20% to A-55%: B-45%; Rf=0.63; run time=8.3 min). Yield: 14.9 mg (28%). Yellow powder. LCMS purity: 100% (LCMS, Rf=0.58, run time=2 min). EI MS m/z: pos. 400.1 (MH+).

(thietan-3-yl)methyl N-{[2-(2,6-dioxopiperidin-3-yl)-3-oxo-2,3-dihydro-1H-isoindol-5-yl]methyl}carbamate (Compound 60) was obtained by Synthetic procedure 1 with using 40 mg (0,129 mmol) of 3-[6-(aminomethyl)-1-oxo-2,3-dihydro-1H-isoindol-2-yl]piperidine-2,6-dione hydrochloride (Reagent 1), 21 mg (0,202 mmol) of (thietan-3-yl)methanol (Reagent 2), 6.9 mg (0,043 mmol) of CDI, and 38.2 mg (0,296 mmol) of DIPEA. Purified by HPLC (gradient: from A-100%: B-0% to A-75%: B-25%; Rf=0.78; run time=8.5 min). Yield: 31.9 mg (58.8%). White powder. LCMS purity: 100% (LCMS, Rf=0.37, run time=2.5 min). EI MS m/z: pos. 404.2 (MH+).

[(1R,5S,6R)-3-oxabicyclo[3.1.0]hexan-6-yl]methyl N-{[2-(2,6-dioxopiperidin-3-yl)-3-oxo-2,3-dihydro-1H-isoindol-5-yl]methyl}carbamate (Compound 61) was obtained by Synthetic procedure 1 with using 38 mg (0,123 mmol) of 3-[6-(aminomethyl)-1-oxo-2,3-dihydro-1H-isoindol-2-yl]piperidine-2,6-dione hydrochloride (Reagent 1), 20 mg (0,175 mmol) of [(1R,5S,6R)-3-oxabicyclo[3.1.0]hexan-6-yl]methanol (Reagent 2), 6 mg (0,037 mmol) of CDI, and 33.2 mg (0,257 mmol) of DIPEA. Purified by HPLC (gradient: from A-95%: B-5% to A-80%: B-20%; Rf=0.67; run time=8.5 min). Yield: 15.4 mg (31.9%). Yellow powder. LCMS purity: 95.4% (LCMS, Rf=0.49, run time=2 min). EI MS m/z: pos. 414.1 (MH+).

(1R,3s,5S)-6,6-dimethylbicyclo[3.1.0]hexan-3-yl N-{[2-(2,6-dioxopiperidin-3-yl)-3-oxo-2,3-dihydro-1H-isoindol-5-yl]methyl}carbamate (Compound 62) was obtained by Synthetic procedure 1 with using 48 mg (0,155 mmol) of 3-[6-(aminomethyl)-1-oxo-2,3-dihydro-1H-isoindol-2-yl]piperidine-2,6-dione hydrochloride (Reagent 1), 27 mg (0,214 mmol) of (1R,3s,5S)-6,6-dimethylbicyclo[3.1.0]hexan-3-ol (Reagent 2), 7 mg (0,043 mmol) of CDI, and 40.6 mg (0,314 mmol) of DIPEA. Purified by HPLC (gradient: from A-70%: B-30% to A-50%: B-50%; Rf=0.63; run time=8.5 min). Yield: 13.2 mg (21.8%). Beige powder. LCMS purity: 100% (LCMS, Rf=0.45, run time=2.5 min). EI MS m/z: pos. 426.0 (MH+).

{7,7-dimethylbicyclo[2.2.1]heptan-1-yl}methyl N-{[2-(2,6-dioxopiperidin-3-yl)-3-oxo-2,3-dihydro-1H-isoindol-5-yl]methyl}carbamate (Compound 63) was obtained by Synthetic procedure 1 with using 36 mg (0,116 mmol) of 3-[6-(aminomethyl)-1-oxo-2,3-dihydro-1H-isoindol-2-yl]piperidine-2,6-dione hydrochloride (Reagent 1), 24 mg (0,156 mmol) of {7,7-dimethylbicyclo[2.2.1]heptan-1-yl}methanol (Reagent 2), 5.8 mg (0,036 mmol) of CDI, and 29.5 mg (0,228 mmol) of DIPEA. Purified by HPLC (gradient: from A-65%: B-35% to A-45%: B-55%; Rf=0.56; run time=8.5 min). Yield: 15.7 mg (33.4%). White powder. LCMS purity: 98.3% (LCMS, Rf=0.67, run time=2 min). EI MS m/z: pos. 454.0 (MH+).

(2-methyloxan-2-yl)methyl N-{[2-(2,6-dioxopiperidin-3-yl)-3-oxo-2,3-dihydro-1H-isoindol-5-yl]methyl}carbamate (Compound 64) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 20 mg (0,154 mmol) of (2-methyloxan-2-yl)methanol (Reagent 2), 4.5 mg (0,028 mmol) of CDI, and 29.1 mg (0,225 mmol) of DIPEA. Purified by HPLC (gradient: from A-85%: B-15% to A-60%: B-40%; Rf=0.51; run time=8.5 min). Yield: 17.8 mg (40.5%). Yellow powder. LCMS purity: 100% (LCMS, Rf=0.44, run time=2 min). EI MS m/z: pos. 430.2 (MH+).

3-(1-methylcyclopropyl)propyl N-{[2-(2,6-dioxopiperidin-3-yl)-3-oxo-2,3-dihydro-1H-isoindol-5-yl]methyl}carbamate (Compound 65) was obtained by Synthetic procedure 1 with using 45 mg (0,145 mmol) of 3-[6-(aminomethyl)-1-oxo-2,3-dihydro-1H-isoindol-2-yl]piperidine-2,6-dione hydrochloride (Reagent 1), 23 mg (0,201 mmol) of 3-(1-methylcyclopropyl)propan-1-ol (Reagent 2), 7.2 mg (0,044 mmol) of CDI, and 38.2 mg (0,296 mmol) of DIPEA. Purified by HPLC (gradient: from A-75%: B-25% to A-60%: B-40%; Rf=0.67; run time=8.5 min). Yield: 19.1 mg (34.4%). Beige powder. LCMS purity: 100% (LCMS, Rf=0.63, run time=2 min). EI MS m/z: pos. 414.1 (MH+).

(3-methoxycyclobutyl)methyl N-{[2-(2,6-dioxopiperidin-3-yl)-3-oxo-2,3-dihydro-1H-isoindol-5-yl]methyl}carbamate (Compound 66) was obtained by Synthetic procedure 1 with using 46 mg (0,149 mmol) of 3-[6-(aminomethyl)-1-oxo-2,3-dihydro-1H-isoindol-2-yl]piperidine-2,6-dione hydrochloride (Reagent 1), 24 mg (0,207 mmol) of (3-methoxycyclobutyl)methanol (Reagent 2), 6.9 mg (0,043 mmol) of CDI, and 39.2 mg (0,303 mmol) of DIPEA. Purified by HPLC (gradient: from A-90%: B-10% to A-75%: B-25%; Rf=0.54; run time=8.5 min). Yield: 25.1 mg (43.9%). Beige powder. LCMS purity: 91.2% (LCMS, Rf=0.52, run time=2 min). EI MS m/z: pos. 416.0 (MH+).

(3-ethoxycyclobutyl)methyl N-{[2-(2,6-dioxopiperidin-3-yl)-3-oxo-2,3-dihydro-1H-isoindol-5-yl]methyl}carbamate (Compound 67) was obtained by Synthetic procedure 1 with using 41 mg (0,132 mmol) of 3-[6-(aminomethyl)-1-oxo-2,3-dihydro-1H-isoindol-2-yl]piperidine-2,6-dione hydrochloride (Reagent 1), 26 mg (0.2 mmol) of (3-ethoxycyclobutyl)methanol (Reagent 2), 6.7 mg (0,041 mmol) of CDI, and 37.9 mg (0,293 mmol) of DIPEA. Purified by HPLC (gradient: from A-85%: B-15% to A-70%: B-30%; Rf=0.52; run time=8.5 min). Yield: 12.6 mg (22%). Yellow powder. LCMS purity: 95% (LCMS, Rf=0.45, run time=2 min). EI MS m/z: pos. 430.2 (MH+).

rac-[(2R,5R)-5-methyloxolan-2-yl]methyl N-{[2-(2,6-dioxopiperidin-3-yl)-3-oxo-2,3-dihydro-1H-isoindol-5-yl]methyl}carbamate (Compound 68) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 22 mg (0,189 mmol) of rac-[(2R,5R)-5-methyloxolan-2-yl]methanol (Reagent 2), 6.1 mg (0,038 mmol) of CDI, and 35.9 mg (0,278 mmol) of DIPEA. Purified by HPLC (gradient: from A-90%: B-10% to A-75%: B-25%; Rf=0.59; run time=8.5 min). Yield: 18.5 mg (35.3%). Yellow powder. LCMS purity: 97.7% (LCMS, Rf=0.35, run time=2.5 min). EI MS m/z: pos. 416.0 (MH+).

3-cyclopropyl-3-methylbutyl N-{[2-(2,6-dioxopiperidin-3-yl)-3-oxo-2,3-dihydro-1H-isoindol-5-yl]methyl}carbamate (Compound 69) was obtained by Synthetic procedure 1 with using 36 mg (0,116 mmol) of 3-[6-(aminomethyl)-1-oxo-2,3-dihydro-1H-isoindol-2-yl]piperidine-2,6-dione hydrochloride (Reagent 1), 20 mg (0,156 mmol) of 3-cyclopropyl-3-methylbutan-1-ol (Reagent 2), 4.8 mg (0.03 mmol) of CDI, and 29.6 mg (0,229 mmol) of DIPEA. Purified by HPLC (gradient: from A-75%: B-25% to A-50%: B-50%; Rf=0.67; run time=8.5 min). Yield: 16.4 mg (36.9%). Yellow powder. LCMS purity: 100% (LCMS, Rf=0.66, run time=2 min). EI MS m/z: pos. 428.2 (MH+).

rac-[(1R,2R,4R)-bicyclo[2.2.2]oct-5-en-2-yl]methyl N-{[2-(2,6-dioxopiperidin-3-yl)-3-oxo-2,3-dihydro-1H-isoindol-5-yl]methyl}carbamate (Compound 70) was obtained by Synthetic procedure 1 with using 38 mg (0,123 mmol) of 3-[6-(aminomethyl)-1-oxo-2,3-dihydro-1H-isoindol-2-yl]piperidine-2,6-dione hydrochloride (Reagent 1), 26 mg (0,188 mmol) of rac-[(1R,2R,4R)-bicyclo[2.2.2]oct-5-en-2-yl]methanol (Reagent 2), 6.4 mg (0,039 mmol) of CDI, and 35.7 mg (0,276 mmol) of DIPEA. Purified by HPLC (gradient: from A-70%: B-30% to A-55%: B-45%; Rf=0.56; run time=8.5 min). Yield: 21.8 mg (39.7%). Yellow powder. LCMS purity: 100% (LCMS, Rf=0.65, run time=2 min). EI MS m/z: pos. 438.1 (MH+).

(1R)-1-{spiro[2.2]pentan-1-yl}ethyl N-{[2-(2,6-dioxopiperidin-3-yl)-3-oxo-2,3-dihydro-1H-isoindol-5-yl]methyl}carbamate (Compound 71) was obtained by Synthetic procedure 1 with using 35 mg (0,113 mmol) of 3-[6-(aminomethyl)-1-oxo-2,3-dihydro-1H-isoindol-2-yl]piperidine-2,6-dione hydrochloride (Reagent 1), 22 mg (0,196 mmol) of (1R)-1-{spiro[2.2]pentan-1-yl}ethan-1-ol (Reagent 2), 6.7 mg (0,041 mmol) of CDI, and 37.2 mg (0,288 mmol) of DIPEA. Purified by HPLC (gradient: from A-80%: B-20% to A-60%: B-40%; Rf=0.71; run time=8.2 min). Yield: 3.5 mg (6.5%). Yellow sticky oil. LCMS purity: 100% (LCMS, Rf=0.59, run time=2 min). EI MS m/z: pos. 412.2 (MH+).

{4-methyl-2-oxabicyclo[2.1.1]hexan-1-yl}methyl N-{[2-(2,6-dioxopiperidin-3-yl)-3-oxo-2,3-dihydro-1H-isoindol-5-yl]methyl}carbamate (Compound 72) was obtained by Synthetic procedure 1 with using 46 mg (0,149 mmol) of 3-[6-(aminomethyl)-1-oxo-2,3-dihydro-1H-isoindol-2-yl]piperidine-2,6-dione hydrochloride (Reagent 1), 27 mg (0,211 mmol) of {4-methyl-2-oxabicyclo[2.1.1]hexan-1-yl}methanol (Reagent 2), 7.5 mg (0,046 mmol) of CDI, and 39.9 mg (0,309 mmol) of DIPEA. Purified by HPLC (gradient: from A-90%: B-10% to A-75%: B-25%; Rf=0.57; run time=8.5 min). Yield: 18.7 mg (31.2%). Yellow powder. LCMS purity: 97.7% (LCMS, Rf=0.52, run time=2 min). EI MS m/z: pos. 428.1 (MH+).

rac-[(1R,2R)-2-(trifluoromethyl)cyclopentyl]methyl N-{[2-(2,6-dioxopiperidin-3-yl)-3-oxo-2,3-dihydro-1H-isoindol-5-yl]methyl}carbamate (Compound 73) was obtained by Synthetic procedure 1 with using 39 mg (0,126 mmol) of 3-[6-(aminomethyl)-1-oxo-2,3-dihydro-1H-isoindol-2-yl]piperidine-2,6-dione hydrochloride (Reagent 1), 30 mg (0,178 mmol) of rac-[(1R,2R)-2-(trifluoromethyl)cyclopentyl]methanol (Reagent 2), 5.5 mg (0,034 mmol) of CDI, and 33.8 mg (0,262 mmol) of DIPEA. Purified by HPLC (gradient: from A-75%: B-25% to A-60%: B-40%; Rf=0.67; run time=8.5 min). Yield: 24.1 mg (43.3%). Yellow powder. LCMS purity: 100% (LCMS, Rf=0.63, run time=2 min). EI MS m/z: pos. 468.1 (MH+).

(1-fluorocyclobutyl)methyl N-{[2-(2,6-dioxopiperidin-3-yl)-3-oxo-2,3-dihydro-1H-isoindol-5-yl]methyl}carbamate (Compound 74) was obtained by Synthetic procedure 1 with using 63 mg (0,203 mmol) of 3-[6-(aminomethyl)-1-oxo-2,3-dihydro-1H-isoindol-2-yl]piperidine-2,6-dione hydrochloride (Reagent 1), 27 mg (0,259 mmol) of (1-fluorocyclobutyl)methanol (Reagent 2), 8.8 mg (0,054 mmol) of CDI, and 30.7 mg (0,238 mmol) of DIPEA. Purified by HPLC (gradient: from A-75%: B-25% to A-50%: B-50%). Yield: 36.5 mg (41.9%). White powder. LCMS purity: 98.6% (LCMS, Rf=0.53, run time=2 min). EI MS m/z: pos. 404.2 (MH+). 1H NMR purity: 95%.

rac-[(1R,2S)-2-fluorocyclopropyl]methyl N-{[2-(2,6-dioxopiperidin-3-yl)-3-oxo-2,3-dihydro-1H-isoindol-5-yl]methyl}carbamate (Compound 75) was obtained by Synthetic procedure 1 with using 54 mg (0,174 mmol) of 3-[6-(aminomethyl)-1-oxo-2,3-dihydro-1H-isoindol-2-yl]piperidine-2,6-dione hydrochloride (Reagent 1), 22 mg (0,244 mmol) of rac-[(1R,2S)-2-fluorocyclopropyl]methanol (Reagent 2), 8.5 mg (0,052 mmol) of CDI, and 46.3 mg (0,358 mmol) of DIPEA. Purified by HPLC (gradient: from A-85%: B-15% to A-65%: B-35%; Rf=0.45; run time=8.5 min). Yield: 32.3 mg (51%). Yellow powder. LCMS purity: 95.6% (LCMS, Rf=0.51, run time=2 min). EI MS m/z: pos. 390.0 (MH+).

rac-[(2R,3S)-3-methyloxolan-2-yl]methyl N-{[2-(2,6-dioxopiperidin-3-yl)-3-oxo-2,3-dihydro-1H-isoindol-5-yl]methyl}carbamate (Compound 76) was obtained by Synthetic procedure 1 with using 39 mg (0,126 mmol) of 3-[6-(aminomethyl)-1-oxo-2,3-dihydro-1H-isoindol-2-yl]piperidine-2,6-dione hydrochloride (Reagent 1), 20 mg (0,172 mmol) of rac-[(2R,3S)-3-methyloxolan-2-yl]methanol (Reagent 2), 6 mg (0,037 mmol) of CDI, and 32.6 mg (0,252 mmol) of DIPEA. Purified by HPLC (gradient: from A-90%: B-10% to A-75%: B-25%; Rf=0.53; run time=8.5 min). Yield: 15.1 mg (31.7%). Yellow powder. LCMS purity: 94.7% (LCMS, Rf=0.41, run time=2 min). EI MS m/z: pos. 416.2 (MH+).

{[1,1′-bi(cyclopropane)]-1-yl}methyl N-{[2-(2,6-dioxopiperidin-3-yl)-3-oxo-2,3-dihydro-1H-isoindol-5-yl]methyl}carbamate (Compound 77) was obtained by Synthetic procedure 1 with using 45 mg (0,145 mmol) of 3-[6-(aminomethyl)-1-oxo-2,3-dihydro-1H-isoindol-2-yl]piperidine-2,6-dione hydrochloride (Reagent 1), 22 mg (0,196 mmol) of {[1,1′-bi(cyclopropane)]-1-yl}methanol (Reagent 2), 6.7 mg (0,041 mmol) of CDI, and 37.2 mg (0,288 mmol) of DIPEA. Purified by HPLC (gradient: from A-75%: B-25% to A-55%: B-45%; Rf=0.53; run time=8.5 min). Yield: 20.6 mg (38.3%). Brown powder. LCMS purity: 100% (LCMS, Rf=0.59, run time=2 min). EI MS m/z: pos. 412.2 (MH+).

2-(2,2-difluorocyclopropyl)ethyl N-{[2-(2,6-dioxopiperidin-3-yl)-3-oxo-2,3-dihydro-1H-isoindol-5-yl]methyl}carbamate (Compound 78) was obtained by Synthetic procedure 1 with using 36 mg (0,116 mmol) of 3-[6-(aminomethyl)-1-oxo-2,3-dihydro-1H-isoindol-2-yl]piperidine-2,6-dione hydrochloride (Reagent 1), 21 mg (0,172 mmol) of 2-(2,2-difluorocyclopropyl)ethan-1-ol (Reagent 2), 6 mg (0,037 mmol) of CDI, and 32.6 mg (0,252 mmol) of DIPEA. Purified by HPLC (gradient: from A-90%: B-10% to A-60%: B-40%; Rf=0.62; run time=8.5 min). Yield: 12 mg (24.8%). Yellow powder. LCMS purity: 95.1% (LCMS, Rf=0.44, run time=2.5 min). EI MS m/z: pos. 422.0 (MH+).

(3,3-dimethyloxetan-2-yl)methyl N-{[2-(2,6-dioxopiperidin-3-yl)-3-oxo-2,3-dihydro-1H-isoindol-5-yl]methyl}carbamate (Compound 79) was obtained by Synthetic procedure 1 with using 54 mg (0,174 mmol) of 3-[6-(aminomethyl)-1-oxo-2,3-dihydro-1H-isoindol-2-yl]piperidine-2,6-dione hydrochloride (Reagent 1), 28 mg (0,241 mmol) of (3,3-dimethyloxetan-2-yl)methanol (Reagent 2), 7.9 mg (0,049 mmol) of CDI, and 45.7 mg (0,354 mmol) of DIPEA. Purified by HPLC (gradient: from A-90%: B-10% to A-65%: B-35%; Rf=0.54; run time=8.5 min). Yield: 28.4 mg (42.5%). Yellow powder. LCMS purity: 98.4% (LCMS, Rf=0.41, run time=2 min). EI MS m/z: pos. 416.2 (MH+).

[1-(cyclopropylmethyl)cyclopropyl]methyl N-{[2-(2,6-dioxopiperidin-3-yl)-3-oxo-2,3-dihydro-1H-isoindol-5-yl]methyl}carbamate (Compound 80) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 22 mg (0,174 mmol) of [1-(cyclopropylmethyl)cyclopropyl]methanol (Reagent 2), 5.7 mg (0,035 mmol) of CDI, and 33 mg (0,255 mmol) of DIPEA. Purified by HPLC (gradient: from A-75%: B-25% to A-55%: B-45%; Rf=0.62; run time=8.5 min). Yield: 16.9 mg (34.2%). Beige powder. LCMS purity: 100% (LCMS, Rf=0.63, run time=2 min). EI MS m/z: pos. 426.1 (MH+).

(3-methyl-1,1-dioxo-1lambda6-thietan-3-yl)methyl N-{[2-(2,6-dioxopiperidin-3-yl)-3-oxo-2,3-dihydro-1H-isoindol-5-yl]methyl}carbamate (Compound 81) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 27 mg (0.18 mmol) of 3-(hydroxymethyl)-3-methyl-1lambda6-thietane-1,1-dione (Reagent 2), 6 mg (0,037 mmol) of CDI, and 34.1 mg (0,264 mmol) of DIPEA. Purified by HPLC (gradient: from A-100%: B-0% to A-75%: B-25%; Rf=0.65; run time=8.5 min). Yield: 19.5 mg (36.2%). Beige powder. LCMS purity: 100% (LCMS, Rf=0.36, run time=2 min). EI MS m/z: pos. 450.2 (MH+).

(1,1-dioxo-1lambda6-thiolan-2-yl)methyl N-{[2-(2,6-dioxopiperidin-3-yl)-3-oxo-2,3-dihydro-1H-isoindol-5-yl]methyl}carbamate (Compound 82) was obtained by Synthetic procedure 1 with using 33 mg (0,107 mmol) of 3-[6-(aminomethyl)-1-oxo-2,3-dihydro-1H-isoindol-2-yl]piperidine-2,6-dione hydrochloride (Reagent 1), 25 mg (0,166 mmol) of 2-(hydroxymethyl)-1lambda6-thiolane-1,1-dione (Reagent 2), 5.8 mg (0,036 mmol) of CDI, and 31.6 mg (0,245 mmol) of DIPEA. Purified by HPLC (gradient: from A-100%: B-0% to A-80%: B-20%; Rf=0.56; run time=8.5 min). Yield: 14.5 mg (29.1%). Beige powder. LCMS purity: 92.9% (LCMS, Rf=0.34, run time=2 min). EI MS m/z: pos. 450.2 (MH+).

(4-methyloxan-4-yl)methyl N-{[2-(2,6-dioxopiperidin-3-yl)-3-oxo-2,3-dihydro-1H-isoindol-5-yl]methyl}carbamate (Compound 83) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 24 mg (0,184 mmol) of (4-methyloxan-4-yl)methanol (Reagent 2), 6.1 mg (0,038 mmol) of CDI, and 34.9 mg (0.27 mmol) of DIPEA. Purified by HPLC (gradient: from A-90%: B-10% to A-65%: B-35%; Rf=0.55; run time=8.5 min). Yield: 27.6 mg (52.3%). Yellow powder. LCMS purity: 96.5% (LCMS, Rf=0.42, run time=2 min). EI MS m/z: pos. 430.2 (MH+).

3-tert-butylcyclobutyl N-{[2-(2,6-dioxopiperidin-3-yl)-3-oxo-2,3-dihydro-1H-isoindol-5-yl]methyl}carbamate (Compound 84) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 22 mg (0,172 mmol) of 3-tert-butylcyclobutan-1-ol (Reagent 2), 6.1 mg (0,038 mmol) of CDI, and 32.5 mg (0,251 mmol) of DIPEA. Purified by HPLC (gradient: from A-70%: B-30% to A-55%: B-45%; Rf=0.66; run time=8.5 min). Yield: 21.5 mg (44%). White powder. LCMS purity: 100% (LCMS, Rf=0.67, run time=2 min). EI MS m/z: pos. 428.1 (MH+).

(1-methylcyclohexyl)methyl N-{[2-(2,6-dioxopiperidin-3-yl)-3-oxo-2,3-dihydro-1H-isoindol-5-yl]methyl}carbamate (Compound 85) was obtained by Synthetic procedure 1 with using 37 mg (0,119 mmol) of 3-[6-(aminomethyl)-1-oxo-2,3-dihydro-1H-isoindol-2-yl]piperidine-2,6-dione hydrochloride (Reagent 1), 21 mg (0,164 mmol) of (1-methylcyclohexyl)methanol (Reagent 2), 5.7 mg (0,035 mmol) of CDI, and 31 mg (0.24 mmol) of DIPEA. Purified by HPLC (gradient: from A-65%: B-35% to A-45%: B-55%; Rf=0.88; run time=8.5 min). Yield: 13.3 mg (28.5%). Yellow powder. LCMS purity: 100% (LCMS, Rf=0.64, run time=2 min). EI MS m/z: pos. 428.1 (MH+).

{2,2-difluorospiro[2.2]pentan-1-yl}methyl N-{[2-(2,6-dioxopiperidin-3-yl)-3-oxo-2,3-dihydro-1H-isoindol-5-yl]methyl}carbamate (Compound 86) was obtained by Synthetic procedure 1 with using 42 mg (0,136 mmol) of 3-[6-(aminomethyl)-1-oxo-2,3-dihydro-1H-isoindol-2-yl]piperidine-2,6-dione hydrochloride (Reagent 1), 26 mg (0,194 mmol) of {2,2-difluorospiro[2.2]pentan-1-yl}methanol (Reagent 2), 6 mg (0,037 mmol) of CDI, and 36.7 mg (0,284 mmol) of DIPEA. Purified by HPLC (gradient: from A-95%: B-5% to A-70%: B-30%; Rf=0.5; run time=8.5 min). Yield: 18.7 mg (33.4%). Yellow powder. LCMS purity: 100% (LCMS, Rf=0.48, run time=2 min). EI MS m/z: pos. 434.2 (MH+).

[(4S)-2,2-dimethyl-1,3-dioxolan-4-yl]methyl N-{[2-(2,6-dioxopiperidin-3-yl)-3-oxo-2,3-dihydro-1H-isoindol-5-yl]methyl}carbamate (Compound 87) was obtained by Synthetic procedure 1 with using 33 mg (0,107 mmol) of 3-[6-(aminomethyl)-1-oxo-2,3-dihydro-1H-isoindol-2-yl]piperidine-2,6-dione hydrochloride (Reagent 1), 29 mg (0,219 mmol) of [(4R)-2,2-dimethyl-1,3-dioxolan-4-yl]methanol (Reagent 2), 7.3 mg (0,045 mmol) of CDI, and 31.2 mg (0,241 mmol) of DIPEA. Purified by HPLC (gradient: from A-90%: B-10% to A-75%: B-25%; Rf=0.55; run time=8.5 min). Yield: 21 mg (44.4%). Blue powder. LCMS purity: 100% (LCMS, Rf=0.5, run time=2 min). EI MS m/z: pos. 432.2 (MH+).

(4,4-difluorocyclohexyl)methyl N-{[2-(2,6-dioxopiperidin-3-yl)-3-oxo-2,3-dihydro-1H-isoindol-5-yl]methyl}carbamate (Compound 88) was obtained by Synthetic procedure 1 with using 41 mg (0,132 mmol) of 3-[6-(aminomethyl)-1-oxo-2,3-dihydro-1H-isoindol-2-yl]piperidine-2,6-dione hydrochloride (Reagent 1), 27 mg (0.18 mmol) of (4,4-difluorocyclohexyl)methanol (Reagent 2), 5.8 mg (0,036 mmol) of CDI, and 34.1 mg (0,264 mmol) of DIPEA. Purified by HPLC (gradient: from A-80%: B-20% to A-65%: B-35%; Rf=0.66; run time=8.5 min). Yield: 23.8 mg (44.2%). Yellow powder. LCMS purity: 98.3% (LCMS, Rf=0.59, run time=2 min). EI MS m/z: pos. 450.2 (MH+).

4-(propan-2-yl)cyclohexyl N-{[2-(2,6-dioxopiperidin-3-yl)-3-oxo-2,3-dihydro-1H-isoindol-5-yl]methyl}carbamate (Compound 89) was obtained by Synthetic procedure 1 with using 47 mg (0,152 mmol) of 3-[6-(aminomethyl)-1-oxo-2,3-dihydro-1H-isoindol-2-yl]piperidine-2,6-dione hydrochloride (Reagent 1), 29 mg (0,204 mmol) of 4-(propan-2-yl)cyclohexan-1-ol (Reagent 2), 6.9 mg (0,043 mmol) of CDI, and 38.6 mg (0,299 mmol) of DIPEA. Purified by HPLC (gradient: from A-65%: B-35% to A-40%: B-60%; Rf=0.68; run time=6.5 min). Yield: 15.2 mg (25.3%). Beige powder. LCMS purity: 95.3% (LCMS, Rf=0.69, run time=2 min). EI MS m/z: pos. 442.2 (MH+).

7-oxaspiro[3.5]nonan-2-yl N-{[2-(2,6-dioxopiperidin-3-yl)-3-oxo-2,3-dihydro-1H-isoindol-5-yl]methyl}carbamate (Compound 90) was obtained by Synthetic procedure 1 with using 39 mg (0,126 mmol) of 3-[6-(aminomethyl)-1-oxo-2,3-dihydro-1H-isoindol-2-yl]piperidine-2,6-dione hydrochloride (Reagent 1), 26 mg (0,183 mmol) of 7-oxaspiro[3.5]nonan-2-ol (Reagent 2), 6 mg (0,037 mmol) of CDI, and 34.7 mg (0,268 mmol) of DIPEA. Purified by HPLC (gradient: from A-90%: B-10% to A-75%: B-25%; Rf=0.66; run time=8.5 min). Yield: 20.8 mg (38.7%). Yellow powder. LCMS purity: 98.8% (LCMS, Rf=0.53, run time=2 min). EI MS m/z: pos. 442.2 (MH+).

(1-methanesulfonylazetidin-3-yl)methyl N-{[2-(2,6-dioxopiperidin-3-yl)-3-oxo-2,3-dihydro-1H-isoindol-5-yl]methyl}carbamate (Compound 91) was obtained by Synthetic procedure 1 with using 51 mg (0,165 mmol) of 3-[6-(aminomethyl)-1-oxo-2,3-dihydro-1H-isoindol-2-yl]piperidine-2,6-dione hydrochloride (Reagent 1), 41 mg (0,248 mmol) of (1-methanesulfonylazetidin-3-yl)methanol (Reagent 2), 8.1 mg (0.05 mmol) of CDI, and 47 mg (0,364 mmol) of DIPEA. Purified by HPLC (gradient: from A-100%: B-0% to A-75%: B-25%; Rf=0.54; run time=8.5 min). Yield: 41.2 mg (53.6%). White powder. LCMS purity: 100% (LCMS, Rf=0.35, run time=2 min). EI MS m/z: pos. 465.2 (MH+).

rac-(1R,2R,4R)-5,5-difluorobicyclo[2.2.1]heptan-2-yl N-{[2-(2,6-dioxopiperidin-3-yl)-3-oxo-2,3-dihydro-1H-isoindol-5-yl]methyl}carbamate (Compound 92) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 26 mg (0,175 mmol) of rac-(1R,2R,4R)-5,5-difluorobicyclo[2.2.1]heptan-2-ol (Reagent 2), 6.1 mg (0,038 mmol) of CDI, and 33.3 mg (0,258 mmol) of DIPEA. Purified by HPLC (gradient: from A-75%: B-25% to A-60%: B-40%; Rf=0.49; run time=8.5 min). Yield: 18.2 mg (34.8%). Yellow powder. LCMS purity: 100% (LCMS, Rf=0.56, run time=2 min). EI MS m/z: pos. 448.2 (MH+).

{dispiro[3.0.3\hat{ }{5}.1\hat{ }{4}]nonan-2-yl}methyl N-{[2-(2,6-dioxopiperidin-3-yl)-3-oxo-2,3-dihydro-1H-isoindol-5-yl]methyl}carbamate (Compound 93) was obtained by Synthetic procedure 1 with using 33 mg (0,107 mmol) of 3-[6-(aminomethyl)-1-oxo-2,3-dihydro-1H-isoindol-2-yl]piperidine-2,6-dione hydrochloride (Reagent 1), 24 mg (0,158 mmol) of {dispiro[3.0.3 \hat{ }{5}.1 \hat{ }{4}]nonan-2-yl}methanol (Reagent 2), 5.4 mg (0,033 mmol) of CDI, and 29.9 mg (0,231 mmol) of DIPEA. Purified by HPLC (gradient: from A-65%: B-35% to A-45%: B-55%; Rf=0.62; run time=8.5 min). Yield: 15.7 mg (33.1%). Yellow powder. LCMS purity: 100% (LCMS, Rf=0.59, run time=6 min). EI MS m/z: pos. 452.2 (MH+).

{2-oxaspiro[4.4]nonan-3-yl}methyl N-{[2-(2,6-dioxopiperidin-3-yl)-3-oxo-2,3-dihydro-1H-isoindol-5-yl]methyl}carbamate (Compound 94) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 25 mg (0.16 mmol) of {2-oxaspiro[4.4]nonan-3-yl}methanol (Reagent 2), 5.2 mg (0,032 mmol) of CDI, and 30.3 mg (0,234 mmol) of DIPEA. Purified by HPLC (gradient: from A-85%: B-15% to A-65%: B-35%; Rf=0.7; run time=8.5 min). Yield: 13.4 mg (27.6%). Yellow powder. LCMS purity: 100% (LCMS, Rf=0.42, run time=2.5 min). EI MS m/z: pos. 456.0 (MH+).

{6,6-difluorospiro[2.5]octan-1-yl}methyl N-{[2-(2,6-dioxopiperidin-3-yl)-3-oxo-2,3-dihydro-1H-isoindol-5-yl]methyl}carbamate (Compound 95) was obtained by Synthetic procedure 1 with using 33 mg (0,107 mmol) of 3-[6-(aminomethyl)-1-oxo-2,3-dihydro-1H-isoindol-2-yl]piperidine-2,6-dione hydrochloride (Reagent 1), 29 mg (0,165 mmol) of {6,6-difluorospiro[2.5]octan-1-yl}methanol (Reagent 2), 5.2 mg (0,032 mmol) of CDI, and 31.2 mg (0,241 mmol) of DIPEA. Purified by HPLC (gradient: from A-75%: B-25% to A-50%: B-50%; Rf=0.59; run time=8.5 min). Yield: 14.5 mg (27.8%). Yellow powder. LCMS purity: 100% (LCMS, Rf=0.52, run time=2 min). EI MS m/z: pos. 476.2 (MH+).

(3-{[(tert-butoxy)carbonyl](methyl)amino}cyclobutyl)methyl N-{[2-(2,6-dioxopiperidin-3-yl)-3-oxo-2,3-dihydro-1H-isoindol-5-yl]methyl}carbamate (Compound 96) was obtained by Synthetic procedure 1 with using 34 mg (0.11 mmol) of 3-[6-(aminomethyl)-1-oxo-2,3-dihydro-1H-isoindol-2-yl]piperidine-2,6-dione hydrochloride (Reagent 1), 35 mg (0,163 mmol) of tert-butyl N-[3-(hydroxymethyl)cyclobutyl]-N-methylcarbamate (Reagent 2), 6.1 mg (0,038 mmol) of CDI, and 30.8 mg (0,238 mmol) of DIPEA. Purified by HPLC (gradient: from A-75%: B-25% to A-55%: B-45%; Rf=0.97; run time=8.2 min). Yield: 17.2 mg (30.8%). Yellow powder. LCMS purity: 100% (LCMS, Rf=0.64, run time=2 min). EI MS m/z: pos. 415.2, 537.2.

(1-cyclopropyl-3-methylazetidin-3-yl)methyl N-{[2-(2,6-dioxopiperidin-3-yl)-3-oxo-2,3-dihydro-1H-isoindol-5-yl]methyl}carbamate; trifluoroacetic acid (Compound 97) was obtained by Synthetic procedure 1 with using 53 mg (0,171 mmol) of 3-[6-(aminomethyl)-1-oxo-2,3-dihydro-1H-isoindol-2-yl]piperidine-2,6-dione hydrochloride (Reagent 1), 36 mg (0,255 mmol) of (1-cyclopropyl-3-methylazetidin-3-yl)methanol (Reagent 2), 8.5 mg (0,052 mmol) of CDI, and 48.3 mg (0,374 mmol) of DIPEA. Purified by HPLC (gradient: from A-100%: B-0% to A-85%: B-15%; Rf=0.57; run time=8.5 min). Yield: 22 mg (29.4%). Light brown sticky oil. LCMS purity: 95.5% (LCMS, Rf=0.3, run time=2.5 min). EI MS m/z: pos. 441.2 (MH+).

2-(3-methylazetidin-1-yl)ethyl N-{[2-(2,6-dioxopiperidin-3-yl)-3-oxo-2,3-dihydro-1H-isoindol-5-yl]methyl}carbamate; trifluoroacetic acid (Compound 98) was obtained by Synthetic procedure 1 with using 55 mg (0,178 mmol) of 3-[6-(aminomethyl)-1-oxo-2,3-dihydro-1H-isoindol-2-yl]piperidine-2,6-dione hydrochloride (Reagent 1), 29 mg (0,252 mmol) of 2-(3-methylazetidin-1-yl)ethan-1-ol (Reagent 2), 7.8 mg (0,048 mmol) of CDI, and 47.7 mg (0,369 mmol) of DIPEA. Purified by HPLC (gradient: from A-100%: B-0% to A-75%: B-25%; Rf=0.61; run time=6.5 min). Yield: 7.5 mg (10.8%). Yellow sticky oil. LCMS purity: 96% (LCMS, Rf=0.36, run time=2 min). EI MS m/z: pos. 415.2 (MH+).

(1-ethylazetidin-2-yl)methyl N-{[2-(2,6-dioxopiperidin-3-yl)-3-oxo-2,3-dihydro-1H-isoindol-5-yl]methyl}carbamate; trifluoroacetic acid (Compound 99) was obtained by Synthetic procedure 1 with using 61 mg (0,197 mmol) of 3-[6-(aminomethyl)-1-oxo-2,3-dihydro-1H-isoindol-2-yl]piperidine-2,6-dione hydrochloride (Reagent 1), 32 mg (0,278 mmol) of (1-ethylazetidin-2-yl)methanol (Reagent 2), 9.3 mg (0,057 mmol) of CDI, and 52.7 mg (0,408 mmol) of DIPEA. Purified by HPLC (gradient: from A-100%: B-0% to A-80%: B-20%; Rf=0.46; run time=8.5 min). Yield: 8.1 mg (10.6%). Brown sticky oil. LCMS purity: 90.2% (LCMS, Rf=0.27, run time=2 min). EI MS m/z: pos. 415.2 (MH+).

(1-ethylazetidin-3-yl)methyl N-{[2-(2,6-dioxopiperidin-3-yl)-3-oxo-2,3-dihydro-1H-isoindol-5-yl]methyl}carbamate; trifluoroacetic acid (Compound 100) was obtained by Synthetic procedure 1 with using 59 mg (0.19 mmol) of 3-[6-(aminomethyl)-1-oxo-2,3-dihydro-1H-isoindol-2-yl]piperidine-2,6-dione hydrochloride (Reagent 1), 42 mg (0,277 mmol) of (1-ethylazetidin-3-yl)methanol hydrochloride (Reagent 2), 9.6 mg (0,059 mmol) of CDI, 52.5 mg (0,406 mmol) of DIPEA, and 47.7 mg (0,369 mmol) of DIPEA. Purified by HPLC (gradient: from A-100%: B-0% to A-80%: B-20%; R_{f}=0.42; run time=8.2 min). Yield: 16.1 mg (21%). Yellow sticky oil. LCMS purity: 94.7% (LCMS, R_{f}=0.36, run time=2 min). EI MS m/z: pos. 415.2 (MH⁺).

### Example 15. Synthesis of Compound 101

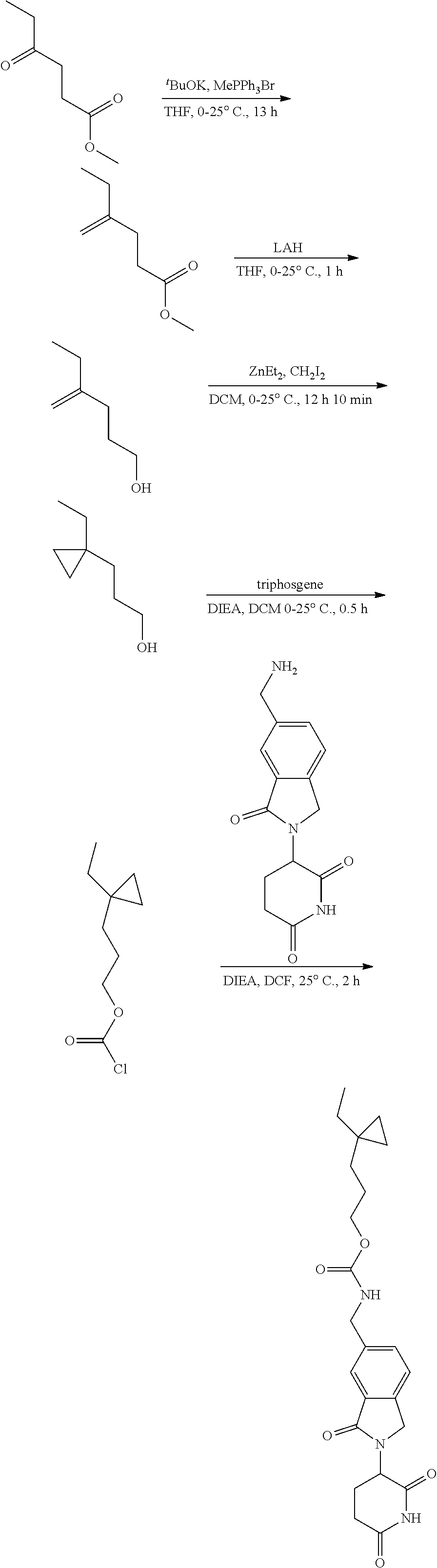

### Step 1. Procedure for Preparation of Compound 2—methyl 4-methylenehexanoate

To the solution of bromo(methyl)triphenylphosphorane (3.22 g, 9.02 mmol, 1.30 eq) in tetrahydrofuran (10.0 mL) was added potassium tert-butoxide (1.17 g, 10.4 mmol, 1.50 eq) which was dissolved in tetrahydrofuran (10.0 mL) at 0° C. under nitrogen atmosphere. After 0.5 h, to the mixture was added methyl 4-oxohexanoate (1.00 g, 6.94 mmol, 1.00 eq). After 0.5 h, the reaction was stirred at 25° C. for 12 h. The mixture was extracted with dichloromethane (2×10 mL). The combined organic layers were washed with brine (10 mL), and dried over anhydrous sodium sulfate, filtered and concentrated to give a residue. The residue was purified by column chromatography (SiO2, petroleum ether/ethyl acetate=50/1) to afford methyl 4-methylenehexanoate (680 mg, 4.78 mmol, 68% yield) as colorless oil.

1H NMR (400 MHz, CDCl3) δ=4.76 (s, 1H), 4.71 (s, 1H), 3.68 (s, 3H), 2.51-2.44 (m, 2H), 2.40-2.32 (m, 2H), 2.05 (q, J=7.4 Hz, 2H), 1.04 (t, J=7.4 Hz, 3H).

### Step 2. Procedure for Preparation of 4-methylenehexan-1-ol

To the solution of methyl 4-methylenehexanoate (680 mg, 4.78 mmol, 1.00 eq) in tetrahydrofuran (5.00 mL) was added lithium aluminium hydride (272 mg, 7.17 mmol, 1.50 eq) at 0° C. under nitrogen atmosphere. Then the reaction was stirred at 25° C. for 1 h. The reaction was quenched by addition water (2 mL), further added aqueous sodium hydroxide (3 M, 2 mL), extracted by dichloromethane (2×10 mL). The combined organic layers were washed with brine (10 mL), and dried over anhydrous sodium sulfate, filtered and concentrate to give a residue. The residue was purified by column chromatography (SiO2, petroleum ether/ethyl acetate=20/5) to afford 4-methylenehexan-1-ol (436 mg, 3.82 mmol, 79% yield) as colorless oil.

1H NMR (400 MHz, CDCl3) δ=4.75 (s, 2H), 3.67 (t, J=6.5 Hz, 2H), 2.16-2.03 (m, 4H), 1.77-1.68 (m, 2H), 1.47 (br s, 1H), 1.04 (t, J=7.4 Hz, 3H).

### Step 3. Procedure for Preparation of 3-(1-ethylcyclopropyl)propan-1-ol

To the solution of 4-methylenehexan-1-ol (240 mg, 2.10 mmol, 1.00 eq) in dichloromethane (5.00 mL) was added diethylzinc (1.00 M, 10.5 mL, 5.00 eq) at 0° C. under nitrogen atmosphere. After 10 min, diiodomethane (2.81 g, 10.5 mmol, 847 uL, 5.00 eq) was added into the mixture. Then the reaction was stirred at 25° C. for 12 h. The mixture was extracted with dichloromethane (2×10 mL). The combined organic layers were washed with brine (10 mL), and dried over anhydrous sodium sulfate, filtered and concentrate to give a residue. The residue was purified by column chromatography (SiO2, petroleum ether/ethyl acetate=10/1) to afford to afford 3-(1-ethylcyclopropyl)propan-1-ol (140 mg, 873.5 umol, 41% yield, 80% purity) as yellow oil.

1H NMR (400 MHz, CDCl3) δ=3.65 (t, J=6.6 Hz, 2H), 1.66-1.59 (m, 2H), 1.32-1.25 (m, 4H), 0.89 (t, J=7.4 Hz, 3H), 0.23 (br d, J=4.6 Hz, 4H).

### Step 4. Procedure for Preparation of Compound 5—3-(1-ethylcyclopropyl)propyl carbonochloridate

To the solution of 3-(1-ethylcyclopropyl)propan-1-ol (50.0 mg, 390 umol, 1.00 eq) in dichloromethane (0.500 mL) was added bis(trichloromethyl) carbonate (115.7 mg, 390 umol, 1.00 eq) at 0° C. Then the N,N-diisopropylethylamine (151 mg, 1.17 mmol, 203 uL, 3.00 eq) was added into the mixture. Then the reaction was stirred at 25° C. for 0.5 h. The reaction mixture was concentrated under reduced pressure to afford 3-(1-ethylcyclopropyl)propyl carbonochloridate (74.4 mg, crude) as an orange solid.

### Step 5. Procedure for Preparation of 3-(1-ethylcyclopropyl)propyl ((2-(2,6-dioxopiperidin-3-yl)-3-oxoisoindolin-5-yl)methyl)carbamate

To the solution of 3-(1-ethylcyclopropyl)propyl carbonochloridate (74.4 mg, 390 umol, 1.00 eq) in dimethylformamide (1.00 mL) were added 3-(6-(aminomethyl)-1-oxoisoindolin-2-yl)piperidine-2,6-dione (121 mg, 390 umol, 1.00 eq, hydrochloride) and N,N-diisopropylethylamine (151 mg, 1.17 mmol, 203 uL, 3.00 eq). Then the reaction was stirred at 25° C. for 2 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 38%-68%, 9 min) and lyophilized to afford 3-(1-ethylcyclopropyl)propyl ((2-(2,6-dioxopiperidin-3-yl)-3-oxoisoindolin-5-yl)methyl)carbamate (11.98 mg, 28.02 umol, 5% yield, 98% purity) as a yellow solid.

1H NMR (400 MHz, DMSO-d6) δ=10.98 (s, 1H), 7.74 (br t, J=5.9 Hz, 1H), 7.60 (s, 1H), 7.56-7.47 (m, 2H), 5.11 (dd, J=5.1, 13.3 Hz, 1H), 4.46-4.39 (m, 1H), 4.33-4.23 (m, 3H), 3.95 (br t, J=6.6 Hz, 2H), 2.96-2.86 (m, 1H), 2.60 (br d, J=17.5 Hz, 1H), 2.45-2.35 (m, 1H), 2.03-1.97 (m, 1H), 1.64-1.48 (m, 2H), 1.27-1.19 (m, 4H), 0.86-0.81 (m, 3H), 0.19 (br d, J=1.6 Hz, 4H).

1H NMR (400 MHz, DMSO-d6, T=80° C.) δ=10.72 (br s, 1H), 7.63 (s, 1H), 7.55-7.49 (m, 2H), 7.42 (br s, 1H), 5.07 (dd, J=5.2, 13.1 Hz, 1H), 4.47-4.32 (m, 2H), 4.28 (d, J=6.1 Hz, 2H), 3.98 (t, J=6.6 Hz, 2H), 2.94-2.84 (m, 1H), 2.66-2.60 (m, 1H), 2.41 (dq, J=4.6, 13.0 Hz, 1H), 2.07-2.01 (m, 1H), 1.63-1.54 (m, 2H), 1.29-1.23 (m, 4H), 0.86-0.82 (m, 3H), 0.20 (br d, J=6.6 Hz, 4H).

### MS (ESI) m/z 428.5 [M+H]⁺.

### Example 16. Synthesis of Compound 102

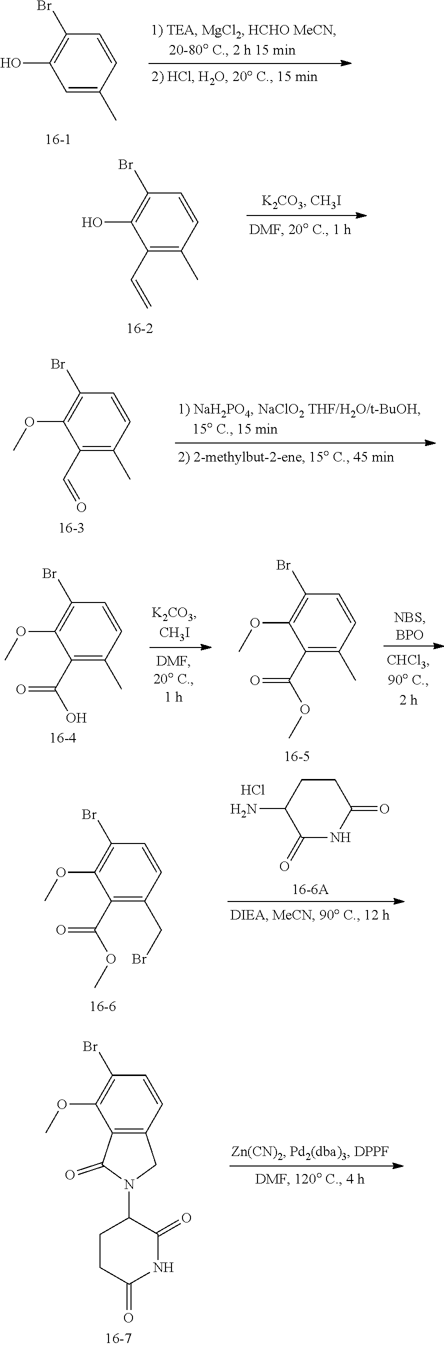

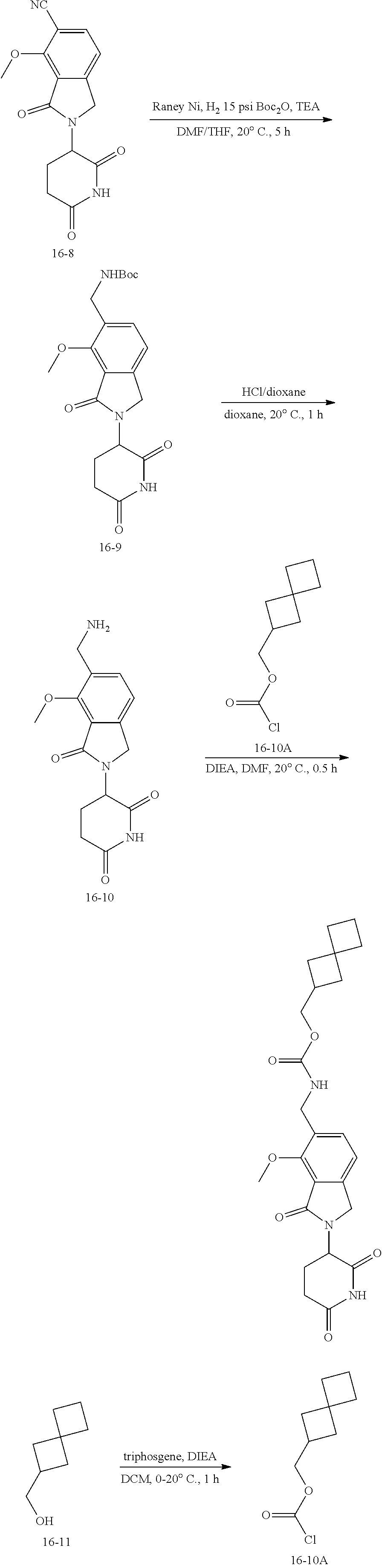

### Step 1. Procedure for Preparation of Compound 2—3-bromo-2-hydroxy-6-methylbenzaldehyde

To a solution of 2-bromo-5-methylphenol (5.00 g, 26.7 mmol, 1.00 eq) in acetonitrile (100 mL) were added triethylamine (10.8 g, 107 mmol, 14.9 mL, 4.00 eq) and magnesium chloride (3.82 g, 40.1 mmol, 1.65 mL, 1.50 eq). The mixture was stirred at 20° C. for 15 min, then paraformaldehyde (5.62 g, 187 mmol, 5.15 mL, 7.00 eq) was added, the mixture was stirred at 80° C. for another 2 h. The reaction mixture was poured into hydrochloric acid (3.00 M, 200 mL, 22.4 eq) and stirred for 15 min. The aqueous phase was extracted with ethyl acetate (3×100 mL). The combined organic layers were washed with brine (80 mL), dried over anhydrous sodium sulfate, filtered and concentrated under reduced pressure to afford 3-bromo-2-hydroxy-6-methylbenzaldehyde (5.50 g, 25.6 mmol, 96% yield) as orange oil. ¹H NMR (400 MHz, CDCl₃-d) δ=12.52 (s, 1H), 10.27 (s, 1H), 7.63 (d, J=8.0 Hz, 1H), 6.68-6.64 (m, 1H), 2.60 (s, 3H).

### Step 2. Procedure for Preparation of Compound 3—3-bromo-2-methoxy-6-methylbenzaldehyde

To a solution of 3-bromo-2-hydroxy-6-methylbenzaldehyde (5.50 g, 25.6 mmol, 1.00 eq) in dimethylformamide (50.0 mL) were added potassium carbonate (7.07 g, 51.2 mmol, 2.00 eq) and iodomethane (3.67 g, 25.8 mmol, 1.61 mL, 1.01 eq). The reaction mixture was stirred at 20° C. for 1 h. The reaction mixture was poured into water (300 mL), extracted with ethyl acetate (3×150 mL). The combined organic layers were washed with brine (200 mL), dried over anhydrous sodium sulfate, filtered and concentrated under reduced pressure to give a residue. The residue was purified by column chromatography (SiO₂, petroleum ether/ethyl acetate=1/0 to 50/1) to afford 3-bromo-2-methoxy-6-methylbenzaldehyde (4.10 g, 17.9 mmol, 70% yield) as yellow oil.

¹H NMR (400 MHz, CDCl₃-d) δ=10.47 (s, 1H), 7.58 (d, J=8.1 Hz, 1H), 6.87 (d, J=8.3 Hz, 1H), 3.90 (s, 3H), 2.50 (s, 3H).

### Step 3. Procedure for Preparation of Compound 4—3-bromo-2-methoxy-6-methylbenzoic acid

To a solution of 3-bromo-2-methoxy-6-methylbenzaldehyde (4.10 g, 17.9 mmol, 1.00 eq) in tetrahydrofuran (32.0 mL), water (16.0 mL) and tertiary butanol (8.00 mL) were added sodium dihydrogen phosphate (8.59 g, 71.6 mmol, 4.00 eq) and sodium chlorite (6.48 g, 71.6 mmol, 4.00 eq). The reaction mixture was stirred at 15° C. for 15 min. Then 2-methylbut-2-ene (8.79 g, 125 mmol, 13.3 mL, 7.00 eq) was added and the mixture was stirred at 15° C. for another 45 min. The reaction mixture was poured into water (100 mL) and adjust pH=9˜10 with 1 M sodium hydroxide. The aqueous phase was extracted with ethyl acetate (3×100 mL). The aqueous phase was adjust pH=5˜6 with 1 M hydrochloric acid. The aqueous phase was extracted with ethyl acetate (3×100 mL). The combined organic layers were washed with brine (200 mL), dried over anhydrous sodium sulfate, filtered and concentrated under reduced pressure to afford 3-bromo-2-methoxy-6-methylbenzoic acid (3.03 g, 12.4 mmol, 69% yield) as a white solid.

1H NMR (400 MHz, DMSO-d6) δ=7.56 (d, J=8.1 Hz, 1H), 7.00 (d, J=8.3 Hz, 1H), 3.78 (s, 3H), 2.23 (s, 3H).

### Step 4. Procedure for Preparation of Compound 5—methyl 3-bromo-2-methoxy-6-methylbenzoate

To a solution of 3-bromo-2-methoxy-6-methylbenzoic acid (3.03 g, 12.4 mmol, 1.00 eq) in dimethylformamide (30.0 mL) were added potassium carbonate (3.42 g, 24.7 mmol, 2.00 eq) and iodomethane (1.75 g, 12.4 mmol, 770 uL, 1.00 eq). The reaction mixture was stirred at 20° C. for 1 h. The reaction mixture was poured into water (300 mL), extracted with ethyl acetate (3×150 mL). The combined organic layers were washed with brine (200 mL), dried over anhydrous sodium sulfate, filtered and concentrated under reduced pressure to afford methyl 3-bromo-2-methoxy-6-methyl-benzoate (3.10 g, 12.0 mmol, 97% yield) as yellow oil.

1H NMR (400 MHz, DMSO-d₆) δ=7.62 (d, J=8.3 Hz, 1H), 7.03 (dd, J=0.6, 8.3 Hz, 1H), 3.87 (s, 3H), 3.77 (s, 3H), 2.19 (s, 3H).

### Step 5. Procedure for Preparation of Compound 6—methyl 3-bromo-6-(bromomethyl)-2-methoxybenzoate

To a solution of methyl 3-bromo-2-methoxy-6-methyl-benzoate (3.10 g, 12.0 mmol, 1.00 eq) in trichloromethane (30.0 mL) were added dibenzoyl peroxide (580 mg, 2.39 mmol, 0.200 eq) and N-Bromosuccinimide (2.34 g, 13.2 mmol, 1.10 eq). The reaction mixture was stirred at 90° C. for 2 h. The reaction mixture was filtered and the filtrate was concentrated under reduced pressure to give a residue. The residue was purified by column chromatography (SiO₂, petroleum ether/ethyl acetate=1/0 to 20/1) to afford methyl 3-bromo-6-(bromomethyl)-2-methoxybenzoate (3.17 g, 9.38 mmol, 78% yield) as yellow oil.

¹H NMR (400 MHz, CDCl₃-d) δ=7.58 (d, J=8.3 Hz, 1H), 7.07 (d, J=8.4 Hz, 1H), 4.47 (s, 2H), 3.99 (s, 3H), 3.92 (s, 3H).

### Step 6. Procedure for Preparation of Compound 7—3-(6-bromo-7-methoxy-1-oxoisoindolin-2-yl)piperidine-2,6-dione

To a solution of methyl 3-bromo-6-(bromomethyl)-2-methoxybenzoate (3.17 g, 9.38 mmol, 1.00 eq) in acetonitrile (30.0 mL) were added N,N-diisopropylethylamine (3.64 g, 28.1 mmol, 4.90 mL, 3.00 eq) and 3-aminopiperidine-2,6-dione (1.85 g, 11.3 mmol, 1.20 eq, hydrochloride). The reaction mixture was stirred at 90° C. for 12 h. The reaction mixture was concentrated under reduced pressure to give a residue. The residue was triturated with ethyl acetate (10 mL) and 1 M hydrochloric acid (5 mL) at 20° C. for 30 min and then filtered. The filter cake was triturated with water (6 mL) and ethyl acetate (3 mL) at 20° C. for 30 min and then filtered. The second filter cake was concentrated under reduced pressure to afford 3-(6-bromo-7-methoxy-1-oxoisoindolin-2-yl)piperidine-2,6-dione (1.84 g, 5.21 mmol, 56% yield) as a blue solid.

1H NMR (400 MHz, DMSO-d6) δ=11.01 (s, 1H), 7.86 (d, J=8.0 Hz, 1H), 7.29 (d, J=8.1 Hz, 1H), 5.08 (dd, J=5.1, 13.3 Hz, 1H), 4.46-4.35 (m, 1H), 4.33-4.23 (m, 1H), 3.97 (s, 3H), 2.97-2.83 (m, 1H), 2.65-2.56 (m, 1H), 2.38 (dq, J=4.3, 13.2 Hz, 1H), 2.05-1.95 (m, 1H).

### Step 7. Procedure for Preparation of Compound 8-2-(2,6-dioxopiperidin-3-yl)-4-methoxy-3-oxoisoindoline-5-carbonitrile

To a solution of 3-(6-bromo-7-methoxy-1-oxoisoindolin-2-yl)piperidine-2,6-dione (800 mg, 2.27 mmol, 1.00 eq) in dimethylformamide (8.00 mL) were added zinc cyanide (532 mg, 4.53 mmol, 288 uL, 2.00 eq), 1,1-bis(diphenylphosphino)ferrocene (251 mg, 453 umol, 0.200 eq) and tris(dibenzylideneacetone)dipalladium(0) (207 mg, 227 umol, 0.100 eq). The reaction mixture was stirred at 120° C. for 4 h under nitrogen atmosphere. The reaction mixture was filtered. The filtrate was diluted with water (40 mL) and extracted with ethyl acetate (3×30 mL). The combined organic layers were washed with water (60 mL), dried over anhydrous sodium sulfate, filtered and concentrated under reduced pressure to give a residue. The residue was purified by reverse phase chromatography (C18, 80 g; condition: water/acetonitrile=100:0 to 88:12, 0.1% formic acid) and lyophilized to afford 2-(2,6-dioxopiperidin-3-yl)-4-methoxy-3-oxoisoindoline-5-carbonitrile (275 mg, 919 umol, 41% yield) as a yellow solid.

¹H NMR (400 MHz, DMSO-d₆) δ=11.02 (br s, 1H), 8.00 (d, J=7.9 Hz, 1H), 7.48 (d, J=7.9 Hz, 1H), 5.09 (dd, J=5.0, 13.3 Hz, 1H), 4.58-4.49 (m, 1H), 4.46-4.38 (m, 1H), 4.20 (s, 3H), 2.95-2.84 (m, 1H), 2.64-2.58 (m, 1H), 2.44-2.33 (m, 1H), 2.05-1.97 (m, 1H).

### Step 8. Procedure for Preparation of Compound 9—tert-butyl ((2-(2,6-dioxopiperidin-3-yl)-4-methoxy-3-oxoisoindolin-5-yl)methyl)carbamate

To a solution of 2-(2,6-dioxopiperidin-3-yl)-4-methoxy-3-oxoisoindoline-5-carbonitrile (270 mg, 902 umol, 1.00 eq) in tetrahydrofuran (27.0 mL) and dimethylformamide (1.00 mL) were added triethylamine (913 mg, 9.02 mmol, 1.26 mL, 10.0 eq), di-tert-butyldicarbonate (591 mg, 2.71 mmol, 622 uL, 3.00 eq) and Raney nickel (77.3 mg, 902 umol, 1.00 eq). The reaction mixture was stirred at 20° C. for 5 h under 15 psi of hydrogen atmosphere. The reaction mixture was filtered and the filtrate was concentrated under reduced pressure to give a residue. The residue was purified by reverse phase chromatography (C18, 80 g; condition: water/acetonitrile=100:0 to 0:100, 0.1% formic acid) and lyophilized to afford tert-butyl ((2-(2,6-dioxopiperidin-3-yl)-4-methoxy-3-oxoisoindolin-5-yl)methyl)carbamate (300 mg, 602 umol, 67% yield, 81% purity) as a yellow solid

1H NMR (400 MHz, DMSO-d6) δ=10.97 (s, 1H), 7.46 (d, J=7.8 Hz, 1H), 7.36-7.29 (m, 1H), 7.27 (d, J=7.8 Hz, 1H), 5.06 (dd, J=5.1, 13.3 Hz, 1H), 4.44-4.35 (m, 1H), 4.32-4.23 (m, 1H), 4.19 (br d, J=6.0 Hz, 2H), 4.00-3.92 (m, 3H), 2.93-2.87 (m, 1H), 2.60 (br d, J=16.3 Hz, 1H), 2.44-2.31 (m, 1H), 2.04-1.96 (m, 1H), 1.39 (s, 9H).

### Step 9. Procedure for Preparation of Compound 10—3-(6-(aminomethyl)-7-methoxy-1-oxoisoindolin-2-yl)piperidine-2,6-dione

To a solution of tert-butyl ((2-(2,6-dioxopiperidin-3-yl)-4-methoxy-3-oxoisoindolin-5-yl)methyl)carbamate (300 mg, 602 umol, 81% purity, 1.00 eq) in dioxane (3.00 mL) was added hydrochloric acid/dioxane (4.00 M, 3.00 mL, 16.1 eq). The reaction mixture was stirred at 20° C. for 1 h. The reaction mixture was concentrated under reduced pressure to afford 3-(6-(aminomethyl)-7-methoxy-1-oxoisoindolin-2-yl)piperidine-2,6-dione (250 mg, 596 umol, 99% yield, 81% purity, hydrochloride) as a yellow solid.

### MS (ESI) m/z 607.4 [2M+H]⁺.

### Step 10. Procedure for Preparation of Compound 10A—Spiro[3.3]Heptan-2-Ylmethyl carbonochloridate

To a solution of spiro[3.3]heptan-2-ylmethanol (30.0 mg, 238 umol, 1.00 eq) in dichloromethane (0.300 mL) was added bis(trichloromethyl) carbonate (70.5 mg, 238 umol, 1.00 eq) and N,N-diisopropylethylamine (61.5 mg, 475 umol, 82.8 uL, 2.00 eq) at 0° C. The reaction mixture was stirred at 20° C. for 1 h. The reaction mixture was concentrated under reduced pressure to spiro[3.3]heptan-2-ylmethyl carbonochloridate (44.0 mg, crude) as a yellow solid.

### Step 11. Procedure for Preparation of spiro[3.3]heptan-2-ylmethyl ((2-(2,6-dioxopiperidin-3-yl)-4-methoxy-3-oxoisoindolin-5-yl)methyl)carbamate

To a solution of 3-(6-(aminomethyl)-7-methoxy-1-oxoisoindolin-2-yl)piperidine-2,6-dione (90.0 mg, 215 umol, 81% purity, 1.00 eq, hydrochloride) in dimethylformamide (1.00 mL) were added N,N-diisopropylethylamine (55.5 mg, 429 umol, 74.7 uL, 2.00 eq) and spiro[3.3]heptan-2-ylmethyl carbonochloridate (40.5 mg, 215 umol, 1.00 eq). The reaction mixture was stirred at 20° C. for 0.5 h. The reaction mixture was filtered. The filtrate was purified by reverse phase chromatography (C18, 40 g; condition: water/acetonitrile=100:0 to 52:48, 0.1% formic acid) and lyophilized to afford spiro[3.3]heptan-2-ylmethyl ((2-(2,6-dioxopiperidin-3-yl)-4-methoxy-3-oxoisoindolin-5-yl)methyl) carbamate (13.37 mg, 29.06 umol, 14% yield, 99% purity) as a white solid.

1H NMR (400 MHz, DMSO-d6) δ=10.97 (br s, 1H), 7.59 (br t, J=5.9 Hz, 1H), 7.46 (d, J=7.8 Hz, 1H), 7.32-7.21 (m, 1H), 5.07 (dd, J=5.1, 13.3 Hz, 1H), 4.52-4.34 (m, 1H), 4.32-4.19 (m, 3H), 3.97 (s, 3H), 3.90 (d, J=6.8 Hz, 2H), 2.97-2.84 (m, 1H), 2.60 (td, J=2.0, 15.3 Hz, 1H), 2.44-2.33 (m, 2H), 2.06-1.91 (m, 5H), 1.91-1.84 (m, 2H), 1.79-1.58 (m, 4H).

1H NMR (400 MHz, DMSO-d6, T=80° C.) δ=10.70 (br s, 1H), 7.49 (d, J=7.8 Hz, 1H), 7.30-7.10 (m, 2H), 5.02 (dd, J=5.3, 13.0 Hz, 1H), 4.44-4.35 (m, 1H), 4.35-4.24 (m, 3H), 4.01 (s, 3H), 3.92 (d, J=6.8 Hz, 2H), 2.88 (ddd, J=5.4, 13.4, 17.4 Hz, 1H), 2.66-2.60 (m, 1H), 2.45-2.33 (m, 2H), 2.08-1.94 (m, 5H), 1.92-1.85 (m, 2H), 1.81-1.68 (m, 4H).

### MS (ESI) m/z 456.3 [M+H]⁺.

### Example 17. Synthesis of Compound 103

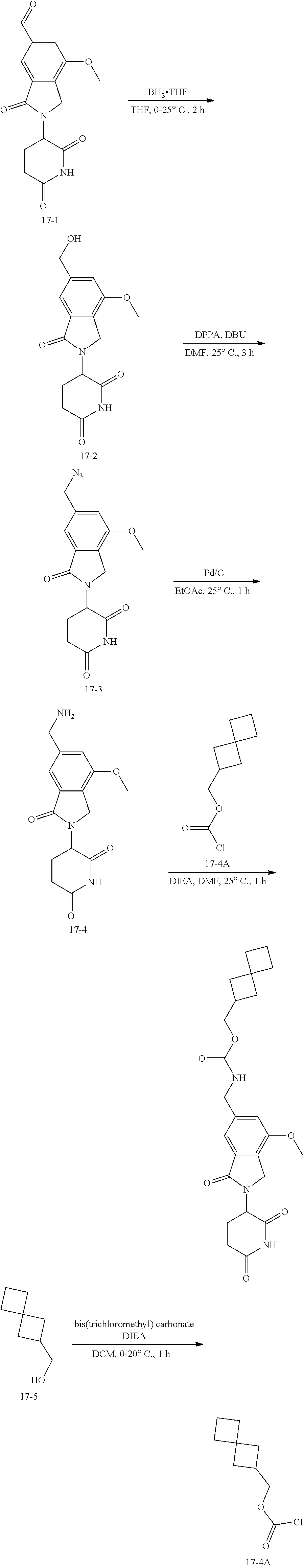

### Step 1. Procedure for Preparation of Compound 4A—spiro[3.3]heptan-2-ylmethyl carbonochloridate

To a solution of spiro[3.3]heptan-2-ylmethanol (30.0 mg, 238 umol, 1.00 eq) in dichloromethane (2.00 mL) were added bis(trichloromethyl) carbonate (113 mg, 380 umol, 1.60 eq) and N,N-diisopropylethylamine (61.5 mg, 475 umol, 82.8 uL, 2.00 eq) at 0° C. The mixture was stirred at 25° C. for 1 h. The reaction mixture was concentrated under reduced pressure to afford spiro[3.3]heptan-2-ylmethyl carbonochloridate (40.0 mg, crude) as yellow solid.

### Step 2. Procedure for Preparation of Compound 2—3-(6-(hydroxymethyl)-4-methoxy-1-oxoisoindolin-2-yl)piperidine-2,6-dione

To a solution of 2-(2,6-dioxo-3-piperidyl)-7-methoxy-3-oxo-isoindoline-5-carbaldehyde (400 mg, 1.32 mmol, 1.00 eq) in tetrahydrofuran (1.50 mL) was added borane dimethyl sulfide complex (1.00 M, 199 uL, 1.50 eq) at 0° C. under nitrogen atmosphere. The mixture was stirred at 25° C. for 2 h. The reaction mixture was quenched by addition water (5.00 mL) slowly at 25° C. and then concentrated under reduced pressure to give a residue. The residue was purified by chromatography (C18, 40 g; condition: water/acetonitrile=100:0 to 0:100, 0.1% formic acid condition) and lyophilized to afford 3-(6-(hydroxymethyl)-4-methoxy-1-oxoisoindolin-2-yl)piperidine-2,6-dione (150 mg, 493 umol, 37% yield) as a white solid.

1H NMR (400 MHz, DMSO-d₆) δ=10.94 (br s, 1H), 7.29 (s, 1H), 7.20 (s, 1H), 5.33 (br d, J=1.3 Hz, 1H), 5.08 (dd, J=5.1, 13.4 Hz, 1H), 4.61 (br s, 2H), 4.41-4.30 (m, 1H), 4.25-4.15 (m, 1H), 3.89 (s, 3H), 2.98-2.84 (m, 1H), 2.70-2.58 (m, 1H), 2.41-2.28 (m, 1H), 2.03-1.94 (m, 1H)

### Step 3. Procedure for Preparation of Compound 3—3-(6-(azidomethyl)-4-methoxy-1-oxoisoindolin-2-yl)piperidine-2,6-dione

To a solution of 3-(6-(hydroxymethyl)-4-methoxy-1-oxoisoindolin-2-yl)piperidine-2,6-dione (120 mg, 394 umol, 1.00 eq) in N,N-dimethylformamide (2.00 mL) were added diphenylphosphinyl azide (326 mg, 1.18 mmol, 256 uL, 3.00 eq) and 2,3,4,6,7,8,9,10-octahydropyrimido[1,2-a]azepine (180 mg, 1.18 mmol, 178 uL, 3.00 eq) at 25° C. The mixture was stirred at 25° C. for 3 h. The reaction mixture was concentrated under reduced pressure to give a residue. The residue was purified by reverse phase chromatography (C18, 40 g; condition: water/acetonitrile=100:0 to 0:100, 0.1% formic acid condition) and lyophilized to afford 3-(6-(azidomethyl)-4-methoxy-1-oxoisoindolin-2-yl)piperidine-2,6-dione (70.0 mg, 213 umol, 54% yield) as a white solid.

1H NMR (400 MHz, DMSO-d₆) δ=10.96 (s, 1H), 7.35 (s, 1H), 7.28 (s, 1H), 5.10 (dd, J=5.3, 13.2 Hz, 1H), 4.57 (s, 2H), 4.45-4.34 (m, 1H), 4.29-4.19 (m, 1H), 3.92 (s, 3H), 3.03-2.84 (m, 1H), 2.72-2.56 (m, 1H), 2.40-2.32 (m, 1H), 2.08-1.94 (m, 1H)

### Step 4. Procedure for Preparation of Compound 4—3-(6-(aminomethyl)-4-methoxy-1-oxoisoindolin-2-yl)piperidine-2,6-dione

To a solution of 3-(6-(azidomethyl)-4-methoxy-1-oxoisoindolin-2-yl)piperidine-2,6-dione (30.0 mg, 91.1 umol, 1.00 eq) in ethyl acetate (5.00 mL) was added in palladium on activated carbon (5.00 mg, 9.11 umol, 10% purity, 0.100 eq). Then the mixture was degassed and purged with hydrogen atmosphere for 3 times. The mixture was stirred at 25° C. for 1 h under hydrogen atmosphere. The reaction mixture was filtered and the filtrate was concentrated under reduced pressure to afford 3-(6-(aminomethyl)-4-methoxy-1-oxoisoindolin-2-yl)piperidine-2,6-dione (27.0 mg, crude) as a white solid.

### MS (ESI) m/z 303.9 [M+H]⁺.

Note: The product was in dead time.

### Step 5. Procedure for Preparation of spiro[3.3]heptan-2-ylmethyl ((2-(2,6-dioxopiperidin-3-yl)-7-methoxy-3-oxoisoindolin-5-yl)methyl)carbamate

To a solution of 3-(6-(aminomethyl)-4-methoxy-1-oxoisoindolin-2-yl)piperidine-2,6-dione (27.0 mg, 89.0 umol, 1.00 eq) in dichloromethane (2.00 mL) were added N,N-diisopropylethylamine (12.7 mg, 97.9 umol, 17.1 uL, 1.10 eq) and spiro[3.3]heptan-2-ylmethyl carbonochloridate (25.2 mg, 134 umol, 1.50 eq) at 25° C. The mixture was stirred at 25° C. for 1 h. The reaction mixture was concentrated under reduced pressure to give a residue. The residue was purified by reverse phase chromatography (C18, 40 g; condition: water/acetonitrile=100:0 to 0:100, 0.1% formic acid condition) and lyophilized to afford spiro[3.3]heptan-2-ylmethyl ((2-(2,6-dioxopiperidin-3-yl)-7-methoxy-3-oxoisoindolin-5-yl)methyl)carbamate (5.65 mg, 12.40 umol, 14% yield, 99% purity, formate) as white solid.

1H NMR (400 MHz, DMSO-d6) δ=10.97 (br s, 1H), 8.43 (s, 1H), 7.76 (br t, J=6.1 Hz, 1H), 7.21 (s, 1H), 7.14 (s, 1H), 5.09 (dd, J=5.1, 13.3 Hz, 1H), 4.43-4.12 (m, 4H), 3.93 (d, J=6.9 Hz, 2H), 3.88 (s, 3H), 2.97-2.83 (m, 1H), 2.70-2.57 (m, 1H), 2.44-2.32 (m, 2H), 2.09-1.84 (m, 7H), 1.81-1.67 (m, 4H)

### MS (ESI) m/z 456.0 [M+H]⁺.

### Example 18. Synthesis of Compound 104

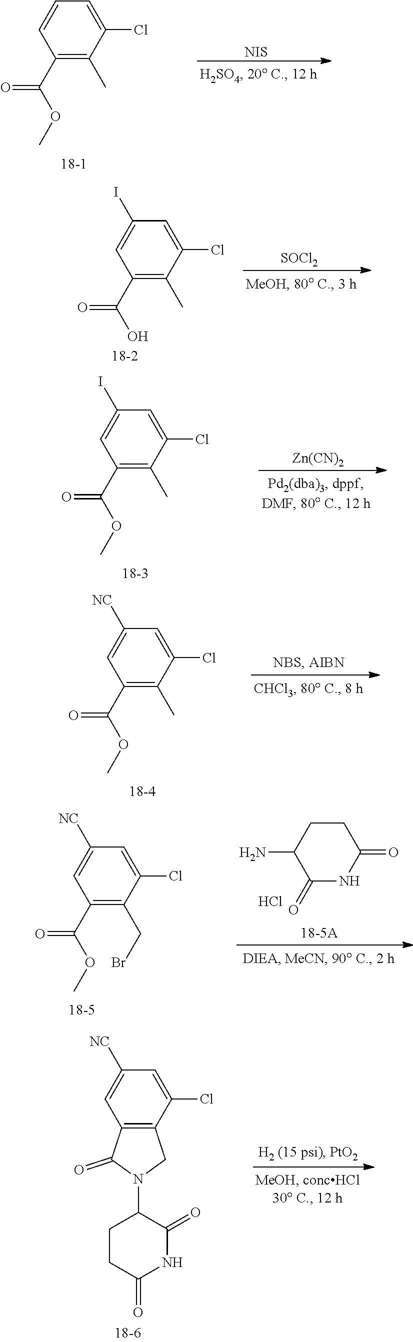

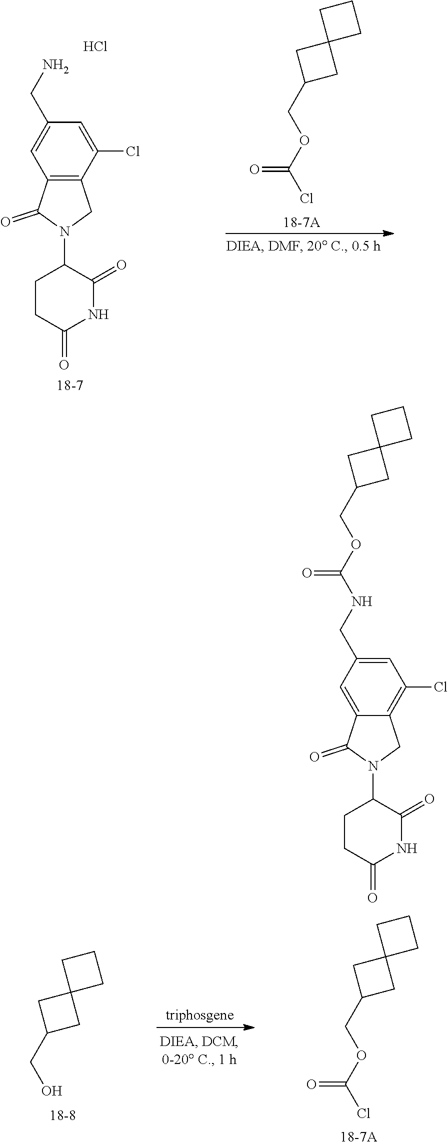

### Step 1. Procedure for Preparation of Compound 2—3-chloro-5-iodo-2-methylbenzoic acid

To a solution of methyl 3-chloro-2-methyl-benzoate (5.00 g, 27.0 mmol, 1.00 eq) in sulfuric acid (40.0 mL) was added N-lodosuccinimide (6.09 g, 27.0 mmol, 1.00 eq). The mixture was stirred at 20° C. for 12 h. The reaction mixture was added dropwise in water (100 mL) at 0° C., and extracted with ethyl acetate (3×50 mL). The combined organic layers were with dried over sodium sulfate, filtered and concentrated under reduced pressure to give a residue. The residue was purified by column chromatography (SiO2, petroleum ether/ethyl acetate=10/1 to 5/1) to afford 3-chloro-5-iodo-2-methylbenzoic acid (3.30 g, 11.1 mmol, 41% yield) as a white solid.

1H NMR (400 MHz, CDCl₃) δ=8.21 (d, J=1.6 Hz, 1H), 7.92 (d, J=1.5 Hz, 1H), 2.63 (s, 3H).

### Step 2. Procedure for Preparation of Compound 3—methyl 3-chloro-5-iodo-2-methylbenzoate

To a solution of 3-chloro-5-iodo-2-methyl-benzoic acid (2.90 g, 9.78 mmol, 1.00 eq) in methanol (30.0 mL) was added thionyl chloride (2.33 g, 19.5 mmol, 1.42 mL, 2.00 eq). The mixture was stirred at 80° C. for 3 h. The mixture was diluted with saturated sodium bicarbonate solution (150 mL), extracted with ethyl acetate (3×50 mL). The combined organic layers were washed with brine (100 mL), dried over anhydrous sodium sulfate, filtered and concentrated under reduced pressure to afford methyl 3-chloro-5-iodo-2-methylbenzoate (2.60 g, 8.37 mmol, 85% yield) as a white solid.

1H NMR (400 MHz, CDCl3) δ=8.02 (d, J=1.6 Hz, 1H), 7.85 (d, J=1.6 Hz, 1H), 3.91 (s, 3H), 2.55 (s, 3H).

### Step 3. Procedure for Preparation of Compound 4—methyl 3-chloro-5-cyano-2-methylbenzoate

To a solution of methyl 3-chloro-5-iodo-2-methylbenzoate (2.60 g, 8.37 mmol, 1.00 eq) in dimethylformamide (20.0 mL) were added zinc cyanide (589 mg, 5.02 mmol, 318 uL, 0.600 eq), tris(dibenzylideneacetone)dipalladium(0) (383 mg, 418 umol, 0.0500 eq) and 1,1′-Bis(diphenylphosphino)ferrocene (557 mg, 1.00 mmol, 0.120 eq). The mixture was stirred at 80° C. for 12 h under nitrogen atmosphere. The mixture was filtered and the filtrate was quenched with water (100 mL), extracted with ethyl acetate (3×50 mL). The combined organic layers were washed with brine (300 mL), and dried over anhydrous sodium sulfate, filtered and concentrated under reduced pressure to give a residue. The residue was purified by column chromatography (SiO2, petroleum ether/ethyl acetate=10/1 to 0/1) to afford methyl 3-chloro-5-cyano-2-methylbenzoate (1.00 g, 4.77 mmol, 56% yield) as a light yellow solid.

1H NMR (400 MHz, CDCl3) δ=8.02 (d, J=1.6 Hz, 1H), 7.79 (d, J=1.6 Hz, 1H), 3.95 (s, 3H), 2.69 (s, 3H).

### Step 4. Procedure for Preparation of Compound 5—methyl 2-(bromomethyl)-3-chloro-5-cyanobenzoate

To a solution of methyl 3-chloro-5-cyano-2-methylbenzoate (1.00 g, 4.77 mmol, 1.00 eq) in trichloromethane (15.0 mL) were added N-Bromosuccinimide (849 mg, 4.77 mmol, 1.00 eq) and (E)-2,2′-(diazene-1,2-diyl)bis(2-methylpropanenitrile) (783 mg, 4.77 mmol, 1.00 eq). The mixture was stirred at 80° C. at 2 h. N-Bromosuccinimide (84.9 mg, 477 umol, 0.100 eq) was added into the mixture. The mixture was stirred at 80° C. at 2 h. N-Bromosuccinimide (169 mg, 954 umol, 0.200 eq) was added into the mixture. The mixture was stirred at 80° C. at 2 h. N-Bromosuccinimide (169 mg, 954 umol, 0.200 eq) was added into the mixture. The mixture was stirred at 80° C. at 2 h. The mixture was concentrated under reduced pressure to give a residue. The residue was dissolved with water (30 mL), extracted with ethyl acetate (3×30 mL). The combined organic layers were washed with brine (50 mL), and dried over anhydrous sodium sulfate, filtered and concentrated under reduced pressure to afford methyl 2-(bromomethyl)-3-chloro-5-cyanobenzoate (2.60 g, crude) as a yellow solid.

1H NMR (400 MHz, CDCl₃) δ=8.16 (d, J=1.5 Hz, 1H), 7.86 (d, J=1.5 Hz, 1H), 5.10 (s, 2H), 4.00 (s, 3H).

### Step 5. Procedure for Preparation of Compound 6—7-chloro-2-(2,6-dioxopiperidin-3-yl)-3-oxoisoindoline-5-carbonitrile

To a solution of methyl 2-(bromomethyl)-3-chloro-5-cyanobenzoate (2.60 g, 7.21 mmol, 1.00 eq) in acetonitrile (30.0 mL) were added 3-aminopiperidine-2,6-dione (1.19 g, 7.21 mmol, 1.00 eq, hydrochloride) and N,N-diisopropylethylamine (2.80 g, 21.6 mmol, 3.77 mL, 3.00 eq). The mixture was stirred at 90° C. for 2 h. The mixture was concentrated under reduced pressure to give a residue. The residue was triturated with the mixture (ethyl acetate:water=2:1, 10 mL) to afford 7-chloro-2-(2,6-dioxopiperidin-3-yl)-3-oxoisoindoline-5-carbonitrile (270 mg, 889 umol, 12% yield) as a deep purple solid.

1H NMR (400 MHz, DMSO-d6) δ=11.04 (s, 1H), 8.35 (d, J=1.1 Hz, 1H), 8.24 (d, J=1.0 Hz, 1H), 5.16 (dd, J=5.1, 13.2 Hz, 1H), 4.66-4.57 (m, 1H), 4.49-4.41 (m, 1H), 2.95-2.87 (m, 1H), 2.62 (br s, 1H), 2.47-2.41 (m, 1H), 2.05-1.99 (m, 1H).

### Step 6. Procedure for Preparation of Compound 7—3-(6-(aminomethyl)-4-chloro-1-oxoisoindolin-2-yl)piperidine-2,6-dione

To a solution of 7-chloro-2-(2,6-dioxopiperidin-3-yl)-3-oxoisoindoline-5-carbonitrile (280 mg, 921 umol, 1.00 eq) in methanol (28.0 mL) was added platinum dioxide (62.8 mg, 276 umol, 0.300 eq), concentrated hydrochloric acid (0.840 mL) and hydrogen (1.86 mg, 921 umol, 1.00 eq). The mixture was stirred at 30° C. for 12 h under hydrogen atmosphere (15 psi). The mixture was filtered and the filtrated was concentrated under reduced pressure to give a crude product. The crude product was triturated with ethanol (3 mL) to afford 3-(6-(aminomethyl)-4-chloro-1-oxoisoindolin-2-yl)piperidine-2,6-dione (160 mg, 464 umol, 50% yield, hydrochloride) as a light red solid.

1H NMR (400 MHz, DMSO-d₆) δ=11.01 (s, 1H), 8.56 (br s, 3H), 7.91 (d, J=6.9 Hz, 2H), 5.14 (br dd, J=4.9, 13.2 Hz, 1H), 4.56-4.46 (m, 1H), 4.40-4.31 (m, 1H), 4.17 (br s, 2H), 2.99-2.84 (m, 1H), 2.60 (br d, J=17.4 Hz, 1H), 2.46-2.40 (m, 1H), 2.07-1.98 (m, 1H).

### Step 7. Procedure for Preparation of Compound 7A—spiro[3.3]heptan-2-ylmethyl carbonochloridate

To a solution of spiro[3.3]heptan-2-ylmethanol (40.0 mg, 316 umol, 1.00 eq) in dichloromethane (1.00 mL) were added bis(trichloromethyl) carbonate (94.0 mg, 316 umol, 1.00 eq) and N,N-diisopropylethylamine (81.9 mg, 633 umol, 110 uL, 2.00 eq) at 0° C. The mixture was stirred at 20° C. for 1 h. The mixture was concentrated under reduced pressure to afford spiro[3.3]heptan-2-ylmethyl carbonochloridate (59.0 mg, crude) as orange oil.

### Step 8. Procedure for Preparation of spiro[3.3]heptan-2-ylmethyl ((7-chloro-2-(2,6-dioxopiperidin-3-yl)-3-oxoisoindolin-5-yl)methyl)carbamate

To a solution of 3-(6-(aminomethyl)-4-chloro-1-oxoisoindolin-2-yl)piperidine-2,6-dione (50.0 mg, 145 umol, 1.00 eq, hydrochloride) in dimethylformamide (0.500 mL) were added spiro[3.3]heptan-2-ylmethyl carbonochloridate (54.8 mg, 290 umol, 2.00 eq) and N,N-diisopropylethylamine (56.3 mg, 435 umol, 75.9 uL, 3.00 eq). The mixture was stirred at 20° C. for 0.5 h. The mixture was filtered. The filtrate was purified by reverse phase column chromatography (C18, 40 g; condition: water/acetonitrile=1/0 to 0/1, 0.1% formic acid) and lyophilized to afford spiro[3.3]heptan-2-ylmethyl ((7-chloro-2-(2,6-dioxopiperidin-3-yl)-3-oxoisoindolin-5-yl)methyl)carbamate (15.96 mg, 34.3 umol, 23% yield, 99% purity) as a yellow solid.

¹H NMR (400 MHz, DMSO-d₆) δ=10.99 (br s, 1H), 7.79 (br t, J=6.3 Hz, 1H), 7.59 (d, J=3.6 Hz, 2H), 5.13 (dd, J=5.1, 13.3 Hz, 1H), 4.51-4.43 (m, 1H), 4.34-4.22 (m, 3H), 3.92 (br d, J=6.8 Hz, 2H), 2.97-2.85 (m, 1H), 2.63-2.56 (m, 1H), 2.44 (br d, J=3.8 Hz, 1H), 2.37 (br d, J=7.3 Hz, 1H), 2.06-1.92 (m, 5H), 1.90-1.84 (m, 2H), 1.79-1.67 (m, 4H)

### MS (ESI) m/z 460.1 [M+H]⁺.

### Example 19. Synthesis of Compound 105

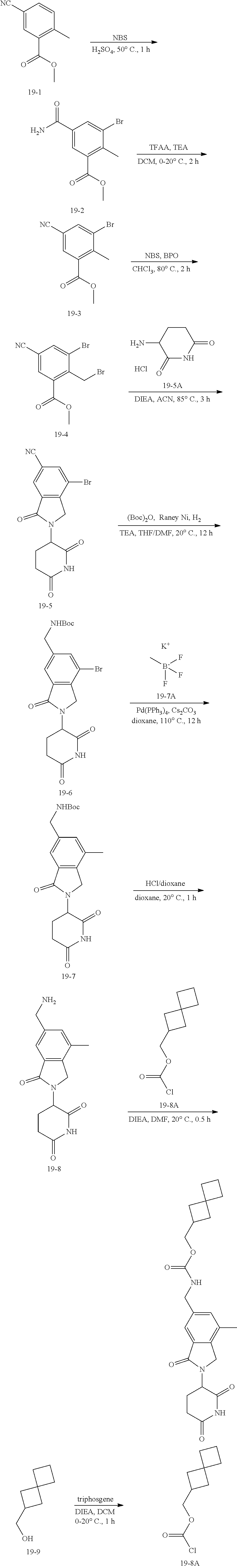

### Step 1. Procedure for Preparation of Compound 2—methyl 3-bromo-5-carbamoyl-2-methylbenzoate

To a solution of methyl 5-cyano-2-methylbenzoate (5.00 g, 28.5 mmol, 1.00 eq) in sulfuric acid (30.0 mL) was added N-Bromosuccinimide (5.59 g, 31.4 mmol, 1.10 eq). The reaction mixture was stirred at 50° C. for 1 h. The reaction mixture was added to the ice water (150 mL) and then extracted with ethyl acetate (3×100 mL). The combined organic layers were dried over anhydrous sodium sulfate, filtered and concentrated under reduced pressure to give a residue. The residue was purified by column chromatography (SiO2, petroleum ether/ethyl acetate=1/0 to 1/1) to afford methyl 3-bromo-5-carbamoyl-2-methylbenzoate (7.17 g, 26.4 mmol, 92% yield) as a white solid.

1H NMR (400 MHz, CDCl₃) δ=8.18 (s, 1H), 8.17 (s, 1H), 6.41-5.55 (m, 2H), 3.94 (s, 3H), 2.69 (s, 3H).

### Step 2. Procedure for Preparation of Compound 3—methyl 3-bromo-5-cyano-2-methylbenzoate

To a solution of methyl 3-bromo-5-carbamoyl-2-methylbenzoate (7.17 g, 26.4 mmol, 1.00 eq) in dichloromethane (70.0 mL) were added triethylamine (9.33 g, 92.2 mmol, 12.8 mL, 3.50 eq) and trifluoroacetic anhydride (7.19 g, 34.3 mmol, 4.76 mL, 1.30 eq) at 0° C. The reaction mixture was stirred at 20° C. for 2 h. The reaction mixture was concentrated under reduced pressure to give a residue. The residue was purified by column chromatography (SiO2, petroleum ether/ethyl acetate=1/0 to 10/1) to afford methyl 3-bromo-5-cyano-2-methylbenzoate (4.00 g, 15.7 mmol, 60% yield) as a white solid.

1H NMR (400 MHz, CDCl₃) δ=8.05 (d, J=1.5 Hz, 1H), 7.98 (d, J=1.5 Hz, 1H), 3.95 (s, 3H), 2.71 (s, 3H).

### Step 3. Procedure for Preparation of Compound 4—methyl 3-bromo-2-(bromomethyl)-5-cyanobenzoate

To a solution of methyl 3-bromo-5-cyano-2-methylbenzoate (4.00 g, 15.7 mmol, 1.00 eq) in trichloromethane (40.0 mL) were added dibenzoyl peroxide (572 mg, 2.36 mmol, 0.150 eq) and N-Bromosuccinimide (3.08 g, 17.3 mmol, 1.10 eq). The reaction mixture was stirred at 80° C. for 2 h. The reaction mixture was filtered and the filtrate was concentrated under reduced pressure to give a residue. The residue was purified by column chromatography (SiO2, petroleum ether/ethyl acetate=1/0 to 10/1) to afford methyl 3-bromo-2-(bromomethyl)-5-cyanobenzoate (4.51 g, 13.5 mmol, 86% yield) as a yellow solid.

1H NMR (400 MHz, CDCl₃) δ=8.19 (d, J=1.4 Hz, 1H), 8.04 (d, J=1.4 Hz, 1H), 5.13 (s, 2H), 4.00 (s, 3H).

### Step 4. Procedure for Preparation of Compound 5—7-bromo-2-(2,6-dioxopiperidin-3-yl)-3-oxoisoindoline-5-carbonitrile

To a solution of methyl 3-bromo-2-(bromomethyl)-5-cyanobenzoate (4.51 g, 13.5 mmol, 1.00 eq) in acetonitrile (45.0 mL) were added N,N-diisopropylethylamine (5.23 g, 40.5 mmol, 7.05 mL, 2.99 eq) and 3-aminopiperidine-2,6-dione (2.68 g, 16.3 mmol, 1.20 eq, hydrochloride). The reaction mixture was stirred at 85° C. for 3 h. The reaction mixture was concentrated under reduced pressure to give a residue. The residue was triturated with a mixture (ethyl acetate: water-3:2, 40 mL) at 20° C. for 12 h and then filtered. The filter cake was concentrated under reduced pressure to afford 7-bromo-2-(2,6-dioxopiperidin-3-yl)-3-oxoisoindoline-5-carbonitrile (4.36 g, 12.5 mmol, 92% yield) as a bluish violet solid.

1H NMR (400 MHz, DMSO-d6) δ=11.03 (s, 1H), 8.77-8.44 (m, 1H), 8.27 (d, J=1.0 Hz, 1H), 5.17 (dd, J=5.1, 13.3 Hz, 1H), 4.59-4.49 (m, 1H), 4.43-4.33 (m, 1H), 2.91 (ddd, J=5.4, 13.5, 17.5 Hz, 1H), 2.60 (br d, J=17.6 Hz, 1H), 2.49-2.42 (m, 1H), 2.07-1.97 (m, 1H).

### Step 5. Procedure for Preparation of Compound 6—tert-butyl ((7-bromo-2-(2,6-dioxopiperidin-3-yl)-3-oxoisoindolin-5-yl)methyl)carbamate

To a solution of 7-bromo-2-(2,6-dioxopiperidin-3-yl)-3-oxoisoindoline-5-carbonitrile (1.00 g, 2.87 mmol, 1.00 eq) in tetrahydrofuran (50.0 mL) and dimethylformamide (10.0 mL) were added triethylamine (2.91 g, 28.7 mmol, 4.00 mL, 10.0 eq), di-tert-butyldicarbonate (1.88 g, 8.62 mmol, 1.98 mL, 3.00 eq) and Raney nickel (246 mg, 2.87 mmol, 1.00 eq). The reaction mixture was stirred at 20° C. for 12 h under 15 psi of hydrogen atmosphere. The reaction mixture was filtered and the filtrate was concentrated under reduced pressure to give a residue. The residue was purified by reverse phase chromatography (C18, 120 g; condition: water/acetonitrile=100:0 to 60:40, 0.1% formic acid) and lyophilized to afford tert-butyl ((7-bromo-2-(2,6-dioxopiperidin-3-yl)-3-oxoisoindolin-5-yl)methyl)carbamate (900 mg, 1.99 mmol, 69% yield) as a white solid.

1H NMR (400 MHz, DMSO-d6) δ=11.00 (br s, 1H), 7.72 (s, 1H), 7.63 (s, 1H), 7.55 (br t, J=6.0 Hz, 1H), 5.13 (dd, J=5.1, 13.3 Hz, 1H), 4.44-4.35 (m, 1H), 4.28-4.14 (m, 3H), 2.98-2.84 (m, 1H), 2.59 (td, J=2.1, 15.2 Hz, 1H), 2.48-2.40 (m, 1H), 2.06-1.97 (m, 1H), 1.39 (s, 9H).

### Step 6. Procedure for Preparation of Compound 7—tert-butyl ((2-(2,6-dioxopiperidin-3-yl)-7-methyl-3-oxoisoindolin-5-yl)methyl)carbamate

To a solution of tert-butyl ((7-bromo-2-(2,6-dioxopiperidin-3-yl)-3-oxoisoindolin-5-yl)methyl)carbamate (200 mg, 442 umol, 1.00 eq) in dioxane (5.00 mL) were added potassium trifluoro(methyl)borate (270 mg, 2.21 mmol, 5.00 eq), tetrakis[triphenylphosphine]palladium(0) (51.1 mg, 44.2 umol, 0.100 eq) and cesium carbonate (288 mg, 884 umol, 2.00 eq). The reaction mixture was stirred at 110° C. for 12 h under nitrogen atmosphere. The reaction mixture was filtered and the filtrate was concentrated under reduced pressure to give a residue. The residue was purified by reverse phase chromatography (C18, 80 g; condition: water/acetonitrile=100:0 to 58:42, 0.1% formic acid) and lyophilized to afford tert-butyl ((2-(2,6-dioxopiperidin-3-yl)-7-methyl-3-oxoisoindolin-5-yl)methyl)carbamate (110 mg, 284 umol, 64% yield) as a yellow solid.

1H NMR (400 MHz, DMSO-d6) δ=10.98 (br s, 1H), 7.50-7.44 (m, 1H), 7.43 (s, 1H), 7.29 (s, 1H), 5.12 (br dd, J=3.7, 13.1 Hz, 1H), 4.44-4.34 (m, 1H), 4.27-4.13 (m, 3H), 2.98-2.86 (m, 1H), 2.61 (br d, J=17.3 Hz, 1H), 2.40 (br s, 1H), 2.31 (s, 3H), 2.02 (br s, 1H), 1.39 (s, 9H).

### Step 7. Procedure for Preparation of Compound 8—3-(6-(aminomethyl)-4-methyl-1-oxoisoindolin-2-yl)piperidine-2,6-dione

To a solution of tert-butyl ((2-(2,6-dioxopiperidin-3-yl)-7-methyl-3-oxoisoindolin-5-yl)methyl)carbamate (140 mg, 361 umol, 1.00 eq) in dioxane (2.00 mL) was added hydrochloric acid/dioxane (4.00 M, 2.00 mL, 22.1 eq). The reaction mixture was stirred at 20° C. for 1 h. The reaction mixture was concentrated under reduced pressure to afford 3-(6-(aminomethyl)-4-methyl-1-oxoisoindolin-2-yl)piperidine-2,6-dione (100 mg, 309 umol, 85% yield, hydrochloride) as a yellow solid.

1H NMR (400 MHz, DMSO-d6) δ=11.00 (s, 1H), 8.36 (br d, J=4.8 Hz, 2H), 7.71 (s, 1H), 7.53 (s, 1H), 5.13 (dd, J=5.1, 13.3 Hz, 1H), 4.46 (br d, J=17.4 Hz, 1H), 4.32-4.27 (m, 1H), 4.15-4.07 (m, 2H), 2.99-2.86 (m, 1H), 2.62 (br d, J=17.4 Hz, 1H), 2.42 (br d, J=8.5 Hz, 1H), 2.34 (s, 3H), 2.06-1.99 (m, 1H).

### Step 8. Procedure for Preparation of Compound 8A—spiro[3.3]heptan-2-ylmethyl carbonochloridate

To a solution of spiro[3.3]heptan-2-ylmethanol (40.0 mg, 317 umol, 1.00 eq) in dichloromethane (1.00 mL) was added bis(trichloromethyl) carbonate (94.1 mg, 317 umol, 1.00 eq) and N,N-diisopropylethylamine (81.9 mg, 634 umol, 110 uL, 2.00 eq) at 0° C. The reaction mixture was stirred at 20° C. for 1 h. The reaction mixture was concentrated under reduced pressure to spiro[3.3]heptan-2-ylmethyl carbonochloridate (60.0 mg, crude) as yellow oil.

### Step 9. Procedure for Preparation of spiro[3.3]heptan-2-ylmethyl ((2-(2,6-dioxopiperidin-3-yl)-7-methyl-3-oxoisoindolin-5-yl)methyl)carbamate

To a solution of 3-(6-(aminomethyl)-4-methyl-1-oxoisoindolin-2-yl)piperidine-2,6-dione (50.0 mg, 154 umol, 1.00 eq, hydrochloride) in dimethylformamide (0.500 mL) were added N,N-diisopropylethylamine (59.9 mg, 463 umol, 80.7 uL, 3.00 eq) and spiro[3.3]heptan-2-ylmethyl carbonochloridate (58.3 mg, 309 umol, 2.00 eq). The reaction mixture was stirred at 20° C. for 0.5 h. The reaction mixture was filtered. The filtrate was purified by reverse phase chromatography (C18, 40 g; condition: water/acetonitrile=100:0 to 40:60, 0.1% formic acid) and lyophilized to afford spiro[3.3]heptan-2-ylmethyl ((2-(2,6-dioxopiperidin-3-yl)-7-methyl-3-oxoisoindolin-5-yl)methyl)carbamate (15.46 mg, 34.82 umol, 23% yield, 99% purity) as a white solid.

1H NMR (400 MHz, DMSO-d₆) δ=11.02-10.96 (m, 1H), 7.73 (br t, J=6.0 Hz, 1H), 7.42 (s, 1H), 7.30 (s, 1H), 5.13 (dd, J=5.1, 13.4 Hz, 1H), 4.44-4.35 (m, 1H), 4.28-4.17 (m, 3H), 3.91 (d, J=6.9 Hz, 2H), 2.92 (ddd, J=5.3, 13.6, 17.4 Hz, 1H), 2.60 (td, J=2.0, 15.3 Hz, 1H), 2.45-2.34 (m, 2H), 2.30 (s, 3H), 2.06-1.91 (m, 5H), 1.90-1.83 (m, 2H), 1.80-1.62 (m, 4H).

### MS (ESI) m/z 462.2 [M+Na]⁺.

### Example 20. Synthesis of Compound 106

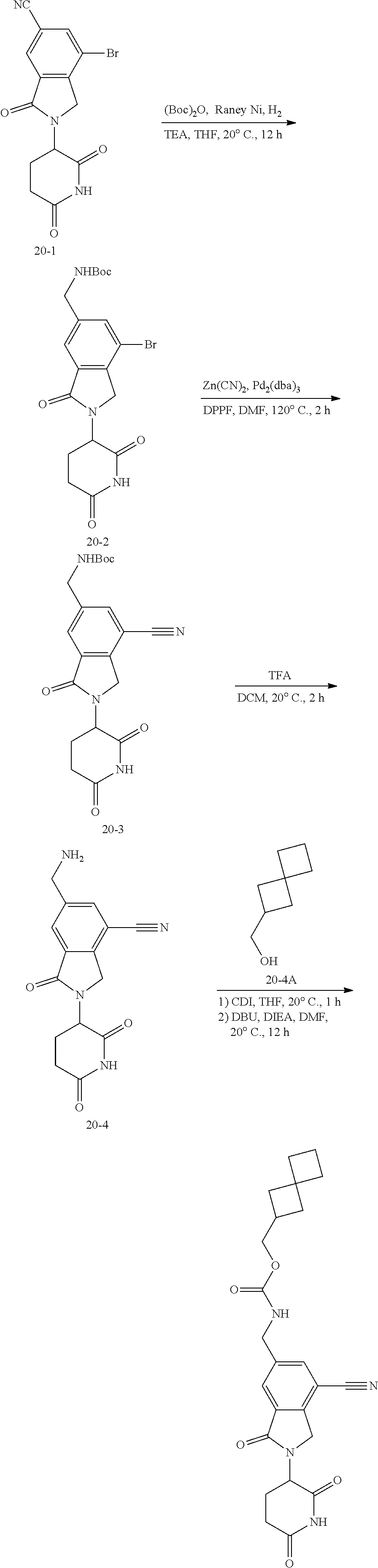

### Step 1. Procedure for Preparation of Compound 2—tert-butyl ((7-bromo-2-(2,6-dioxopiperidin-3-yl)-3-oxoisoindolin-5-yl)methyl)carbamate

To a solution of 7-bromo-2-(2,6-dioxopiperidin-3-yl)-3-oxoisoindoline-5-carbonitrile (500 mg, 1.44 mmol, 1.00 eq) in tetrahydrofuran (100 mL) were added triethylamine (1.45 g, 14.4 mmol, 2.00 mL, 10.0 eq), di-tert-butyldicarbonate (940 mg, 4.31 mmol, 990 uL, 3.00 eq) and Raney nickel (123 mg, 1.44 mmol, 1.00 eq). The reaction mixture was stirred at 20° C. for 12 h under 15 psi of hydrogen atmosphere. The reaction mixture was filtered and the filtrate was concentrated under reduced pressure to give a residue. The residue was purified by reverse phase chromatography (C18, 80 g; condition: water/acetonitrile=100:0 to 50:50, 0.1% formic acid) and lyophilized to afford tert-butyl ((7-bromo-2-(2,6-dioxopiperidin-3-yl)-3-oxoisoindolin-5-yl)methyl)carbamate (410 mg, 906 umol, 63% yield) as a white solid.

1H NMR (400 MHz, DMSO-d6) δ=10.98 (br s, 1H), 7.72 (s, 1H), 7.63 (s, 1H), 7.53 (br t, J=5.9 Hz, 1H), 5.12 (dd, J=5.1, 13.3 Hz, 1H), 4.44-4.33 (m, 1H), 4.29-4.15 (m, 3H), 2.97-2.85 (m, 1H), 2.64-2.56 (m, 1H), 2.48-2.40 (m, 1H), 2.06-1.97 (m, 1H), 1.39 (s, 9H).

### Step 2. Procedure for Preparation of Compound 3—tert-butyl ((7-cyano-2-(2,6-dioxopiperidin-3-yl)-3-oxoisoindolin-5-yl)methyl)carbamate

To a solution of tert-butyl ((7-bromo-2-(2,6-dioxopiperidin-3-yl)-3-oxoisoindolin-5-yl)methyl)carbamate (200 mg, 442 umol, 1.00 eq) in dimethylformamide (2.00 mL) were added zinc cyanide (200 mg, 1.70 mmol, 108 uL, 3.85 eq), 1,1-bis(diphenylphosphino)ferrocene (49.0 mg, 88.4 umol, 0.200 eq) and tris(dibenzylideneacetone)dipalladium(0) (40.5 mg, 44.2 umol, 0.100 eq). The reaction mixture was stirred at 120° C. for 2 h under nitrogen atmosphere. The reaction mixture was filtered. The filtrate was diluted with water (50 mL) and extracted with ethyl acetate (3×30 mL). The combined organic layers were washed with water (50 mL), dried over anhydrous sodium sulfate, filtered and concentrated under reduced pressure to give a residue. The residue was purified by reverse phase chromatography (C18, 80 g; condition: water/acetonitrile=100:0 to 50:50, 0.1% formic acid) and lyophilized to afford tert-butyl ((7-cyano-2-(2,6-dioxopiperidin-3-yl)-3-oxoisoindolin-5-yl)methyl)carbamate (160 mg, 402 umol, 91% yield) as a white solid.

1H NMR (400 MHz, DMSO-d6) δ=11.02 (s, 1H), 7.96 (s, 1H), 7.91 (s, 1H), 7.59 (br t, J=6.1 Hz, 1H), 5.14 (dd, J=5.1, 13.3 Hz, 1H), 4.74-4.65 (m, 1H), 4.56-4.47 (m, 1H), 4.27 (br d, J=6.0 Hz, 2H), 2.97-2.85 (m, 1H), 2.63-2.57 (m, 1H), 2.47-2.41 (m, 1H), 2.06-1.96 (m, 1H), 1.39 (s, 9H).

### Step 3. Procedure for Preparation of Compound 4—6-(aminomethyl)-2-(2,6-dioxopiperidin-3-yl)-1-oxoisoindoline-4-carbonitrile

To a solution of tert-butyl ((7-cyano-2-(2,6-dioxopiperidin-3-yl)-3-oxoisoindolin-5-yl)methyl)carbamate (160 mg, 402 umol, 1.00 eq) in dichloromethane (5.00 mL) was added trifluoroacetic acid (770 mg, 6.75 mmol, 0.500 mL, 16.8 eq). The reaction mixture was stirred at 20° C. for 1 h. The reaction mixture was concentrated under reduced pressure to give a residue. The residue was diluted with water (15 mL) and then lyophilized to afford 6-(aminomethyl)-2-(2,6-dioxopiperidin-3-yl)-1-oxoisoindoline-4-carbonitrile (230 mg, crude, trifluoroacetate) as a yellow solid.

1H NMR (400 MHz, DMSO-d6) δ=11.03 (s, 1H), 8.26 (br s, 2H), 8.22 (s, 1H), 8.20 (s, 1H), 5.15 (dd, J=5.1, 13.2 Hz, 1H), 4.80-4.71 (m, 1H), 4.57 (d, J=18.3 Hz, 1H), 4.24 (br d, J=3.0 Hz, 2H), 2.97-2.85 (m, 1H), 2.62 (br d, J=17.4 Hz, 1H), 2.48-2.42 (m, 1H), 2.06-1.99 (m, 1H).

### Step 4. Procedure for Preparation of spiro[3.3]heptan-2-ylmethyl ((7-cyano-2-(2,6-dioxopiperidin-3-yl)-3-oxoisoindolin-5-yl)methyl)carbamate

To a solution of spiro[3.4]octan-2-ylmethanol (30.0 mg, 238 um, 1.00 eq) in tetrahydrofuran (0.500 mL) was added di(1H-imidazol-1-yl)methanone (57.8 mg, 357 umol, 1.50 eq). The reaction mixture was stirred at 20° C. for 1 h. The resulting solution was added to a mixture of 6-(aminomethyl)-2-(2,6-dioxopiperidin-3-yl)-1-oxoisoindoline-4-carbonitrile (80.0 mg, 194 umol, 1.00 eq, trifluoroacetate), 2,3,4,6,7,8,9,10-octahydropyrimido[1,2-a]azepine (29.5 mg, 194 umol, 29.3 uL, 1.00 eq) and N,N-diisopropylethylamine (25.1 mg, 194 umol, 33.8 uL, 1.00 eq) in dimethylformamide (1.00 mL). The reaction mixture was stirred at 20° C. for 12 h. The reaction mixture was concentrated under reduced pressure to give a residue. The residue was dissolved in dimethylformamide (1 mL) and then filtered. The filtrate was purified by reverse phase chromatography (C18, 40 g; condition: water/acetonitrile=100:0 to 50:50, 0.1% formic acid) and lyophilized to afford spiro[3.3]heptan-2-ylmethyl ((7-cyano-2-(2,6-dioxopiperidin-3-yl)-3-oxoisoindolin-5-yl)methyl)carbamate (10.75 mg, 23.62 umol, 12% yield, 99% purity) as a white solid.

1H NMR (400 MHz, DMSO-d6) δ=11.02 (s, 1H), 7.97 (s, 1H), 7.92 (s, 1H), 7.83 (br t, J=6.1 Hz, 1H), 5.15 (dd, J=5.1, 13.3 Hz, 1H), 4.69 (d, J=18.1 Hz, 1H), 4.50 (d, J=17.9 Hz, 1H), 4.32 (br d, J=5.9 Hz, 2H), 3.92 (d, J=6.8 Hz, 2H), 2.96-2.85 (m, 1H), 2.63-2.58 (m, 1H), 2.47-2.34 (m, 2H), 2.07-1.91 (m, 5H), 1.90-1.83 (m, 2H), 1.77-1.53 (m, 4H).

1H NMR (400 MHz, DMSO-d6, T=80° C.) δ=10.90-10.64 (m, 1H), 7.94 (br s, 1H), 7.93 (br s, 1H), 7.65-7.41 (m, 1H), 5.11 (dd, J=5.1, 13.0 Hz, 1H), 4.72-4.63 (m, 1H), 4.58-4.49 (m, 1H), 4.33 (d, J=5.9 Hz, 2H), 3.94 (d, J=6.8 Hz, 2H), 2.97-2.83 (m, 1H), 2.61 (br s, 1H), 2.46-2.42 (m, 1H), 2.41-2.34 (m, 1H), 2.10-2.00 (m, 3H), 1.97 (t, J=7.3 Hz, 2H), 1.91-1.84 (m, 2H), 1.80-1.74 (m, 2H), 1.74-1.66 (m, 2H).

### MS (ESI) m/z 473.2 [M+Na]⁺.

### Example 21. Synthesis of Compound 107

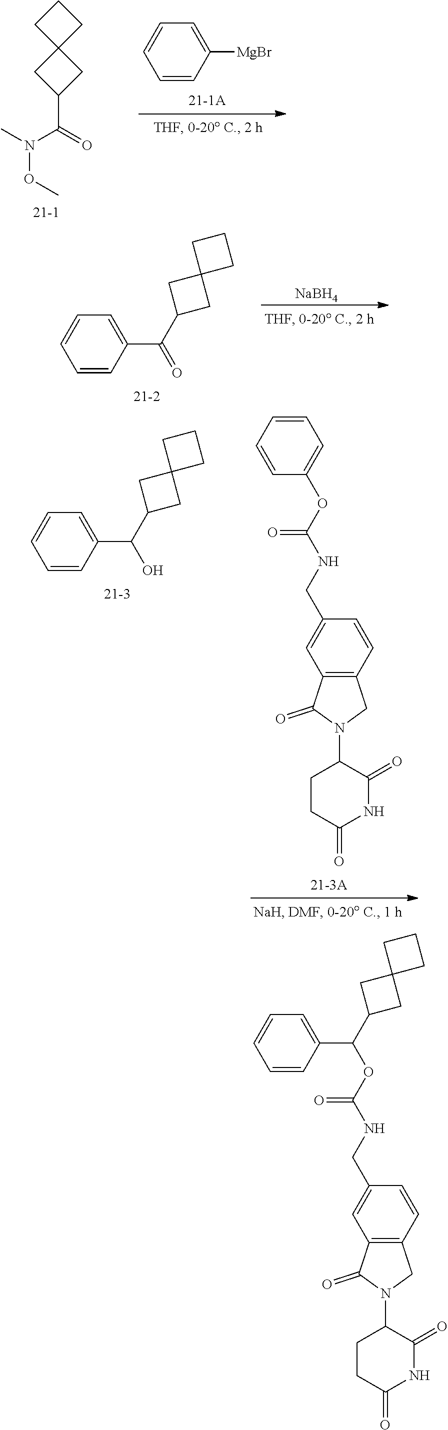

### Step 1. Procedure for Preparation of Compound 2—phenyl(spiro[3.3]heptan-2-yl)methanone

To a solution of N-methoxy-N-methylspiro[3.3]heptane-2-carboxamide (100 mg, 546 umol, 1.00 eq) in tetrahydrofuran (1.00 mL) was added phenylmagnesium bromide (2.80 M, 292 uL, 1.50 eq) at 0° C. The reaction mixture was stirred at 20° C. for 2 h. The mixture was quenched with saturated ammonium chloride aqueous solution (10.0 mL) and extracted with ethyl acetate (3×20 mL). The combined organic layers were washed with brine (2×20 mL), dried over anhydrous sodium sulfate, filtered and concentrated under reduced pressure to afford phenyl(spiro[3.3]heptan-2-yl)methanone (352 mg, 1.24 mmol, 12% yield) as light yellow oil.

1H NMR (400 MHz, CDCl3-d) δ=7.90 (d, J=8.0 Hz, 2H), 7.58-7.50 (m, 1H), 7.49-7.42 (m, 2H), 3.84 (quin, J=8.5 Hz, 1H), 2.42-2.36 (m, 2H), 2.34-2.28 (m, 2H), 2.13 (t, J=7.3 Hz, 2H), 1.97-1.90 (m, 2H), 1.88-1.79 (m, 2H).

### Step 2. Procedure for Preparation of Compound 3—phenyl(spiro[3.3]heptan-2-yl)methanol

To a solution of phenyl(spiro[3.3]heptan-2-yl)methanone (95.0 mg, 474 umol, 1.00 eq) in tetrahydrofuran (1.00 mL) was added sodium borohydride (35.9 mg, 949 umol, 2.00 eq) at 0° C. The reaction mixture was stirred at 20° C. for 2 h. The mixture was quenched with saturated ammonium chloride (10.0 mL) and extracted with ethyl acetate (3×20 mL). The combined organic layers were washed with brine (2×20 mL), dried over anhydrous sodium sulfate, filtered and concentrated under reduced pressure to afford phenyl(spiro[3.3]heptan-2-yl)methanol (87.0 mg, 430 umol, 91% yield) as light yellow oil.

1H NMR (400 MHz, CDCl3-d) δ=7.36-7.33 (m, 1H), 7.32 (d, J=0.6 Hz, 2H), 7.30 (br s, 1H), 7.29 (br d, J=1.8 Hz, 1H), 4.52 (d, J=8.0 Hz, 1H), 2.45 (sxt, J=8.2 Hz, 1H), 2.13 (ddd, J=3.9, 7.6, 11.2 Hz, 1H), 2.03-1.97 (m, 2H), 1.97-1.90 (m, 2H), 1.90-1.84 (m, 2H), 1.83-1.78 (m, 2H), 1.78-1.70 (m, 2H).

### Step 3. Procedure for preparation of phenyl(spiro[3.3]heptan-2-yl)methyl ((2-(2,6-dioxopiperidin-3-yl)-3-oxoisoindolin-5-yl)methyl)carbamate

To a solution of phenyl ((2-(2,6-dioxopiperidin-3-yl)-3-oxoisoindolin-5-yl)methyl)carbamate (97.0 mg, 247 umol, 1.00 eq) and phenyl(spiro[3.3]heptan-2-yl)methanol (50.0 mg, 247 umol, 1.00 eq) in dimethylformamide (1.00 mL) was added sodium hydride (19.8 mg, 494 umol, 60% purity, 2.00 eq) at 0° C. The reaction mixture was stirred at 20° C. for 1 h. The reaction mixture was filtered. The filtrate was purified by reverse phase chromatography (C18, 80 g; condition: water/acetonitrile=100:0 to 0:100) and lyophilized to afford phenyl(spiro[3.3]heptan-2-yl)methyl ((2-(2,6-dioxopiperidin-3-yl)-3-oxoisoindolin-5-yl)methyl) carbamate (14.64 mg, 28.90 umol, 12% yield, 99% purity) as a white solid.

1H NMR (400 MHz, DMSO-d6) δ=10.97 (br s, 1H), 7.88 (br t, J=5.2 Hz, 1H), 7.69-7.57 (m, 1H), 7.55-7.42 (m, 2H), 7.37-7.28 (m, 2H), 7.27 (br d, J=7.1 Hz, 2H), 7.20 (br s, 1H), 5.46 (br d, J=8.0 Hz, 1H), 5.10 (br dd, J=4.3, 12.6 Hz, 1H), 4.45-4.37 (m, 1H), 4.35-4.19 (m, 3H), 2.97-2.85 (m, 1H), 2.60 (br d, J=17.5 Hz, 1H), 2.39 (br d, J=13.5 Hz, 1H), 2.04 -1.95 (m, 2H), 1.95-1.90 (m, 2H), 1.88 (br d, J=14.9 Hz, 2H), 1.85-1.78 (m, 2H), 1.78-1.73 (m, 2H), 1.72 (br s, 2H).

Special NMR (400 MHz, DMSO-d6, T=80° C.) δ=10.71 (br s, 1H), 7.63 (s, 1H), 7.61-7.39 (m, 3H), 7.34-7.28 (m, 2H), 7.28-7.19 (m, 3H), 5.49 (d, J=7.8 Hz, 1H), 5.07 (dd, J=5.1, 13.0 Hz, 1H), 4.47-4.40 (m, 1H), 4.37-4.32 (m, 1H), 4.29 (br d, J=5.9 Hz, 2H), 2.89 (ddd, J=5.5, 13.4, 17.5 Hz, 1H), 2.66-2.59 (m, 1H), 2.43-2.35 (m, 1H), 2.07-2.01 (m, 1H), 1.95 (br t, J=7.2 Hz, 3H), 1.85 (br d, J=7.6 Hz, 4H), 1.83-1.68 (m, 4H).

### MS (ESI) m/z 502.0 [M+H]⁺.

### Example 22. Synthesis of Compound 108

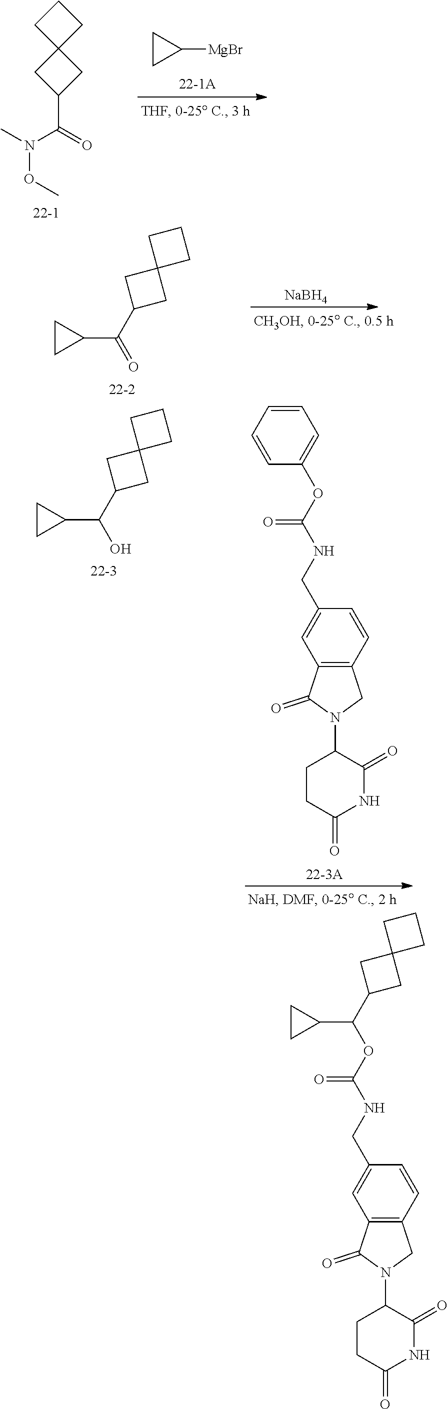

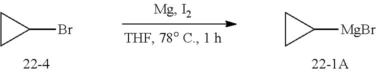

### Step 1. Procedure for Preparation of Compound 1A—cyclopropylmagnesium bromide

To a solution of magnesium dihydride (321 mg, 13.2 mmol, 1.20 eq), iodide (2.79 mg, 11.0 umol, 0.00100 eq) in tetrahydrofuran (8.00 mL) was added bromocyclopropane (1.33 g, 11.0 mmol, 881 uL, 1.00 eq) at 78° C. under nitrogen atmosphere. Then the reaction was stirred at 78° C. for 1 h. The solution of bromo(cyclopropyl)magnesium (1.60 g, crude) in tetrahydrofuran (8.00 mL) was obtained as a gray liquid.

Note: The reaction mixture was used into the next step without further purification.

### Step 2. Procedure for Preparation of Compound 2—cyclopropyl(spiro[3.3]heptan-2-yl)methanone

To the solution of bromo(cyclopropyl)magnesium (1.60 g, crude) in tetrahydrofuran (8.00 mL) was added N-methoxy-N-methylspiro[3.3]heptane-2-carboxamide (500 mg, 2.73 mmol, 1.00 eq) under nitrogen atmosphere at 0° C. Then the reaction was stirred at 25° C. for 3 h. The mixture was quenched by addition of saturated aqueous ammonium chloride (20 mL), then extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=50/1) to afford cyclopropyl(spiro[3.3]heptan-2-yl)methanone (400 mg, 2.44 mmol, 89% yield) as colorless oil.

1H NMR (400 MHz, CDCl₃) δ=3.22 (quin, J=8.6 Hz, 1H), 2.21 (dt, J=1.6, 8.7 Hz, 4H), 2.07-2.02 (m, 2H), 1.89-1.77 (m, 5H), 0.98 (quin, J=3.8 Hz, 2H), 0.85-0.82 (m, 2H).

### Step 3. Procedure for Preparation of Compound 3—cyclopropyl(spiro[3.3]heptan-2-yl)methanol

To a solution of cyclopropyl(spiro[3.3]heptan-2-yl)methanone (400 mg, 2.44 mmol, 1.00 eq) in methanol (5.00 mL) were added sodium borohydride (250 mg, 6.61 mmol, 2.72 eq) at 0° C. Then the reaction was stirred at 25° C. for 0.5 h. The reaction was quenched by addition of water (10 mL), then extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=10/1 to 5/1) to afford cyclopropyl(spiro[3.3]heptan-2-yl)methanol (290 mg, 1.74 mmol, 72% yield) as colorless oil.

1H NMR (400 MHz, CDCl3) δ=2.78 (t, J=7.8 Hz, 1H), 2.38-2.27 (m, 1H), 2.10-2.00 (m, 4H), 1.89-1.77 (m, 6H), 1.44 (br s, 1H), 0.83-0.74 (m, 1H), 0.52-0.40 (m, 2H), 0.28-0.20 (m, 2H).

### Step 4. Procedure for preparation of cyclopropyl(spiro[3.3]heptan-2-yl)methyl ((2-(2,6-dioxopiperidin-3-yl)-3-oxoisoindolin-5-yl)methyl)carbamate

To the solution of cyclopropyl(spiro[3.3]heptan-2-yl)methanol (50.0 mg, 301 umol, 1.00 eq) and phenyl ((2-(2,6-dioxopiperidin-3-yl)-3-oxoisoindolin-5-yl)methyl)carbamate (118 mg, 301 umol, 1.00 eq) in dimethylformamide (0.500 mL) were added sodium hydride (18.0 mg, 451 umol, 60% purity, 1.50 eq) at 0° C. under nitrogen atmosphere. The mixture was stirred at 25° C. for 2 h. After filtration via filter paper, the organic layer was added water (5 mL) and extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=1/1 to 1/2) to afford cyclopropyl(spiro[3.3]heptan-2-yl)methyl ((2-(2,6-dioxopiperidin-3-yl)-3-oxoisoindolin-5-yl)methyl)carbamate (22.58 mg, 45.59 umol, 15% yield, 94% purity) as a white solid.

1H NMR (400 MHz, DMSO-d) δ=10.97 (s, 1H), 7.68 (br t, J=6.1 Hz, 1H), 7.61 (s, 1H), 7.57-7.48 (m, 2H), 5.11 (dd, J=5.0, 13.3 Hz, 1H), 4.46-4.23 (m, 4H), 4.05 (t, J=7.8 Hz, 1H), 2.95-2.85 (m, 1H), 2.60 (br d, J=17.5 Hz, 1H), 2.44 (br s, 2H), 2.03-1.93 (m, 5H), 1.88-1.79 (m, 3H), 1.79-1.70 (m, 3H), 0.88-0.79 (m, 1H), 0.49-0.32 (m, 2H), 0.29-0.18 (m, 2H).

### MS (ESI) m/z 466.0 [M+H]⁺.

### Example 23. Synthesis of Compound 109

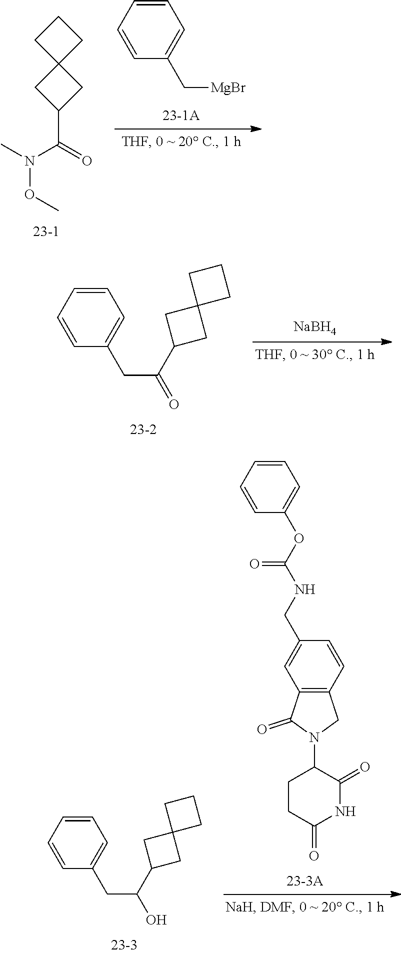

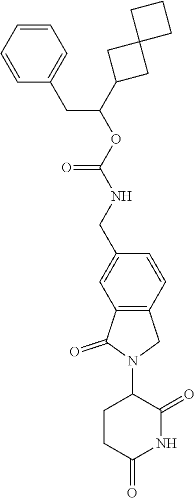 [SMILES: O=C1CCC(N2Cc3ccc(CNC(=O)OC(Cc4ccccc4)C4CC5(CCC5)C4)cc3C2=O)C(=O)N1]

### Step 1. Procedure for Preparation of Compound 2—2-phenyl-1-(spiro[3.3]heptan-2-yl)ethenone

To a solution of N-methoxy-N-methylspiro[3.3]heptane-2-carboxamide (300 mg, 1.64 mmol, 1.00 eq) in tetrahydrofuran (5.00 mL) was added benzylmagnesium bromide (1.00 M, 2.46 mL, 1.50 eq) at 0° C. under nitrogen atmosphere. Then the mixture was stirred at 20° C. for 1 h under nitrogen atmosphere. The mixture was quenched with saturated ammonium chloride (20.0 mL), extracted with ethyl acetate (3×20 mL). The combined organic layers washed with brine (30.0 mL), and dried over anhydrous sodium sulfate, filtered and concentrated under reduced pressure to give a residue. The residue was purified by column chromatography (SiO2, petroleum ether/ethyl acetate=10/0 to 10/1) to afford 2-phenyl-1-(spiro[3.3]heptan-2-yl)ethanone (250 mg, 1.17 mmol, 71% yield) as colorless oil.

1H NMR (400 MHz, CDCl3) δ=7.35-7.29 (m, 2H), 7.27 (m, 1H), 7.21-7.17 (m, 2H), 3.64 (s, 2H), 3.18 (t, J=8.6 Hz, 1H), 2.22-2.15 (m, 2H), 2.10-2.04 (m, 2H), 2.00 (t, J=7.0 Hz, 2H), 1.89-1.83 (m, 2H), 1.82-1.75 (m, 2H).

### Step 2. Procedure for Preparation of Compound 3—2-phenyl-1-(spiro[3.3]heptan-2-yl)ethanol

To a solution of 2-phenyl-1-(spiro[3.3]heptan-2-yl)ethanone (250 mg, 1.17 mmol, 1.00 eq) in tetrahydrofuran (3.00 mL) was added sodium borohydride (88.2 mg, 2.33 mmol, 2.00 eq) at 0° C. The mixture was stirred at 30° C. for 1 h. The mixture was quenched with saturated ammonium chloride (10 mL), extracted with ethyl acetate (3×10.0 mL). The combined organic layers washed with brine (20.0 mL), and dried over anhydrous sodium sulfate, filtered and concentrated under reduced pressure to afford 2-phenyl-1-(spiro[3.3]heptan-2-yl) ethanol (200 mg, 924 umol, 79% yield) as a yellow solid.

1H NMR (400 MHz, CDCl3) δ=7.35-7.29 (m, 2H), 7.26-7.20 (m, 3H), 3.68 (dt, J=3.9, 8.0 Hz, 1H), 2.76 (dd, J=3.9, 13.6 Hz, 1H), 2.52 (dd, J=8.3, 13.7 Hz, 1H), 2.26-2.15 (m, 1H), 2.12-2.06 (m, 1H), 2.05-1.98 (m, 3H), 1.92-1.85 (m, 3H), 1.84-1.80 (m, 2H), 1.79-1.73 (m, 2H).

### Step 3. Procedure for preparation of 2-phenyl-1-(spiro[3.3]heptan-2-yl)ethyl ((2-(2,6-dioxopiperidin-3-yl)-3-oxoisoindolin-5-yl)methyl)carbamate

To a solution of phenyl ((2-(2,6-dioxopiperidin-3-yl)-3-oxoisoindolin-5-yl)methyl)carbamate (150 mg, 381 umol, 1.00 eq) and 2-phenyl-1-(spiro[3.3]heptan-2-yl)ethanol (82.4 mg, 381 umol, 1.00 eq) in dimethylformamide (1.50 mL) was added sodium hydride (30.5 mg, 762 umol, 60% purity, 2.00 eq) at 0° C. The mixture was stirred at 20° C. for 1 h. The mixture was quenched with formic acid (0.5 mL) at 0° C. The residue was purified by reverse phase column chromatography (C18, 80 g; condition: water/acetonitrile=1/0 to 0/1) and lyophilized to afford 2-phenyl-1-(spiro[3.3]heptan-2-yl)ethyl ((2-(2,6-dioxopiperidin 3-yl)-3-oxoisoindolin-5-yl)methyl)carbamate (43.9 mg, 84.3 umol, 22% yield, 99% purity) as a white solid.

1H NMR (400 MHz, DMSO-d6) δ=10.97 (s, 1H), 7.69 (br t, J=6.0 Hz, 1H), 7.59 (s, 1H), 7.52 (br d, J=7.8 Hz, 1H), 7.40 (br d, J=7.8 Hz, 1H), 7.30-7.21 (m, 2H), 7.20-7.13 (m, 3H), 5.12 (br dd, J=5.1, 13.3 Hz, 1H), 4.83-4.73 (m, 1H), 4.46-4.40 (m, 1H), 4.35-4.27 (m, 1H), 4.23 (br t, J=6.1 Hz, 2H), 2.96-2.86 (m, 1H), 2.75-2.65 (m, 2H), 2.60 (br d, J=18.3 Hz, 1H), 2.41 (br d, J=4.1 Hz, 1H), 2.22 (br dd, J=7.8, 15.8 Hz, 1H), 1.93 (br d, J=4.5 Hz, 5H), 1.83 (br t, J=7.9 Hz, 3H), 1.77-1.70 (m, 2H), 1.67 (br d, J=9.9 Hz, 1H).

### MS (ESI) m/z 538.1 [M+Na]⁺.

### Example 24. Synthesis of Compound 110

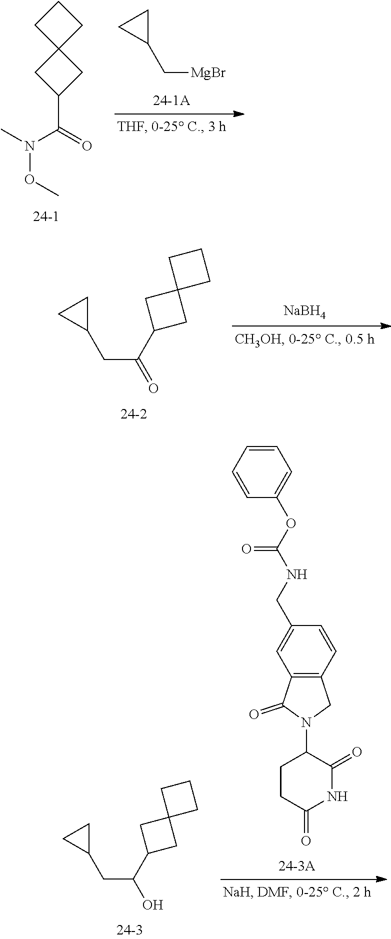

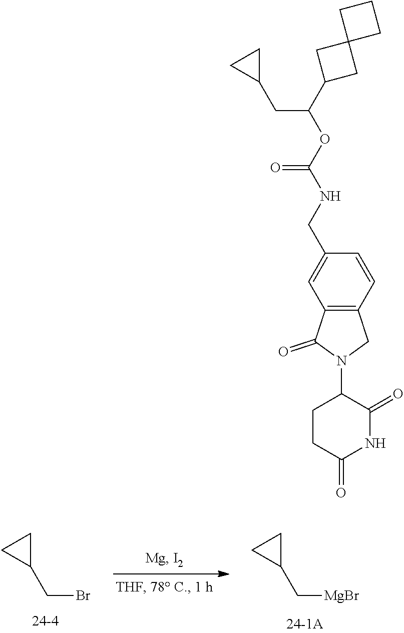

### Step 1. Procedure for Preparation of Compound 4A—cyclopropylmagnesium bromide

To a solution of magnesium dihydride (321 mg, 13.2 mmol, 1.20 eq), iodide (2.79 mg, 11.0 umol, 0.00100 eq) in tetrahydrofuran (8.00 mL) was added (bromomethyl)cyclopropane (1.25 g, 11.0 mmol, 886 uL, 1.00 eq) at 78° C. under nitrogen atmosphere. Then the reaction was stirred at 78° C. for 1 h. The solution of bromo(cyclopropyl)magnesium (1.75 g, crude) in tetrahydrofuran (8.00 mL) was obtained as a gray liquid.

Note: The reaction mixture was used into the next step without further purification.

### Step 2. Procedure for Preparation of Compound 2—2-cyclopropyl-1-(spiro[3.3]heptan-2-yl)ethenone

To the solution of (cyclopropylmethyl)magnesium bromide (1.75 g, crude) in tetrahydrofuran (8.00 mL) was added N-methoxy-N-methylspiro[3.3]heptane-2-carboxamide (500 mg, 2.73 mmol, 1.00 eq) under nitrogen atmosphere at 0° C. Then the reaction was stirred at 25° C. for 3 h. The mixture was quenched by of 20 mL of saturated ammonium chloride aqueous (20 mL), then extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=50/1) to afford 2-cyclopropyl-1-(spiro[3.3]heptan-2-yl)ethanone (378 mg, 2.12 mmol, 78% yield) as colorless oil.

1H NMR (400 MHz, CDCl3) δ=5.86-5.75 (m, 1H), 5.05-4.95 (m, 2H), 3.14-3.04 (m, 1H), 2.46-2.42 (m, 2H), 2.34-2.29 (m, 2H), 2.18-2.11 (m, 4H), 2.03 (t, J=7.2 Hz, 2H), 1.90-1.85 (m, 2H), 1.83-1.75 (m, 2H)

### Step 3. Procedure for Preparation of Compound 3—2-cyclopropyl-1-(spiro[3.3]heptan-2-yl)ethanol

To a solution of 2-cyclopropyl-1-(spiro[3.3]heptan-2-yl)ethanone (378 mg, 2.12 mmol, 1.00 eq) in methanol (5.00 mL) was added sodium borohydride (160 mg, 4.24 mmol, 2.00 eq) at 0° C. Then the reaction was stirred at 25° C. for 0.5 h. The reaction was quenched by addition of saturated ammonium chloride aqueous (10 mL), then extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=10/1 to 5/1) to afford 2-cyclopropyl-1-(spiro[3.3]heptan-2-yl)ethanol (330 mg, 1.83 mmol, 86% yield) as colorless oil.

1H NMR (400 MHz, CDCl3) δ=5.90-5.77 (m, 1H), 5.08-4.94 (m, 2H), 3.45 (dt, J=3.4, 8.1 Hz, 1H), 2.20-1.98 (m, 7H), 1.87-1.75 (m, 5H), 1.68 (dd, J=8.6, 10.8 Hz, 1H), 1.54-1.45 (m, 1H), 1.42-1.31 (m, 2H).

### Step 4. Procedure for preparation of 2-cyclopropyl-1-(spiro[3.3]heptan-2-yl)ethyl ((2-(2,6-dioxopiperidin-3-yl)-3-oxoisoindolin-5-yl)methyl)carbamate

To the solution of 2-cyclopropyl-1-(spiro[3.3]heptan-2-yl)ethanol (50.0 mg, 277 umol, 1.00 eq) and phenyl ((2-(2,6-dioxopiperidin-3-yl)-3-oxoisoindolin-5-yl)methyl)carbamate (109 mg, 277 umol, 1.00 eq) in dimethylformamide (0.50 mL) was added sodium hydride (16.6 mg, 416 umol, 60% purity, 1.50 eq) at 0° C. under nitrogen atmosphere. The mixture was stirred at 25° C. for 2 h. After filtration via filter paper, the organic layer was added water (10 mL) and then extracted with ethyl acetate (2×10 mL). The combined organic layers were washed with brine (2×10 mL), and dried over anhydrous sodium sulfate, filtered and concentrate to give a residue. The residue was purified by column chromatography (SiO2, petroleum ether/ethyl acetate=1/1 to 1/2) to afford 2-cyclopropyl-1-(spiro[3.3]heptan-2-yl)ethyl ((2-(2,6-dioxopiperidin-3-yl)-3-oxoisoindolin-5-yl)methyl)carbamate (17.81 mg, 34.91 umol, 12% yield, 94% purity) as a white solid.

1H NMR (400 MHz, DMSO-d) δ=10.97 (s, 1H), 7.70 (br t, J=5.7 Hz, 1H), 7.61 (s, 1H), 7.57-7.47 (m, 2H), 5.83-5.68 (m, 1H), 5.11 (br dd, J=4.9, 13.2 Hz, 1H), 5.04-4.82 (m, 2H), 4.65-4.57 (m, 1H), 4.46-4.30 (m, 2H), 4.29-4.23 (m, 2H), 2.97-2.85 (m, 1H), 2.60 (br d, J=16.9 Hz, 1H), 2.43-2.34 (m, 1H), 2.32-2.17 (m, 1H), 2.07-1.86 (m, 7H), 1.84-1.64 (m, 6H), 1.53-1.37 (m, 2H).

### MS (ESI) m/z 480.2 [M+H]⁺.

### Example 25. Synthesis of Compound 111

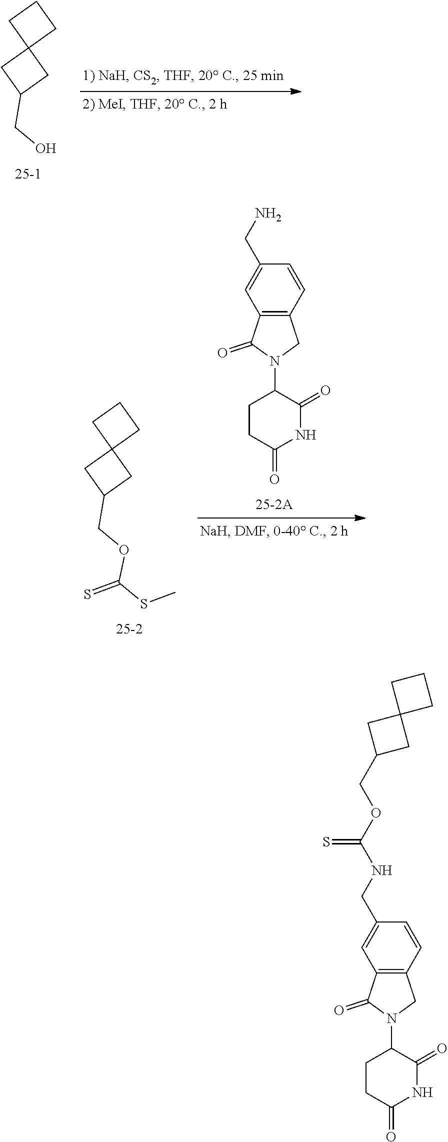

### Step 1. Procedure for Preparation of Compound 2—S-methyl O-(spiro[3.3]heptan-2-ylmethyl) carbonodithioate

To a solution of spiro[3.3]heptan-2-ylmethanol (50.0 mg, 396 umol, 1.00 eq) in tetrahydrofuran (1.00 mL) was added sodium hydride (19.0 mg, 475 umol, 60% purity, 1.20 eq). The mixture was stirred at 20° C. for 20 min. Then, carbon disulfide (45.3 mg, 594 umol, 35.9 uL, 1.50 eq) was added into the mixture. The reaction mixture was stirred at 20° C. for 5 min. The solution of O-(spiro[3.3]heptan-2-ylmethyl)S-hydrogen carbonodithioate (80.0 mg, 395 umol, 99% yield) in tetrahydrofuran (1.00 mL) was obtained as colorless liquid, and the solution was used into the next step without further purification.

To a solution of O-(spiro[3.3]heptan-2-ylmethyl)S-hydrogen carbonodithioate (80.0 mg, 395 umol, 1.00 eq) in tetrahydrofuran (1.00 mL) was added iodomethane (67.3 mg, 474 umol, 29.5 uL, 1.20 eq). The reaction mixture was stirred at 20° C. for 2 h. The reaction mixture was filtered and concentrated under reduced pressure to give a residue. The residue was purified by column chromatography (SiO2, petroleum ether/ethyl acetate=1/0 to 5/1) and concentrated under reduced pressure to afford S-methyl O-(spiro[3.3]heptan-2-ylmethyl) carbonodithioate (70.0 mg, 323 umol, 81% yield) as colorless oil.

1H NMR (400 MHz, CDCl3-d) δ=4.46 (d, J=6.9 Hz, 2H), 2.55 (td, J=7.4, 15.1 Hz, 1H), 2.48 (s, 3H), 2.12-2.04 (m, 2H), 1.98-1.91 (m, 2H), 1.89-1.82 (m, 2H), 1.80-1.65 (m, 4H).

### Step 2. Procedure for preparation of O-(spiro[3.3]heptan-2-ylmethyl) ((2-(2,6-dioxopiperidin-3-yl)-3-oxoisoindolin-5-yl)methyl)carbamothioate

To a solution of S-methyl O-(spiro[3.3]heptan-2-ylmethyl) carbonodithioate (50.0 mg, 231 umol, 1.00 eq) in dimethylformamide (1.00 mL) was added 3-(6-(aminomethyl)-1-oxoisoindolin-2-yl)piperidine-2,6-dione (63.1 mg, 231 umol, 1.00 eq) and sodium hydride (11.1 mg, 462 umol, 2.00 eq) at 0° C. The reaction mixture was stirred at 40° C. for 2 h. The reaction mixture was quenched by adding ice cold water (10 mL), extracted with ethyl acetate (2×10 mL). The combined organic layers were washed with water (10 mL), brine (10 mL), dried over sodium sulfate and concentrated under reduced pressure to give a residue. The residue was purified by Prep-HPLC (column: Phenomenex Luna C18 150*25 mm*10 um; mobile phase: [water(formic acid)-acetonitrile]; B %: 45%-75%, 10 min) and lyophilized to afford 0-(spiro[3.3]heptan-2-ylmethyl) ((2-(2,6-dioxopiperidin-3-yl)-3-oxoisoindolin-5-yl)methyl)carbamothioate (23.42 mg, 52.51 umol, 22% yield, 99% purity) as a white solid.

1H NMR (400 MHz, DMSO-d6) δ=10.98 (br s, 1H), 9.79-9.61 (m, 1H), 7.64-7.43 (m, 3H), 5.17-5.07 (m, 1H), 4.71 (d, J=5.8 Hz, 1H), 4.48-4.37 (m, 2H), 4.35-4.30 (m, 2H), 4.30-4.24 (m, 1H), 2.97-2.85 (m, 1H), 2.63-2.56 (m, 1H), 2.48-2.44 (m, 1H), 2.43-2.37 (m, 1H), 2.11-2.01 (m, 2H), 2.00-1.95 (m, 2H), 1.90 (br t, J=8.2 Hz, 2H), 1.81-1.70 (m, 4H), 1.69-1.51 (m, 1H).

### MS (ESI) m/z 463.9 [M+Na]⁺.

### Example 26. Synthesis of Compound 112

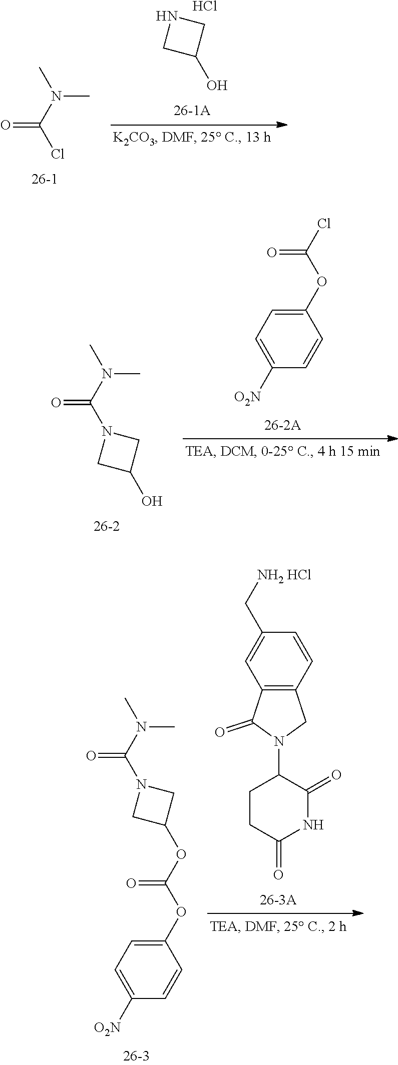

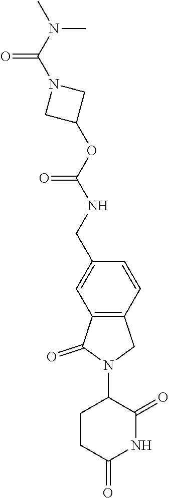 [SMILES: CN(C)C(=O)N1CC(OC(=O)NCc2ccc3c(c2)C(=O)N(C2CCC(=O)NC2=O)C3)C1]

### Step 1. Procedure for Preparation of Compound 2—3-hydroxy-N,N-dimethylazetidine-1-carboxamide

To a solution of azetidin-3-ol (2.04 g, 18.6 mmol, 2.00 eq, hydrochloride) in dimethylformamide (15.0 mL) was added potassium carbonate (2.57 g, 18.6 mmol, 2.00 eq). After stirring at 25° C. for 1 h, dimethylcarbamic chloride (1.00 g, 9.3 mmol, 1.00 eq) was added into the mixture. Then the reaction was stirred at 25° C. for 12 h. The mixture was diluted with water (20 mL), extracted with dichloromethane/isopropanol (3/1, 3×20 mL). The combined organic layers were washed with brine (2×10 mL), and dried over anhydrous sodium sulfate, filtered and concentrate to give a residue. The residue was purified by column chromatography (SiO2, petroleum ether/ethyl acetate=0/1) to afford 3-hydroxy-N,N-dimethylazetidine-1-carboxamide (940 mg, 6.52 mmol, 70% yield) as colorless oil.

1H NMR (400 MHz, CDCl3) δ=4.55 (br d, J=3.4 Hz, 1H), 4.17 (dd, J=6.8, 9.5 Hz, 2H), 3.90-3.81 (m, 3H), 2.84 (s, 6H).

### Step 2. Procedure for Preparation of Compound 3—1-(dimethylcarbamoyl)azetidin-3-yl (4-nitrophenyl) carbonate

To the solution of 3-hydroxy-N,N-dimethylazetidine-1-carboxamide (50.0 mg, 347 umol, 1.00 eq) in dichloromethane (1.00 mL) was added triethylamine (175 mg, 1.73 mmol, 5.00 eq). The mixture was stirred at 25° C. for 15 min and then cooled to 0° C. Then 4-nitrophenyl carbonochloridate (245 mg, 1.21 mmol, 3.50 eq) was added into the mixture at 0° C. and then the reaction was stirred at 25° C. for 4 h. The reaction mixture was concentrated under reduced pressure to give a residue. The residue was purified by column chromatography (SiO2, petroleum ether/ethyl acetate=5/1 to 0/1) to afford 1-(dimethylcarbamoyl)azetidin-3-yl (4-nitrophenyl) carbonate (20.0 mg, 45.3 umol, 13% yield, 70% purity) as colorless oil.

1H NMR (400 MHz, CDCl3) δ=8.33-8.27 (m, 2H), 7.42-7.36 (m, 2H), 5.27 (tt, J=4.2, 6.7 Hz, 1H), 4.40-4.35 (m, 2H), 4.14-4.10 (m, 2H), 2.90 (s, 6H).

### Step 3. Procedure for preparation of 1-(dimethylcarbamoyl)azetidin-3-yl ((2-(2,6-dioxopiperidin-3-yl)-3-oxoisoindolin-5-yl)methyl)carbamate

To the solution of 1-(dimethylcarbamoyl)azetidin-3-yl (4-nitrophenyl) carbonate (20.0 mg, 45.3 umol, 70% purity, 1.00 eq), 3-(6-(aminomethyl)-1-oxoisoindolin-2-yl)piperidine-2,6-dione (14.0 mg, 45.3 umol, 1.00 eq, hydrochloride) in dimethylformamide (1.00 mL) was added triethylamine (9.16 mg, 90.5 umol, 2.00 eq). Then the reaction was stirred at 25° C. for 2 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 4%-34%, 10 min) and lyophilized to afford 1-(dimethylcarbamoyl)azetidin-3-yl((2-(2,6-dioxopiperidin-3-yl)-3-oxoisoindolin-5-yl)methyl) carbamate (15.58 mg, 31.51 umol, 70% yield, 99% purity, formate) as a white solid.

1H NMR (400 MHz, DMSO-d6) δ=10.97 (br d, J=2.5 Hz, 1H), 8.46 (br s, 1H), 8.03 (br s, 1H), 7.61 (s, 1H), 7.57-7.50 (m, 2H), 5.11 (br dd, J=4.6, 13.3 Hz, 1H), 5.01 (br d, J=4.6 Hz, 1H), 4.46-4.40 (m, 1H), 4.33-4.27 (m, 3H), 4.15 (br t, J=7.9 Hz, 2H), 3.78-3.73 (m, 2H), 2.91 (br t, J=13.4 Hz, 1H), 2.74 (s, 6H), 2.60 (br d, J=13.9 Hz, 1H), 2.45-2.37 (m, 1H), 2.04-1.97 (m, 1H).

### MS (ESI) m/z 444.0 [M+H]⁺.

### Example 27. Synthesis of Compound 113

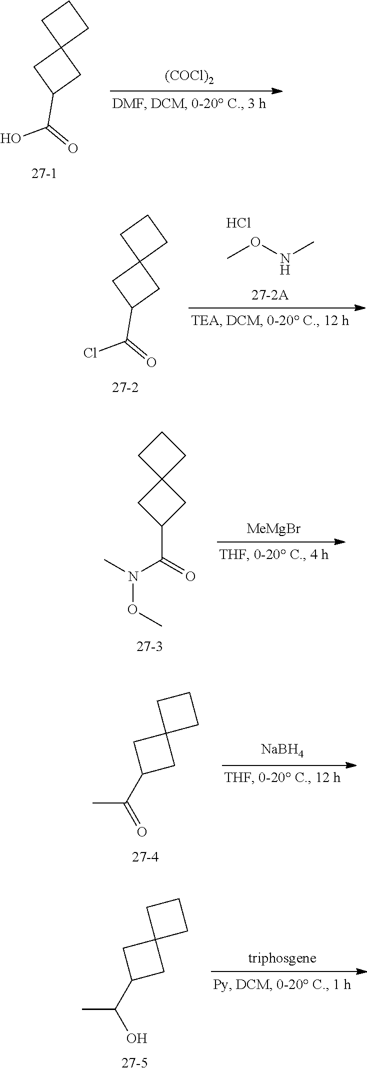

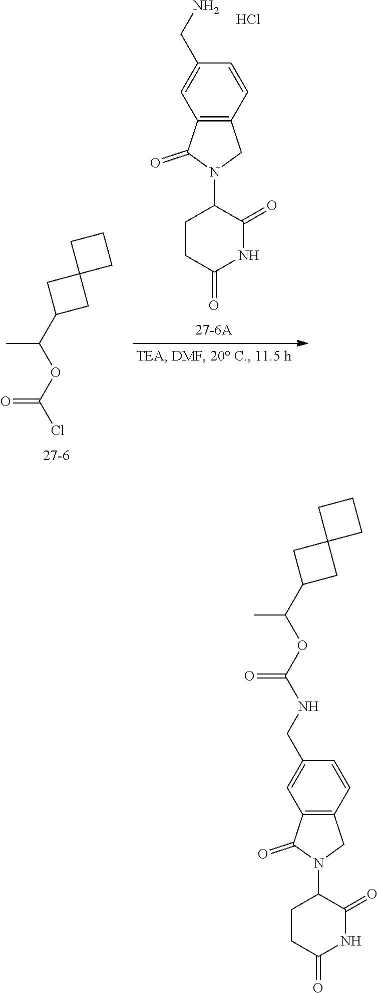

### Step 1. Procedure for Preparation of Compound 2—spiro[3.3]heptane-2-carbonyl chloride

To a solution of spiro[3.3]heptane-2-carboxylic acid (2.00 g, 14.3 mmol, 1.00 eq) in dichloromethane (10.0 mL) was added oxalyl dichloride (1.99 g, 15.7 mmol, 1.37 mL, 1.10 eq) and dimethylformamide (10.4 mg, 143 umol, 11.0 uL, 0.0100 eq) at 0° C. The reaction mixture was stirred at 20° C. for 3 h. The reaction mixture was concentrated under reduced pressure to afford spiro[3.3]heptane-2-carbonyl chloride (2.26 g, 14.2 mmol, 100% yield) as colorless oil.

### Step 2. Procedure for Preparation of Compound 3—N-methoxy-N-methylspiro[3.3]heptane-2-carboxamide

To a solution of spiro[3.3]heptane-2-carbonyl chloride (2.26 g, 14.3 mmol, 1.00 eq) in dichloromethane (10.0 mL) was added N,O-dimethylhydroxylamine (1.67 g, 17.1 mmol, 1.20 eq, hydrochloride) and triethylamine (4.33 g, 42.7 mmol, 5.95 mL, 3.00 eq) at 0° C. The reaction mixture was stirred at 20° C. for 12 h. The reaction mixture was added water (50 mL) and extracted with ethyl acetate (3×50 mL). The combined organic layers were dried over anhydrous sodium sulfate, filtered and concentrated under reduced pressure to afford N-methoxy-N-methylspiro[3.3]heptane-2-carboxamide (2.50 g, 13.6 mmol, 96% yield) as yellow oil.

1H NMR (400 MHz, CDCl3) δ=3.57 (s, 3H), 3.25 (br d, J=8.9 Hz, 1H), 3.09 (s, 3H), 2.26-2.15 (m, 2H), 2.13-2.04 (m, 2H), 1.99 (t, J=7.2 Hz, 2H), 1.88-1.80 (m, 2H), 1.78-1.68 (m, 2H).

### Step 3. Procedure for Preparation of Compound 4—1-(spiro[3.3]heptan-2-yl)ethenone

To a solution of N-methoxy-N-methylspiro[3.3]heptane-2-carboxamide (1.00 g, 5.46 mmol, 1.00 eq) in tetrahydrofuran (10.0 mL) was added bromo(methyl)magnesium (3 M, 2.73 mL, 1.50 eq) at 0° C. The reaction mixture was stirred at 20° C. for 4 h. The reaction mixture was quenched with hydrochloric acid (1 N, 20 mL), and extracted with ethyl acetate (3×50 mL). The combined organic layers were washed with brine (50 mL), dried over anhydrous sodium sulfate, filtered and concentrated under reduced pressure to give a residue. The residue was purified by chromatography on silica gel (petroleum ether/ethyl acetate=1/0 to 100/4) and concentrated under reduced pressure to afford 1-(spiro[3.3]heptan-2-yl)ethanone (600 mg, 4.34 mmol, 80% yield) as colorless oil.

1H NMR (400 MHz, CDCl3) δ=3.08 (quin, J=8.6 Hz, 1H), 2.17 (s, 2H), 2.15 (s, 2H), 2.07 (s, 3H), 2.04-2.00 (m, 2H), 1.91-1.85 (m, 2H), 1.83-1.74 (m, 2H).

### Step 4. Procedure for Preparation of Compound 5—1-(spiro[3.3]heptan-2-yl)ethanol

To a solution of 1-(spiro[3.3]heptan-2-yl)ethanone (200 mg, 1.45 mmol, 1.00 eq) in methanol (1.00 mL) was added sodium borohydride (109 mg, 2.89 mmol, 2.00 eq) at 0° C. The reaction mixture was stirred at 20° C. for 12 h. The reaction mixture was concentrated under reduced pressure, added into saturated ammonium chloride (10 mL) and extracted with ethyl acetate (3×30 mL). The organic layers were washed with brine (3×20 mL), dried over anhydrous sodium sulfate, filtered and under reduced pressure to afford 1-spiro[3.3]heptan-2-ylethanol (120 mg, 856 umol, 59% yield) as colorless oil.

1H NMR (400 MHz, CDCl₃) δ=3.55 (quin, J=6.5 Hz, 1H), 2.05-1.97 (m, 2H), 1.96-1.90 (m, 3H), 1.82-1.76 (m, 2H), 1.74-1.66 (m, 3H), 1.62-1.54 (m, 1H), 1.00 (d, J=6.1 Hz, 3H).

### Step 5. Procedure for Preparation of Compound 6—1-(spiro[3.3]heptan-2-yl)ethyl carbonochloridate

To a solution of 1-spiro[3.3]heptan-2-ylethanol (50.0 mg, 357 umol, 1.00 eq) in dichloromethane (1.00 mL) was added pyridine (56.0 mg, 713 umol, 57.6 uL, 2.00 eq) and bis(trichloromethyl) carbonate (106 mg, 357 umol, 1.00 eq) at 0° C. The reaction mixture was stirred at 20° C. for 1 h. The reaction mixture was concentrated under reduced pressure to afford 1-(spiro[3.3]heptan-2-yl)ethyl carbonochloridate (72.0 mg, crude) as a white solid.

### Step 6. Procedure for preparation of 1-(spiro[3.3]heptan-2-yl)ethyl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (55.0 mg, 178 umol, 1.00 eq, hydrochloride) in dimethylformamide (0.500 mL) was added 1-(spiro[3.3]heptan-2-yl)ethyl carbonochloridate (72.0 mg, 355 umol, 2.00 eq) and pyridine (42.1 mg, 533 umol, 43.0 uL, 3.00 eq). The reaction mixture was stirred at 20° C. for 12 h. The reaction mixture was filtered. The filtrate was purified by Prep-HPLC (column: Phenomenex luna C18 150*25 mm*10 um; mobile phase: [water (formic acid)-acetonitrile]; B %: 41%-71%, 10 min) and lyophilized to give residue. The residue was triturated with petroleum ether (5 mL) at 20° C. for 1 h to afford 1-(spiro[3.3]heptan-2-yl)ethyl ((2-(2,6-dioxopiperidin-3-yl)-3-oxoisoindolin-5-yl)methyl)carbamate (6.65 mg, 14.83 umol, 35% yield, 98% purity) as a yellow solid.

1H NMR (400 MHz, DMSO-d6) δ=10.99 (s, 1H), 7.68 (br t, J=6.3 Hz, 1H), 7.61 (s, 1H), 7.58-7.53 (m, 1H), 7.52-7.47 (m, 1H), 5.11 (dd, J=5.0, 13.3 Hz, 1H), 4.70-4.54 (m, 1H), 4.49-4.40 (m, 1H), 4.35-4.19 (m, 3H), 2.98-2.81 (m, 1H), 2.63 (br s, 1H), 2.43-2.35 (m, 1H), 2.18 (quin, J=7.9 Hz, 1H), 2.06-1.90 (m, 5H), 1.88-1.82 (m, 2H), 1.80-1.74 (m, 2H), 1.73-1.63 (m, 2H), 1.04 (br d, J=6.1 Hz, 3H).

1H NMR (400 MHz, DMSO-d₆, T=80° C.) δ=10.73 (br s, 1H), 7.63 (s, 1H), 7.58-7.47 (m, 2H), 7.35 (br d, J=2.1 Hz, 1H), 5.07 (dd, J=5.2, 13.1 Hz, 1H), 4.67-4.58 (m, 1H), 4.50-4.41 (m, 1H), 4.39-4.32 (m, 1H), 4.29 (d, J=6.3 Hz, 2H), 2.90 (ddd, J=5.5, 13.4, 17.5 Hz, 1H), 2.69-2.63 (m, 1H), 2.45-2.37 (m, 1H), 2.26-2.16 (m, 1H), 2.08-1.94 (m, 5H), 1.88-1.82 (m, 2H), 1.77 (q, J=6.9 Hz, 3H), 1.72-1.65 (m, 1H), 1.05 (d, J=6.1 Hz, 3H.)

### MS (ESI) m/z 462.3 [M+Na]⁺.

### Example 28. Synthesis of Compound 115

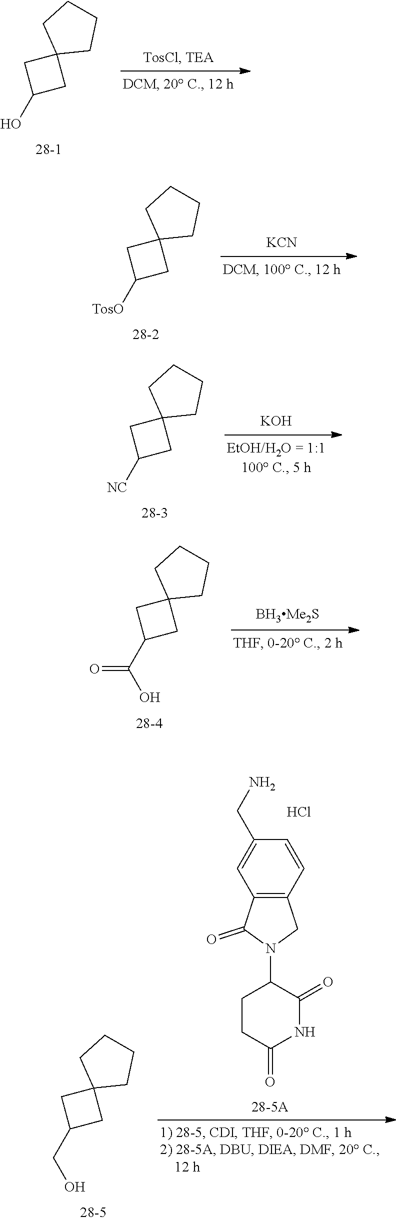

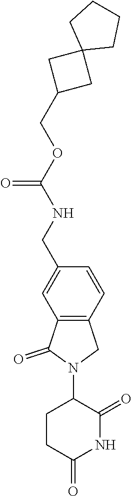 [SMILES: O=C1CCC(N2Cc3ccc(CNC(=O)OCC4CC5(CCCC5)C4)cc3C2=O)C(=O)N1]

### Step 1. Procedure for Preparation of Compound 2—spiro[3.4]octan-2-yl 4-methylbenzenesulfonate

To a solution of spiro[3.4]octan-2-ol (500 mg, 3.96 mmol, 1.00 eq) in dichloromethane (5.00 mL) were added triethylamine (802 mg, 7.92 mmol, 1.10 mL, 2.00 eq) and 4-methylbenzene-1-sulfonyl chloride (755 mg, 3.96 mmol, 1.00 eq). The reaction mixture was stirred at 20° C. for 12 h. The reaction mixture was filtered and the filtrate was concentrated under reduced pressure to give a residue. The residue was purified by column chromatography (SiO2, petroleum ether/ethyl acetate=1/0 to 20/1) to afford spiro[3.4]octan-2-yl 4-methylbenzenesulfonate (690 mg, 2.46 mmol, 62% yield) as yellow oil.

1H NMR (400 MHz, CDCl₃) δ=7.79 (d, J=8.1 Hz, 2H), 7.34 (d, J=8.1 Hz, 2H), 4.80 (quin, J=7.4 Hz, 1H), 2.46 (s, 3H), 2.18-2.10 (m, 2H), 2.10-2.03 (m, 2H), 1.59-1.48 (m, 8H).

### Step 2. Procedure for Preparation of Compound 3—spiro[3.4]octane-2-carbonitrile

To a solution of spiro[3.4]octan-2-yl 4-methylbenzenesulfonate (690 mg, 2.46 mmol, 1.00 eq) in dimethylsulfoxide (6.00 mL) was added potassium cyanide (321 mg, 4.92 mmol, 211 uL, 2.00 eq). The reaction mixture was stirred at 100° C. for 12 h. The reaction mixture was added to water (50 mL) to quench, and then the mixture was extracted with ethyl acetate (2×30 mL). The organic layers were combined, dried over anhydrous sodium sulfate, filtered and concentrated under reduced pressure to a residue. The residue was purified by column chromatography (SiO2, petroleum ether/ethyl acetate=1/0 to 50/1) to afford spiro[3.4]octane-2-carbonitrile (110 mg, 814 umol, 33% yield) as yellow oil.

1H NMR (400 MHz, CDCl₃) δ=3.00 (quin, J=8.5 Hz, 1H), 2.31-2.22 (m, 4H), 1.69-1.64 (m, 2H), 1.62-1.54 (m, 6H).

### Step 3. Procedure for Preparation of Compound 4—spiro[3.4]octane-2-carboxylic acid

To a solution of spiro[3.4]octane-2-carbonitrile (110 mg, 814 umol, 1.00 eq) in ethanol (1.00 mL) and water (1.00 mL) was added potassium hydroxide (183 mg, 3.25 mmol, 4.00 eq). The reaction mixture was stirred at 100° C. for 5 h. The reaction mixture was adjusted pH to 4 with 1 N hydrochloric acid, and then the mixture was extracted with ethyl acetate (3×10 mL). The combined organic phases were dried with anhydrous sodium sulfate, filtered and concentrated under reduced pressure to afford spiro[3.4]octane-2-carboxylic acid (60.0 mg, 389 umol, 48% yield) as yellow oil.

1H NMR (400 MHz, CDCl₃) δ=3.08 (quin, J=8.7 Hz, 1H), 2.26-2.18 (m, 2H), 2.13-2.07 (m, 2H), 1.67-1.62 (m, 2H), 1.61-1.50 (m, 6H).

### Step 4. Procedure for Preparation of Compound 5—spiro[3.4]octan-2-ylmethanol

To a solution of spiro[3.4]octane-2-carboxylic acid (60.0 mg, 389 umol, 1.00 eq) in tetrahydrofuran (1.00 mL) was added borane dimethyl sulfide complex (10.0 M, 77.8 uL, 2.00 eq) at 0° C. The reaction mixture was stirred at 20° C. for 2 h. The reaction mixture was added to methanol (10 mL) to quench. Then the mixture was concentrated under reduced pressure to afford spiro[3.4]octan-2-ylmethanol (40.0 mg, 285 umol, 73% yield) as yellow oil.

1H NMR (400 MHz, CDCl₃) δ=3.59 (d, J=6.9 Hz, 2H), 2.48-2.35 (m, 1H), 1.96-1.88 (m, 2H), 1.66-1.60 (m, 4H), 1.59-1.50 (m, 6H).

### Step 5. Procedure for Preparation of spiro[3.4]octan-2-ylmethyl ((2-(2,6-dioxopiperidin-3-yl)-3-oxoisoindolin-5-yl)methyl)carbamate

To a solution of spiro[3.4]octan-2-ylmethanol (30.0 mg, 214 umol, 1.00 eq) in tetrahydrofuran (0.500 mL) was added di(1H-imidazol-1-yl)methanone (34.7 mg, 214 umol, 1.00 eq) at 0° C. The reaction mixture was stirred at 20° C. for 1 h. The resulting solution was added to a mixture of 3-(6-(aminomethyl)-1-oxoisoindolin-2-yl)piperidine-2,6-dione (65.0 mg, 210 umol, 1.00 eq, hydrochloride), 2,3,4,6,7,8,9,10-octahydropyrimido[1,2-a]azepine (32.0 mg, 210 umol, 31.6 uL, 1.00 eq) and N,N-diisopropylethylamine (27.1 mg, 210 umol, 36.6 uL, 1.00 eq) in dimethylformamide (0.500 mL). The reaction mixture was stirred at 20° C. for 12 h. The reaction mixture was concentrated under reduced pressure to give a residue. The residue was dissolved in dimethylformamide (1.5 mL) and then filtered. The filtrate was purified by Prep-HPLC (column: Phenomenex luna C18 150*25 mm*10 um; mobile phase: [water (formic acid)-acetonitrile]; B %: 40%-70%, 9 min) and lyophilized to afford spiro[3.4]octan-2-ylmethyl ((2-(2,6-dioxopiperidin-3-yl)-3-oxoisoindolin-5-yl)methyl)carbamate (12.32 mg, 27.47 umol, 13% yield, 98% purity) as an off-white solid.

1H NMR (400 MHz, DMSO-d6) δ=10.98 (s, 1H), 7.76 (br t, J=6.0 Hz, 1H), 7.60 (s, 1H), 7.57-7.52 (m, 1H), 7.52-7.48 (m, 1H), 5.11 (dd, J=5.0, 13.4 Hz, 1H), 4.47-4.39 (m, 1H), 4.34-4.23 (m, 3H), 3.97-3.88 (m, 2H), 2.97-2.85 (m, 1H), 2.62-2.57 (m, 1H), 2.47-2.41 (m, 1H), 2.41-2.34 (m, 1H), 2.05-1.95 (m, 1H), 1.92-1.80 (m, 2H), 1.68-1.61 (m, 2H), 1.58 (br d, J=6.0 Hz, 2H), 1.54-1.43 (m, 6H).

1H NMR (400 MHz, DMSO-d6, T=80° C.) δ=10.73 (br d, J=4.8 Hz, 1H), 7.63 (s, 1H), 7.56-7.49 (m, 2H), 7.49-7.34 (m, 1H), 5.07 (dd, J=5.1, 13.2 Hz, 1H), 4.47-4.41 (m, 1H), 4.37-4.31 (m, 1H), 4.28 (d, J=6.1 Hz, 2H), 3.96 (d, J=6.6 Hz, 2H), 2.95-2.84 (m, 1H), 2.66-2.60 (m, 1H), 2.47-2.36 (m, 2H), 2.09-2.00 (m, 1H), 1.88 (br t, J=9.7 Hz, 2H), 1.69-1.63 (m, 2H), 1.59 (br d, J=5.4 Hz, 2H), 1.56-1.46 (m, 6H).

### MS (ESI) m/z 440.2 [M+H]⁺.

### Example 29. Synthesis of Compound 116

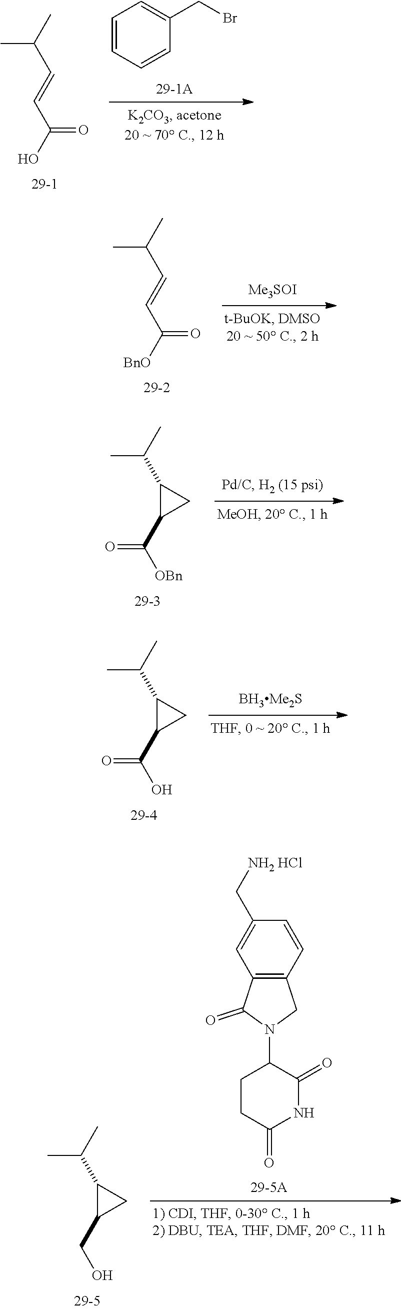

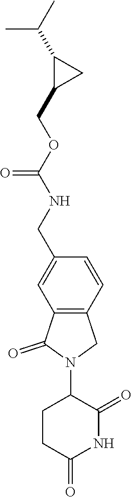

### Step 1. Procedure for Preparation of Compound 2—(E)-benzyl 4-methylpent-2-enoate

To a solution of (E)-4-methylpent-2-enoic acid (3.00 g, 26.2 mmol, 3.13 mL, 1.00 eq) in acetone (30.0 mL) was added potassium carbonate (7.26 g, 52.5 mmol, 2.00 eq). The mixture was stirred at 20° C. for 0.5 h. A solution of (bromomethyl)benzene (4.49 g, 26.2 mmol, 3.12 mL, 1.00 eq) in acetone (10 mL) was added into the mixture. The mixture was stirred at 70° C. for 11.5 h. The mixture was filtered and the filtrate was concentrated under reduced pressure to give a residue. The residue was purified by column chromatography (SiO2, petroleum ether/ethyl acetate=10/1 to 0/1) to afford (E)-benzyl 4-methylpent-2-enoate (3.60 g, 17.6 mmol, 67% yield) as yellow oil.

1H NMR (400 MHz, CDCl3) δ=7.41-7.32 (m, 5H), 7.02 (dd, J=6.6, 15.8 Hz, 1H), 5.84 (dd, J=1.5, 15.8 Hz, 1H), 5.19 (s, 2H), 2.47 (ddd, J=1.5, 6.8, 13.5 Hz, 1H), 1.08 (s, 3H), 1.07 (s, 3H).

### Step 2. Procedure for Preparation of Compound 3—(1R,2S)-benzyl 2-isopropylcyclopropanecarboxylate

To a solution of trimethylsulfoxoniumiodide (3.88 g, 17.6 mmol, 1.20 eq) in dimethylsulfoxide (20.0 mL) was added potassium tert-butoxide (1.98 g, 17.6 mmol, 1.20 eq) at 20° C. The mixture was stirred at 20° C. for 1 h. The mixture was added into a mixture of (E)-benzyl 4-methylpent-2-enoate (3.00 g, 14.6 mmol, 1.00 eq) in dimethylsulfoxide (10.0 mL) at 50° C. under nitrogen atmosphere. The mixture was stirred at 50° C. for 1 h under nitrogen atmosphere. The mixture was quenched with water (20 mL), extracted with ethyl acetate (3×20 mL). The combined organic layers were washed with brine (3×20 mL), dried over anhydrous sodium sulfate, filtered and concentrated under reduced pressure to give a residue. The residue was purified by column chromatography (SiO2, petroleum ether/ethyl acetate=10/1 to 5/1) to afford (1R,2S)-benzyl 2-isopropylcyclopropanecarboxylate (240 mg, 1.10 mmol, 7% yield) as yellow oil.

1H NMR (400 MHz, CDCl3) δ=7.39-7.35 (m, 5H), 5.12 (d, J=4.1 Hz, 2H), 1.47 (td, J=4.3, 8.3 Hz, 1H), 1.28 (ddd, J=2.1, 4.1, 6.4 Hz, 1H), 1.18 (td, J=4.3, 8.7 Hz, 1H), 1.10-1.04 (m, 1H), 1.01-0.97 (m, 6H), 0.77 (ddd, J=4.1, 6.4, 8.2 Hz, 1H).

### Step 3. Procedure for Preparation of Compound 4—(1R,2S)-2-isopropylcyclopropanecarboxylic acid

To a solution of (1R,2S)-benzyl 2-isopropylcyclopropanecarboxylate (240 mg, 1.10 mmol, 1.00 eq) in methanol (2.50 mL) was added palladium on activated carbon (20.0 mg, 10% purity). The mixture was stirred at 20° C. for 1 h under 15 psi of hydrogen atmosphere. The mixture was filtered and the filtrate was concentrated under reduced pressure to afford (1R,2S)-2-isopropylcyclopropanecarboxylic acid (100 mg, 780 umol, 70% yield) as colorless oil.

1H NMR (400 MHz, CDCl₃) δ=1.40 (td, J=4.2, 8.2 Hz, 1H), 1.33-1.25 (m, 1H), 1.20 (td, J=4.3, 8.8 Hz, 1H), 1.05 (td, J=6.1, 8.2 Hz, 1H), 1.01-0.97 (m, 6H), 0.85-0.79 (m, 1H).

### Step 4. Procedure for Preparation of Compound 5—((1R,2S)-2-isopropylcyclopropyl)methanol

To a solution of (1R,2S)-2-isopropylcyclopropanecarboxylic acid (50.0 mg, 390 umol, 1.00 eq) in tetrahydrofuran (1.00 mL) was added borane dimethyl sulfide complex (10.0 M, 78.0 uL, 2.00 eq) at 0° C. The mixture was stirred at 20° C. for 1 h. The mixture was quenched with methanol (5 mL) and concentrated under reduced pressure to afford ((1R,2S)-2-isopropylcyclopropyl)methanol (20.0 mg, 175 umol, 44% yield) as colorless oil.

1H NMR (400 MHz, CDCl3) δ=3.43 (br s, 2H), 1.33-1.18 (m, 1H), 0.97 (br s, 3H), 0.88 (br d, J=4.6 Hz, 2H), 0.42 (br s, 2H), 0.36 (br d, J=5.9 Hz, 3H).

1H NMR (400 MHz, CDCl₃, T=50° C.) δ=3.44 (br t, J=6.1 Hz, 2H), 1.28 (s, 1H), 0.98 (br s, 3H), 0.89 (td, J=5.7, 11.7 Hz, 2H), 0.44 (br s, 2H), 0.39-0.32 (m, 3H)

### Step 5. Procedure for preparation of ((1R,2S)-2-isopropylcyclopropyl)methyl ((2-(2,6-dioxopiperidin-3-yl)-3-oxoisoindolin-5-yl)methyl)carbamate

To a solution of ((1R,2S)-2-isopropylcyclopropyl)methanol (30.0 mg, 262 umol, 1.00 eq) in tetrahydrofuran (0.500 mL) was added di(1H-imidazol-1-yl)methanone (42.6 mg, 262 umol, 1.00 eq) at 0° C. The mixture was stirred at 30° C. for 1 h. Then the mixture was added into a solution of 3-(6-(aminomethyl)-1-oxoisoindolin-2-yl)piperidine-2,6-dione (74.3 mg, 240 umol, 1.00 eq, hydrochloride), 1,8-diazabicyclo[5.4.0]undec-7-ene (36.5 mg, 240 umol, 36.1 uL, 1.00 eq) and triethylamine (24.2 mg, 240 umol, 33.4 uL, 1.00 eq) in tetrahydrofuran (0.300 mL) and dimethylformamide (0.300 mL). The mixture was stirred at 20° C. for 11 h. The mixture was concentrated under reduced pressure to give a residue. The residue was purified by Prep-HPLC (column: Phenomenex luna C18 150*25 mm*10 um; mobile phase: [water (formic acid)-acetonitrile]; B %: 36%-66%, 10 min) and lyophilized to afford ((1R,2S)-2-isopropylcyclopropyl)methyl((2-(2,6-dioxopiperidin-3-yl)-3-oxoisoindolin-5-yl)methyl)carbamate (5.39 mg, 12.91 umol, 5% yield, 99% purity) as a yellow solid.

1H NMR (400 MHz, DMSO-d6) δ=10.98 (br s, 1H), 7.76 (br t, J=6.1 Hz, 1H), 7.61 (s, 1H), 7.57-7.47 (m, 2H), 5.11 (dd, J=5.1, 13.3 Hz, 1H), 4.46-4.39 (m, 1H), 4.33-4.24 (m, 3H), 3.80 (d, J=6.6 Hz, 2H), 2.97-2.85 (m, 1H), 2.64-2.56 (m, 1H), 2.39 (dd, J=4.4, 12.9 Hz, 1H), 2.04-1.96 (m, 1H), 0.90 (s, 6H), 0.88-0.81 (m, 2H), 0.48 (br d, J=3.4 Hz, 1H), 0.40-0.29 (m, 2H).

### MS (ESI) m/z 414.1 [M+H]⁺.

### Example 30. Synthesis of Compound 117

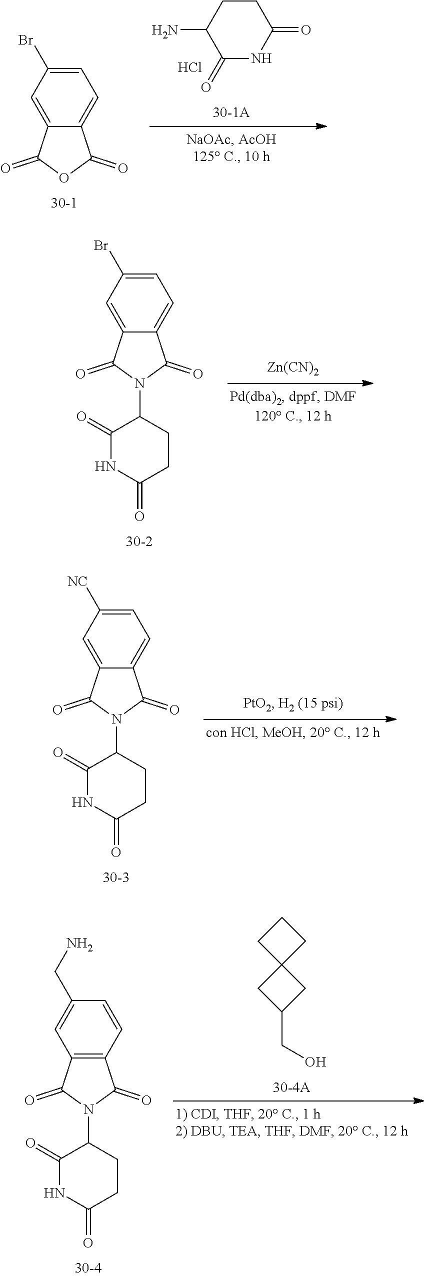

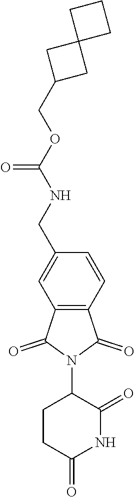 [SMILES: O=C1CCC(N2C(=O)c3ccc(CNC(=O)OCC4CC5(CCC5)C4)cc3C2=O)C(=O)N1]

### 4.1. Procedure for Compound 2—5-bromo-2-(2,6-dioxopiperidin-3-yl)isoindoline-1,3-dione

A mixture of 5-bromoisobenzofuran-1,3-dione (10.0 g, 44.1 mmol, 1.00 eq), 3-aminopiperidine-2,6-dione hydrochloride (7.25 g, 44.1 mmol, 1.00 eq) and sodium acetate (3.61 g, 44.4 mmol, 1.00 eq) in acetic acid (150 mL) was heated at 125° C. for 10 h. The reaction mixture was cooled to 25° C. and concentrate under reduced pressure to give a residue. The residue was triturated with water (170 mL) at 20° C. for 1 h and then filtered. The filter cake was concentrated under reduced pressure to afford 5-bromo-2-(2,6-dioxopiperidin-3-yl)isoindoline-1,3-dione (13.7 g, 40.0 mmol, 91% yield, 98% purity) as a purple solid.

1H NMR (400 MHz, DMSO-d6) δ=11.15 (s, 1H), 8.25-8.03 (m, 2H), 7.86 (d, J=7.9 Hz, 1H), 5.16 (dd, J=5.4, 12.8 Hz, 1H), 2.92-2.80 (m, 1H), 2.68-2.54 (m, 2H), 2.14-1.96 (m, 1H).

### Step 2. Procedure for Compound 3-2-(2,6-dioxopiperidin-3-yl)-1,3-dioxoisoindoline-5-carbonitrile

To a solution of 5-bromo-2-(2,6-dioxopiperidin-3-yl)isoindoline-1,3-dione (7.00 g, 20.8 mmol, 1.00 eq) in N,N-dimethylformamide (70.0 mL) were added zinc cyanide (4.88 g, 41.5 mmol, 2.00 eq), 1,1′-bis(diphenylphosphino)ferrocene (1.15 g, 2.08 mmol, 0.100 eq) and bis(dibenzylideneacetone)palladium (1.19 g, 2.08 mmol, 0.100 eq). The reaction mixture was stirred at 120° C. for 12 h. The mixture was triturated with water (200 mL). The mixture was filtered and the filter cake was concentrated under reduced pressure to give a residue. The residue was triturated with ethyl alcohol (30 mL) and then filtered. The filter cake was concentrated under reduced pressure to afford 2-(2,6-dioxo-3-piperidyl)-1,3-dioxo-isoindoline-5-carbonitrile (4.00 g, 14.1 mmol, 68% yield) as a gray solid.

1H NMR (400 MHz, CDCl3) δ=11.24-10.78 (m, 1H), 8.29 (br d, J=7.5 Hz, 1H), 8.04 (br d, J=7.5 Hz, 1H), 7.87 (s, 1H), 5.13 (br dd, J=5.1, 12.7 Hz, 1H), 2.85 (br s, 1H), 2.62-2.49 (m, 2H), 2.03-1.96 (m, 1H).

### Step 3. Procedure for Compound 4—5-(aminomethyl)-2-(2,6-dioxopiperidin-3-yl)isoindoline-1,3-dione

To a solution of 2-(2,6-dioxopiperidin-3-yl)-1,3-dioxoisoindoline-5-carbonitrile (1.00 g, 3.53 mmol, 1.00 eq) in methanol (100 mL) was added concentrated hydrochloric acid (3.00 mL), platinum(iv)oxide (241 mg, 1.06 mmol, 0.300 eq). The reaction mixture was stirred at 20° C. for 12 h under 15 psi of hydrogen atmosphere. The reaction mixture was added 1N of hydrochloric acid (40 mL) and filtered. The filtrate was concentrated under reduced pressure to give a residue. The residue was purified by Prep-HPLC (column: Phenomenex Luna C18 200*40 mm*10 um; mobile phase: [water (hydrochloric acid)-acetonitrile]; B %: 1%-20%, 10 min) and lyophilized to afford 5-(aminomethyl)-2-(2,6-dioxopiperidin-3-yl)isoindoline-1,3-dione (30.0 mg, 104 umol, 3% yield) as a white solid.

### MS (ESI) m/z 288.1 [M+H]⁺.

### Step 4. Procedure for spiro[3.3]heptan-2-ylmethyl ((2-(2,6-dioxopiperidin-3-yl)-1,3-dioxoisoindolin-5-yl)methyl)carbamate

To a solution of spiro[3.3]heptan-2-ylmethanol (15.0 mg, 119 umol, 1.00 eq) in tetrahydrofuran (0.100 mL) was added di(1H-imidazol-1-yl)methanone (38.6 mg, 238 umol, 2.00 eq). The reaction mixture was stirred at 20° C. for 1 h. Then the mixture was added into a solution of 5-(aminomethyl)-2-(2,6-dioxopiperidin-3-yl)isoindoline-1,3-dione (29.4 mg, 102 umol, 1.13 eq), 2,3,4,6,7,8,9,10-octahydropyrimido[1,2-a]azepine (90.8 umol, 13.7 uL, 1.00 eq) and triethylamine (90.8 umol, 12.6 uL, 1.00 eq) in tetrahydrofuran (1.00 mL) and dimethyformamide (1.00 mL). The reaction mixture was stirred at 20° C. for 12 h. The reaction was concentrated under reduced pressure and filtered. The filtrate was purified by Prep-HPLC (column: Waters xbridge 150*25 mm 10 um; mobile phase: [water (ammonium bicarbonate)-acetonitrile]; B %: 33%-63%, 8 min). The mixture was added 2 drop formic acid and lyophilized to afford spiro[3.3]heptan-2-ylmethyl ((2-(2,6-dioxopiperidin-3-yl)-1,3-dioxoisoindolin-5-yl) methyl)carbamate (10.25 mg, 23.09 umol, 25% yield, 99% purity) as a white solid.

1H NMR (400 MHz, DMSO-d6) δ=11.32-10.87 (m, 1H), 7.94-7.81 (m, 2H), 7.79-7.70 (m, 2H), 5.15 (dd, J=5.4, 12.9 Hz, 1H), 4.35 (br d, J=6.1 Hz, 2H), 3.92 (br d, J=6.6 Hz, 2H), 2.96-2.82 (m, 1H), 2.68-2.53 (m, 2H), 2.36 (br dd, J=7.2, 14.8 Hz, 1H), 2.11-2.00 (m, 3H), 1.97 (br t, J=7.2 Hz, 2H), 1.90-1.80 (m, 2H), 1.77-1.51 (m, 4H).

1H NMR (400 MHz, DMSO-d6, T=80° C.) δ=11.03-10.59 (m, 1H), 7.87 (d, J=7.6 Hz, 1H), 7.80-7.73 (m, 2H), 7.63-7.39 (m, 1H), 5.11 (dd, J=5.4, 12.8 Hz, 1H), 4.36 (d, J=6.1 Hz, 2H), 3.94 (d, J=6.8 Hz, 2H), 2.97-2.82 (m, 1H), 2.66-2.56 (m, 2H), 2.38 (td, J=7.5, 14.7 Hz, 1H), 2.13-2.07 (m, 1H), 2.05 (br d, J=10.5 Hz, 2H), 2.00 (br s, 2H), 1.92-1.85 (m, 2H), 1.81-1.67 (m, 4H).

### MS (ESI) m/z 462.1 [M+Na]⁺.

### Example 31. Synthesis of Compound 118

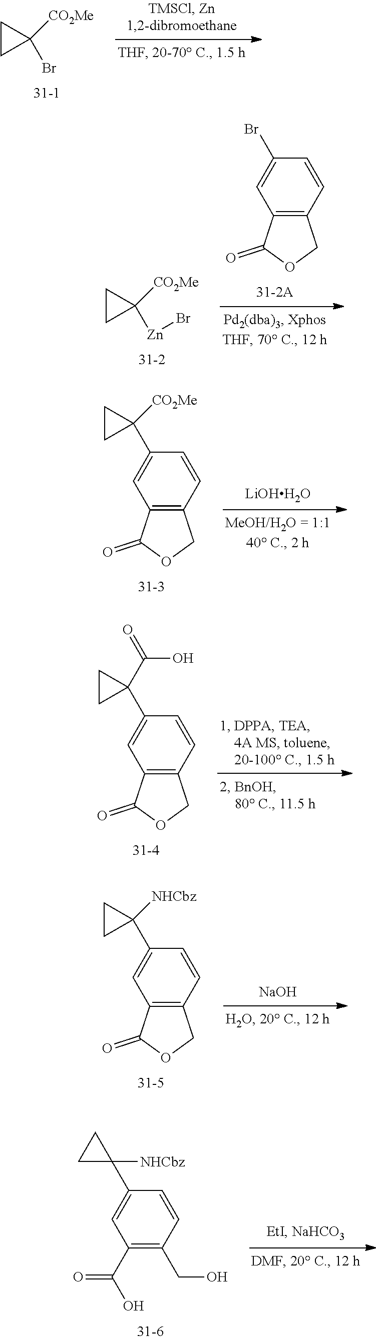

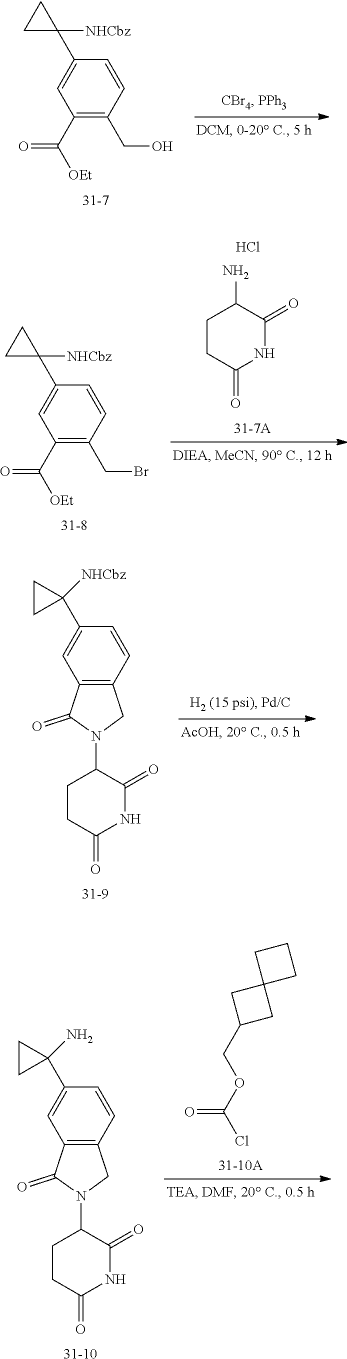

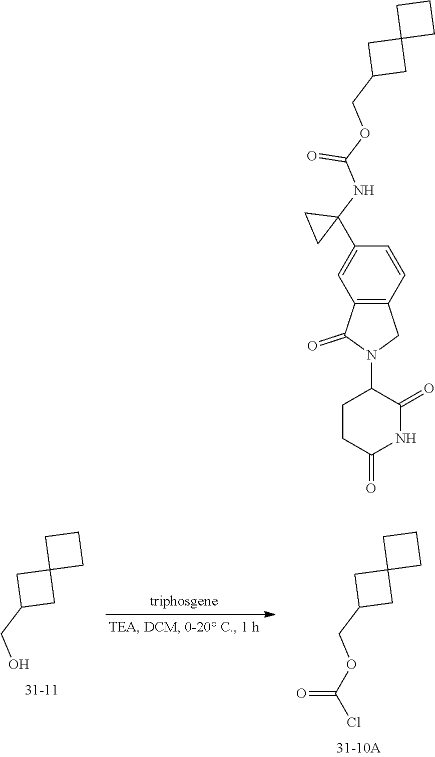 [SMILES: O=C1CCC(N2Cc3ccc(C4(NC(=O)OCC5CC6(CCC6)C5)CC4)cc3C2=O)C(=O)N1 ; OCC1CC2(CCC2)C1 ; O=C(Cl)OCC1CC2(CCC2)C1]

### Step 1. Procedure for Preparation of Compound 2—(1-methoxycarbonyl)cyclopropyl)zinc(II) bromide

To a solution of zinc (12.3 g, 188 mmol, 2.00 eq) in tetrahydrofuran (160 mL) were added trimethylchlorosilane (1.02 g, 9.38 mmol, 1.19 mL, 0.100 eq) and 1,2-dibromoethane (3.53 g, 18.8 mmol, 1.42 mL, 0.200 eq) at 20° C. under nitrogen atmosphere. The mixture was stirred at 20° C. for 30 min. Then methyl 1-bromocyclopropanecarboxylate (16.8 g, 93.9 mmol, 1.00 eq) was added to the mixture and the reaction mixture was stirred at 70° C. for 1 h under nitrogen atmosphere to afford (1-(methoxycarbonyl)cyclopropyl)zinc(II) bromide (22.9 g, 93.9 mmol, 100% yield) as colorless oil.

Note: The reaction mixture was directly used to next step.

### Step 2. Procedure for Preparation of Compound 3—methyl 1-(3-oxo-1,3-dihydroisobenzofuran-5-yl)cyclopropanecarboxylate

To a solution of 6-bromoisobenzofuran-1(3H)-one (10.0 g, 46.9 mmol, 1.00 eq), (1-(methoxycarbonyl)cyclopropyl)zinc(II) bromide (22.9 g, 93.8 mmol, 2.00 eq) in tetrahydrofuran (160 mL) were added tris(dibenzylideneacetone)dipalladium(0) (430 mg, 469 umol, 0.0100 eq) and 2-(dicyclohexylphosphino)-2,4,6-tri-i-propyl-1,1-biphenyl (448 mg, 939 umol, 0.0200 eq). The reaction mixture was stirred at 70° C. for 12 h under nitrogen atmosphere. The reaction mixture was filtered and the filtrate was concentrated under reduced pressure to give a residue. The residue was poured into water (150 mL) and extracted with ethyl acetate (2×100 mL). The combined organic layers were washed with brine (200 mL), dried over anhydrous sodium sulfate, filtered and concentrated under reduced pressure to give another residue. The residue was purified by column chromatography (SiO₂, petroleum ether/ethyl acetate=1/0 to 1/1) to afford methyl 1-(3-oxo-1,3-dihydroisobenzofuran-5-yl)cyclopropanecarboxylate (7.10 g, 30.6 mmol, 65% yield) as a yellow solid.

¹H NMR (400 MHz, CDCl₃) δ=7.89 (d, J=1.0 Hz, 1H), 7.71 (dd, J=1.6, 7.9 Hz, 1H), 7.45 (dd, J=0.6, 7.9 Hz, 1H), 5.32 (s, 2H), 3.64 (s, 3H), 1.73-1.68 (m, 2H), 1.26-1.22 (m, 2H).

### Step 3. Procedure for Preparation of Compound 4—1-(3-oxo-1,3-dihydroisobenzofuran-5-yl)cyclopropanecarboxylic acid

To a solution of methyl 1-(3-oxo-1,3-dihydroisobenzofuran-5-yl)cyclopropanecarboxylate (7.10 g, 30.6 mmol, 1.00 eq) in methanol (70.0 mL) and water (70.0 mL) was added lithium hydroxide monohydrate (5.13 g, 122 mmol, 4.00 eq). The reaction mixture was stirred at 40° C. for 2 h under nitrogen atmosphere. The reaction mixture was concentrated under reduced pressure to give a residue. The residue was added 1M hydrochloric acid till pH=4 and then extracted with ethyl acetate (2×85 mL). The combined organic layers were dried over anhydrous sodium sulfate, filtered and concentrated under reduced pressure to give a crude product. The crude product was triturated with a mixture (methanol:ethyl acetate=2:1, 90 mL) at 20° C. for 3 h and filtered. The filter cake was concentrated under reduced pressure to afford 1-(3-oxo-1,3-dihydroisobenzofuran-5-yl) cyclopropanecarboxylic acid (5.74 g, 26.3 mmol, 86% yield) as a white solid.

¹H NMR (400 MHz, DMSO-d₆) δ=7.72 (d, J=1.3 Hz, 1H), 7.24-7.17 (m, 1H), 7.17-7.09 (m, 1H), 4.48 (s, 2H), 1.43-1.34 (m, 2H), 1.00 (q, J=3.5 Hz, 2H).

### Step 4. Procedure for Preparation of Compound 5—benzyl (1-(3-oxo-1,3-dihydroisobenzofuran-5-yl)cyclopropyl)carbamate

To a solution of 1-(3-oxo-1,3-dihydroisobenzofuran-5-yl)cyclopropanecarboxylic acid (4.80 g, 22.0 mmol, 1.00 eq) in toluene (50.0 mL) were added diphenylphosphoryl azide (7.26 g, 26.4 mmol, 5.72 mL, 1.20 eq), triethylamine (2.67 g, 26.4 mmol, 3.67 mL, 1.20 eq) and 4 A MS (5.00 g). The mixture was stirred at 20° C. for 0.5 h and then warmed to 100° C. for 1 h under nitrogen atmosphere. Then phenylmethanol (3.57 g, 33.0 mmol, 3.43 mL, 1.50 eq) was added to the mixture and the reaction mixture was stirred at 80° C. for 11.5 h. The reaction mixture was filtered and the filtrate was concentrated under reduced pressure to give a residue. The residue was purified by reverse phase chromatography (C18, 330 g; condition: water/acetonitrile=100:0 to 0:100, 0.1% formic acid) and lyophilized to afford benzyl (1-(3-oxo-1,3-dihydroisobenzofuran-5-yl)cyclopropyl)carbamate (1.40 g, 4.33 mmol, 20% yield) as a yellow solid.

¹H NMR (400 MHz, CDCl₃) δ=7.85-7.66 (m, 1H), 7.61 (br d, J=7.9 Hz, 1H), 7.42 (br d, J=7.9 Hz, 1H), 7.37 (s, 4H), 7.27-7.14 (m, 1H), 5.63-5.38 (m, 1H), 5.29 (s, 2H), 5.11 (s, 2H), 1.39 (br s, 2H), 1.35-1.29 (m, 2H).

### Step 5. Procedure for Preparation of Compound 6—5-(1-(((benzyloxy)carbonyl)amino)cyclopropyl)-2-(hydroxymethyl)benzoic acid

To a solution of benzyl (1-(3-oxo-1,3-dihydroisobenzofuran-5-yl)cyclopropyl)carbamate (1.40 g, 4.33 mmol, 1.00 eq) in water (10.0 mL) was added sodium hydroxide (1.73 g, 43.3 mmol, 10.0 eq). The reaction mixture was stirred at 20° C. for 12 h. The reaction mixture was adjusted pH to 5-6 with 1 N hydrochloric acid, and then the mixture was extracted with ethyl acetate (3×10 mL). The combined organic phases were dried with anhydrous sodium sulfate, filtered and concentrated under reduced pressure to afford 5-(1-(((benzyloxy)carbonyl)amino)cyclopropyl)-2-(hydroxymethyl)benzoic acid (1.40 g, 4.10 mmol, 95% yield) as a white solid.

¹H NMR (400 MHz, DMSO-d₆) δ=13.23-12.24 (m, 1H), 8.23 (s, 1H), 7.74 (d, J=1.5 Hz, 1H), 7.59 (d, J=8.1 Hz, 1H), 7.39-7.36 (m, 1H), 7.36-7.29 (m, 5H), 5.02 (s, 2H), 4.78 (s, 2H), 1.17 (br d, J=1.1 Hz, 4H).

### Step 6. Procedure for Preparation of Compound 7—ethyl 5-(1-(((benzyloxy)carbonyl)amino)cyclopropyl)-2-(hydroxymethyl)benzoate

To a solution of 5-(1-(((benzyloxy)carbonyl)amino)cyclopropyl)-2-(hydroxymethyl)benzoic acid (1.40 g, 4.10 mmol, 1.00 eq) in dimethylformamide (10.0 mL) were added sodium bicarbonate (1.03 g, 12.3 mmol, 479 uL, 3.00 eq) and iodoethane (1.28 g, 8.20 mmol, 656 uL, 2.00 eq). The reaction mixture was stirred at 20° C. for 12 h. The reaction mixture was added to water (100 mL) and extracted with ethyl acetate (3×100 mL). The combined organic phases were dried with anhydrous sodium sulfate, filtered and concentrated under reduced pressure to afford ethyl 5-(1-(((benzyloxy)carbonyl)amino)cyclopropyl)-2-(hydroxymethyl) benzoate (1.50 g, 4.06 mmol, 99% yield) as yellow oil.

¹H NMR (400 MHz, CDCl₃) δ=7.91-7.78 (m, 1H), 7.50-7.41 (m, 1H), 7.40-7.30 (m, 5H), 7.27-7.11 (m, 1H), 5.59-5.34 (m, 1H), 5.10 (s, 2H), 4.74 (s, 2H), 4.39 (q, J=7.1 Hz, 2H), 1.41 (t, J=7.1 Hz, 3H), 1.36-1.31 (m, 2H), 1.29-1.25 (m, 2H).

### Step 7. Procedure for Preparation of Compound 8—ethyl 5-(1-(((benzyloxy)carbonyl)amino)cyclopropyl)-2-(bromomethyl)benzoate

To a solution of carbon tetrabromide (539 mg, 1.62 mmol, 1.20 eq) and triphenylphosphine (426 mg, 1.62 mmol, 1.20 eq) in dichloromethane (5.00 mL) was added ethyl 5-(1-(((benzyloxy)carbonyl)amino)cyclopropyl)-2-(hydroxymethyl) benzoate (500 mg, 1.35 mmol, 1.00 eq) in dichloromethane (2.00 mL) at 0° C. The reaction mixture was stirred at 20° C. for 5 h. The reaction mixture was concentrated under reduced pressure to give a residue. The residue was purified by column chromatography (SiO₂, petroleum ether/ethyl acetate=1/0 to 3/1) to afford ethyl 5-(1-(((benzyloxy)carbonyl)amino)cyclopropyl)-2-(bromomethyl)benzoate (110 mg, 255 umol, 19% yield) as a yellow solid.

¹H NMR (400 MHz, CDCl₃) δ=7.85-7.70 (m, 1H), 7.41-7.12 (m, 7H), 5.51 (br s, 1H), 5.10 (s, 2H), 4.92 (s, 2H), 4.41 (q, J=7.0 Hz, 2H), 1.42 (t, J=7.0 Hz, 3H), 1.35-1.27 (m, 4H).

### Step 8. Procedure for Preparation of Compound 9—benzyl (1-(2-(2,6-dioxopiperidin-3-yl)-3-oxoisoindolin-5-yl)cyclopropyl)carbamate

To a solution of ethyl 5-(1-(((benzyloxy)carbonyl)amino)cyclopropyl)-2-(bromomethyl)benzoate (110 mg, 255 umol, 1.00 eq) in acetonitrile (5.00 mL) was added N,N-diisopropylethylamine (98.7 mg, 763 umol, 133 uL, 3.00 eq) and 3-aminopiperidine-2,6-dione (39.1 mg, 305 umol, 1.20 eq, hydrochloride). The reaction mixture was stirred at 90° C. for 12 h. The reaction mixture was concentrated under reduced pressure to give a residue. The residue was triturated with a mixture (ethyl acetate:water=3:2, 2.5 mL) at 20° C. for 1 h and then filtered. The filter cake was concentrated under reduced pressure to afford benzyl (1-(2-(2,6-dioxopiperidin-3-yl)-3-oxoisoindolin-5-yl)cyclopropyl)carbamate (76.0 mg, 175 umol, 69% yield) as a blue solid.

¹H NMR (400 MHz, CDCl₃) δ=7.95-7.83 (m, 1H), 7.81-7.62 (m, 1H), 7.59-7.51 (m, 1H), 7.43-7.31 (m, 6H), 5.53 (br s, 1H), 5.23 (dd, J=4.6, 13.1 Hz, 1H), 5.10 (s, 2H), 4.51-4.43 (m, 1H), 4.36-4.28 (m, 1H), 2.98-2.91 (m, 1H), 2.90-2.80 (m, 1H), 2.42-2.32 (m, 1H), 2.28-2.17 (m, 1H), 1.36 (br s, 2H), 1.33 (br s, 2H).

### Step 9. Procedure for Preparation of Compound 10—3-(6-(1-aminocyclopropyl)-1-oxoisoindolin-2-yl)piperidine-2,6-dione

To a solution of benzyl (1-(2-(2,6-dioxopiperidin-3-yl)-3-oxoisoindolin-5-yl)cyclopropyl)carbamate (35.0 mg, 80.8 umol, 1.00 eq) in acetic acid (1.00 mL) was added palladium on activated carbon (30.0 mg, 10% purity). The mixture was stirred at 20° C. for 0.5 h under hydrogen atmosphere (15 Psi). The reaction mixture was filtered and the filtrated was concentrated under reduced pressure to afford 3-(6-(1-aminocyclopropyl)-1-oxoisoindolin-2-yl)piperidine-2,6-dione (20.0 mg, 66.8 umol, 83% yield) as a blue solid.

¹H NMR (400 MHz, MeOD) δ=7.89 (d, J=1.3 Hz, 1H), 7.72 (dd, J=1.7, 7.9 Hz, 1H), 7.67-7.61 (m, 1H), 5.16 (dd, J=5.3, 13.4 Hz, 1H), 4.58-4.44 (m, 2H), 2.97-2.86 (m, 1H), 2.83-2.75 (m, 1H), 2.56-2.45 (m, 1H), 2.21-2.15 (m, 1H), 1.36-1.33 (m, 2H), 1.30-1.27 (m, 2H).

### Step 10. Procedure for Preparation of Compound 10A—spiro[3.3]heptan-2-ylmethyl carbonochloridate

To a solution of spiro[3.3]heptan-2-ylmethanol (25.0 mg, 198 umol, 1.00 eq) in dichloromethane (0.500 mL) was added bis(trichloromethyl) carbonate (58.8 mg, 198 umol, 1.00 eq) and triethylamine (40.1 mg, 396 umol, 55.2 uL, 2.00 eq) at 0° C. The reaction mixture was stirred at 20° C. for 1 h. The reaction mixture was concentrated under reduced pressure to afford spiro[3.3]heptan-2-ylmethyl carbonochloridate (35.0 mg, crude) as a white solid.

### Step 11. Procedure for Preparation of spiro[3.3]heptan-2-ylmethyl (1-(2-(2,6-dioxopiperidin-3-yl)-3-oxoisoindolin-5-yl)cyclopropyl)carbamate

To a solution of 3-(6-(1-aminocyclopropyl)-1-oxoisoindolin-2-yl)piperidine-2,6-dione (20.0 mg, 66.8 umol, 1.00 eq) in dimethylformamide (0.500 mL) were added triethylamine (20.3 mg, 201 umol, 27.9 uL, 3.00 eq) and spiro[3.3]heptan-2-ylmethyl carbonochloridate (25.2 mg, 134 umol, 2.00 eq). The reaction mixture was stirred at 20° C. for 0.5 h. The reaction mixture was filtered. The filtrate was purified by Prep-HPLC (column: Phenomenex Luna C18 150*25 mm*10 um; mobile phase: [water (formic acid)-acetonitrile]; B %: 39%-69%, 9 min) and lyophilized to afford spiro[3.3]heptan-2-ylmethyl (1-(2-(2,6-dioxopiperidin-3-yl)-3-oxoisoindolin-5-yl)cyclopropyl)carbamate (2.15 mg, 4.71 umol, 7% yield, 99% purity) as a white solid.

¹H NMR (400 MHz, DMSO-d₆) δ=10.97 (br s, 1H), 8.09 (s, 1H), 7.56-7.47 (m, 2H), 7.38 (br d, J=8.3 Hz, 1H), 5.11 (dd, J=4.9, 13.3 Hz, 1H), 4.45-4.36 (m, 1H), 4.33-4.23 (m, 1H), 3.89-3.78 (m, 2H), 2.91 (ddd, J=5.1, 13.1, 17.8 Hz, 1H), 2.63-2.57 (m, 1H), 2.42-2.30 (m, 2H), 2.05-1.93 (m, 4H), 1.87 (br t, J=7.1 Hz, 2H), 1.80-1.46 (m, 5H), 1.18 (br s, 4H).

¹H NMR (400 MHz, DMSO-d₆, T=80° C.) δ=10.85-10.53 (m, 1H), 7.84-7.66 (m, 1H), 7.59 (s, 1H), 7.53-7.47 (m, 1H), 7.46-7.41 (m, 1H), 5.10-5.02 (m, 1H), 4.45-4.37 (m, 1H), 4.36-4.28 (m, 1H), 3.88 (d, J=6.6 Hz, 2H), 2.95-2.84 (m, 1H), 2.66-2.59 (m, 1H), 2.46-2.37 (m, 1H), 2.37-2.31 (m, 1H), 2.07-2.03 (m, 1H), 2.02-1.93 (m, 4H), 1.85 (br d, J=6.8 Hz, 2H), 1.79-1.72 (m, 2H), 1.72-1.64 (m, 2H), 1.20 (br d, J=5.0 Hz, 4H).

### MS (ESI) m/z 451.9 [M+H]⁺.

### Example 32. Synthesis of Compound 119

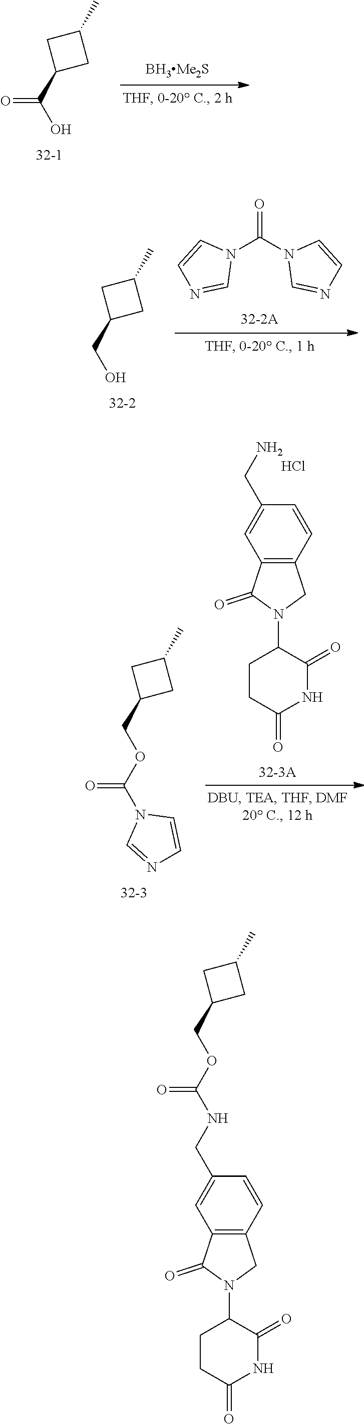

### Step 1. Procedure for Preparation of Compound 2—(1r,3r)-3-methylcyclobutyl)methanol

To a solution of 1r,3r)-3-methylcyclobutanecarboxylic acid (100 mg, 876 umol, 1.00 eq) in tetrahydrofuran (1.00 mL) was added borane dimethyl sulfide complex (10.0 M, 175 uL, 2.00 eq) at 0° C. The reaction mixture was stirred at 20° C. for 2 h. The reaction mixture was quenched by methanol (20 mL) and concentrated under reduced pressure to afford ((1r, 3r)-3-methylcyclobutyl)methanol (77.0 mg, crude) as yellow oil.

¹H NMR (400 MHz, CDCl₃) δ=3.65 (d, J=7.3 Hz, 2H), 2.51-2.42 (m, 1H), 2.42-2.33 (m, 1H), 1.93-1.86 (m, 2H), 1.73-1.65 (m, 2H), 1.12 (d, J=6.9 Hz, 3H).

### Step 2. Procedure for Preparation of Compound 3—(1r,3r)-3-methylcyclobutyl)methyl 1H-imidazole-1-carboxylate

To a solution of (1r,3r)-3-methylcyclobutyl)methanol (30.0 mg, 300 umol, 1.00 eq) in tetrahydrofuran (0.300 mL) was added di(1H-imidazol-1-yl)methanone (73.0 mg, 449 umol, 1.50 eq) at 0° C. The reaction mixture was stirred at 20° C. for 1 h. The reaction mixture was used into next step directly.

### Step 3. Procedure for preparation of (1r,3r)-3-methylcyclobutyl)methyl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (87.7 mg, 283 umol, 1.00 eq, hydrochloride) in tetrahydrofuran (0.500 mL) and dimethylformamide (0.500 mL) were added 2,3,4,6,7,8,9,10-octahydropyrimido[1,2-a]azepine (43.1 mg, 283 umol, 42.6 uL, 1.00 eq), triethylamine (43.0 mg, 425 umol, 59.0 uL, 1.00 eq) and (1r,3r)-3-methylcyclobutyl)methyl 1H-imidazole-1-carboxylate (previous prepared). The reaction mixture was stirred at 20° C. for 12 h. The reaction mixture was concentrated under reduced pressure to give a residue. The residue was diluted with dimethylformamide (1 mL) and then filtered. The filtrate was purified by Prep-HPLC (column: Phenomenex luna C18 150*25 mm*10 um; mobile phase: [water (formic acid)-acetonitrile]; B %: 36%-66%, 9 min) and lyophilized to afford (1r,3r)-3-methylcyclobutyl)methyl ((2-(2,6-dioxopiperidin-3-yl)-3-oxoisoindolin-5-yl)methyl)carbamate (22.31 mg, 55.29 umol, 20% yield, 99% purity) as an off-white solid.

¹H NMR (400 MHz, DMSO-d₆) δ=10.99 (br s, 1H), 7.79 (br t, J=6.1 Hz, 1H), 7.60 (s, 1H), 7.57-7.48 (m, 2H), 5.11 (dd, J=5.2, 13.3 Hz, 1H), 4.47-4.39 (m, 1H), 4.33-4.23 (m, 3H), 4.00 (d, J=7.1 Hz, 2H), 2.96-2.85 (m, 1H), 2.59 (br dd, J=2.2, 15.1 Hz, 1H), 2.53 (br d, J=2.0 Hz, 1H), 2.41 (br d, J=4.5 Hz, 1H), 2.36 (br d, J=8.8 Hz, 1H), 2.00 (br dd, J=5.2, 10.6 Hz, 1H), 1.92-1.80 (m, 2H), 1.69-1.57 (m, 2H), 1.06 (d, J=6.8 Hz, 3H).

¹H NMR (400 MHz, DMSO-d₆, T=80° C.) δ=10.89-10.59 (m, 1H), 7.63 (s, 1H), 7.56-7.51 (m, 2H), 7.50 (s, 1H), 5.07 (dd, J=5.2, 12.9 Hz, 1H), 4.47-4.41 (m, 1H), 4.37-4.31 (m, 1H), 4.28 (d, J=6.4 Hz, 2H), 4.01 (d, J=7.0 Hz, 2H), 2.95-2.84 (m, 1H), 2.66-2.61 (m, 1H), 2.47-2.42 (m, 1H), 2.39 (br s, 1H), 2.38-2.34 (m, 1H), 2.07-2.01 (m, 1H), 1.93-1.85 (m, 2H), 1.68-1.59 (m, 2H), 1.08 (d, J=6.9 Hz, 3H).

### MS (ESI) m/z 399.8 [M+H]⁺.

### Example 33. Synthesis of Compound 120

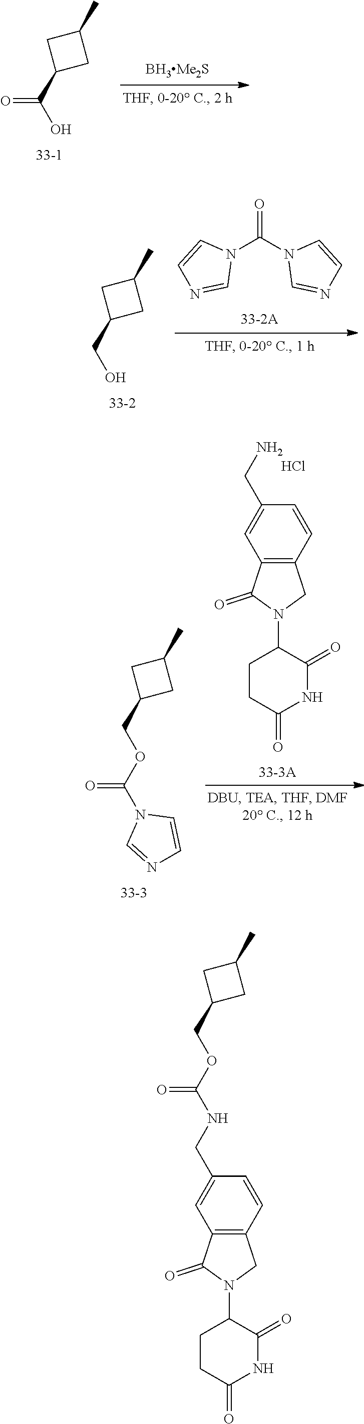

### Step 1. Procedure for Preparation of Compound 2—((1s,3s)-3-methylcyclobutyl)methanol

To a solution of (1s,3s)-3-methylcyclobutanecarboxylic acid (100 mg, 876 umol, 1.00 eq) in tetrahydrofuran (1.00 mL) was added borane dimethyl sulfide complex (10 M, 175 uL, 2.00 eq) at 0° C. The reaction mixture was stirred at 20° C. for 2 h. The reaction mixture was quenched by methanol (20 mL) and concentrated under reduced pressure to afford ((1s,3s)-3-methylcyclobutyl)methanol (76.0 mg, crude) as yellow oil.

¹H NMR (400 MHz, CDCl₃-d) δ=3.54 (d, J=6.5 Hz, 2H), 2.37-2.30 (m, 1H), 2.30-2.22 (m, 1H), 2.22-2.18 (m, 1H), 2.18-2.13 (m, 1H), 1.35-1.31 (m, 1H), 1.30-1.26 (m, 1H), 1.03 (d, J=6.6 Hz, 3H).

### Step 2. Procedure for Preparation of Compound 3—((1s,3s)-3-methylcyclobutyl)methyl 1H-imidazole-1-carboxylate

To a solution of ((1s,3s)-3-methylcyclobutyl)methanol (30 mg, 300 umol, 1.00 eq) in tetrahydrofuran (0.300 mL) was added di(1H-imidazol-1-yl)methanone (73.0 mg, 449 umol, 1.50 eq) at 0° C. The reaction mixture was stirred at 20° C. for 1 h. The reaction mixture was concentrated under reduced pressure to afford ((1s,3s)-3-methylcyclobutyl)methyl 1H-imidazole-1-carboxylate (55.0 mg, crude) as a white solid.

### Step 3. Procedure for preparation of ((1s,3s)-3-methylcyclobutyl)methyl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (87.7 mg, 283 umol, 1.00 eq, hydrochloride) in tetrahydrofuran (0.500 mL) and dimethylformamide (0.500 mL) were added 2,3,4,6,7,8,9,10-octahydropyrimido[1,2-a]azepine (43.1 mg, 283 umol, 42.68 uL, 1.00 eq), triethylamine (43.0 mg, 425 umol, 59.0 uL, 1.00 eq) and 3-methylcyclobutyl)methyl 1H-imidazole-1-carboxylate (55.0 mg, 283 umol, 1.00 eq). The reaction mixture was stirred at 20° C. for 12 h. The reaction mixture was concentrated under reduced pressure to give a residue. The residue was diluted with dimethylformamide (1 mL) and then filtered. The filtrate was purified by Prep-HPLC (column: Phenomenex luna C18 150*25 mm*10 um; mobile phase: [water (formic acid)-acetonitrile]; B %: 36%-66%, 9 min) and lyophilized to afford ((1s,3s)-3-methylcyclobutyl)methyl ((2-(2,6-dioxopiperidin-3-yl)-3-oxoisoindolin-5-yl)methyl)carbamate (34.65 mg, 85.88 umol, 30% yield, 99% purity) as an off-white solid.

¹H NMR (400 MHz, DMSO-d₆) δ=10.98 (br s, 1H), 7.77 (br t, J=5.9 Hz, 1H), 7.60 (s, 1H), 7.57-7.48 (m, 2H), 5.11 (dd, J=5.0, 13.3 Hz, 1H), 4.47-4.39 (m, 1H), 4.33-4.24 (m, 3H), 3.89 (br d, J=6.4 Hz, 2H), 2.96-2.85 (m, 1H), 2.60 (br d, J=17.5 Hz, 1H), 2.46-2.38 (m, 1H), 2.37-2.32 (m, 1H), 2.21 (dt, J=7.5, 14.8 Hz, 1H), 2.15-2.05 (m, 2H), 2.03-1.97 (m, 1H), 1.38-1.23 (m, 2H), 0.99 (br d, J=6.5 Hz, 3H).

¹H NMR (400 MHz, DMSO-d₆, T=80° C.) δ=10.69 (br s, 1H), 7.63 (s, 1H), 7.56-7.48 (m, 2H), 7.43 (br s, 1H), 5.06 (dd, J=5.3, 13.0 Hz, 1H), 4.49-4.38 (m, 1H), 4.38-4.31 (m, 1H), 4.29 (d, J=6.1 Hz, 2H), 3.91 (d, J=6.3 Hz, 2H), 2.94-2.84 (m, 1H), 2.67-2.59 (m, 1H), 2.41 (br d, J=8.5 Hz, 1H), 2.37-2.31 (m, 1H), 2.28-2.18 (m, 1H), 2.17-2.08 (m, 2H), 2.08-2.01 (m, 1H), 1.38-1.25 (m, 2H), 1.00 (d, J=6.4 Hz, 3H).

### MS (ESI) m/z 400.1 [M+H]⁺.

### Example 34. Synthesis of Compound 121

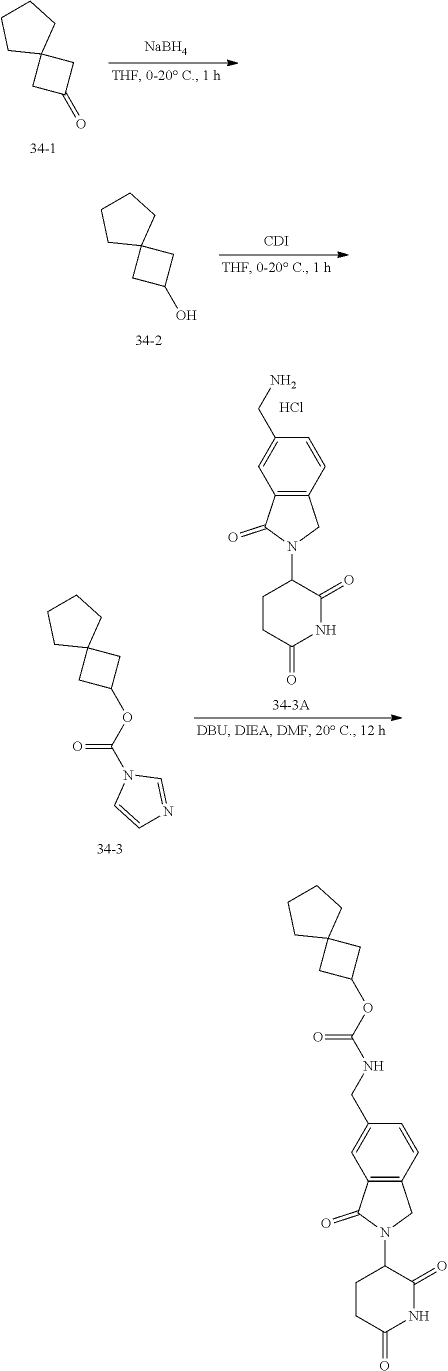

### Step 1. Procedure for Preparation of Compound 2—spiro[3.4]octan-2-ol

To a solution of spiro[3.4]octan-2-one (680 mg, 5.48 mmol, 1.00 eq) in tetrahydrofuran (7.00 mL) was added sodium borohydride (414 mg, 11.0 mmol, 2.00 eq) at 0° C. The reaction mixture was stirred at 20° C. for 1 h. The reaction mixture was quenched with saturated ammonium chloride (30 mL) and then extracted with ethyl acetate (3×20 mL). The organic layers were washed with brine (40 mL), dried over anhydrous sodium sulfate, filtered and concentrated under reduced pressure to afford spiro[3.4]octan-2-ol (630 mg, 4.99 mmol, 91% yield) as yellow oil.

¹H NMR (400 MHz, CDCl₃) δ=4.24 (quin, J=7.3 Hz, 1H), 2.28-2.19 (m, 2H), 2.01 (br d, J=2.6 Hz, 1H), 1.85-1.77 (m, 2H), 1.60-1.49 (m, 8H).

### Step 2. Procedure for Preparation of Compound 3—spiro[3.4]octan-2-yl 1H-imidazole-1-carboxylate

To a solution of spiro[3.4]octan-2-ol (20.0 mg, 159 umol, 1.00 eq) in tetrahydrofuran (0.200 mL) was added di(1H-imidazol-1-yl)methanone (38.6 mg, 238 umol, 1.50 eq) at 0° C. The reaction mixture was stirred at 20° C. for 1 h to afford spiro[3.4]octan-2-yl 1H-imidazole-1-carboxylate which was used into next step directly.

### Step 3. Procedure for Preparation of spiro[3.4]octan-2-yl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (45.0 mg, 145 umol, 1.00 eq, hydrochloride) in dimethylformamide (0.500 mL) were added 2,3,4,6,7,8,9,10-octahydropyrimido[1,2-a] azepine (22.1 mg, 145 umol, 21.9 uL, 1.00 eq), N,N-diisopropylethylamine (18.8 mg, 145 umol, 25.3 uL, 1.00 eq) and spiro[3.4]octan-2-yl 1H-imidazole-1-carboxylate in tetrahydrofuran (0.200 mL). The reaction mixture was stirred at 20° C. for 12 h. The reaction mixture was concentrated under reduced pressure to give a residue. The residue was purified by Prep-HPLC (column: Phenomenex luna C18 150*25 mm*10 um; mobile phase: [water (formic acid)-acetonitrile]; B %: 35%-65%, 9 min) and lyophilized to afford spiro[3.4]octan-2-yl ((2-(2,6-dioxopiperidin-3-yl)-3-oxoisoindolin-5-yl)methyl)carbamate (14.42 mg, 33.55 umol, 23% yield, 99% purity) as an off-white solid.

¹H NMR (400 MHz, DMSO-d₆) δ=10.97 (s, 1H), 7.75 (br t, J=5.5 Hz, 1H), 7.59 (s, 1H), 7.57-7.53 (m, 1H), 7.52-7.47 (m, 1H), 5.11 (dd, J=5.0, 13.4 Hz, 1H), 4.87-4.77 (m, 1H), 4.47-4.38 (m, 1H), 4.33-4.27 (m, 1H), 4.25 (d, J=6.3 Hz, 2H), 2.96-2.85 (m, 1H), 2.64-2.57 (m, 1H), 2.41 (br s, 1H), 2.23-2.17 (m, 2H), 2.03-1.97 (m, 1H), 1.88 (br t, J=9.4 Hz, 2H), 1.58-1.46 (m, 8H).

### MS (ESI) m/z 425.9 [M+H]⁺.

### Example 35. Synthesis of Compound 122

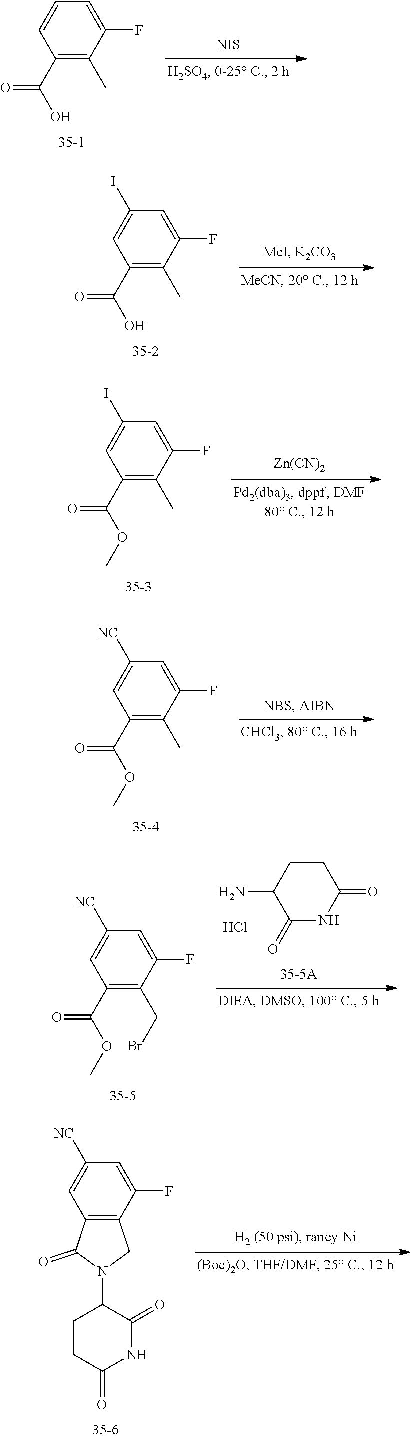

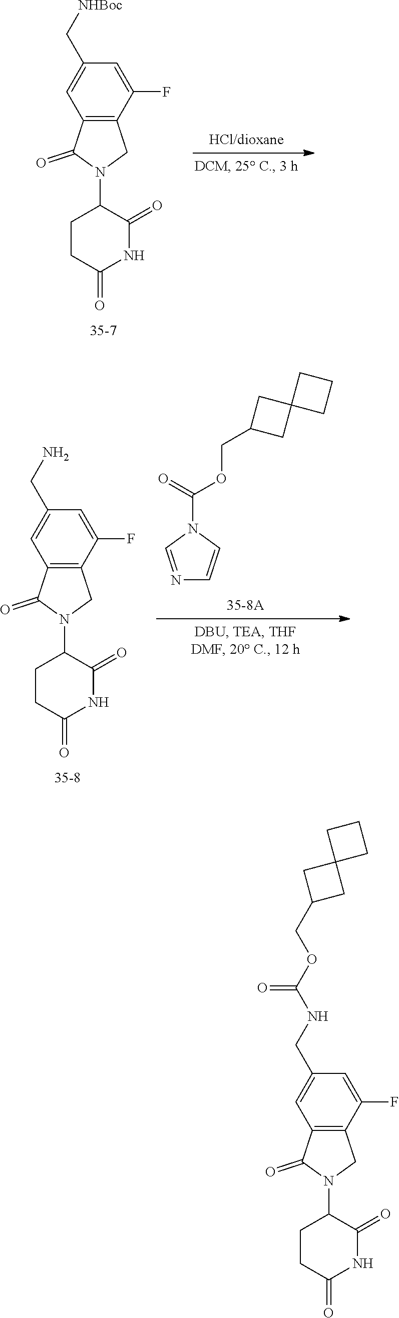

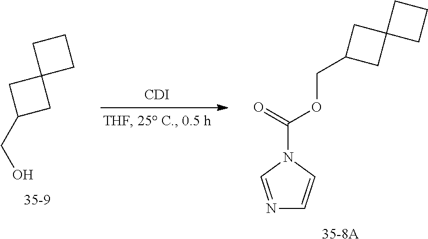

### Step 1. Procedure for Preparation of Compound 2—3-fluoro-5-iodo-2-methylbenzoic acid

To a solution of 3-fluoro-2-methylbenzoic acid (20.0 g, 130 mmol, 1.00 eq) in sulfuric acid (90.0 mL) were added 1-iodopyrrolidine-2,5-dione (26.3 g, 117 mmol, 0.900 eq) at 0° C. The reaction mixture was stirred at 25° C. for 2 h. The reaction mixture was poured into ice water and the resulting mixture was extracted with ethyl acetate (3×500 mL). The combined organic layers were washed with brine (300 mL), dried over anhydrous sodium sulfate, filtered, and concentrated under reduced pressure to give a residue. The residue was purified by reversed-phase HPLC (0.1% Formic acid, column: 100 mm*H350 mm Welch Ultimate XB_C18 20-40 μm; 120 A, B %: 10-55% 45 min; 55% 20 min) to afford 3-fluoro-5-iodo-2-methyl-benzoic acid (5.80 g, 15.5 mmol, 12.0% yield, 75% purity) as a white solid and 3-fluoro-5-iodo-2-methyl-benzoic acid (5.08 g, 17.8 mmol, 13.7% yield, 98% purity) as a white solid. ¹H NMR (400 MHz, CDCl₃) δ=8.16 (s, 1H), 7.60 (dd, J=1.6, 8.6 Hz, 1H), 2.50 (d, J=2.3 Hz, 3H).

### Step 2. Procedure for Preparation of Compound 3—3-fluoro-5-iodo-2-methylbenzoic acid

To a solution of 3-fluoro-5-iodo-2-methyl-benzoic acid (5.80 g, 20.7 mmol, 1.00 eq) in acetonitrile (60.0 mL) was added potassium carbonate (8.59 g, 62.1 mmol, 3.00 eq) and iodomethane (2.94 g, 20.7 mmol, 1.29 mL, 1.00 eq). The reaction was stirred at 20° C. for 12 h. The reaction mixture was filtered and the filtrate was concentrated under reduced pressure to afford methyl 3-fluoro-5-iodo-2-methyl-benzoate (6.00 g, 20.4 mmol, 99% yield) as colorless oil.

¹H NMR (400 MHz, CDCl₃) δ=8.00 (s, 1H), 7.53 (dd, J=1.4, 8.6 Hz, 1H), 3.91 (s, 3H), 2.44 (d, J=2.3 Hz, 3H).

### Step 3. Procedure for Preparation of Compound 4—methyl 5-cyano-3-fluoro-2-methylbenzoate

To a solution of methyl 3-fluoro-5-iodo-2-methyl-benzoate (4.26 g, 14.5 mmol, 1.00 eq) and dicyanozinc (1.02 g, 8.68 mmol, 551 uL, 0.600 eq) in dimethylformamide (50.0 mL) were added tris(dibenzylideneacetone)dipalladium(0) (663 mg, 723 umol, 0.0500 eq) and 1,1-bis(diphenylphosphino)ferrocene (963 mg, 1.74 mmol, 0.120 eq). The reaction mixture was stirred at 80° C. for 12 h under nitrogen atmosphere. The reaction mixture was quenched with water (100 mL) and extracted with ethyl acetate (3×150 mL). The combined organic layers were washed with brine (3×150 mL), dried over sodium sulfate, filtered and concentrated under reduced pressure to give a residue. The residue was purified by column chromatography (SiO₂, petroleum ether: ethyl acetate=100/1 to 0/1) to afford methyl 5-cyano-3-fluoro-2-methyl-benzoate (2.48 g, crude) as a white solid.

¹H NMR (400 MHz, CDCl₃) δ=8.01 (s, 1H), 7.47 (br d, J=8.8 Hz, 1H), 3.95 (s, 3H), 2.58 (d, J=2.1 Hz, 3H).

### Step 4. Procedure for Preparation of Compound 5—methyl 2-(bromomethyl)-5-cyano-3-fluorobenzoate

A mixture of methyl 5-cyano-3-fluoro-2-methyl-benzoate (1.00 g, 5.18 mmol, 1.00 eq), N-bromosuccinimide (1.01 g, 5.69 mmol, 1.10 eq) and 2,2-azobis(2-methylpropionitrile) (85.0 mg, 518 umol, 0.100 eq) in trichlorofluoromethane (10.0 mL) was stirred at 80° C. for 16 h. The resulting mixture was extracted with ethyl acetate (3×25 mL). The combined organic layers were washed with brine (20 mL), dried over anhydrous sodium sulfate, filtered, and the filtrate was concentrated under reduced pressure to give a residue. The residue was purified by column chromatography (SiO₂, petroleum ether/ethyl acetate=100/1 to 50/1) and concentrated under reduced pressure to afford methyl 2-(bromomethyl)-5-cyano-3-fluoro-benzoate (1.20 g, crude) as a white solid.

¹H NMR (400 MHz, CDCl₃) δ=8.10 (s, 1H), 7.56 (dd, J=1.6, 8.8 Hz, 1H), 4.99 (d, J=1.9 Hz, 2H), 4.01 (s, 3H).

### Step 5. Procedure for Preparation of Compound 6-2-(2,6-dioxopiperidin-3-yl)-7-fluoro-3-oxoisoindoline-5-carbonitrile

To a solution of methyl 2-(bromomethyl)-5-cyano-3-fluoro-benzoate (600 mg, 2.21 mmol, 1.00 eq) and 3-aminopiperidine-2,6-dione (436 mg, 2.17 mmol, 9.82^{e-1} eq, hydrochloride) in dimethylsulfoxide (20.0 mL) was added N,N-diisopropylethylamine (1.14 g, 8.82 mmol, 1.54 mL, 4.00 eq). Then the mixture was stirred at 100° C. for 5 h. The resulting mixture was extracted with ethyl acetate (3×25 mL). The combined organic layers were washed with brine (10 mL), dried over anhydrous sodium sulfate, filtered and concentrated under reduced pressure to give a residue. The residue was purified by column chromatography (SiO₂, petroleum ether: ethyl acetate=100/1 to 30/1) and concentrated under reduced pressure to afford 2-(2,6-dioxo-3-piperidyl)-7-fluoro-3-oxo-isoindoline-5-carbonitrile (1.00 g, crude) as a white solid.

¹H NMR (400 MHz, DMSO-d₆) δ=11.01 (s, 1H), 8.27-7.98 (m, 2H), 5.11 (dd, J=5.0, 13.3 Hz, 1H), 4.77-4.59 (m, 1H), 4.54-4.45 (m, 1H), 2.94-2.81 (m, 1H), 2.56 (br d, J=17.3 Hz, 1H), 2.43-2.38 (m, 1H), 2.02-1.92 (m, 1H).

### Step 6. Procedure for Preparation of Compound 7—tert-butyl ((2-(2,6-dioxopiperidin-3-yl)-7-fluoro-3-oxoisoindolin-5-yl)methyl)carbamate

A mixture of 2-(2,6-dioxo-3-piperidyl)-7-fluoro-3-oxo-isoindoline-5-carbonitrile (1.00 g, 3.48 mmol, 1.00 eq) in tetrahydrofuran (12.0 mL) and dimethyl formamide (12.0 mL) was added di-tert-butyldicarbonate (3.80 g, 17.4 mmol, 4.00 mL, 5.00 eq) and Raney nickel (298 mg, 3.48 mmol, 1.00 eq). Then the mixture was stirred at 25° C. for 12 h under hydrogen (50 Psi). The mixture was filtered. The filtrate was purified by column chromatography (SiO₂, petroleum ether: ethyl acetate=100/1 to 1/1) and concentrated under reduced pressure to afford tert-butyl ((2-(2,6-dioxopiperidin-3-yl)-7-fluoro-3-oxoisoindolin-5-yl)methyl)carbamate (270 mg, 642 umol, 18% yield, 93% purity) as a white solid.

¹H NMR (400 MHz, DMSO-d₆) δ=11.01 (s, 1H), 7.56 (br t, J=5.8 Hz, 1H), 7.48 (s, 1H), 7.33 (br d, J=9.8 Hz, 1H), 5.11 (br dd, J=5.1, 13.2 Hz, 1H), 4.54 (br d, J=17.0 Hz, 1H), 4.37 (br d, J=16.9 Hz, 1H), 4.23 (br d, J=5.6 Hz, 2H), 2.91-2.89 (m, 1H), 2.73 (br s, 1H), 2.44-2.40 (m, 1H), 2.01 (br d, J=6.0 Hz, 1H), 1.39 (s, 9H)

### Step 7. Procedure for Preparation of Compound 8—3-(6-(aminomethyl)-4-fluoro-1-oxoisoindolin-2-yl)piperidine-2,6-dione

To a solution of tert-butyl ((2-(2,6-dioxopiperidin-3-yl)-7-fluoro-3-oxoisoindolin-5-yl)methyl)carbamate (250 mg, 639 umol, 1.00 eq) in dichloromethane (3.00 mL) was added hydrochloric acid/ethyl acetate (4.00 M, 2.00 mL, 12.5 eq). The reaction was stirred at 25° C. for 3 h. The reaction mixture was concentrated under reduced pressure to afford 3-(6-(aminomethyl)-4-fluoro-1-oxoisoindolin-2-yl)piperidine-2,6-dione (234 mg, crude, hydrochloride) as a white solid.

¹H NMR (400 MHz, DMSO-d₆) δ=11.02 (s, 1H), 8.46 (br d, J=1.1 Hz, 3H), 7.78 (s, 1H), 7.66 (d, J=9.9 Hz, 1H), 5.12 (dd, J=5.1, 13.3 Hz, 1H), 4.66-4.56 (m, 1H), 4.46-4.38 (m, 1H), 4.18 (br d, J=5.5 Hz, 2H), 3.00-2.84 (m, 1H), 2.61 (br d, J=17.9 Hz, 1H), 2.46-2.40 (m, 1H), 2.07-1.98 (m, 1H).

### Step 8. Procedure for Preparation of Compound 8A—spiro[3.3]heptan-2-ylmethyl 1H-imidazole-1-carboxylate

To a solution of spiro[3.3]heptan-2-ylmethanol (45.0 mg, 357 umol, 1.00 eq) in tetrahydrofuran (1.00 mL) were added di(1H-imidazol-1-yl)methanone (116 mg, 713 umol, 2.00 eq). The reaction mixture was stirred at 25° C. for 0.5 h. The reaction mixture was concentrated under reduced pressure to afford spiro[3.3]heptan-2-ylmethyl 1H-imidazole-1-carboxylate (76.0 mg, crude) as colorless liquid.

### Step 9. Procedure for Preparation of spiro[3.3]heptan-2-ylmethyl ((2-(2,6-dioxopiperidin-3-yl)-7-fluoro-3-oxoisoindolin-5-yl)methyl)carbamate

To a solution of 3-(6-(aminomethyl)-4-fluoro-1-oxoisoindolin-2-yl)piperidine-2,6-dione (100 mg, 343 umol, 1.00 eq) in tetrahydrofuran (1.00 mL) and dimethylformamide (1.00 mL) were added 2,3,4,6,7,8,9,10-octahydropyrimido[1,2-a]azepine (52.3 mg, 343 umol, 51.8 uL, 1.00 eq), triethylamine (34.7 mg, 343 umol, 47.8 uL, 1.00 eq) and spiro[3.3]heptan-2-ylmethyl 1H-imidazole-1-carboxylate (76.0 mg, 343 umol, 1.00 eq). The reaction mixture was stirred at 20° C. for 12 h. The reaction mixture was filtered. Then, the filtrate was purified by Prep-HPLC (column: Phenomenex Luna C18 100*30 mm*5 um; mobile phase: [water (formic acid)-acetonitrile]; B %: 33%-63%, 8 min) and lyophilized to afford spiro[3.3]heptan-2-ylmethyl ((2-(2,6-dioxopiperidin-3-yl)-7-fluoro-3-oxoisoindolin-5-yl)methyl)carbamate (41.17 mg, 91.91 umol, 27% yield, 99% purity) as a white solid.

¹H NMR (400 MHz, DMSO-d₆) δ=10.99 (s, 1H), 7.79 (br t, J=6.1 Hz, 1H), 7.47 (s, 1H), 7.33 (d, J=10.0 Hz, 1H), 5.10 (dd, J=5.1, 13.4 Hz, 1H), 4.57-4.47 (m, 1H), 4.38-4.32 (m, 1H), 4.26 (br d, J=6.1 Hz, 2H), 3.90 (d, J=6.8 Hz, 2H), 2.96-2.82 (m, 1H), 2.60 (br s, 1H), 2.43-2.33 (m, 2H), 2.04-1.91 (m, 5H), 1.88-1.83 (m, 2H), 1.75-1.64 (m, 4H).

### MS (ESI) m/z.444.2 [M+H]⁺.

### Example 36. Synthesis of Compound 123

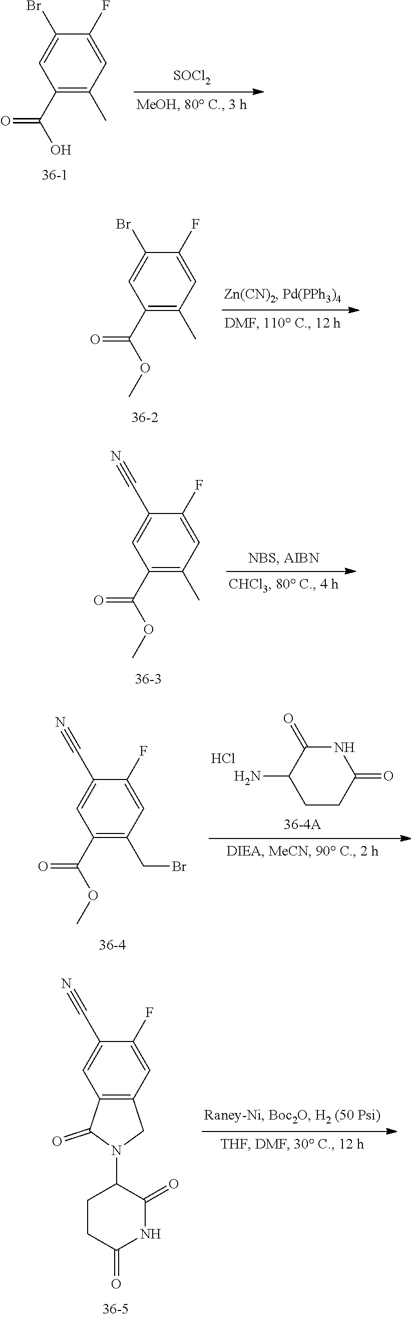

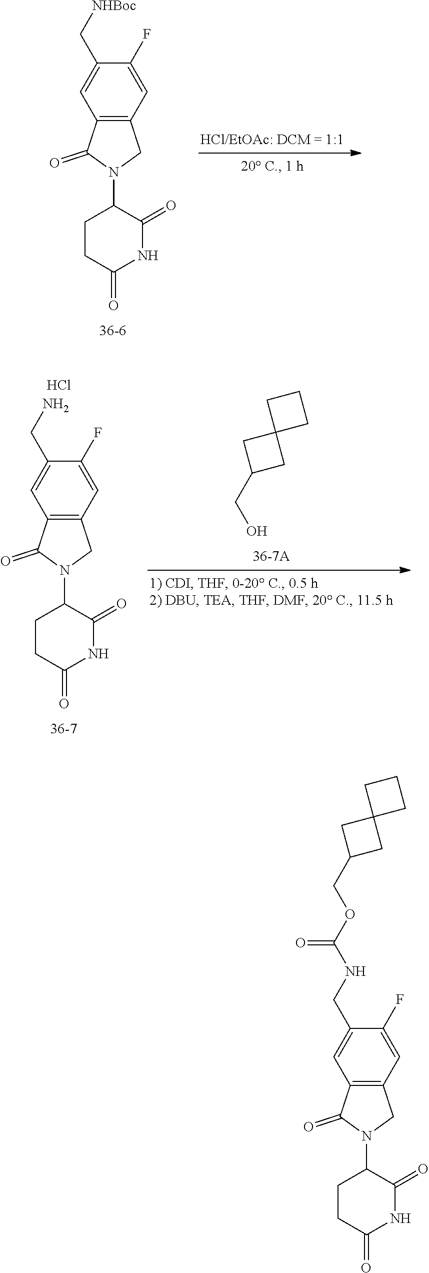

### Step 1. Procedure for Preparation of Compound 2—methyl 5-bromo-4-fluoro-2-methylbenzoate

To a solution of 5-bromo-4-fluoro-2-methylbenzoic acid (1.80 g, 7.72 mmol, 1.00 eq) in methanol (18.0 mL) was added thionyl chloride (1.84 g, 15.4 mmol, 1.12 mL, 2.00 eq). The mixture was stirred at 80° C. for 3 h. The mixture was diluted with saturated sodium bicarbonate solution (50 mL), extracted with ethyl acetate (3×30 mL). The combined organic layers were washed with brine (100 mL), dried over anhydrous sodium sulfate, filtered and concentrated under reduced pressure to afford methyl 5-bromo-4-fluoro-2-methylbenzoate (1.60 g, 6.48 mmol, 83% yield) as a yellow solid.

¹H NMR (400 MHz, CDCl₃-d) δ=8.17 (d, J=7.1 Hz, 1H), 7.02 (d, J=9.3 Hz, 1H), 3.90 (s, 3H), 2.58 (s, 3H).

### Step 2. Procedure for Preparation of Compound 3—methyl 5-cyano-4-fluoro-2-methylbenzoate

To a solution of methyl 5-bromo-4-fluoro-2-methylbenzoate (1.00 g, 4.05 mmol, 1.00 eq) in dimethylformamide (10.0 mL) were added zinc cyanide (950 mg, 8.10 mmol, 513 uL, 2.00 eq) and tetrakis[triphenylphosphine]palladium(0) (467 mg, 404 umol, 0.100 eq). The mixture was stirred at 110° C. for 12 h under nitrogen atmosphere. The mixture was filtered and the filtrate was quenched with water (50 mL), extracted with ethyl acetate (3×40 mL). The combined organic layers were washed with saturated sodium bicarbonate solution (150 mL), and dried over anhydrous sodium sulfate, filtered and concentrated under reduced pressure to give a residue. The residue was purified by column chromatography (SiO₂, petroleum ether/ethyl acetate=10/0 to 10/1) to afford methyl 5-cyano-4-fluoro-2-methylbenzoate (580 mg, 3.00 mmol, 74% yield) as colorless oil.

¹H NMR (400 MHz, CDCl₃-d) δ=8.26 (d, J=6.8 Hz, 1H), 7.13 (d, J=9.6 Hz, 1H), 3.93 (s, 3H), 2.70 (s, 3H).

### Step 3. Procedure for Preparation of Compound 4—methyl 2-(bromomethyl)-5-cyano-4-fluorobenzoate

To a solution of methyl 5-cyano-4-fluoro-2-methylbenzoate (580 mg, 3.00 mmol, 1.00 eq) in trichloromethane (7.00 mL) were added N-Bromosuccinimide (587 mg, 3.30 mmol, 1.10 eq) and azodiisobutyronitrile (49.3 mg, 300 umol, 0.100 eq). The mixture was stirred at 80° C. for 4 h. The mixture was concentrated under reduced pressure to give a residue. The residue was dissolved with water (20 mL), extracted with ethyl acetate (3×20 mL). The combined organic layers were washed with brine (50 mL), and dried over anhydrous sodium sulfate, filtered and concentrated under reduced pressure to afford methyl 2-(bromomethyl)-5-cyano-4-fluorobenzoate (880 mg, 2.59 mmol, 86% yield, 80% purity) as yellow oil.

¹H NMR (400 MHz, CDCl₃-d) δ=8.32 (d, J=6.5 Hz, 1H), 7.40 (d, J=9.3 Hz, 1H), 4.95 (s, 2H), 3.98 (s, 3H).

### Step 4. Procedure for Preparation of Compound 5-2-(2,6-dioxopiperidin-3-yl)-6-fluoro-3-oxoisoindoline-5-carbonitrile

To a solution of methyl 2-(bromomethyl)-5-cyano-4-fluorobenzoate (1.00 g, 2.94 mmol, 80% purity, 1.00 eq) in acetonitrile (10.0 mL) were added 3-aminopiperidine-2,6-dione hydrochloride (483 mg, 2.94 mmol, 1.00 eq) and N,N-diisopropylethylamine (1.14 g, 8.82 mmol, 1.54 mL, 3.00 eq). Then mixture was stirred at 90° C. for 2 h. The mixture was concentrated under reduced pressure to give a crude product. The crude product was triturated with ethyl alcohol (10 mL) to afford 2-(2,6-dioxopiperidin-3-yl)-6-fluoro-3-oxoisoindoline-5-carbonitrile (425 mg, 1.33 mmol, 45% yield, 90% purity) as a black solid.

¹H NMR (400 MHz, DMSO-d₆) δ=11.02 (s, 1H), 8.34 (d, J=5.9 Hz, 1H), 7.86 (d, J=8.9 Hz, 1H), 5.13 (dd, J=5.0, 13.3 Hz, 1H), 4.62-4.43 (m, 2H), 2.96-2.85 (m, 1H), 2.62-2.59 (m, 1H), 2.40 (br dd, J=4.4, 13.1 Hz, 1H), 2.04-1.98 (m, 1H).

### Step 5. Procedure for Preparation of Compound 6—tert-butyl ((2-(2,6-dioxopiperidin-3-yl)-6-fluoro-3-oxoisoindolin-5-yl)methyl)carbamate

To a solution of 2-(2,6-dioxopiperidin-3-yl)-6-fluoro-3-oxoisoindoline-5-carbonitrile (500 mg, 1.57 mmol, 90% purity, 1.00 eq) in tetrahydrofuran (2.50 mL) and dimethylformamide (2.50 mL) were added di-tert-butyldicarbonate (1.71 g, 7.83 mmol, 1.80 mL, 5.00 eq) and raney nickel (134 mg, 1.57 mmol, 1.00 eq). The mixture was stirred at 30° C. for 12 h under hydrogen atmosphere (50 psi). The mixture was filtered and the filtrate was diluted with water (30 mL), extracted with ethyl acetate (3×20 mL). The combined organic layers washed with brine (3×20 mL), and dried over anhydrous sodium sulfate, filtered and concentrated under reduced pressure to give a residue. The residue was purified by column chromatography (SiO₂, petroleum ether/ethyl acetate=5/1 to 0/1) to afford tert-butyl ((2-(2,6-dioxopiperidin-3-yl)-6-fluoro-3-oxoisoindolin-5-yl)methyl)carbamate (180 mg, 459 umol, 29% yield) as a brown solid.

¹H NMR (400 MHz, DMSO-d₆) δ=10.99 (s, 1H), 7.65 (d, J=6.9 Hz, 1H), 7.54 (br t, J=6.0 Hz, 1H), 7.46 (d, J=9.6 Hz, 1H), 5.08 (dd, J=5.1, 13.3 Hz, 1H), 4.48-4.40 (m, 1H), 4.34-4.28 (m, 1H), 4.23 (br d, J=5.9 Hz, 2H), 2.95-2.84 (m, 1H), 2.62-2.56 (m, 1H), 2.39 (dd, J=4.4, 13.1 Hz, 1H), 2.04-1.99 (m, 1H), 1.39 (s, 9H).

### Step 6. Procedure for Preparation of Compound 7—3-(6-(aminomethyl)-5-fluoro-1-oxoisoindolin-2-yl)piperidine-2,6-dione

To a solution of tert-butyl ((2-(2,6-dioxopiperidin-3-yl)-6-fluoro-3-oxoisoindolin-5-yl)methyl)carbamate (180 mg, 459 umol, 1.00 eq) in dichloromethane (1.00 mL) was added hydrochloric acid/ethyl acetate (4.00 M, 1.00 mL, 8.70 eq). The mixture was stirred at 20° C. for 1 h. The mixture was filtered and the filtrate was concentrated under reduced pressure to afford 3-(6-(aminomethyl)-5-fluoro-1-oxoisoindolin-2-yl) piperidine-2,6-dione (120 mg, 366 umol, 80% yield, hydrochloride) as an off-white solid.

¹H NMR (400 MHz, DMSO-d₆) δ=11.01 (s, 1H), 8.39 (br s, 3H), 7.98 (d, J=6.9 Hz, 1H), 7.60 (d, J=9.5 Hz, 1H), 5.10 (dd, J=5.0, 13.4 Hz, 1H), 4.53-4.46 (m, 1H), 4.40-4.33 (m, 1H), 4.21-4.15 (m, 2H), 2.96-2.85 (m, 1H), 2.60 (br d, J=18.1 Hz, 1H), 2.44-2.35 (m, 1H), 2.04-1.99 (m, 1H).

### Step 7. Procedure for Preparation of spiro[3.3]heptan-2-ylmethyl ((2-(2,6-dioxopiperidin-3-yl)-6-fluoro-3-oxoisoindolin-5-yl)methyl)carbamate

To a solution of spiro[3.3]heptan-2-ylmethanol (20.0 mg, 158 umol, 1.00 eq) in tetrahydrofuran (0.500 mL) was added di(1H-imidazol-1-yl)methanone (51.4 mg, 316 umol, 2.00 eq) at 0° C. Then the mixture was stirred at 20° C. for 0.5 h. A mixture of 3-(6-(aminomethyl)-5-fluoro-1-oxoisoindolin-2-yl)piperidine-2,6-dione (51.9 mg, 158 umol, 1.00 eq, hydrochloride), 2,3,4,6,7,8,9,10-octahydropyrimido[1,2-a]azepine (24.1 mg, 158 umol, 23.8 uL, 1.00 eq) and triethylamine (16.0 mg, 158 umol, 22.1 uL, 1.00 eq) in tetrahydrofuran (0.500 mL) and N,N-dimethylformamide (0.500 mL) was added into this mixture. The reaction mixture was stirred at 20° C. for 11.5 h. The mixture was concentrated under reduced pressure to give a residue. The residue was purified by Prep-HPLC (column: Phenomenex luna C18 150*25 mm*10 um; mobile phase: [water (formic acid)-acetonitrile]; B %: 40%-70%, 10.5 min) and lyophilized to afford spiro[3.3]heptan-2-ylmethyl((2-(2,6-dioxopiperidin-3-yl)-6-fluoro-3-oxoisoindolin-5-yl)methyl)carbamate (22.38 mg, 49.4 umol, 31% yield, 98% purity) as an off-white solid.

¹H NMR (400 MHz, DMSO-d₆) δ=10.98 (br s, 1H), 7.79 (br t, J=5.9 Hz, 1H), 7.65 (d, J=6.9 Hz, 1H), 7.47 (d, J=9.6 Hz, 1H), 5.09 (dd, J=5.1, 13.3 Hz, 1H), 4.47-4.41 (m, 1H), 4.34-4.25 (m, 3H), 3.91 (d, J=6.8 Hz, 2H), 2.96-2.85 (m, 1H), 2.59 (td, J=2.0, 15.2 Hz, 1H), 2.42-2.34 (m, 2H), 2.06-1.93 (m, 5H), 1.90-1.84 (m, 2H), 1.79-1.67 (m, 4H).

### MS (ESI) m/z 444.1 [M+H]⁺.

### Example 37. Synthesis of Compounds 124-129

Compound 124: 15.3 mg (21.9%). 2-(spiro[3.3]heptan-2-yl)ethyl ((2-(2,6-dioxopiperidin-3-yl)-3-oxoisoindolin-5-yl)methyl)carbamate. HPLC purity: 100%; Ret_time: 1.326 min; HRMS (ESI) calculated for C₂₄H₂₉N₃O₅: 439.21; observed: 440.1 [M+H]⁺

Compound 125: 17.7 mg (25.3%) (6,6-difluorospiro[3.3]heptan-2-yl)methyl ((2-(2,6-dioxopiperidin-3-yl)-3-oxoisoindolin-5-yl)methyl)carbamate. HPLC purity: 95.56%; Ret_time: 1.030 min; HRMS (ESI) calculated for C₂₃H₂₅F₂N₃O₅: 461.18; observed: 462.2 [M+H]⁺

Compound 126: 12.7 mg (18.1%) spiro[2.3]hexan-5-ylmethyl ((2-(2,6-dioxopiperidin-3-yl)-3-oxoisoindolin-5-yl)methyl)carbamate. HPLC purity: 98.30%; Ret_time: 1.180 min; HRMS (ESI) calculated for C₂₂H₂₅N₃O₅: 411.18; observed: 412.0 [M+H]⁺

Compound 127: 10.7 mg (15.3%) dispiro[3.1.36.14]decan-2-ylmethyl((2-(2,6-dioxopiperidin-3-yl)-3-oxoisoindolin-5-yl)methyl)carbamate. ¹H NMR (400 MHz, Acetonitrile-d₃) δ8.86 (s, 1H), 7.68 (s, 1H), 7.64-7.48 (m, 2H), 6.06 (s, 1H), 5.11 (dd, J=13.4, 5.2 Hz, 1H), 4.49-4.32 (m, 4H), 3.98 (d, J=6.7 Hz, 2H), 2.96-2.65 (m, 2H), 2.49-2.36 (m, 2H), 1.93 (d, J=10.1 Hz, 9H), 1.79 (dq, J=18.2, 9.9, 8.2 Hz, 5H). HPLC purity: 98.26%; Ret_time: 1.414 min; HRMS (ESI) calculated for C₂₆H₃₁N₃O₅: 465.23; observed: 466.2 [M+H]⁺

Compound 128: 10.3 mg (14.7%) 3-cyclobutylpropyl ((2-(2,6-dioxopiperidin-3-yl)-3-oxoisoindolin-5-yl)methyl)carbamate. HPLC purity: 100%; Ret_time: 1.239 min; HRMS (ESI) calculated for C₂₂H₂₇N₃O₅: 413.20; observed: 414.1 [M+H]⁺

Compound 129: 19.7 mg (28.1%) 2-((1R,5S)-6,6-dimethylbicyclo[3.1.1]hept-2-en-2-yl)ethyl ((2-(2,6-dioxopiperidin-3-yl)-3-oxoisoindolin-5-yl)methyl)carbamate. HPLC purity: 99.60%; Ret_time: 1.242 min; HRMS (ESI) calculated for C₂₆H₃₁N₃O₅: 465.23; observed: 464.2 [M−H]⁻

### Example 38. Synthesis of Compound 130

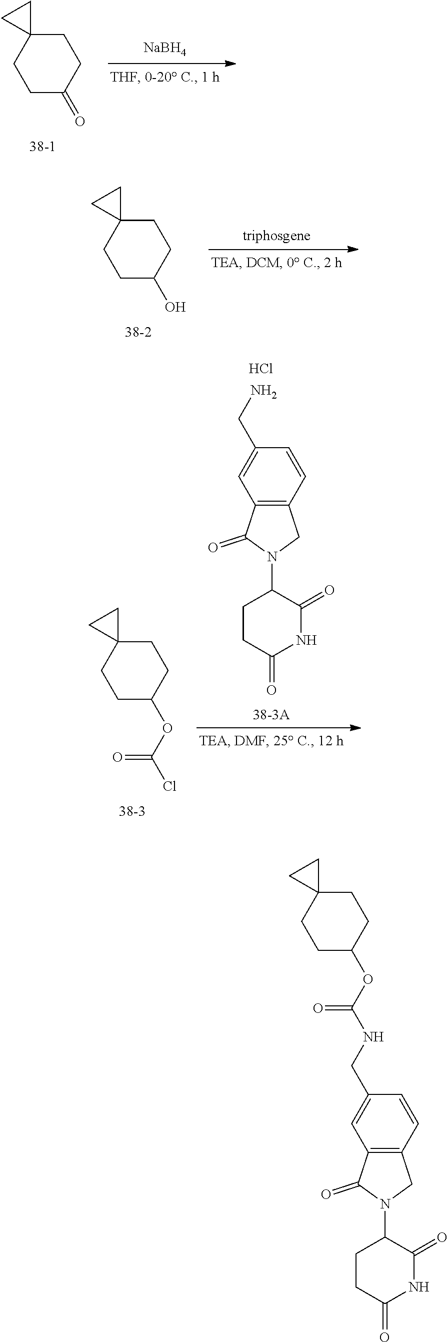

### Step 1. Procedure for Compound 2—spiro[2.5]octan-6-ol

To a solution of spiro[2.5]octan-6-one (200 mg, 1.61 mmol, 1.00 eq) in tetrahydrofuran (2.00 mL) was added sodium borohydride (122 mg, 3.22 mmol, 2.00 eq) at 0° C. The reaction mixture was stirred at 20° C. for 1 h. The reaction mixture was quenched with saturated ammonium chloride (14 mL) and then extracted with ethyl acetate (3×10 mL). The organic layers were washed with brine (10.0 mL), dried over anhydrous sodium sulfate, filtered and concentrated under reduced pressure to afford spiro[2.5]octan-6-ol (180 mg, 1.43 mmol, 89% yield) as yellow oil.

1H NMR (400 MHz, CDCl3-d) δ=3.78-3.67 (m, 1H), 1.91-1.85 (m, 2H), 1.56 (br d, J=13.1 Hz, 2H), 1.52-1.46 (m, 2H), 1.44 (br d, J=3.5 Hz, 1H), 1.10 (br d, J=13.0 Hz, 2H), 0.32-0.28 (m, 2H), 0.25-0.20 (m, 2H).

### Step 2. Procedure for Compound 3—spiro[2.5]octan-6-yl carbonochloridate

To a solution of spiro[2.5]octan-6-ol (40.0 mg, 317 umol, 1.00 eq) in dichloromethane (0.400 mL) was added bis(trichloromethyl) carbonate (94.1 mg, 317 umol, 1.00 eq) and triethylamine (64.2 mg, 634 umol, 88.0 uL, 2.00 eq) at 0° C. The reaction mixture was stirred at 20° C. for 1 h. The reaction mixture was concentrated under reduced pressure to afford spiro[2.5]octan-6-yl carbonochloridate (55.0 mg, crude, hydrochloride) as a yellow solid.

### Step 3. Procedure for spiro[2.5]octan-6-yl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (45.0 mg, 145 umol, 1.00 eq, hydrochloride) in N,N-dimethylformamide (0.500 mL) was added triethylamine (73.5 mg, 726 umol, 101 uL, 5.00 eq) and spiro[2.5]octan-6-yl carbonochloridate (54.8 mg, 291 umol, 2.00 eq). The reaction mixture was stirred at 25° C. for 12 h. The reaction mixture was filtered. The filtrate was purified by Prep-HPLC(column: Phenomenex Luna C8 250*50 mm*10 um; mobile phase: [water(formic acid)-acetonitrile]; B %: 31%-61%, 9 min) and lyophilized to afford spiro[2.5]octan-6-yl ((2-(2,6-dioxopiperidin-3-yl)-3-oxoisoindolin-5-yl)methyl)carbamate (1.80 mg, 4.10 umol, 3% yield, 97% purity) as an off-white solid.

¹H NMR (400 MHz, DMSO-d6) δ=10.98 (s, 1H), 7.77-7.68 (m, 1H), 7.61 (s, 1H), 7.57-7.49 (m, 2H), 5.14-5.07 (m, 1H), 4.63-4.55 (m, 1H), 4.47-4.39 (m, 1H), 4.34-4.24 (m, 3H), 2.95-2.86 (m, 1H), 2.59 (br s, 1H), 2.39 (br d, J=8.4 Hz, 1H), 2.03-1.98 (m, 1H), 1.83-1.75 (m, 2H), 1.53-1.44 (m, 4H), 1.22-1.15 (m, 2H), 0.29-0.19 (m, 4H).

### MS (ESI) m/z.426.1 [M+H]⁺.

### Example 39. Synthesis of Compound 131

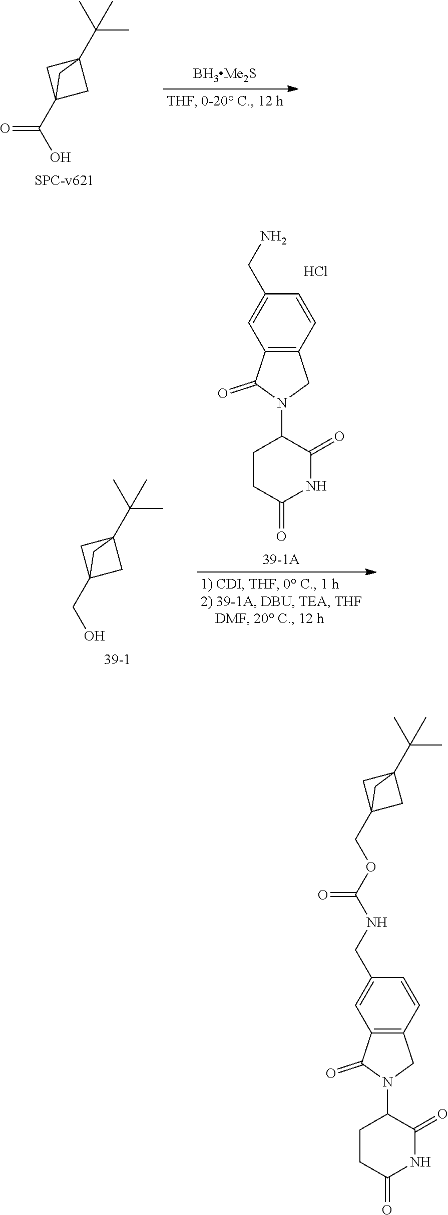 [SMILES: CC(C)(C)C12CC(C(=O)O)(C1)C2 ; CC(C)(C)C12CC(CO)(C1)C2 ; NCc1ccc2c(c1)C(=O)N(C1CCC(=O)NC1=O)C2 ; Cl ; CC(C)(C)C12CC(COC(=O)NCc3ccc4c(c3)C(=O)N(C3CCC(=O)NC3=O)C4)(C1)C2]

### Step 1. Procedure for Preparation of Compound 2 —(3-(tert-butyl)bicyclo[1.1.1]pentan-1-yl)methanol

To a solution of 3-(tert-butyl)bicyclo[1.1.1]pentane-1-carboxylic acid (100 mg, 594 umol, 1.00 eq) in tetrahydrofuran (1.00 mL) was added borane dimethyl sulfide complex (10.0 M, 119 uL, 2.00 eq) at 0° C. The reaction mixture was stirred at 20° C. for 12 h. The reaction mixture was added to methanol (10.0 mL) to quench. Then the mixture was concentrated under reduced pressure to afford (3-(tert-butyl)bicyclo[1.1.1]pentan-1-yl)methanol (77.0 mg, 499 umol, 84% yield) as colorless oil.

¹H NMR (400 MHz, CDCl₃) δ=3.60 (s, 2H), 1.49 (s, 6H), 0.85 (s, 9H).

### Step 2. Procedure for preparation of (3-(tert-butyl)bicyclo[1.1.1]pentan-1-yl)methyl((2-(2,6-dioxopiperidin-3-yl)-3-oxoisoindolin-5-yl)methyl)carbamate

To a solution of (3-(tert-butyl)bicyclo[1.1.1]pentan-1-yl)methanol (35.0 mg, 227 umol, 1.00 eq) in tetrahydrofuran (0.300 mL) was added di(1H-imidazol-1-yl)methanone (73.6 mg, 454 umol, 2.00 eq) at 0° C. The mixture was stirred at 20° C. for 1 h. The resulting solution was added to a mixture of 3-(6-(aminomethyl)-1-oxoisoindolin-2-yl)piperidine-2,6-dione (70.3 mg, 227 umol, 1.00 eq, hydrochloride), 2,3,4,6,7,8,9,10-octahydropyrimido[1,2-a]azepine (34.5 mg, 227 umol, 34.2 uL, 1.00 eq) and triethylamine (23.0 mg, 227 umol, 31.6 uL, 1.00 eq) in tetrahydrofuran (0.300 mL) and dimethylformamide (0.300 mL). The reaction mixture was stirred at 20° C. for 12 h. The reaction mixture was concentrated under reduced pressure to give a residue. The residue was dissolved in dimethylformamide (1.5 mL) and then filtered. The filtrate was purified by Prep-HPLC(column: Phenomenex luna C18 150*25 mm*10 um; mobile phase: [water(formic acid)-acetonitrile]; B %: 40%-70%, 9 min) and lyophilized to afford (3-(tert-butyl)bicyclo[1.1.1]pentan-1-yl)methyl((2-(2,6-dioxopiperidin-3-yl)-3-oxoisoindolin-5-yl)methyl)carbamate (21.05 mg, 45.95 umol, 20% yield, 99% purity) as an off-white solid.

¹H NMR (400 MHz, DMSO-d₆) δ=10.97 (br s, 1H), 7.78 (br t, J=6.1 Hz, 1H), 7.61 (s, 1H), 7.58-7.53 (m, 1H), 7.53-7.47 (m, 1H), 5.11 (dd, J=5.0, 13.4 Hz, 1H), 4.46-4.36 (m, 1H), 4.34-4.21 (m, 3H), 3.98 (s, 2H), 2.98-2.85 (m, 1H), 2.60 (td, J=2.1, 15.3 Hz, 1H), 2.43-2.36 (m, 1H), 2.04-1.93 (m, 1H), 1.44 (s, 5H), 1.27-1.14 (m, 1H), 0.80 (s, 8H), 0.71 (br s, 1H).

¹H NMR (400 MHz, DMSO-d₆, T=80° C.) δ=10.67 (br s, 1H), 7.64 (s, 1H), 7.56-7.49 (m, 2H), 7.49-7.33 (m, 1H), 5.06 (dd, J=5.3, 13.1 Hz, 1H), 4.46-4.40 (m, 1H), 4.37-4.27 (m, 3H), 3.99 (s, 2H), 2.89 (ddd, J=5.5, 13.4, 17.5 Hz, 1H), 2.66-2.60 (m, 1H), 2.45-2.35 (m, 1H), 2.03 (dtd, J=2.6, 5.3, 12.8 Hz, 1H), 1.49-1.39 (m, 6H), 0.81 (s, 9H).

### MS (ESI) m/z 454.2 [M+H]⁺.

### Example 40. Synthesis of Compound 132

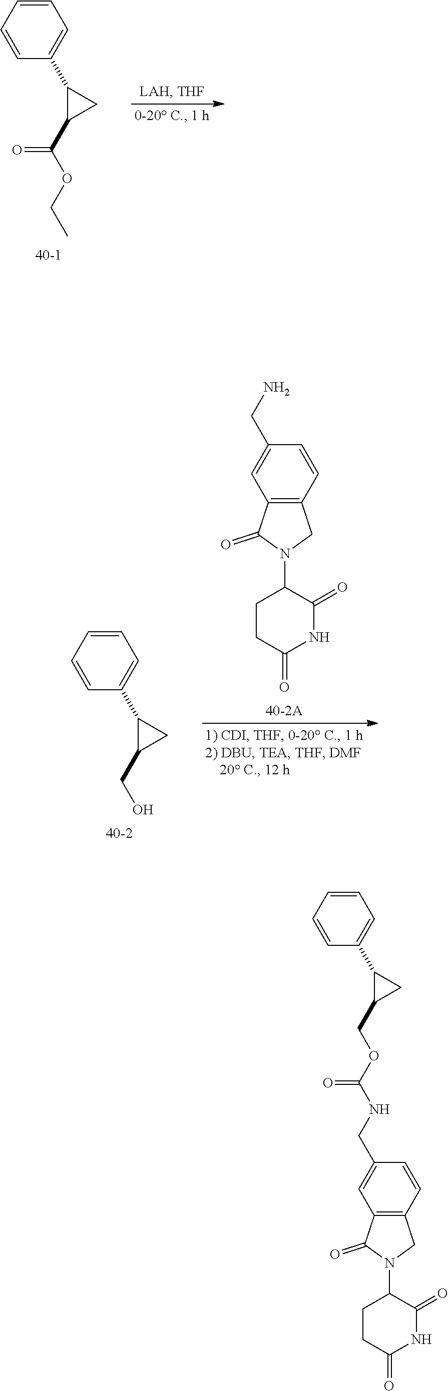

### Step 1. Procedure for Compound 2—((1R,2R)-2-phenylcyclopropyl)methanol

To a mixture of (1R,2R)-ethyl 2-phenylcyclopropanecarboxylate (1.00 g, 5.26 mmol, 1.00 eq) in tetrahydrofuran (10.0 mL) was added lithium aluminum hydride (599 mg, 15.8 mmol, 3.00 eq) at 0° C. The mixture was stirred at 20° C. for another 1 h. The two batches reaction mixture were combined, quenched with sodium sulfate decahydrate (5.00 g) and filtered. The filtrate was concentrated under reduced pressure to afford ((1R,2R)-2-phenylcyclopropyl)methanol (1.50 g, 10.1 mmol, 96% yield) as colorless oil.

¹H NMR (400 MHz, CDCl3) δ=7.33-7.24 (m, 2H), 7.22-7.16 (m, 1H), 7.13-7.06 (m, 2H), 3.75-3.54 (m, 2H), 1.92-1.76 (m, 1H), 1.55-1.40 (m, 1H), 1.06-0.86 (m, 2H).

### Step 2. Procedure for ((1R,2R)-2-phenylcyclopropyl)methyl ((2-(2,6-dioxopiperidin-3-yl)-3-oxoisoindolin-5-yl)methyl)carbamate

To a mixture of ((1R,2R)-2-phenylcyclopropyl)methanol (100 mg, 675 umol, 1.00 eq) in tetrahydrofuran (0.500 mL) was added di(1H-imidazol-1-yl)methanone (219 mg, 1.35 mmol, 2.00 eq) at 0° C. The mixture was stirred at 20° C. for 1 h. The reaction mixture was added into a solution of 3-(6-(aminomethyl)-1-oxoisoindolin-2-yl)piperidine-2,6-dione (209 mg, 675 umol, 1.00 eq, hydrochloride), triethylamine (675 umol, 93.9 uL, 1.00 eq) and 2,3,4,6,7,8,9,10-octahydropyrimido[1,2-a]azepine (675 umol, 102 uL, 1.00 eq) in tetrahydrofuran (0.500 mL) and dimethylformamide (0.500 mL). The reaction mixture was stirred at 20° C. for 12 h. The reaction was concentrated under reduced pressure to give a residue. The residue was dissolved in dimethylformamide (1 mL) and then filtered. The filtrate was purified by Prep-HPLC (column: Phenomenex luna C18 150*25 mm*10 um; mobile phase: [water formic acid)-acetonitrile]; B %: 32%-62%, 10 min) and lyophilized to afford ((1R,2R)-2-phenylcyclopropyl)methyl ((2-(2,6-dioxopiperidin-3-yl)-3-oxoisoindolin-5-yl)methyl)carbamate (40.8 mg, 90.27 umol, 13% yield, 99% purity) as a white solid.

¹H NMR (400 MHz, DMSO-d₆) δ=10.98 (br s, 1H), 7.84 (br t, J=6.1 Hz, 1H), 7.63 (s, 1H), 7.57-7.43 (m, 2H), 7.29-7.18 (m, 2H), 7.16-7.09 (m, 1H), 7.06 (br d, J=7.4 Hz, 2H), 5.12 (dd, J=5.1, 13.3 Hz, 1H), 4.51-4.40 (m, 1H), 4.37-4.21 (m, 3H), 4.09-3.99 (m, 1H), 3.93 (dd, J=7.5, 11.4 Hz, 1H), 2.98-2.86 (m, 1H), 2.66-2.57 (m, 1H), 2.47-2.38 (m, 1H), 2.06-1.97 (m, 1H), 1.95-1.87 (m, 1H), 1.47-1.35 (m, 1H), 1.05-0.75 (m, 2H).

### MS (ESI) m/z 448.2 [M+H]⁺.

### Example 41. Synthesis of Compound 133

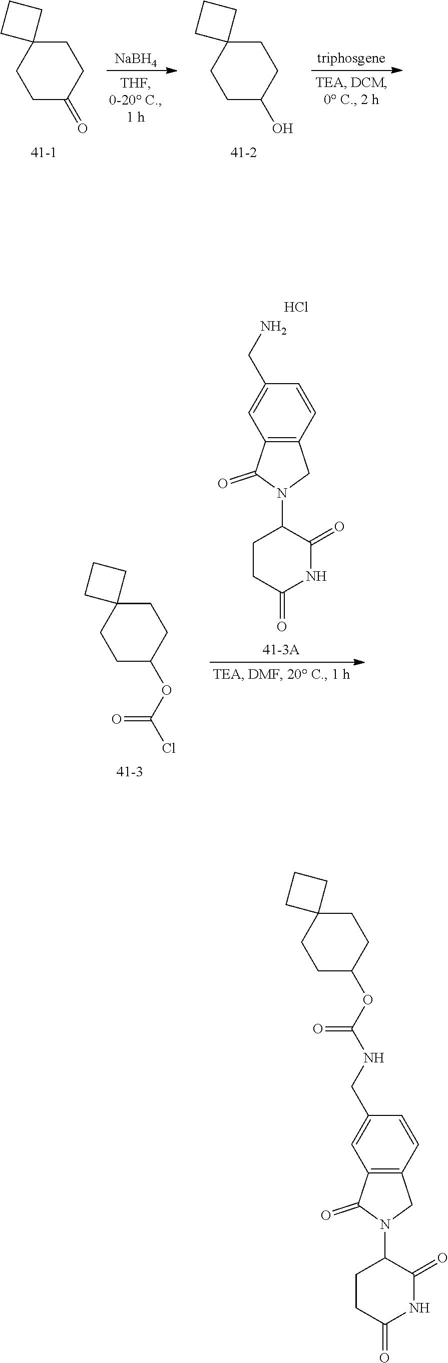

### Step 1. Procedure for Compound 2—spiro[3.5]nonan-7-ol

To a solution of spiro[3.5]nonan-7-one (50.0 mg, 362 umol, 1.00 eq) in tetrahydrofuran (1.00 mL) was added sodium borohydride (27.4 mg, 724 umol, 2.00 eq) at 0° C. The reaction mixture was stirred at 20° C. for 1 h. The reaction mixture was quenched with saturated ammonium chloride (4 mL) and then extracted with ethyl acetate (3×10 mL). The organic layers were washed with brine (10 mL), dried over anhydrous sodium sulfate, filtered and concentrated under reduced pressure to afford spiro[3.5]nonan-7-ol (31.0 mg, 221 umol, 62% yield) as colorless oil.

¹H NMR (400 MHz, CDCl3-d) δ=3.60 (br s, 1H), 1.87-1.81 (m, 2H), 1.76-1.68 (m, 8H), 1.32 (br dd, J=4.7, 9.7 Hz, 4H), 0.90-0.84 (m, 1H).

### Step 2. Procedure for Compound 3—spiro[3.5]nonan-7-yl carbonochloridate

To a solution of spiro[3.5]nonan-7-ol (30.0 mg, 214 umol, 1.00 eq) and bis(trichloromethyl) carbonate (31.8 mg, 107 umol, 0.500 eq) in dichloromethane (0.500 mL) was added dropwise triethylamine (21.7 mg, 214 umol, 29.8 uL, 1.00 eq) at 0° C. The reaction mixture was stirred at 0° C. for 1 h. Bis(trichloromethyl) carbonate (31.8 mg, 107 umol, 0.500 eq) was added to the mixture and the reaction mixture was stirred at 0° C. for 1 h. The reaction mixture was concentrated under reduced pressure to afford spiro[3.5]nonan-7-yl carbonochloridate (44.0 mg, crude) was obtained as a white solid.

### Step 3. Procedure for spiro[3.5]nonan-7-yl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (30.0 mg, 96.9 umol, 1.00 eq, hydrochloride) in dimethylformamide (0.300 mL) was added triethylamine (49.0 mg, 484 umol, 67.4 uL, 5.00 eq) and spiro[3.5]nonan-7-yl carbonochloridate (39.3 mg, 194 umol, 2.00 eq). The reaction mixture was stirred at 20° C. for 1 h. The reaction mixture was filtered. The filtrate was purified by Prep-HPLC(column: Phenomenex Luna C8 250*50 mm*10 um; mobile phase: [water(formic acid)-acetonitrile]; B %: 36%-66%, 9 min) and lyophilized to afford spiro[3.5]nonan-7-yl ((2-(2,6-dioxopiperidin-3-yl)-3-oxoisoindolin-5-yl)methyl)carbamate (2.57 mg, 5.67 umol, 6% yield, 97% purity) as a white solid.

¹H NMR (400 MHz, DMSO-d₆) δ=11.04-10.89 (m, 1H), 7.67 (br t, J=5.6 Hz, 1H), 7.60 (s, 1H), 7.56-7.53 (m, 1H), 7.51-7.48 (m, 1H), 5.14-5.07 (m, 1H), 4.49-4.39 (m, 2H), 4.33-4.22 (m, 3H), 2.96-2.85 (m, 1H), 2.65-2.55 (m, 1H), 2.39 (br d, J=8.5 Hz, 1H), 2.03-1.97 (m, 1H), 1.84-1.77 (m, 2H), 1.72-1.64 (m, 8H), 1.37-1.30 (m, 4H)

### MS (ESI) m/z. 440.1 [M+H]⁺.

### Example 42. Synthesis of Compound 134

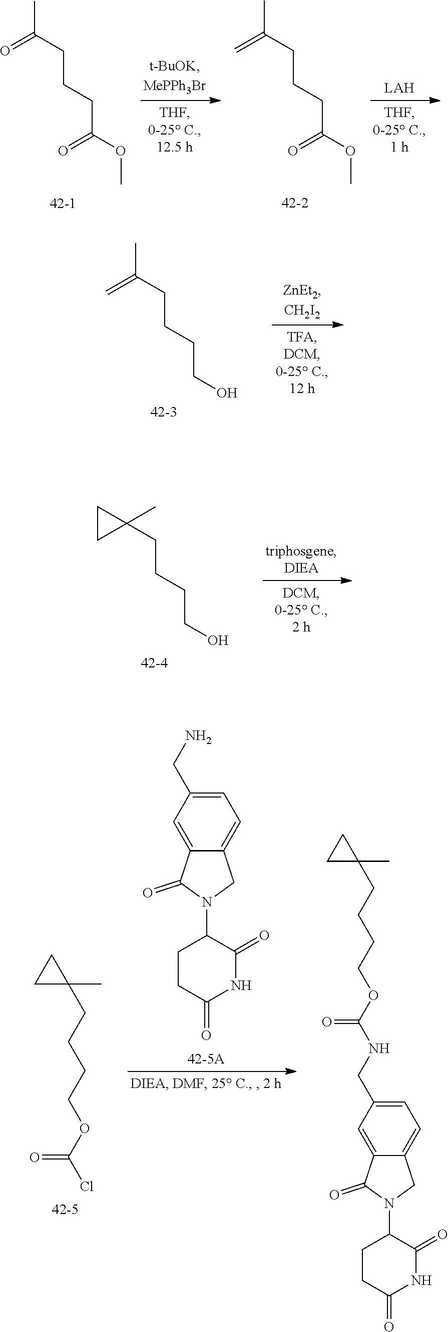

### Step 1. Procedure for Compound 2—methyl 5-methylhex-5-enoate

To a solution of methyl 5-oxohexanoate (32.2 g, 90.2 mmol, 1.30 eq) in tetrahydrofuran (300 mL) was added potassium tert-butoxide (11.7 g, 104 mmol, 1.50 eq) at 0° C. The mixture was stirred at 0° C. for 0.5 h. The mixture was added methyl 5-oxohexanoate (10.0 g, 69.4 mmol, 1.00 eq). The reaction mixture was stirred at 25° C. for 12 h. The reaction mixture was diluted with water (50 mL), extracted with ethyl acetate (3×25 mL). The combined organic layers were dried over anhydrous sodium sulfate, filtered and concentrated under reduced pressure to give a residue. The residue was purified by column chromatography (SiO₂, petroleum ether/ethyl acetate=50/1 to 20/1) to afford methyl 5-methylhex-5-enoate (7.00 g, 49.0 mmol, 70% yield) as colorless oil.

¹H NMR (400 MHz, CDCl₃-d) δ=4.76-4.67 (m, 2H), 3.67 (s, 3H), 2.31 (t, J=7.6 Hz, 2H), 2.05 (t, J=7.6 Hz, 2H), 1.82-1.75 (m, 2H), 1.72 (s, 3H).

### Step 2. Procedure for Preparation of Compound 3—5-methylhex-5-en-1-ol

To a solution of methyl 5-methylhex-5-enoate (1.00 g, 7.03 mmol, 1.00 eq) in tetrahydrofuran (10.0 mL) was added lithium aluminium hydride (400 mg, 10.6 mmol, 1.50 eq) at 0° C. The mixture was stirred at 25° C. for 1 h. The reaction mixture was quenched with saturated sodium sulfate decahydrate (20.0 mL), filtered and the filtrate was concentrated under reduced pressure to afford 5-methylhex-5-en-1-ol (400 mg, 3.50 mmol, 49% yield) as colorless oil.

¹H NMR (400 MHz, CDCl₃-d) δ=4.70 (br d, J=12.3 Hz, 2H), 3.66 (t, J=6.2 Hz, 2H), 2.05 (br t, J=7.2 Hz, 2H), 1.72 (s, 3H), 1.60-1.50 (m, 4H).

### Step 3. Procedure for Compound 4—4-(1-methylcyclopropyl)butan-1-ol

To a solution of 5-methylhex-5-en-1-ol (400 mg, 3.50 mmol, 1.00 eq) in dichloromethane (20.0 mL) were added diethylzinc (1 M, 10.5 mL, 3.00 eq), diiodomethane (2.81 g, 10.5 mmol, 848 uL, 3.00 eq) and trifluoroacetic acid (1.20 g, 10.5 mmol, 778 uL, 3.00 eq) at 0° C. The mixture was stirred at 25° C. for 12 h. The mixture was diluted with water (20 mL), extracted with ethyl acetate (3×25 mL). The combined organic layers were dried over anhydrous sodium sulfate, filtered and concentrated under reduced pressure to give a residue. The residue was purified by column chromatography (SiO₂, petroleum ether/ethyl acetate=10/1 to 3/1) to afford 4-(1-methylcyclopropyl)butan-1-ol (200 mg, crude) as colorless oil.

¹H NMR (400 MHz, CDCl₃-d) δ=3.65 (d, J=6.5 Hz, 2H), 2.07-2.03 (m, 1H), 1.55 (br d, J=6.9 Hz, 2H), 1.47-1.41 (m, 2H), 1.25 (br d, J=8.1 Hz, 2H), 1.01 (s, 3H), 0.22 (br d, J=7.1 Hz, 4H).

### Step 4. Procedure for Preparation of Compound 5—5-methylhex-5-en-1-ol

To a solution of 4-(1-methylcyclopropyl)butan-1-ol (100 mg, 780 umol, 1.00 eq) in dichloromethane (2.00 mL) were added N,N-diisopropylethylamine (201 mg, 1.56 mmol, 271 uL, 2.00 eq) and bis(trichloromethyl) carbonate (278 mg, 936 umol, 1.20 eq) at 0° C. The mixture was stirred at 25° C. for 2 h. The reaction mixture was filtered and the filtrate was concentrated under reduced pressure to afford 4-(1-methylcyclopropyl)butyl carbonochloridate (100 mg, crude) as a yellow solid.

### Step 5. Procedure for 4-(1-methylcyclopropyl)butyl ((2-(2,6-dioxopiperidin-3-yl)-3-oxoisoindolin-5-yl)methyl)carbamate

To a solution of 3-(6-(aminomethyl)-1-oxoisoindolin-2-yl)piperidine-2,6-dione (143 mg, 524 umol, 1.00 eq, hydrochloride) in dimethylformamide (2.00 mL) were added N,N-diisopropylethylamine (203 mg, 1.57 mmol, 274 uL, 3.00 eq) and 4-(1-methylcyclopropyl)butyl carbonochloridate (100 mg, 524 umol, 1.00 eq). The mixture was stirred at 25° C. for 2 h. The mixture was filtered. The residue was purified by reverse phase chromatography (C18, 40 g; condition: water/acetonitrile=100:0 to 0:100, 0.1% formic acid) and lyophilized to afford 4-(1-methylcyclopropyl)butyl ((2-(2,6-dioxopiperidin-3-yl)-3-oxoisoindolin-5-yl)methyl)carbamate (30.89 mg, 72 umol, 13% yield, 99% purity) as an off-white solid.

¹H NMR (400 MHz, DMSO-d₆) δ=10.98 (s, 1H), 7.76 (br t, J=6.0 Hz, 1H), 7.61 (s, 1H), 7.57-7.48 (m, 2H), 5.15-5.07 (m, 1H), 4.47-4.28 (m, 2H), 4.27 (br d, J=6.6 Hz, 2H), 3.96 (t, J=6.6 Hz, 2H), 2.97-2.85 (m, 1H), 2.64-2.55 (m, 1H), 2.45-2.33 (m, 1H), 2.04-1.94 (m, 1H), 1.59-1.48 (m, 2H), 1.43-1.31 (m, 2H), 1.24-1.16 (m, 2H), 0.97 (s, 3H), 0.24-0.10 (m, 4H).

¹H NMR (400 MHz, DMSO-d₆, T=80° C.) δ=10.80-10.68 (m, 1H), 7.63 (s, 1H), 7.55-7.50 (m, 2H), 7.49-7.38 (m, 1H), 5.10-5.03 (m, 1H), 4.46-4.32 (m, 2H), 4.29 (d, J=6.1 Hz, 2H), 3.98 (t, J=6.6 Hz, 2H), 2.95-2.84 (m, 1H), 2.63 (br d, J=17.6 Hz, 1H), 2.46-2.35 (m, 1H), 2.07-2.00 (m, 1H), 1.59-1.52 (m, 2H), 1.43-1.33 (m, 2H), 1.27-1.20 (m, 2H), 0.99 (s, 3H), 0.26-0.17 (m, 4H).

### MS (ESI) m/z 428.1 [M+H]⁺.

### Example 43. Synthesis of Compound 135

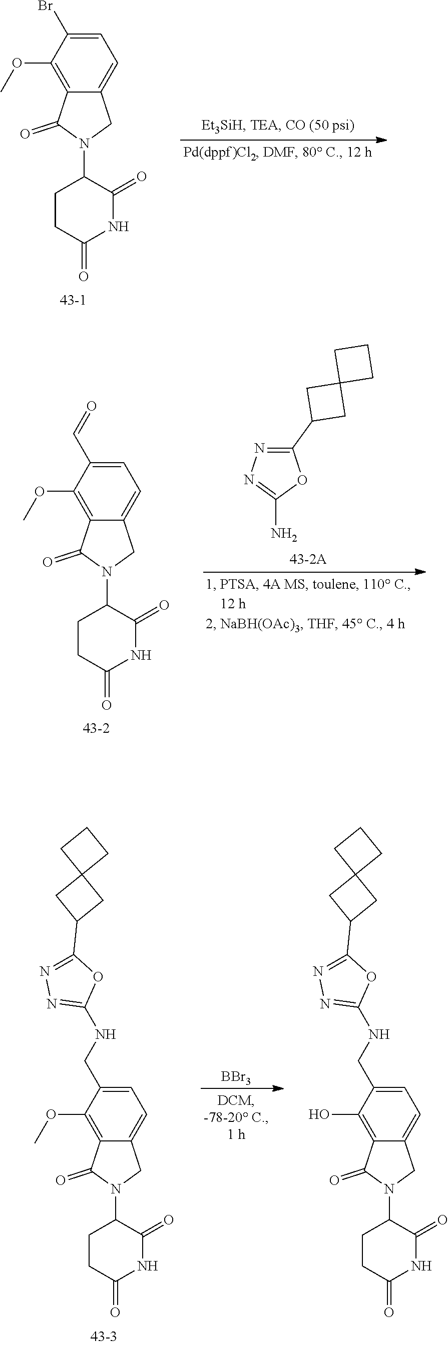 [SMILES: COc1c(Br)ccc2c1C(=O)N(C1CCC(=O)NC1=O)C2 ; COc1c(C=O)ccc2c1C(=O)N(C1CCC(=O)NC1=O)C2 ; Nc1nnc(C2CC3(CCC3)C2)o1 ; COc1c(CNc2nnc(C3CC4(CCC4)C3)o2)ccc2c1C(=O)N(C1CCC(=O)NC1=O)C2 ; "BBr3" ; O=C1CCC(N2Cc3ccc(CNc4nnc(C5CC6(CCC6)C5)o4)c(O)c3C2=O)C(=O)N1]

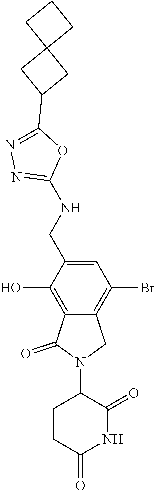 [SMILES: O=C1CCC(N2Cc3c(Br)cc(CNc4nnc(C5CC6(CCC6)C5)o4)c(O)c3C2=O)C(=O)N1]

### Step 1. Procedure for Preparation of Compound 2-2-(2,6-dioxopiperidin-3-yl)-4-methoxy-3-oxoisoindoline-5-carbaldehyde

To a solution of 3-(6-bromo-7-methoxy-1-oxoisoindolin-2-yl)piperidine-2,6-dione (700 mg, 1.98 mmol, 1.00 eq), triethylsilane (691 mg, 5.95 mmol, 950 uL, 3.00 eq), triethylamine (602 mg, 5.95 mmol, 828 uL, 3.00 eq) in dimethylformamide (21.0 mL) was added [1,1-bis(diphenylphosphino)ferrocene] dichloropalladium(II) (145 mg, 198 umol, 0.100 eq). The reaction mixture was stirred at 80° C. for 12 h under 50 psi of carbon monoxide atmosphere. The reaction mixture was filtered and the filtrate was concentrated under reduced pressure to give a residue. The residue was purified by reverse phase chromatography (C18, 120 g; condition: water/acetonitrile=100:0 to 90:10, 0.1% formic acid) and lyophilized to afford 2-(2,6-dioxopiperidin-3-yl)-4-methoxy-3-oxoisoindoline-5-carbaldehyde (270 mg, 893 umol, 45% yield) as a yellow solid.

1H NMR (400 MHz, DMSO-d6) δ=11.01 (s, 1H), 10.38 (s, 1H), 7.96 (d, J=7.8 Hz, 1H), 7.46 (d, J=7.9 Hz, 1H), 5.10 (dd, J=5.1, 13.3 Hz, 1H), 4.56-4.51 (m, 1H), 4.43-4.38 (m, 1H), 4.18 (s, 3H), 2.93-2.87 (m, 1H), 2.63-2.59 (m, 1H), 2.39 (br dd, J=4.4, 13.3 Hz, 1H), 2.01 (br dd, J=5.4, 10.7 Hz, 1H).

### Step 0.2 Procedure for preparation of Compound 3—3-(7-methoxy-1-oxo-6-(((5-(spiro[3.3]heptan-2-yl)-1,3,4-oxadiazol-2-yl)amino)methyl)isoindolin-2-yl)piperidine-2,6-dione

To a solution of 2-(2,6-dioxo-3-piperidyl)-4-methoxy-3-oxo-isoindoline-5-carbaldehyde (270 mg, 893 umol, 1.00 eq) in toluene (15.0 mL) were added 4A MS (270 mg), 4-methylbenzenesulfonic acid (17.0 mg, 89.3 umol, 0.100 eq) and 5-(spiro[3.3]heptan-2-yl)-1,3,4-oxadiazol-2-amine (176 mg, 983 umol, 1.10 eq). The reaction mixture was stirred at 115° C. for 12 h under nitrogen atmosphere. Cooled to 20° C., sodium triacetoxyhydroborate (947 mg, 4.47 mmol, 5.00 eq) and tetrahydrofuran (10.0 mL) were added to the mixture and the reaction mixture was stirred at 45° C. for 4 h under nitrogen atmosphere. The reaction mixture was concentrated under reduced pressure to give a residue. The residue was dissolved in dimethylformamide (8 mL) and then filtered. The filtered was purified by reverse phase chromatography (C18, 80 g; condition: water/acetonitrile=100:0 to 54:46, 0.1% formic acid) and lyophilized to afford 3-(7-methoxy-1-oxo-6-(((5-(spiro[3.3]heptan-2-yl)-1,3,4-oxadiazol-2-yl)amino)methyl)isoindolin-2-yl)piperidine-2,6-dione (90.0 mg, 193 umol, 22% yield) as an off-white solid.

1H NMR (400 MHz, DMSO-d6) δ=10.98 (br s, 1H), 7.89 (t, J=6.1 Hz, 1H), 7.58 (d, J=7.6 Hz, 1H), 7.27 (d, J=7.8 Hz, 1H), 5.07 (dd, J=4.9, 13.3 Hz, 1H), 4.44-4.34 (m, 3H), 4.31-4.22 (m, 1H), 4.01 (s, 3H), 3.40-3.37 (m, 1H), 2.96-2.85 (m, 1H), 2.64-2.56 (m, 1H), 2.45-2.38 (m, 1H), 2.36-2.31 (m, 2H), 2.21-2.14 (m, 2H), 2.07-2.02 (m, 2H), 2.01-1.95 (m, 1H), 1.93-1.86 (m, 2H), 1.81-1.72 (m, 2H).

Step 3. Procedure for preparation of 3-(7-hydroxy-1-oxo-6-(((5-(spiro[3.3]heptan-2-yl)-1,3,4-oxadiazol-2-yl)amino)methyl)isoindolin-2-yl)piperidine-2,6-dione and 3-(4-bromo-7-hydroxy-1-oxo-6-(((5-(spiro[3.3]heptan-2-yl)-1,3,4-oxadiazol-2-yl)amino)methyl)isoindolin-2-yl)piperidine-2,6-dione

To a solution of 3-(7-methoxy-1-oxo-6-(((5-(spiro[3.3]heptan-2-yl)-1,3,4-oxadiazol-2-yl)amino)methyl)isoindolin-2-yl)piperidine-2,6-dione (90.0 mg, 193 umol, 1.00 eq) in dichloromethane (1.50 mL) was added boron tribromide (145 mg, 580 umol, 55.9 uL, 3.00 eq) at −78° C. The reaction mixture was stirred at 20° C. for 1 h. The reaction mixture was quenched with water (0.5 mL) and then concentrated under reduced pressure to give a residue. The residue was purified by Prep-HPLC(column: Phenomenex luna C18 150*25 mm*10 um; mobile phase: [water (formic acid)-acetonitrile]; B %: 30%-60%, 9 min) and lyophilized to afford 3-(7-hydroxy-1-oxo-6-(((5-(spiro[3.3]heptan-2-yl)-1,3,4-oxadiazol-2-yl)amino)methyl)isoindolin-2-yl)piperidine-2,6-dione (16.09 mg, 33.86 umol, 18% yield, 95% purity) as an off-white solid and 3-(4-bromo-7-hydroxy-1-oxo-6-(((5-(spiro[3.3]heptan-2-yl)-1,3,4-oxadiazol-2-yl)amino)methyl)isoindolin-2-yl)piperidine-2,6-dione (9.40 mg, 15.95 umol, 8% yield, 90% purity) as an off-white solid.

1H NMR (400 MHz, DMSO-d6) δ=11.00 (s, 1H), 9.44-9.16 (m, 1H), 7.89 (t, J=5.9 Hz, 1H), 7.48 (d, J=7.6 Hz, 1H), 7.02 (d, J=7.6 Hz, 1H), 5.04 (dd, J=5.2, 13.3 Hz, 1H), 4.46-4.32 (m, 3H), 4.31-4.22 (m, 1H), 3.42-3.35 (m, 1H), 2.97-2.85 (m, 1H), 2.62 (br s, 1H), 2.42-2.36 (m, 1H), 2.35-2.29 (m, 2H), 2.23-2.15 (m, 2H), 2.08-2.03 (m, 2H), 2.02-1.97 (m, 1H), 1.93-1.86 (m, 2H), 1.81-1.72 (m, 2H).

1H NMR (400 MHz, DMSO-d6) δ=11.01 (s, 1H), 9.71 (br s, 1H), 7.95 (br t, J=5.5 Hz, 1H), 7.63 (s, 1H), 5.06 (dd, J=4.9, 13.3 Hz, 1H), 4.41-4.27 (m, 3H), 4.22-4.13 (m, 1H), 3.38 (t, J=8.4 Hz, 1H), 2.98-2.85 (m, 1H), 2.59 (br d, J=17.4 Hz, 1H), 2.47-2.39 (m, 1H), 2.36-2.30 (m, 2H), 2.24-2.16 (m, 2H), 2.07-1.98 (m, 3H), 1.94-1.87 (m, 2H), 1.82-1.72 (m, 2H).

### MS (ESI) m/z 452.1 [M+H]+. MS (ESI) m/z 532.2 [M+H+2]+.

### Example 44. Synthesis of Compound 136

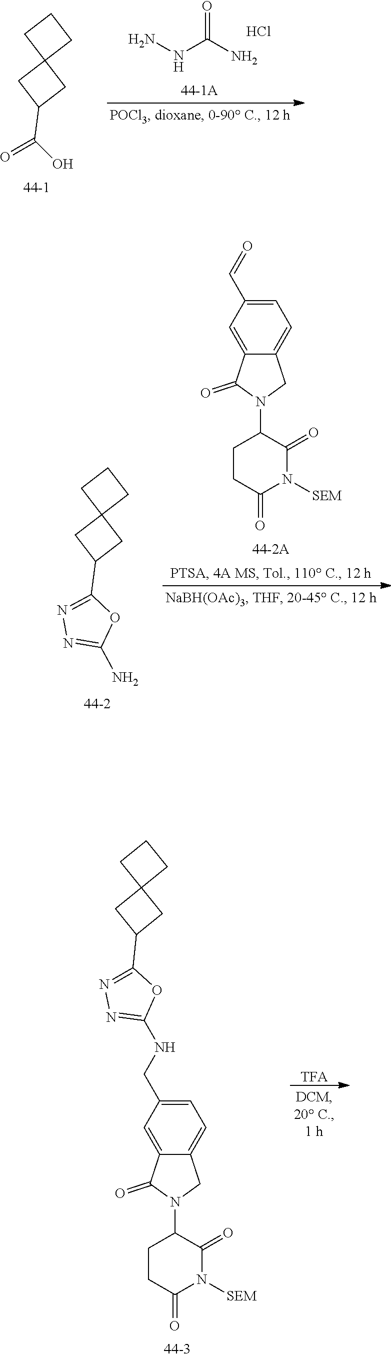

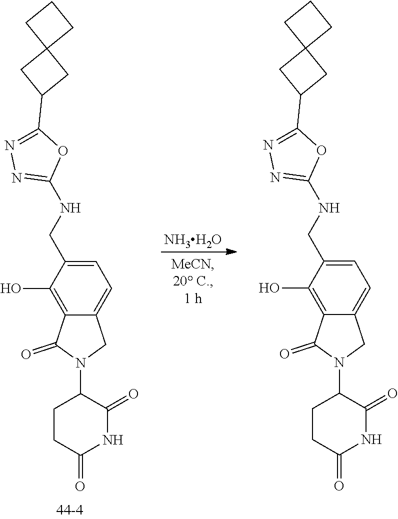 [SMILES: O=C1CCC(N2Cc3ccc(CNc4nnc(C5CC6(CCC6)C5)o4)c(O)c3C2=O)C(=O)N1 ; "NH3·H2O" ; O=C1CCC(N2Cc3ccc(CNc4nnc(C5CC6(CCC6)C5)o4)c(O)c3C2=O)C(=O)N1]

### Step 1. Procedure for Compound 2—5-(spiro[3.3]heptan-2-yl)-1,3,4-oxadiazol-2-amine

To a solution of spiro[3.3]heptane-2-carboxylic acid (200 mg, 1.43 mmol, 1.00 eq), hydrazinecarboxamide (159 mg, 1.43 mmol, 1.00 eq, hydrochloride) in dioxane (2.00 mL) was added phosphorus oxychloride (656 mg, 4.28 mmol, 398 uL, 3.00 eq) at 0° C. The reaction mixture was stirred at 90° C. for 12 h. The reaction mixture was added to water (10 mL). The pH of the mixture was adjust to the range of 8-9 with saturated sodium bicarbonate, then the mixture was extracted with ethyl acetate (3×10 mL). The organic phases were combined, washed with brine (20 mL), dried over sodium sulfate, filtered and concentrated under reduced pressure to give a residue. The residue was purified by reverse phase chromatography (C18, 40 g; condition: water/acetonitrile=100:0 to 0:100, 0.1% ammonia) and lyophilized to afford 5-(spiro[3.3]heptan-2-yl)-1,3,4-oxadiazol-2-amine (80.0 mg, 447 umol, 31% yield) as a white solid.

1H NMR (400 MHz, CDCl3-d) δ=4.90 (br s, 2H), 3.42 (quin, J=8.6 Hz, 1H), 2.42-2.30 (m, 4H), 2.10 (br t, J=7.2 Hz, 2H), 2.00-1.93 (m, 2H), 1.83 (quin, J=7.4 Hz, 2H).

### Step 2. Procedure for Compound 3—3-(1-oxo-6-(((5-(spiro[3.3]heptan-2-yl)-1,3,4-oxadiazol-2-yl)amino)methyl)isoindolin-2-yl)-1-((2-(trimethylsilyl)ethoxy)methyl)piperidine-2,6-dione

To a solution of 5-(spiro[3.3]heptan-2-yl)-1,3,4-oxadiazol-2-amine (10 mg, 55.8 umol, 1.00 eq) in toluene (2.00 mL) were added 4-methylbenzenesulfonic acid (961 ug, 5.58 umol, 0.100 eq), 4A MS (50.0 mg) and 2-(2,6-dioxo-1-((2-(trimethylsilyl)ethoxy)methyl)piperidin-3-yl)-3-oxoisoindoline-5-carbaldehyde (27.0 mg, 67.0 mmol, 1.20 eq). The mixture was stirred at 110° C. for 12 h under nitrogen atmosphere. Cooled to 20° C., sodium triacetoxyhydroborate (59.1 mg, 279 mmol, 5.00 eq) and tetrahydrofuran (1.00 mL) was were added to the mixture and the reaction mixture was stirred at 45° C. for 12 h. The reaction mixture was concentrated under reduced pressure to give a residue. The residue was dissolved in N,N-dimethylformamide (0.5 mL) and then filtered. The filtered was purified by reverse phase chromatography (C18, 40 g; condition: water/acetonitrile=100:0 to 0:100, 0.1% formic acid) and lyophilized to afford 3-(1-oxo-6-(((5-(spiro[3.3]heptan-2-yl)-1,3,4-oxadiazol-2-yl)amino)methyl)isoindolin-2-yl)-1-((2-(trimethylsilyl)ethoxy) methyl)piperidine-2,6-dione (12.0 mg, 21.2 umol, 38% yield) as yellow oil.

1H NMR (400 MHz, CDCl3-d) δ=7.88 (s, 1H), 7.63 (d, J=8.0 Hz, 1H), 7.45 (d, J=7.9 Hz, 1H), 5.26-5.18 (m, 3H), 4.63 (s, 2H), 4.52-4.43 (m, 1H), 4.38-4.30 (m, 1H), 3.67-3.58 (m, 2H), 3.41 (quin, J=8.6 Hz, 1H), 3.08-2.99 (m, 1H), 2.97-2.84 (m, 1H), 2.40-2.28 (m, 5H), 2.24-2.16 (m, 1H), 2.08 (t, J=7.3 Hz, 2H), 1.99-1.93 (m, 2H), 1.86-1.80 (m, 2H), 0.99-0.90 (m, 2H), 0.08 (s, 9H).

### Step 3. Procedure for Compound 3—1-(hydroxymethyl)-3-(1-oxo-6-(((5-(spiro[3.3]heptan-2-yl)-1,3,4-oxadiazol-2-yl)amino)methyl)isoindolin-2-yl)piperidine-2,6-dione

To a solution of 3-(1-oxo-6-(((5-(spiro[3.3]heptan-2-yl)-1,3,4-oxadiazol-2-yl)amino)methyl)isoindolin-2-yl)-1-((2-(trimethylsilyl)ethoxy)methyl)piperidine-2,6-dione (12.0 mg, 21.2 umol, 1.00 eq) in dichloromethane (1.00 mL) was added trifluoroacetic acid (154 mg, 1.35 mmol, 0.100 mL, 63.7 eq). The reaction mixture was stirred at 20° C. for 1 h. The reaction mixture was concentrated under reduced pressure to afford 1-(hydroxymethyl)-3-(1-oxo-6-(((5-(spiro[3.3]heptan-2-yl)-1,3,4-oxadiazol-2-yl)amino)methyl)isoindolin-2-yl)piperidine-2,6-dione (10.0 mg, crude) as yellow oil.

### MS (ESI) m/z.466.0 [M+H]⁺.

### Step 4. Procedure for 3-(1-oxo-6-(((5-(spiro[3.3]heptan-2-yl)-1,3,4-oxadiazol-2-yl)amino)methyl)isoindolin-2-yl)piperidine-2,6-dione

To a solution of 1-(hydroxymethyl)-3-(1-oxo-6-(((5-(spiro[3.3]heptan-2-yl)-1,3,4-oxadiazol-2-yl)amino)methyl)isoindolin-2-yl)piperidine-2,6-dione (10.0 mg, 21.5 umol, 1.00 eq) in acetonitrile (1.00 mL) was added ammonium hydroxide (45.5 mg, 325 umol, 0.500 mL, 25% purity, 15.1 eq). The reaction mixture was stirred at 20° C. for 1 h. The reaction mixture was concentrated under reduced pressure to give a residue (<30° C.). The residue was purified by Prep-HPLC(column: Phenomenex luna C18 150*25 mm*10 um; mobile phase: [water (formic acid)-acetonitrile]; B %: 24%-54%, 9 min) and lyophilized to afford 3-(1-oxo-6-(((5-(spiro[3.3]heptan-2-yl)-1,3,4-oxadiazol-2-yl)amino)methyl)isoindolin-2-yl)piperidine-2,6-dione (2.71 mg, 6.16 umol, 29% yield, 99% purity) as a white solid.

1H NMR (400 MHz, DMSO-d6) δ=10.99 (br s, 1H), 8.06 (t, J=6.3 Hz, 1H), 7.71 (s, 1H), 7.63-7.59 (m, 1H), 7.58-7.55 (m, 1H), 5.12 (dd, J=5.1, 13.3 Hz, 1H), 4.48-4.40 (m, 3H), 4.34-4.28 (m, 1H), 3.40-3.35 (m, 1H), 2.97-2.86 (m, 1H), 2.63-2.60 (m, 1H), 2.40 (dd, J=4.0, 13.0 Hz, 1H), 2.34 (br d, J=1.8 Hz, 2H), 2.23-2.15 (m, 2H), 2.08-2.03 (m, 2H), 2.02-1.96 (m, 1H), 1.93-1.88 (m, 2H), 1.81-1.73 (m, 2H).

### MS (ESI) m/z.436.2 [M+H]⁺.

### Example 45. Synthesis of Compound 137

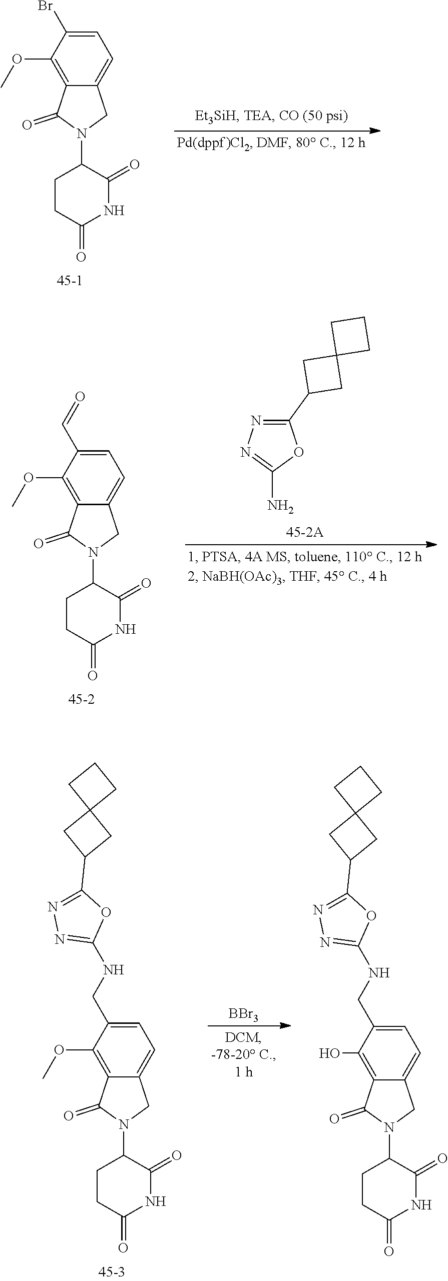 [SMILES: COc1c(Br)ccc2c1C(=O)N(C1CCC(=O)NC1=O)C2 ; COc1c(C=O)ccc2c1C(=O)N(C1CCC(=O)NC1=O)C2 ; Nc1nnc(C2CC3(CCC3)C2)o1 ; COc1c(CNc2nnc(C3CC4(CCC4)C3)o2)ccc2c1C(=O)N(C1CCC(=O)NC1=O)C2 ; "BBr3" ; O=C1CCC(N2Cc3ccc(CNc4nnc(C5CC6(CCC6)C5)o4)c(O)c3C2=O)C(=O)N1]

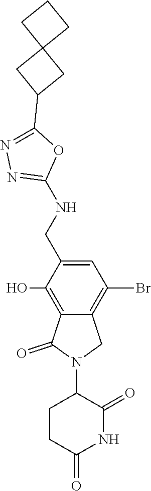 [SMILES: O=C1CCC(N2Cc3c(Br)cc(CNc4nnc(C5CC6(CCC6)C5)o4)c(O)c3C2=O)C(=O)N1]

### Step 1. Procedure for Preparation of Compound 2-2-(2,6-dioxopiperidin-3-yl)-4-methoxy-3-oxoisoindoline-5-carbaldehyde

To a solution of 3-(6-bromo-7-methoxy-1-oxoisoindolin-2-yl)piperidine-2,6-dione (700 mg, 1.98 mmol, 1.00 eq), triethylsilane (691 mg, 5.95 mmol, 950 uL, 3.00 eq), triethylamine (602 mg, 5.95 mmol, 828 uL, 3.00 eq) in dimethylformamide (21.0 mL) was added [1,1-bis(diphenylphosphino)ferrocene] dichloropalladium(II) (145 mg, 198 umol, 0.100 eq). The reaction mixture was stirred at 80° C. for 12 h under 50 psi of carbon monoxide atmosphere. The reaction mixture was filtered and the filtrate was concentrated under reduced pressure to give a residue. The residue was purified by reverse phase chromatography (C18, 120 g; condition: water/acetonitrile=100:0 to 90:10, 0.1% formic acid) and lyophilized to afford 2-(2,6-dioxopiperidin-3-yl)-4-methoxy-3-oxoisoindoline-5-carbaldehyde (270 mg, 893 umol, 45% yield) as a yellow solid.

¹H NMR (400 MHz, DMSO-d₆) δ=11.01 (s, 1H), 10.38 (s, 1H), 7.96 (d, J=7.8 Hz, 1H), 7.46 (d, J=7.9 Hz, 1H), 5.10 (dd, J=5.1, 13.3 Hz, 1H), 4.56-4.51 (m, 1H), 4.43-4.38 (m, 1H), 4.18 (s, 3H), 2.93-2.87 (m, 1H), 2.63-2.59 (m, 1H), 2.39 (br dd, J=4.4, 13.3 Hz, 1H), 2.01 (br dd, J=5.4, 10.7 Hz, 1H).

### Step 2. Procedure for Preparation of Compound 3—3-(7-methoxy-1-oxo-6-(((5-(spiro[3.3]heptan-2-yl)-1,3,4-oxadiazol-2-yl)amino)methyl)isoindolin-2-yl)piperidine-2,6-dione

To a solution of 2-(2,6-dioxo-3-piperidyl)-4-methoxy-3-oxo-isoindoline-5-carbaldehyde (270 mg, 893 umol, 1.00 eq) in toluene (15.0 mL) were added 4A MS (270 mg), 4-methylbenzenesulfonic acid (17.0 mg, 89.3 umol, 0.100 eq) and 5-(spiro[3.3]heptan-2-yl)-1,3,4-oxadiazol-2-amine (176 mg, 983 umol, 1.10 eq). The reaction mixture was stirred at 115° C. for 12 h under nitrogen atmosphere. Cooled to 20° C., sodium triacetoxyhydroborate (947 mg, 4.47 mmol, 5.00 eq) and tetrahydrofuran (10.0 mL) were added to the mixture and the reaction mixture was stirred at 45° C. for 4 h under nitrogen atmosphere. The reaction mixture was concentrated under reduced pressure to give a residue. The residue was dissolved in dimethylformamide (8 mL) and then filtered. The filtered was purified by reverse phase chromatography (C18, 80 g; condition: water/acetonitrile=100:0 to 54:46, 0.1% formic acid) and lyophilized to afford 3-(7-methoxy-1-oxo-6-(((5-(spiro[3.3]heptan-2-yl)-1,3,4-oxadiazol-2-yl)amino)methyl)isoindolin-2-yl)piperidine-2,6-dione (90.0 mg, 193 umol, 22% yield) as an off-white solid.

¹H NMR (400 MHz, DMSO-d₆) δ=10.98 (br s, 1H), 7.89 (t, J=6.1 Hz, 1H), 7.58 (d, J=7.6 Hz, 1H), 7.27 (d, J=7.8 Hz, 1H), 5.07 (dd, J=4.9, 13.3 Hz, 1H), 4.44-4.34 (m, 3H), 4.31-4.22 (m, 1H), 4.01 (s, 3H), 3.40-3.37 (m, 1H), 2.96-2.85 (m, 1H), 2.64-2.56 (m, 1H), 2.45-2.38 (m, 1H), 2.36-2.31 (m, 2H), 2.21-2.14 (m, 2H), 2.07-2.02 (m, 2H), 2.01-1.95 (m, 1H), 1.93-1.86 (m, 2H), 1.81-1.72 (m, 2H).

Step 3. Procedure for preparation of 3-(7-hydroxy-1-oxo-6-(((5-(spiro[3.3]heptan-2-yl)-1,3,4-oxadiazol-2-yl)amino)methyl)isoindolin-2-yl)piperidine-2,6-dione and 3-(4-bromo-7-hydroxy-1-oxo-6-(((5-(spiro[3.3]heptan-2-yl)-1,3,4-oxadiazol-2-yl)amino)methyl)isoindolin-2-yl)piperidine-2,6-dione

To a solution of 3-(7-methoxy-1-oxo-6-(((5-(spiro[3.3]heptan-2-yl)-1,3,4-oxadiazol-2-yl)amino)methyl)isoindolin-2-yl)piperidine-2,6-dione (90.0 mg, 193 umol, 1.00 eq) in dichloromethane (1.50 mL) was added boron tribromide (145 mg, 580 umol, 55.9 uL, 3.00 eq) at −78° C. The reaction mixture was stirred at 20° C. for 1 h. The reaction mixture was quenched with water (0.5 mL) and then concentrated under reduced pressure to give a residue. The residue was purified by Prep-HPLC(column: Phenomenex luna C18 150*25 mm*10 um; mobile phase: [water (formic acid)-acetonitrile]; B %: 30%-60%, 9 min) and lyophilized to afford 3-(7-hydroxy-1-oxo-6-(((5-(spiro[3.3]heptan-2-yl)-1,3,4-oxadiazol-2-yl)amino)methyl)isoindolin-2-yl)piperidine-2,6-dione (16.09 mg, 33.86 umol, 18% yield, 95% purity) as an off-white solid and 3-(4-bromo-7-hydroxy-1-oxo-6-(((5-(spiro[3.3]heptan-2-yl)-1,3,4-oxadiazol-2-yl)amino)methyl)isoindolin-2-yl)piperidine-2,6-dione (9.40 mg, 15.95 umol, 8% yield, 90% purity) as an off-white solid.

¹H NMR (400 MHz, DMSO-d₆) δ=11.00 (s, 1H), 9.44-9.16 (m, 1H), 7.89 (t, J=5.9 Hz, 1H), 7.48 (d, J=7.6 Hz, 1H), 7.02 (d, J=7.6 Hz, 1H), 5.04 (dd, J=5.2, 13.3 Hz, 1H), 4.46-4.32 (m, 3H), 4.31-4.22 (m, 1H), 3.42-3.35 (m, 1H), 2.97-2.85 (m, 1H), 2.62 (br s, 1H), 2.42-2.36 (m, 1H), 2.35-2.29 (m, 2H), 2.23-2.15 (m, 2H), 2.08-2.03 (m, 2H), 2.02-1.97 (m, 1H), 1.93-1.86 (m, 2H), 1.81-1.72 (m, 2H).

¹H NMR (400 MHz, DMSO-d₆) δ=11.01 (s, 1H), 9.71 (br s, 1H), 7.95 (br t, J=5.5 Hz, 1H), 7.63 (s, 1H), 5.06 (dd, J=4.9, 13.3 Hz, 1H), 4.41-4.27 (m, 3H), 4.22-4.13 (m, 1H), 3.38 (t, J=8.4 Hz, 1H), 2.98-2.85 (m, 1H), 2.59 (br d, J=17.4 Hz, 1H), 2.47-2.39 (m, 1H), 2.36-2.30 (m, 2H), 2.24-2.16 (m, 2H), 2.07-1.98 (m, 3H), 1.94-1.87 (m, 2H), 1.82-1.72 (m, 2H).

MS (ESI) m/z 452.1 [M+H]⁺. MS (ESI) m/z 532.2 [M+H+2]⁺.

### Example 46. Synthesis of Compound 138

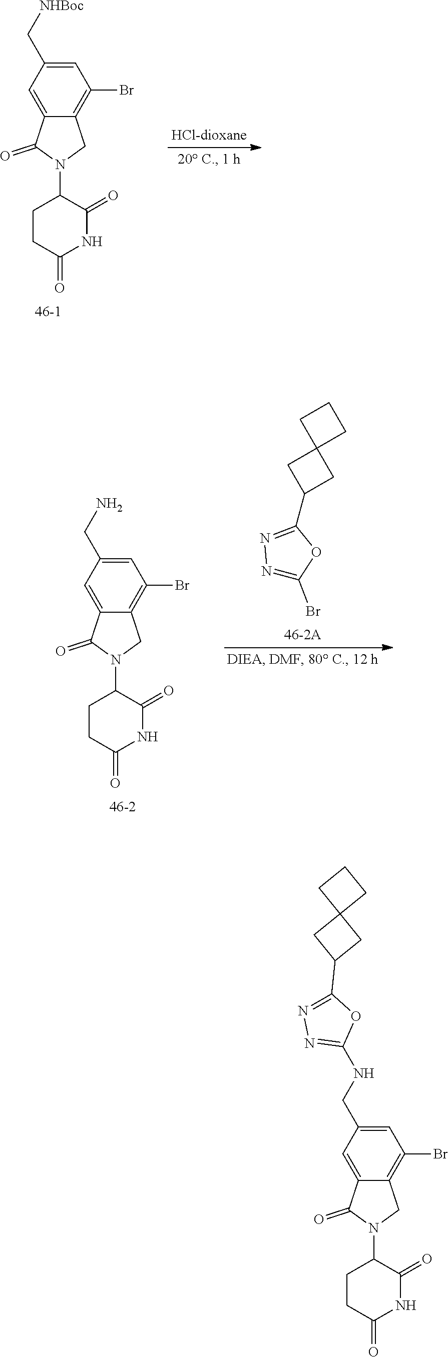 [SMILES: CC(C)(C)OC(=O)NCc1cc(Br)c2c(c1)C(=O)N(C1CCC(=O)NC1=O)C2 ; NCc1cc(Br)c2c(c1)C(=O)N(C1CCC(=O)NC1=O)C2 ; Brc1nnc(C2CC3(CCC3)C2)o1 ; O=C1CCC(N2Cc3c(Br)cc(CNc4nnc(C5CC6(CCC6)C5)o4)cc3C2=O)C(=O)N1]

### Step 1. Procedure for Preparation of Compound 2—3-(6-(aminomethyl)-4-bromo-1-oxoisoindolin-2-yl)piperidine-2,6-dione

To a solution of tert-butyl ((7-bromo-2-(2,6-dioxopiperidin-3-yl)-3-oxoisoindolin-5-yl)methyl)carbamate (120 mg, 265 umol, 1.00 eq) in dioxane (1.00 mL) was added hydrochloric acid/dioxane (4.00 M, 2.00 mL, 30.2 eq). The reaction mixture was stirred at 20° C. for 1 h. The reaction mixture was concentrated under reduced pressure to afford 3-(6-(aminomethyl)-4-bromo-1-oxoisoindolin-2-yl)piperidine-2,6-dione (103 mg, 265 umol, 99% yield, hydrochloride) as a brown solid.

1H NMR (400 MHz, DMSO-d6) δ=11.02 (s, 1H), 8.42 (br s, 3H), 8.02 (s, 1H), 7.93 (s, 1H), 5.14 (dd, J=5.1, 13.3 Hz, 1H), 4.49-4.40 (m, 1H), 4.34-4.24 (m, 1H), 4.17 (q, J=5.5 Hz, 2H), 2.97-2.86 (m, 1H), 2.60 (br d, J=17.1 Hz, 1H), 2.49-2.42 (m, 1H), 2.06-1.98 (m, 1H).

### Step 2. Procedure for preparation of 3-(4-bromo-1-oxo-6-(((5-(spiro[3.3]heptan-2-yl)-1,3,4-oxadiazol-2-yl)amino)methyl)isoindolin-2-yl)piperidine-2,6-dione

To a solution of 3-(6-(aminomethyl)-4-bromo-1-oxoisoindolin-2-yl)piperidine-2,6-dione (100 mg, 257 umol, 1.00 eq, hydrochloride) in dimethylformamide (1.00 mL) were added N,N-diisopropylethylamine (66.5 mg, 515 umol, 89.6 uL, 2.00 eq) and 2-bromo-5-(spiro[3.3]heptan-2-yl)-1,3,4-oxadiazole (50.0 mg, 206 umol, 0.800 eq). The reaction mixture was stirred at 80° C. for 12 h. The reaction mixture was filtered. The filtrate was purified by Prep-HPLC(column: Phenomenex luna C18 150*25 mm*10 um; mobile phase: [water (formic acid)-acetonitrile]; B %: 34%-64%, 9 min) and lyophilized to give a crude product. The crude product was purified by column chromatography (SiO2, petroleum ether/ethyl acetate=1/0 to 0/1) and concentrated under reduced pressure to give yellow oil. The yellow oil was diluted with acetonitrile (1 mL), then added water (15 mL) and lyophilized to afford 3-(4-bromo-1-oxo-6-(((5-(spiro[3.3]heptan-2-yl)-1,3,4-oxadiazol-2-yl)amino)methyl)isoindolin-2-yl)piperidine-2,6-dione (10.95 mg, 20.44 umol, 7.94% yield, 96% purity) as a white solid.

1H NMR (400 MHz, DMSO-d6) δ=11.00 (s, 1H), 8.09 (t, J=6.2 Hz, 1H), 7.84 (s, 1H), 7.73 (s, 1H), 5.13 (dd, J=5.1, 13.4 Hz, 1H), 4.45 (d, J=6.3 Hz, 2H), 4.43-4.36 (m, 1H), 4.27-4.20 (m, 1H), 3.44-3.37 (m, 1H), 2.95-2.85 (m, 1H), 2.61 (br d, J=2.1 Hz, 1H), 2.45-2.41 (m, 1H), 2.35-2.30 (m, 2H), 2.21-2.16 (m, 2H), 2.07-2.02 (m, 2H), 2.00 (br d, J=6.1 Hz, 1H), 1.92-1.87 (m, 2H), 1.81-1.71 (m, 2H).

### MS (ESI) m/z 516.2 [M+H+2]⁺.

### Example 47. Synthesis of Compound 139

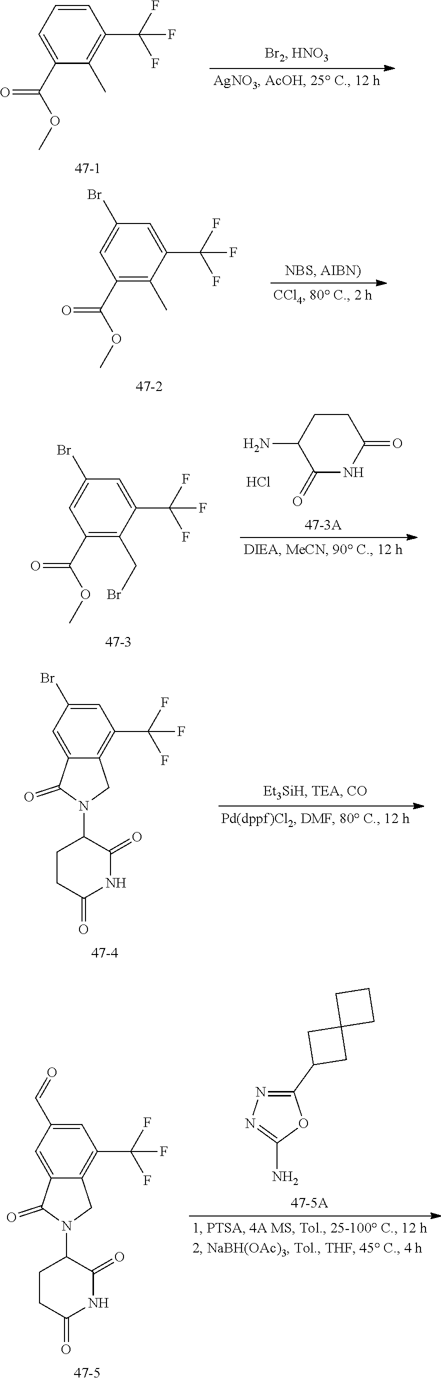

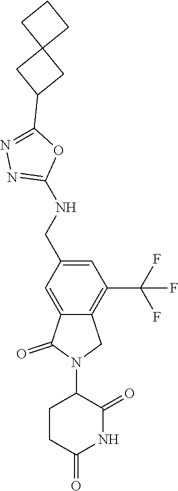 [SMILES: O=C1CCC(N2Cc3c(cc(CNc4nnc(C5CC6(CCC6)C5)o4)cc3C(F)(F)F)C2=O)C(=O)N1]

### Step 1. Procedure for compound 2—methyl 5-bromo-2-methyl-3-(trifluoromethyl)benzoate

To a solution of methyl 2-methyl-3-(trifluoromethyl)benzoate (5.00 g, 22.9 mmol, 1.00 eq) in acetic acid (20.0 mL) was added bromine (4.03 g, 25.2 mmol, 1.30 mL, 1.10 eq) and nitric acid (1.55 g, 24.1 mmol, 1.11 mL, 98% purity, 1.05 eq) at 25° C. Then silver nitrate (2.50 M, 9.63 mL, 1.05 eq) was added into the mixture over 30 min. And the mixture was stirred for 12 h at 25° C. The mixture was basified to 7 with 1 M sodium hydroxide. The mixture was diluted with water (200 mL) and extracted with ethyl acetate (100.0 mL*3). The combined organic layers were washed with brine (200 mL), dried over sodium sulfate and filtered. The filtrate was concentrated under reduced pressure and purified by column chromatography (SiO2, petroleum ether) to afford methyl 5-bromo-2-methyl-3-(trifluoromethyl)benzoate (5.10 g, crude) as yellow oil.

1H NMR (400 MHz, CDCl3) δ=8.07 (d, J=1.9 Hz, 1H), 7.90 (d, J=2.0 Hz, 1H), 3.95-3.94 (m, 3H), 2.60 (d, J=1.5 Hz, 3H)

### Step 2. Procedure for compound 3—methyl 5-bromo-2-(bromomethyl)-3-(trifluoromethyl)benzoate

To a solution of methyl 5-bromo-2-methyl-3-(trifluoromethyl)benzoate (3.00 g, 10.1 mmol, 1.00 eq) in tetrachloromethane (25.0 mL) was added N-bromosuccinimide (1.80 g, 10.1 mmol, 1.00 eq) and (E)-2,2′-(diazene-1,2-diyl)bis(2-methylpropanenitrile) (166 mg, 1.01 mmol, 0.10 eq) under nitrogen atmosphere. The mixture was stirred at 80° C. for 2 h. The mixture was concentrated under reduced pressure to give a residue. The residue was purified by column chromatography (SiO2, petroleum ether/ethyl acetate=50:1) to afford methyl 5-bromo-2-(bromomethyl)-3-(trifluoromethyl)benzoate (2.90 g, crude) as yellow oil.

1H NMR (400 MHz, CDCl3) δ=8.22-8.17 (m, 1H), 7.97-7.94 (m, 1H), 5.05 (s, 2H), 4.00 (s, 3H)

### Step 3. Procedure for compound 4—3-(6-bromo-1-oxo-4-(trifluoromethyl)isoindolin-2-yl)piperidine-2,6-dione

To a solution of methyl 5-bromo-2-(bromomethyl)-3-(trifluoromethyl)benzoate (2.76 g, 7.35 mmol, 1.10 eq) in acetonitrile (30.0 mL) was added N,N-diisopropylethylamine (2.59 g, 20.1 mmol, 3.49 mL, 3.00 eq) and 3-aminopiperidine-2,6-dione hydrochloride (1.10 g, 6.68 mmol, 1.00 eq, hydrochloric acid) at 25° C. And the mixture was stirred at 90° C. for 12 h. The reaction mixture was and concentrated under reduced pressure to give a residue. The residue was purified by column chromatography (SiO2, petroleum ether/ethyl acetate=3:1) to afford 3-(6-bromo-1-oxo-4-(trifluoromethyl)isoindolin-2-yl)piperidine-2,6-dione (309 mg, 774 umol, 11% yield, 98% purity) as a blue solid.

1H NMR (400 MHz, DMSO-d6) δ=11.03 (s, 1H), 8.28-8.16 (m, 2H), 5.20-5.10 (m, 1H), 4.71-4.57 (m, 1H), 4.55-4.43 (m, 1H), 2.98-2.83 (m, 1H), 2.63-2.58 (m, 1H), 2.42-2.24 (m, 1H), 2.06-1.99 (m, 1H)

### Step 4. Procedure for compound 5-2-(2,6-dioxopiperidin-3-yl)-3-oxo-7-(trifluoromethyl)isoindoline-5-carbaldehyde

To a solution of 3-(6-bromo-1-oxo-4-(trifluoromethyl)isoindolin-2-yl)piperidine-2,6-dione (309 mg, 790 umol, 1.00 eq) in N,N-dimethyl formamide (10.0 mL) was added [1,1′-bis(diphenylphosphino)ferrocene]dichloropalladium(II) (57.8 mg, 79.0 umol, 0.10 eq) and triethylsilane (276 mg, 2.37 mmol, 379 uL, 3.00 eq) and triethylamine (240 mg, 2.37 mmol, 330 uL, 3.00 eq). The mixture was degassed and purged with carbon monoxide (50 psi). The mixture was stirred at 80° C. for 12 h. The mixture was quenched with water (50.0 mL) and extracted with ethyl acetate (100 mL*3). The combined organic layer was washed with water (50.0 mL), dried over sodium sulfate, filtered and concentrated under reduced pressure to give a residue. The residue was purified by column chromatography (SiO2, petroleum ether/ethyl acetate=2:1) to afford 2-(2,6-dioxopiperidin-3-yl)-3-oxo-7-(trifluoromethyl)isoindoline-5-carbaldehyde (138 mg, 389 umol, 49% yield, 96% purity) as a yellow solid.

1H NMR (400 MHz, DMSO-d6) δ=11.09-10.95 (m, 1H), 10.27-10.10 (m, 1H), 8.54-8.44 (m, 1H), 8.09-7.92 (m, 1H), 5.24-5.10 (m, 1H), 4.84-4.49 (m, 2H), 2.98-2.89 (m, 1H), 2.64-2.56 (m, 1H), 2.53 (br s, 1H), 2.10-2.00 (m, 1H)

### Step 5. Procedure for 3-cyclopropylprop-2-yn-1-yl (1-(4-(2,6-dioxopiperidin-3-yl)-3,5-difluorophenyl)azetidin-3-yl)carbamate

To a solution of 2-(2,6-dioxopiperidin-3-yl)-3-oxo-7-(trifluoromethyl)isoindoline-5-carbaldehyde (138 mg, 406 umol, 1.00 eq) in toluene (16.0 mL) was added 5-(spiro[3.3]heptan-2-yl)-1,3,4-oxadiazol-2-amine (87.2 mg, 487 umol, 1.20 eq), p-toluene sulfonic acid (6.98 mg, 40.6 umol, 0.10 eq) and 4 A MS (138 mg) at 25° C. The mixture was stirred at 110° C. for 12 h. The mixture was cooled to 25° C. And then sodium triacetoxyhydroborate (429 mg, 2.03 mmol, 5.00 eq) in tetrahydrofuran (5.00 mL) was added into the mixture. The mixture was stirred at 45° C. for 4 h. The residue was purified by prep-HPLC (column: Phenomenex luna C18 150*25 mm*10 um; mobile phase: [water(formic acid)-acetonitrile]; B %: 34%-64%, 9 min) and lyophilized to afford 3-(1-oxo-6-(((5-(spiro[3.3]heptan-2-yl)-1,3,4-oxadiazol-2-yl)amino)methyl)-4-(trifluoromethyl)isoindolin-2-yl)piperidine-2,6-dione as a white solid.

1H NMR (400 MHz, DMSO-d6) δ=11.12-10.85 (m, 1H), 8.14 (t, J=6.1 Hz, 1H), 7.99 (br d, J=12.5 Hz, 2H), 5.15 (dd, J=5.2, 13.2 Hz, 1H), 4.68-4.60 (m, 1H), 4.54 (br d, J=6.3 Hz, 2H), 4.52-4.46 (m, 1H), 3.42-3.36 (m, 1H), 2.97-2.84 (m, 1H), 2.64-2.57 (m, 1H), 2.44 (br s, 2H), 2.35-2.30 (m, 2H), 2.22-2.14 (m, 2H), 2.06-2.02 (m, 2H), 1.93-1.85 (m, 2H), 1.81-1.71 (m, 2H)

### MS (ESI) m/z 504.2 [M+H]⁺.

### Example 48. Synthesis of Compound 140

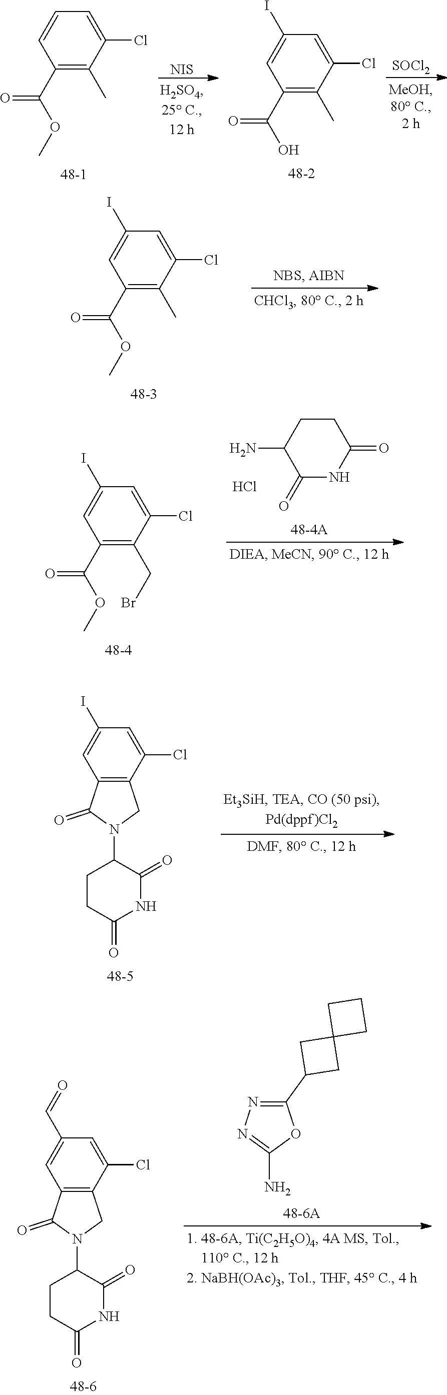

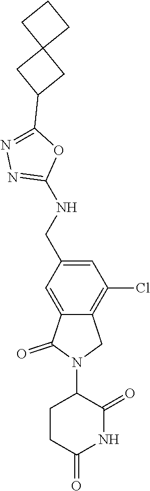 [SMILES: O=C1CCC(N2Cc3c(Cl)cc(CNc4nnc(C5CC6(CCC6)C5)o4)cc3C2=O)C(=O)N1]

### Step 1. Procedure for Preparation of Compound 2—3-chloro-5-iodo-2-methylbenzoic acid

To a solution of methyl 3-chloro-2-methylbenzoate (3.00 g, 16.2 mmol, 1.00 eq) in sulfuric acid (24.0 mL) was added 1-iodopyrrolidine-2,5-dione (3.66 g, 16.2 mmol, 1.00 eq). The reaction was stirred at 25° C. for 12 h. The reaction was added dropwise in water (100 mL) at 0° C. Then the mixture was extracted with ethyl acetate (3×100 mL) and the combined organic layers were washed with brine (100 mL), dried over sodium sulfate, filtered and concentrated under reduced pressure to give a residue. The residue was purified by column chromatography (SiO2, petroleum ether/ethyl acetate=10/1 to 5/1) to afford 3-chloro-5-iodo-2-methylbenzoic acid (1.35 g, 4.55 mmol, 28.02% yield) as a white solid.

1H NMR (400 MHz, CDCl3) δ=8.12 (d, J=1.6 Hz, 1H), 7.83 (d, J=1.6 Hz, 1H), 2.55 (s, 3H).

### Step 2. Procedure for Preparation of Compound 3—methyl 3-chloro-5-iodo-2-methylbenzoate

To a solution of 3-chloro-5-iodo-2-methylbenzoic acid (1.35 g, 4.55 mmol, 1.00 eq) in methanol (15.0 mL) was added thionyl chloride (1.08 g, 9.11 mmol, 660 uL, 2.00 eq). The reaction was stirred at 80° C. for 2 h. The reaction was quenched with sodium bicarbonate (100 mL) and extracted with ethyl acetate (3×100 mL) and the combined organic layers were washed with brine (100 mL), dried over sodium sulfate, filtered and concentrated under reduced pressure to afford methyl 3-chloro-5-iodo-2-methylbenzoate (1.00 g, 3.22 mmol, 71% yield) as a white solid.

1H NMR (400 MHz, CDCl3) δ=7.94 (d, J=1.2 Hz, 1H), 7.76 (d, J=1.2 Hz, 1H), 3.83 (s, 3H), 2.46 (s, 3H).

### Step 3. Procedure for Preparation of Compound 4—methyl 2-(bromomethyl)-3-chloro-5-iodobenzoate

To a solution of methyl 3-chloro-5-iodo-2-methylbenzoate (1.00 g, 3.22 mmol, 1.00 eq) in trichloromethane (10.0 mL) was added N-Bromosuccinimide (630 mg, 3.54 mmol, 1.10 eq) and (E)-2,2′-(diazene-1,2-diyl)bis(2-methylpropanenitrile) (528 mg, 3.22 mmol, 1.00 eq). The reaction was stirred at 80° C. for 2 h. The reaction was diluted with water (100 mL) and extracted with ethyl acetate (3×100 mL). The combined organic layers were washed with brine (100 mL), dried over sodium sulfate, filtered and concentrated under reduced pressure to afford methyl 2-(bromomethyl)-3-chloro-5-iodobenzoate (1.20 g, 3.08 mmol, 96% yield) as a white solid.

1H NMR (400 MHz, CDCl3) δ=8.10 (s, 1H), 7.86 (s, 1H), 4.97 (s, 2H), 3.89 (s, 3H).

### Step 4. Procedure for Preparation of Compound 5-3-(4-chloro-6-iodo-1-oxoisoindolin-2-yl)piperidine-2,6-dione

To a solution of methyl 2-(bromomethyl)-3-chloro-5-iodobenzoate (1.20 g, 3.08 mmol, 1.00 eq) and 3-aminopiperidine-2,6-dione hydrochloride (608 mg, 3.70 mmol, 1.20 eq) in acetonitrile (10.0 mL) was added N,N-diisopropylethylamine (1.19 g, 9.24 mmol, 1.61 mL, 3.00 eq). The reaction was stirred at 90° C. for 2 h. The reaction was concentrated under reduced pressure to give a residue. The residue was triturated with ethyl acetate (10.0 mL) and water (10.0 mL) to afford 3-(4-chloro-6-iodo-1-oxoisoindolin-2-yl)piperidine-2,6-dione (500 mg, 1.24 mmol, 40% yield) as a white solid.

1H NMR (400 MHz, DMSO-d6) δ=11.02 (s, 1H), 8.13 (s, 1H), 8.03 (s, 1H), 5.13 (dd, J=5.2, 13.2 Hz, 1H), 4.51-4.40 (m, 1H), 4.35-4.24 (m, 1H), 2.96-2.86 (m, 1H), 2.62 (br s, 1H), 2.45 (br dd, J=4.4, 13.2 Hz, 1H), 2.07-1.97 (m, 1H).

### Step 5. Procedure for Preparation of Compound 6-7-chloro-2-(2,6-dioxopiperidin-3-yl)-3-oxoisoindoline-5-carbaldehyde

To a solution of 3-(4-chloro-6-iodo-1-oxoisoindolin-2-yl)piperidine-2,6-dione (500 mg, 1.24 mmol, 1.00 eq) in dimethylformamide (4.00 mL) was added triethylamine (375 mg, 3.71 mmol, 516 uL, 3.00 eq), triethylsilane (431 mg, 3.71 mmol, 592 uL, 3.00 eq) and [1,1-bis(diphenylphosphino)ferrocene]dichloropalladium(II) (90.4 mg, 123 umol, 0.100 eq). The reaction was stirred at 80° C. for 12 h under carbon monoxide atmosphere. The reaction was filtered to give a filtrate. The filtrate was purified by reversed-phase HPLC (0.1% formic acid condition) to afford 7-chloro-2-(2,6-dioxopiperidin-3-yl)-3-oxoisoindoline-5-carbaldehyde (260 mg, 847 umol, 67% yield) as a white solid.

1H NMR (400 MHz, DMSO-d6) δ=11.03 (br d, J=9.2 Hz, 1H), 10.12 (s, 1H), 8.23 (d, J=9.6 Hz, 2H), 5.18 (br dd, J=4.8, 12.8 Hz, 1H), 4.68-4.58 (m, 1H), 4.49-4.44 (m, 1H), 2.96-2.90 (m, 1H), 2.64 (br d, J=2.0 Hz, 1H), 2.46 (br s, 1H), 2.06-2.02 (m, 1H).

### Step 6. Procedure for preparation of 3-(4-chloro-1-oxo-6-(((5-(spiro[3.3]heptan-2-yl)-1,3,4-oxadiazol-2-yl)amino)methyl)isoindolin-2-yl)piperidine-2,6-dione

To a solution of 7-chloro-2-(2,6-dioxopiperidin-3-yl)-3-oxoisoindoline-5-carbaldehyde (160 mg, 521 umol, 1.00 eq) and 5-(spiro[3.3]heptan-2-yl)-1,3,4-oxadiazol-2-amine (112 mg, 626 umol, 1.20 eq) in toluene (2.00 mL) was added 4A molecular sieve (100 mg) and tetraethoxytitanium (11.9 mg, 52.1 umol, 10.8 uL, 0.100 eq). The reaction was stirred at 110° C. for 12 h. After cooled down, a solution sodium triacetoxyhydroborate (552 mg, 2.61 mmol, 5.00 eq) in tetrahydrofuran (2.00 mL) was added into the resulting solution. The reaction was stirred at 45° C. for 4 h. The reaction was concentrated under reduced pressure and filtered to give a residue. The residue was purified by Prep-HPLC (column: Phenomenex luna C18 150*25 mm*10 um; mobile phase: [water(formic acid)-acetonitrile]; B %: 35%-55%, 9 min) to afford 3-(4-chloro-1-oxo-6-(((5-(spiro[3.3]heptan-2-yl)-1,3,4-oxadiazol-2-yl)amino)methyl)isoindolin-2-yl)piperidine-2,6-dione (8.69 mg, 11.7 umol, 2% yield, 97% purity) as a white solid.

1H NMR (400 MHz, DMSO-d6) δ=11.00 (br d, J=3.6 Hz, 1H), 8.10 (br t, J=5.2 Hz, 1H), 7.71 (s, 2H), 5.13 (br dd, J=4.0, 13.2 Hz, 1H), 4.53-4.41 (m, 3H), 4.36-4.27 (m, 1H), 3.56-3.48 (m, 1H), 2.89 (br d, J=11.6 Hz, 1H), 2.62 (br s, 1H), 2.45 (br s, 1H), 2.36-2.30 (m, 2H), 2.22-2.15 (m, 2H), 2.07-2.03 (m, 2H), 2.02-1.97 (m, 1H), 1.94-1.88 (m, 2H), 1.81-1.74 (m, 2H).

### MS (ESI) m/z 470.2 [M+H]⁺.

### Example 49. Synthesis of Compound 141

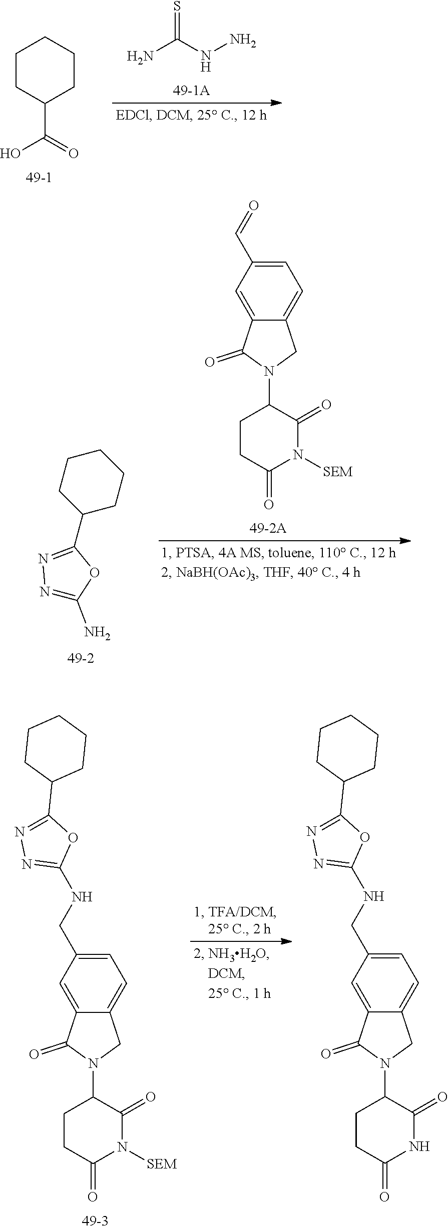 [SMILES: O=C(O)C1CCCCC1 ; NNC(N)=S ; Nc1nnc(C2CCCCC2)o1 ; C[Si](C)(C)CCOCN1C(=O)CCC(N2Cc3ccc(C=O)cc3C2=O)C1=O ; C[Si](C)(C)CCOCN1C(=O)CCC(N2Cc3ccc(CNc4nnc(C5CCCCC5)o4)cc3C2=O)C1=O ; O=C1CCC(N2Cc3ccc(CNc4nnc(C5CCCCC5)o4)cc3C2=O)C(=O)N1]

### 4.1. Procedure for Preparation of Compound 2-5-cyclohexyl-1,3,4-oxadiazol-2-amine

To a solution of cyclohexanecarboxylic acid (1.00 g, 7.80 mmol, 971 uL, 1.00 eq) in dichloromethane (40.0 mL) were added hydrazinecarbothioamide (1.14 g, 12.5 mmol, 1.60 eq) and 1-(3-dimethylaminopropyl)-3-ethylcarbodiimide hydrochloride (4.49 g, 23.4 mmol, 3.00 eq). The reaction mixture was stirred at 25° C. for 12 h. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) to afford 5-cyclohexyl-1,3,4-oxadiazol-2-amine (300 mg, 1.79 mmol, 23% yield) as a white solid.

### MS (ESI) m/z 167.8 [M+H]⁺.

### Step 2. Procedure for Preparation of Compound 3—3-(6-(((5-cyclohexyl-1,3,4-oxadiazol-2-yl)amino)methyl)-1-oxoisoindolin-2-yl)-1-((2-(trimethylsilyl)ethoxy)methyl)piperidine-2,6-dione

To a solution of 2-(2,6-dioxo-1-((2-(trimethylsilyl)ethoxy)methyl)piperidin-3-yl)-3-oxoisoindoline-5-carbaldehyde (160 mg, 398 umol, 1.00 eq) in toluene (8.00 mL) were added 5-cyclohexyl-1,3,4-oxadiazol-2-amine (73.1 mg, 437 umol, 1.10 eq), 4-methylbenzenesulfonic acid (6.85 mg, 39.8 umol, 0.100 eq) and 4 A MS (160 mg). The mixture was stirred at 110° C. for 12 h. Then sodium triacetoxy borohydride (253 mg, 1.19 mmol, 3.00 eq) in tetrahydrofuran (2.00 mL) was added into the mixture. The mixture was stirred at 40° C. for 4 h. The reaction mixture was filtered and the filtrate was concentrated under reduced pressure to give a residue. The residue was purified by reverse phase chromatography (C18, 40 g; condition: water/acetonitrile=100:0 to 0:100, 0.1% formic acid) to afford 3-(6-(((5-cyclohexyl-1,3,4-oxadiazol-2-yl)amino)methyl)-1-oxoisoindolin-2-yl)-1-((2-(trimethylsilyl)ethoxy)methyl)piperidine-2,6-dione (90.0 mg, 163 umol, 41% yield) as a white solid

1H NMR (400 MHz, DMSO-d6) δ=8.07 (t, J=6.2 Hz, 1H), 7.74 (s, 1H), 7.66-7.56 (m, 2H), 5.25 (dd, J=5.0, 13.4 Hz, 1H), 5.07 (q, J=9.8 Hz, 2H), 4.52-4.44 (m, 3H), 4.34-4.25 (m, 1H), 3.54 (dt, J=5.5, 7.9 Hz, 2H), 3.16-3.01 (m, 1H), 2.83-2.68 (m, 2H), 2.45-2.33 (m, 1H), 2.13-2.05 (m, 1H), 1.98-1.89 (m, 2H), 1.78-1.68 (m, 2H), 1.67-1.56 (m, 1H), 1.50-1.26 (m, 5H), 0.89-0.83 (m, 2H), 0.00 (s, 9H).

### Step 3. Procedure for preparation of 3-(6-(((5-cyclohexyl-1,3,4-oxadiazol-2-yl)amino)methyl)-1-oxoisoindolin-2-yl)piperidine-2,6-dione

To a solution of 3-(6-(((5-cyclohexyl-1,3,4-oxadiazol-2-yl)amino)methyl)-1-oxoisoindolin-2-yl)-1-((2-(trimethylsilyl)ethoxy)methyl)piperidine-2,6-dione (90.0 mg, 163 umol, 1.00 eq) in dichloromethane (3.00 mL) was added trifluoroacetic acid (18.5 mg, 163 umol, 12.0 uL, 1.00 eq). The mixture was stirred at 25° C. for 2 h. Then added ammonium hydroxide (50.0 mg, 397 umol, 54.6 uL, 28% purity, 2.00 eq) was added into the mixture. The mixture was stirred at 25° C. for 1 h. The reaction mixture was filtered and concentrated under reduced pressure to give a residue. The residue was purified by reverse phase chromatography (C18, 40 g; condition: water/acetonitrile=100:0 to 0:100, 0.1% formic acid) to afford 3-(6-(((5-cyclohexyl-1,3,4-oxadiazol-2-yl)amino)methyl)-1-oxoisoindolin-2-yl)piperidine-2,6-dione (29.14 mg, 68.13 umol, 34% yield, 99% purity) as a white solid

1H NMR (400 MHz, DMSO-d6) δ=10.99 (s, 1H), 8.04 (t, J=6.1 Hz, 1H), 7.71 (s, 1H), 7.59 (q, J=7.9 Hz, 2H), 5.12 (dd, J=5.1, 13.4 Hz, 1H), 4.45 (br s, 1H), 4.43 (br d, J=6.4 Hz, 2H), 4.36-4.28 (m, 1H), 3.01-2.84 (m, 1H), 2.77-2.67 (m, 1H), 2.60 (br d, J=17.4 Hz, 1H), 2.45-2.32 (m, 1H), 2.04-1.96 (m, 1H), 1.91 (br d, J=11.4 Hz, 2H), 1.76-1.66 (m, 2H), 1.62 (br d, J=11.5 Hz, 1H), 1.45-1.20 (m, 5H)

### MS (ESI) m/z 424.3 [M+H]⁺.

### Example 50. Synthesis of Compound 142

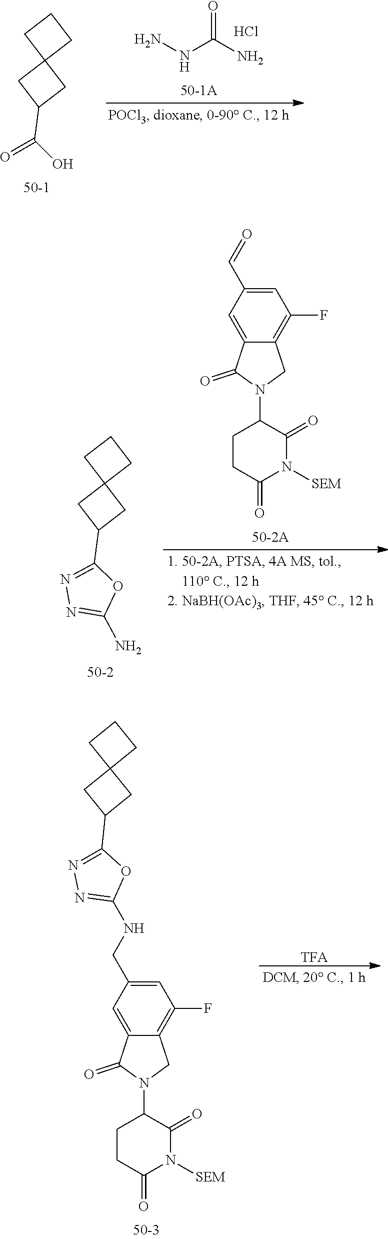

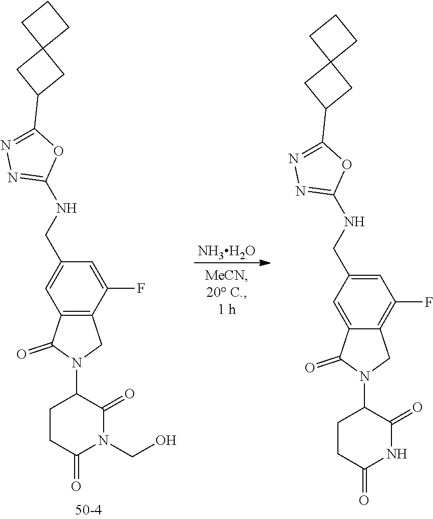 [SMILES: O=C1CCC(N2Cc3c(F)cc(CNc4nnc(C5CC6(CCC6)C5)o4)cc3C2=O)C(=O)N1CO ; "NH3·H2O" ; O=C1CCC(N2Cc3c(F)cc(CNc4nnc(C5CC6(CCC6)C5)o4)cc3C2=O)C(=O)N1]

### Step 1. Procedure for Preparation of Compound 2—5-(spiro[3.3]heptan-2-yl)-1,3,4-oxadiazol-2-amine

To a solution of spiro[3.3]heptane-2-carboxylic acid (1.00 g, 7.13 mmol, 1.00 eq) in dioxane (10.0 mL) were added hydrazinecarboxamide (875 mg, 7.84 mmol, 1.10 eq, hydrochloride) and phosphorus oxychloride (3.28 g, 21.4 mmol, 1.99 mL, 3.00 eq) at 0° C. The reaction mixture was stirred at 90° C. for 12 h. The reaction mixture was added to water (2.00 mL). The pH of the mixture was adjust to the range of 8-9 with saturated sodium bicarbonate, then the mixture was concentrated under reduced pressure to give a residue. The residue was diluted with dimethylformamide (5.00 mL) and then filtered. The filtrate was purified by reverse phase chromatography (C18, 80 g; condition: water/acetonitrile=100:0 to 0:100, 0.1% formic acid) and lyophilized to afford 5-(spiro[3.3]heptan-2-yl)-1,3,4-oxadiazol-2-amine (1.20 g, 6.70 mmol, 94% yield) as a yellow solid.

1H NMR (400 MHz, CDCl3-d) δ=6.84 (s, 2H), 3.38 (br s, 1H), 2.37-2.31 (m, 2H), 2.22-2.15 (m, 2H), 2.06 (t, J=7.3 Hz, 2H), 1.94-1.88 (m, 2H), 1.82-1.73 (m, 2H).

Step 2. Procedure for Preparation of Compound 3—3-(4-fluoro-1-oxo-6-(((5-(spiro[3.3]heptan-2-yl)-1,3,4-oxadiazol-2-yl)amino)methyl)isoindolin-2-yl)-1-((2-(trimethylsilyl)ethoxy) methyl)piperidine-2,6-dione

To a solution of 5-(spiro[3.3]heptan-2-yl)-1,3,4-oxadiazol-2-amine (100 mg, 558 umol, 1.00 eq) in toluene (10.0 mL) were added 4-methylbenzenesulfonic acid (9.61 mg, 55.8 umol, 0.100 eq), 4A MS (20.0 mg) and 2-(2,6-dioxo-1-((2-(trimethylsilyl)ethoxy)methyl)piperidin-3-yl)-7-fluoro-3-oxoisoindoline-5-carbaldehyde (282 mg, 670 umol, 1.20 eq). The mixture was stirred at 110° C. for 12 h under nitrogen atmosphere. Cooled to 20° C., sodium triacetoxyhydroborate (547 mg, 2.58 mmol, 5.00 eq) and tetrahydrofuran (5.00 mL) were added to the mixture and the reaction mixture was stirred at 45° C. for 12 h. The reaction mixture was concentrated under reduced pressure to give a residue. The residue was dissolved in N,N-dimethylformamide (0.5 mL) and then filtered. The filtered was purified by reverse phase chromatography (C18, 40 g; condition: water/acetonitrile=100:0 to 0:100, 0.1% formic acid) and lyophilized to afford 3-(4-fluoro-1-oxo-6-(((5-(spiro[3.3]heptan-2-yl)-1,3,4-oxadiazol-2-yl)amino)methyl)isoindolin-2-yl)-1-((2-(trimethylsilyl)ethoxy) methyl)piperidine-2,6-dione (80.0 mg, 137 umol, 27% yield) as green oil.

1H NMR (400 MHz, CDCl3-d) δ=7.69 (s, 1H), 7.38-7.34 (m, 1H), 5.28-5.24 (m, 1H), 5.23-5.15 (m, 2H), 4.66 (s, 2H), 4.56-4.48 (m, 1H), 4.43-4.34 (m, 1H), 3.76 (br t, J=6.4 Hz, 1H), 3.65-3.60 (m, 2H), 3.45-3.39 (m, 1H), 2.43-2.37 (m, 2H), 2.34-2.28 (m, 2H), 2.09 (br t, J=7.4 Hz, 2H), 2.00-1.94 (m, 2H), 1.87-1.81 (m, 2H), 1.31-1.23 (m, 4H), 0.98-0.92 (m, 2H), 0.04-0.04 (m, 9H).

### Step 3. Procedure for Preparation of Compound 4—3-(4-fluoro-1-oxo-6-(((5-(spiro[3.3]heptan-2-yl)-1,3,4-oxadiazol-2-yl)amino)methyl)isoindolin-2-yl)-1-(hydroxymethyl)piperidine-2,6-dione

To a solution of 3-(4-fluoro-1-oxo-6-(((5-(spiro[3.3]heptan-2-yl)-1,3,4-oxadiazol-2-yl)amino)methyl) isoindolin-2-yl)-1-((2-(trimethylsilyl)ethoxy) methyl)piperidine-2,6-dione (96.0 mg, 164 umol, 1.00 eq) in dichloromethane (3.00 mL) was added trifluoroacetic acid (554 mg, 4.86 mmol, 360 uL, 29.6 eq). The reaction mixture was stirred at 20° C. for 1 h. The reaction mixture was concentrated under reduced pressure to afford 3-(4-fluoro-1-oxo-6-(((5-(spiro[3.3]heptan-2-yl)-1,3,4-oxadiazol-2-yl)amino)methyl)isoindolin-2-yl)-1-(hydroxymethyl)piperidine-2,6-dione (70.0 mg, crude) as green oil.

### MS (ESI) m/z.484.2 [M+H]⁺.

### Step 4. Procedure for 3-(4-fluoro-1-oxo-6-(((5-(spiro[3.3]heptan-2-yl)-1,3,4-oxadiazol-2-yl)amino)methyl)isoindolin-2-yl)piperidine-2,6-dione

To a solution of 3-(4-fluoro-1-oxo-6-(((5-(spiro[3.3]heptan-2-yl)-1,3,4-oxadiazol-2-yl)amino)methyl) isoindolin-2-yl)-1-(hydroxymethyl)piperidine-2,6-dione (70.0 mg, 144.78 umol, 1.00 eq) in acetonitrile (3.00 mL) was added ammonium hydroxide (546 mg, 3.89 mmol, 0.600 mL, 25% purity, 26.9 eq). The reaction mixture was stirred at 20° C. for 1 h. The reaction mixture was concentrated under reduced pressure to give a residue (<30° C.). The residue was purified by Prep-HPLC(column: Phenomenex luna C18 150*25 mm*10 um; mobile phase: [water (formic acid)-acetonitrile]; B %: 28%-58%, 9 min) and lyophilized to afford 3-(4-fluoro-1-oxo-6-(((5-(spiro[3.3]heptan-2-yl)-1,3,4-oxadiazol-2-yl)amino)methyl)isoindolin-2-yl)piperidine-2,6-dione (13.8 mg, 30.13 umol, 21% yield, 99% purity) as a white solid.

1H NMR (400 MHz, DMSO-d6) δ=11.12-10.91 (m, 1H), 8.09 (br t, J=6.1 Hz, 1H), 7.58 (s, 1H), 7.46 (br d, J=9.8 Hz, 1H), 5.11 (br dd, J=5.0, 13.5 Hz, 1H), 4.57-4.46 (m, 2H), 4.45-4.35 (m, 2H), 3.38 (t, J=8.4 Hz, 1H), 2.96-2.86 (m, 1H), 2.62-2.57 (m, 1H), 2.45-2.40 (m, 2H), 2.39-2.33 (m, 2H), 2.22-2.16 (m, 2H), 2.07-2.02 (m, 2H), 1.92-1.87 (m, 2H), 1.80-1.72 (m, 2H).

### MS (ESI) m/z.454.0 [M+H]⁺.

### Example 51. Synthesis of Compound 143

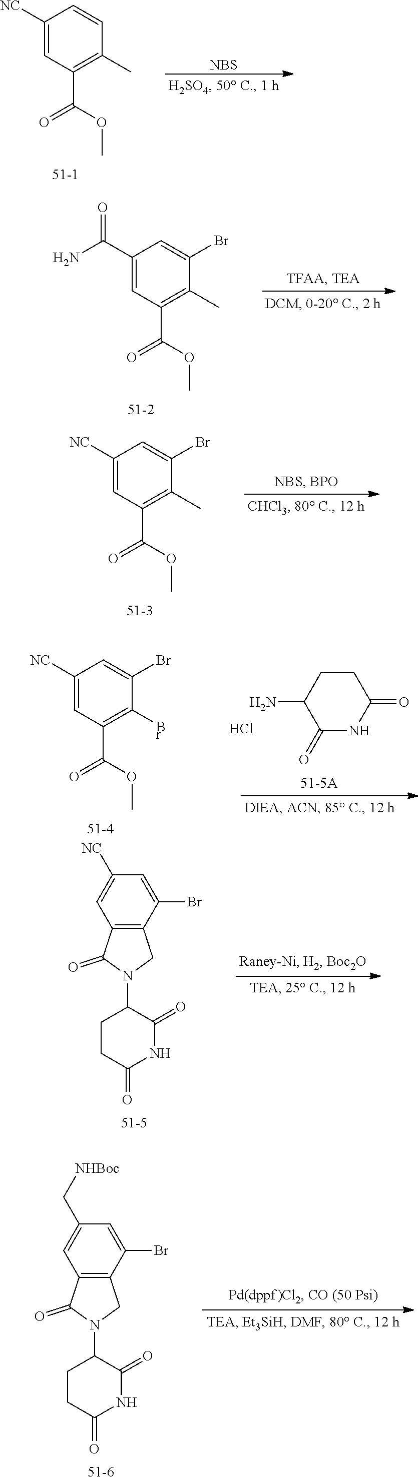

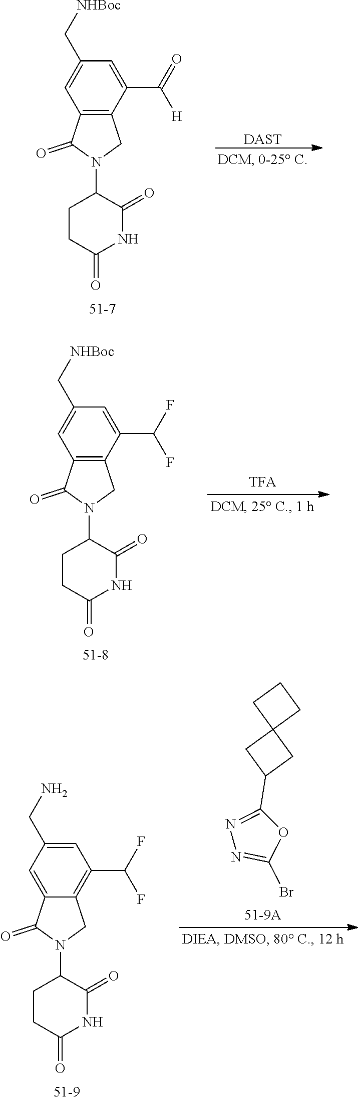

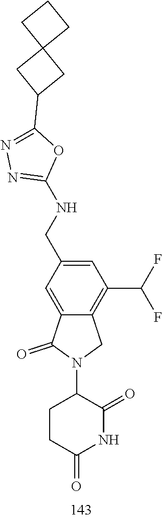 [SMILES: O=C1CCC(N2Cc3c(cc(CNc4nnc(C5CC6(CCC6)C5)o4)cc3C(F)F)C2=O)C(=O)N1]

### Step 1. Procedure for Compound 2—methyl 3-bromo-5-carbamoyl-2-methylbenzoate

To a solution of methyl 4-bromo-3-formylbenzoate (10.0 g, 57.1 mmol, 1.00 eq) in sulfuric acid (50.0 mL) was added N-bromosuccinimide (11.2 g, 62.8 mmol, 1.10 eq). The reaction mixture was stirred at 50° C. for 1 h. The reaction mixture was cooled to 25° C. Ethyl acetate (100 mL) and water (300 mL) were added into the mixture. The aqueous phase was extracted with ethyl acetate (2×300 mL). The combined organic layers were washed with brine (150 mL), dried over sodium sulfate and filtered. The filtrate was concentrated under reduced pressure to give a residue. The residue was purified by column chromatography (SiO2, petroleum ether/ethyl acetate=1/0 to 1/1) to afford methyl 3-bromo-5-carbamoyl-2-methylbenzoate (10.0 g, 36 mmol, 63% yield, 99% purity) as a white solid.

1H NMR (400 MHz, CDCl3-d) δ=8.23-8.11 (m, 2H), 6.23-5.58 (m, 2H), 3.94 (s, 3H), 2.69 (s, 3H).

### Step 2. Procedure for Compound 3—methyl 3-bromo-5-cyano-2-methylbenzoate

To a solution of methyl 3-bromo-5-carbamoyl-2-methylbenzoate (4.3 g, 15.8 mmol, 1.00 eq) in dichloromethane (30.0 mL) were added triethylamine (4.80 g, 47.4 mmol, 6.60 mL, 3.00 eq) and trifluoroacetic anhydride (4.31 g, 20.5 mmol, 2.86 mL, 1.30 eq) at 0° C. The reaction mixture was stirred at 20° C. for 2 h. The mixture was concentrated under reduced pressure to give a residue. The residue was purified by column chromatography (SiO2, petroleum ether/ethyl acetate=10/0 to 6/1) to afford methyl 3-bromo-5-cyano-2-methyl-benzoate (7.00 g, 27 mmol, 87% yield) as a white solid.

1H NMR (400 MHz, DMSO-d6) δ=8.39 (d, J=1.6 Hz, 1H), 8.15 (d, J=1.5 Hz, 1H), 3.87 (s, 3H), 2.57 (s, 3H).

### Step 3. Procedure for Compound 4—methyl 3-bromo-2-(bromomethyl)-5-cyanobenzoate

To a solution of methyl 3-bromo-5-cyano-2-methyl-benzoate (6.00 g, 23.6 mmol, 1.00 eq) in trichloromethane (80.0 mL), benzoyl peroxide (1.72 g, 7.08 mmol, 0.300 eq) and N-bromosuccinimide (4.62 g, 26.0 mmol, 1.10 eq). The reaction mixture was stirred at 80° C. for 12 h. The reaction mixture was filtered and the filtrate was concentrated under reduced pressure to give a residue. The residue was purified by column chromatography on silica gel (petroleum ether/ethyl acetate=30/1) to give methyl 3-bromo-2-(bromomethyl)-5-cyano-benzoate (5.5 g, crude) as a white solid.

1H NMR (400 MHz, DMSO-d6) δ=8.51 (d, J=1.4 Hz, 1H), 8.28 (d, J=1.5 Hz, 1H), 5.00 (s, 2H), 3.91 (s, 3H).

### Step 4. Procedure for Compound 5—7-bromo-2-(2,6-dioxopiperidin-3-yl)-3-oxoisoindoline-5-carbonitrile

To a solution of methyl 3-bromo-2-(bromomethyl)-5-cyano-benzoate (5.5 g, 16.52 mmol, 1.00 eq) in acetonitrile (30.0 mL) was added N,N-diisopropylethylamine (6.40 g, 49.6 mmol, 8.63 mL, 3.00 eq) and 3-aminopiperidine-2,6-dione hydrochloride (4.08 g, 24.8 mmol, 1.50 eq, hydrochloric acid). The mixture was stirred at 85° C. for 12 h. The mixture was concentrated under reduced pressure to give a residue. The residue was triturated with a mixture (ethyl acetate:water-3:2, 80 mL) at 25° C. for 1 h and then filtered to afford 7-bromo-2-(2,6-dioxopiperidin-3-yl)-3-oxoisoindoline-5-carbonitrile (3.50 g, 9.25 mmol, 60% yield, 92% purity) as a white solid.

1H NMR (400 MHz, DMSO-d6) δ=11.04 (s, 1H), 8.46 (s, 1H), 8.27 (s, 1H), 5.16 (dd, J=5.0, 13.3 Hz, 1H), 4.61-4.32 (m, 2H), 2.96-2.86 (m, 1H), 2.63-2.57 (m, 1H), 2.48-2.42 (m, 1H), 2.06-1.98 (m, 1H).

### Step 5. Procedure for Compound 6—tert-butyl ((7-bromo-2-(2,6-dioxopiperidin-3-yl)-3-oxoisoindolin-5-yl)methyl)carbamate

To a solution of 7-bromo-2-(2,6-dioxopiperidin-3-yl)-3-oxoisoindoline-5-carbonitrile (500 mg, 1.44 mmol, 1.00 eq) in tetrahydrofuran (5.00 mL) was added triethylamine (436 mg, 4.31 mmol, 600 uL, 3.00 eq), di-tert-butyl dicarbonate (940 mg, 4.31 mmol, 990 uL, 3.00 eq) and Raney-Ni (100 mg, 1.17 mmol) under nitrogen atmosphere. The mixture was stirred at 25° C. for 12 h under hydrogen atmosphere (15 Psi). The resulting mixture was filtered over Celite and the filtrate was concentrated under reduced pressure. The residue was purified by reversed-phase chromatography (C18, 120 g; condition: water/acetonitrile=100:0 to 60:40, 0.1% formic) and lyophilized to give tert-butyl ((7-bromo-2-(2,6-dioxopiperidin-3-yl)-3-oxoisoindolin-5-yl)methyl)carbamate (300 mg, 530.62 umol, 36.95% yield, 80% purity) as a white solid.

MS (ESI) m/z 395.8 [M-55]+.

### Step 6. Procedure for Compound 7—tert-butyl ((2-(2,6-dioxopiperidin-3-yl)-7-formyl-3-oxoisoindolin-5-yl)methyl)carbamate

To a solution of tert-butyl ((7-bromo-2-(2,6-dioxopiperidin-3-yl)-3-oxoisoindolin-5-yl)methyl)carbamate (350 mg, 774 umol, 1.00 eq) in dimethyl formamide (5.00 mL) was added [1,1′-bis(diphenylphosphino)ferrocene]dichloropalladium(II) (56.6 mg, 77.4 umol, 0.100 eq) and triethylsilane (270 mg, 2.32 mmol, 371 uL, 3.00 eq) and triethylamine (235 mg, 2.32 mmol, 323 uL, 3.00 eq). The mixture was degassed and purged with carbon monoxide (50 Psi). And the mixture was stirred at 80° C. for 12 h. The reaction mixture was cooled to 25° C. The residue was diluted with ethyl acetate (50 mL) and water (50 mL). The aqueous phase was extracted with ethyl acetate (3×50 mL). The organic layer was dried over sodium sulfate concentrated under reduced pressure to give a residue. The residue was purified by reversed-phase chromatography (C18, 120 g; condition: water/acetonitrile=100:0 to 60:40, 0.1% formic) and lyophilized to afford tert-butyl ((2-(2,6-dioxopiperidin-3-yl)-7-formyl-3-oxoisoindolin-5-yl)methyl)carbamate (300 mg, crude) as a white solid.

MS (ESI) m/z 401.8 [M+H]+.

### Step 7. Procedure for Compound 8—tert-butyl ((7-(difluoromethyl)-2-(2,6-dioxopiperidin-3-yl)-3-oxoisoindolin-5-yl)methyl)carbamate

To a solution of tert-butyl ((2-(2,6-dioxopiperidin-3-yl)-7-formyl-3-oxoisoindolin-5-yl)methyl)carbamate (130 mg, 324 umol, 1.00 eq) in dichloromethane (2.00 mL) at 0° C. was added dropwise diethylamine sulfur trifluoride (418 mg, 2.59 mmol, 342 uL, 8.00 eq) under nitrogen atmosphere. The mixture was stirred for 15 min and allowed to warm to 25° C. and was stirred for 4 h. The mixture was concentrated under reduced pressure to give a residue. The residue was purified by column chromatography (SiO2, petroleum ether/ethyl acetate=1/4) to afford tert-butyl N-[[7-(difluoromethyl)-2-(2,6-dioxo-3-piperidyl)-3-oxo-isoindolin-5-yl]methyl]carbamate (50.0 mg, 118 umol, 36% yield) as a white solid.

1H NMR (400 MHz, DMSO-d6) δ=11.01 (s, 1H), 7.76 (s, 1H), 7.68 (s, 1H), 7.58 (br t, J=6.0 Hz, 1H), 7.25 (t, J=55.2 Hz, 1H), 5.14 (dd, J=5.0, 13.4 Hz, 1H), 4.62-4.42 (m, 2H), 4.30-4.18 (m, 2H), 2.96-2.85 (m, 1H), 2.64-2.55 (m, 1H), 2.43 (br d, J=4.1 Hz, 1H), 2.07-2.00 (m, 1H), 1.39 (s, 9H).

### Step 8. Procedure for Compound 9—3-(6-(aminomethyl)-4-(difluoromethyl)-1-oxoisoindolin-2-yl)piperidine-2,6-dione

To a solution of tert-butyl ((7-(difluoromethyl)-2-(2,6-dioxopiperidin-3-yl)-3-oxoisoindolin-5-yl)methyl)carbamate (58.0 mg, 137 umol, 1.00 eq) in dichloromethane (4.00 mL) was added trifluoroacetic acid (1.23 g, 10.8 mmol, 0.800 mL, 78.9 eq). The mixture was stirred at 25° C. for 1 h. The reaction mixture was concentrated under reduced pressure to give 3-(6-(aminomethyl)-4-(difluoromethyl)-1-oxoisoindolin-2-yl)piperidine-2,6-dione (44.0 mg, 136 umol, 99% yield) as colorless oil.

MS (ESI) m/z 323.9 [M+H]+

### Step 9. Procedure for 3-(4-(difluoromethyl)-1-oxo-6-(((5-(spiro[3.3]heptan-2-yl)-1,3,4-oxadiazol-2-yl)amino)methyl)isoindolin-2-yl)piperidine-2,6-dione

To a solution of 3-(6-(aminomethyl)-4-(difluoromethyl)-1-oxoisoindolin-2-yl)piperidine-2,6-dione (44.0 mg, 101 umol, 1.00 eq, trifluoroacetic acid) in dimethylsulfoxide (3.00 mL) was added N,N-diisopropylethylamine (26.0 mg, 201 umol, 35.1 uL, 2.00 eq) and 2-bromo-5-(spiro[3.3]heptan-2-yl)-1,3,4-oxadiazole (24.5 mg, 101 umol, 1.00 eq). The mixture was stirred at 80° C. for 12 h. The reaction mixture was filtered and concentrated under reduced pressure to give a residue. The residue was purified by reversed-phase chromatography (C18, 80 g; condition: water/acetonitrile=100:0 to 0:100, 0.1% formic) and lyophilization to afford 3-(4-(difluoromethyl)-1-oxo-6-(((5-(spiro[3.3]heptan-2-yl)-1,3,4-oxadiazol-2-yl)amino)methyl)isoindolin-2-yl)piperidine-2,6-dione (2.86 mg, 6.18 umol, 6.67% yield) as a white solid.

1H NMR (400 MHz, DMSO-d6) δ=11.00 (s, 1H), 8.12 (t, J=6.1 Hz, 1H), 7.87 (s, 1H), 7.79 (s, 1H), 7.41-7.10 (m, 1H), 5.24-4.98 (m, 1H), 4.61-4.50 (m, 2H), 4.50-4.42 (m, 2H), 3.37 (br s, 1H), 2.94-2.87 (m, 1H), 2.61 (br dd, J=1.9, 3.1 Hz, 1H), 2.43-2.42 (m, 1H), 2.33 (br s, 2H), 2.19 (br dd, J=2.6, 8.4 Hz, 2H), 2.06-2.01 (m, 3H), 1.91-1.87 (m, 2H), 1.79-1.74 (m, 2H).

MS (ESI) m/z 486.2 [M+H]+.

### Example 52. Synthesis of Compound 144

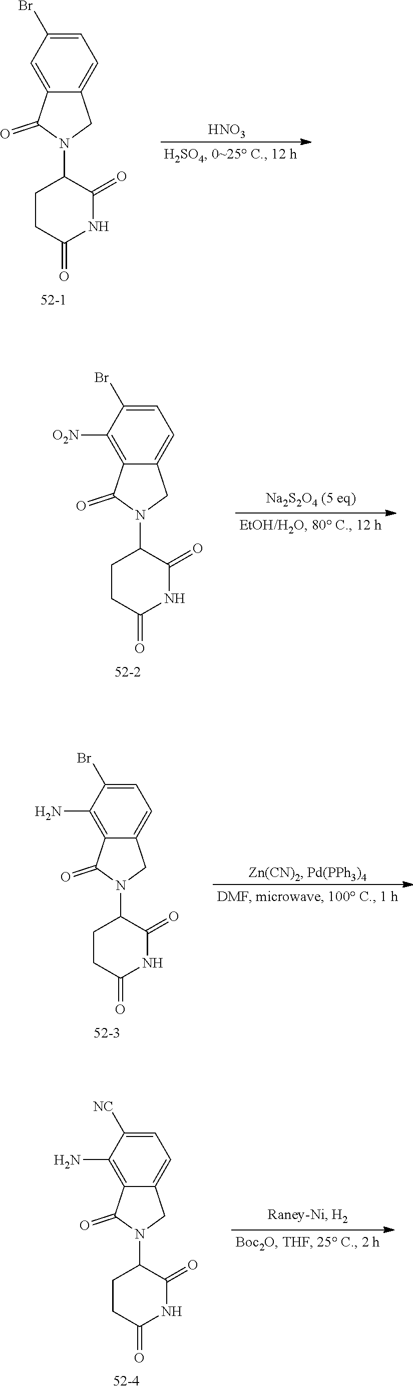

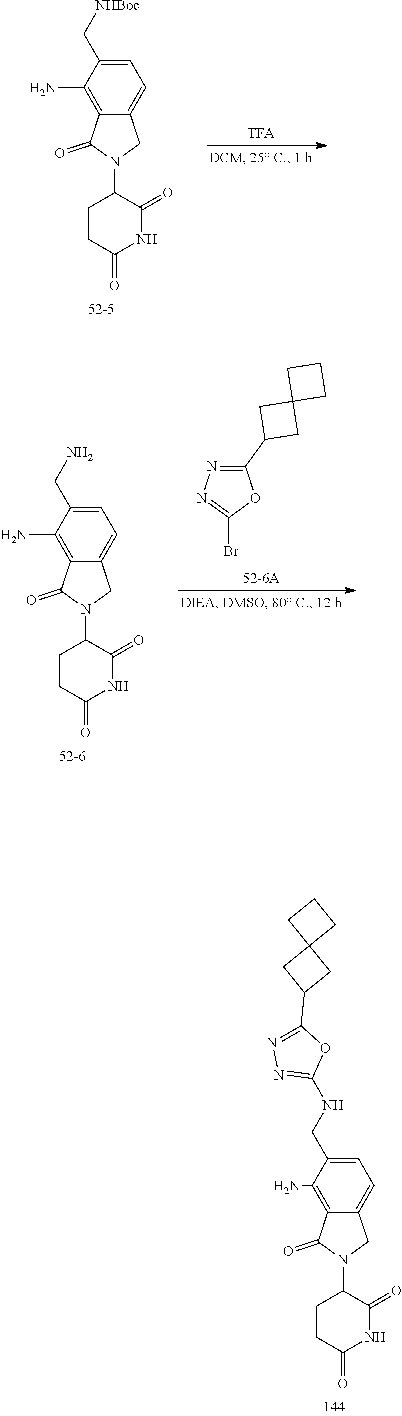 [SMILES: CC(C)(C)OC(=O)NCc1ccc2c(c1N)C(=O)N(C1CCC(=O)NC1=O)C2 ; NCc1ccc2c(c1N)C(=O)N(C1CCC(=O)NC1=O)C2 ; Brc1nnc(C2CC3(CCC3)C2)o1 ; Nc1c(CNc2nnc(C3CC4(CCC4)C3)o2)ccc2c1C(=O)N(C1CCC(=O)NC1=O)C2]

### Step 1. Procedure for Preparation of Compound 2—3-(6-bromo-7-nitro-1-oxoisoindolin-2-yl)piperidine-2,6-dione

To a solution of 3-(6-bromo-1-oxo-isoindolin-2-yl)piperidine-2,6-dione (2.00 g, 6.19 mmol, 1.00 eq) in sulfuric acid (20.0 mL) was added a mixture of nitric acid (3.60 g, 40.0 mmol, 2.57 mL, 70% purity, 6.46 eq) and sulfuric acid (11.8 g, 120 mmol, 6.40 mL, 19.4 eq) at 0° C. The mixture was warmed to 25° C. and stirred at 25° C. for 12 h. The reaction mixture was added ice (100 g) and filtered to give a filter cake, the filter cake was concentrated under reduced pressure to give 3-(6-bromo-7-nitro-1-oxoisoindolin-2-yl)piperidine-2,6-dione (1.80 g, 4.89 mmol, 79% yield) as yellow solid.

1H NMR (400 MHz, DMSO-d6) δ=11.06 (s, 1H), 8.14 (d, J=8.1 Hz, 1H), 7.83 (d, J=8.1 Hz, 1H), 5.09 (dd, J=5.0, 13.3 Hz, 1H), 4.62-4.54 (m, 1H), 4.48-4.42 (m, 1H), 2.95-2.86 (m, 1H), 2.63 (br s, 1H), 2.40 (br dd, J=4.1, 13.2 Hz, 1H), 2.06 (br dd, J=5.5, 11.3 Hz, 1H).

### Step 2. Procedure for Preparation of Compound 3—3-(7-amino-6-bromo-1-oxoisoindolin-2-yl)piperidine-2,6-dione

To a solution of 3-(6-bromo-7-nitro-1-oxo-isoindolin-2-yl)piperidine-2,6-dione (1.80 g, 4.89 mmol, 1.00 eq) in ethanol (40.0 mL) and water (5.00 mL) was added sodium hydrosulfite (4.26 g, 24.4 mmol, 5.32 mL, 5.00 eq). The mixture was stirred at 80° C. for 12 h. The reaction mixture was poured into water (200 mL) and extracted with ethyl acetate (3×90.0 mL). The combined organic phase was washed with brine (100 mL), dried with anhydrous sodium sulfate, filtered and concentrated in vacuum to give 3-(7-amino-6-bromo-1-oxoisoindolin-2-yl)piperidine-2,6-dione (1.00 g, 2.96 mmol, 60% yield) as a white solid

### Step 3. Procedure for Preparation of Compound 4—4-amino-2-(2,6-dioxopiperidin-3-yl)-3-oxoisoindoline-5-carbonitrile

To a solution of 3-(7-amino-6-bromo-1-oxo-isoindolin-2-yl)piperidine-2,6-dione (1.00 g, 2.96 mmol, 1.00 eq) and zinc cyanide (240 mg, 2.04 mmol, 129 uL, 0.700 eq) in dimethylformamide (1.00 mL) was added tetrakis[triphenylphosphine]palladium(0) (341 mg, 295 umol, 0.100 eq). The mixture was stirred at 100° C. for 1 h under microwave conditions. The reaction mixture was poured into water (200 mL) and extracted with ethyl acetate (3×90.0 mL). The combined organic phase was washed with brine (80.0 mL), dried over anhydrous sodium sulfate, filtered and concentrated in vacuum to give a residue. The residue was triturated with ethyl acetate (40.0 mL) at 25° C. for 10 min to afford 4-amino-2-(2,6-dioxopiperidin-3-yl)-3-oxoisoindoline-5-carbonitrile (600 mg, 2.11 mmol, 71% yield) as a yellow solid.

MS (ESI) m/z 285.1 [M+H]+.

### Step 4. Procedure for Preparation of Compound 5—tert-butyl ((4-amino-2-(2,6-dioxopiperidin-3-yl)-3-oxoisoindolin-5-yl)methyl)carbamate

To a solution of 4-amino-2-(2,6-dioxo-3-piperidyl)-3-oxo-isoindoline-5-carbonitrile (600 mg, 2.11 mmol, 1.00 eq) in tetrahydrofuran (20.0 mL) was added di-tert-butyldicarbonate (921 mg, 4.22 mmol, 969 uL, 2.00 eq) and Raney-Ni (18.0 mg, 211 umol, 0.100 eq) under nitrogen atmosphere. The mixture was stirred at 25° C. for 2 h under hydrogen atmosphere. The reaction mixture was filtered to give a filtrate, the filtrate was concentrated to give a residue. The residue was purified by reverse phase chromatography (C18, 120 g; condition: water/acetonitrile=100:0 to 60:40, 0.1% formic) and lyophilized to afford tert-butyl ((4-amino-2-(2,6-dioxopiperidin-3-yl)-3-oxoisoindolin-5-yl)methyl)carbamate (350 mg, 901 umol, 42% yield) as a white solid.

1H NMR (400 MHz, DMSO-d6) δ=10.96 (br s, 1H), 7.39-7.24 (m, 1H), 7.17 (br d, J=7.5 Hz, 1H), 6.66 (br d, J=7.5 Hz, 1H), 6.21-5.97 (m, 2H), 5.00 (br dd, J=5.0, 13.1 Hz, 1H), 4.35-4.26 (m, 1H), 4.21-4.13 (m, 1H), 4.03 (br d, J=5.6 Hz, 2H), 2.96-2.84 (m, 1H), 2.59 (br d, J=16.6 Hz, 1H), 2.42-2.31 (m, 1H), 2.02-1.92 (m, 1H), 1.47-1.34 (m, 9H).

### Step 5. Procedure for Preparation of Compound 5—3-(7-amino-6-(aminomethyl)-1-oxoisoindolin-2-yl)piperidine-2,6-dione

To a solution of tert-butyl ((4-amino-2-(2,6-dioxopiperidin-3-yl)-3-oxoisoindolin-5-yl)methyl)carbamate (100 mg, 257 umol, 1.00 eq) in dichloromethane (5.00 mL) was added methylsulphonicacid (74.2 mg, 772 umol, 55.0 uL, 3.00 eq). The mixture was stirred at 25° C. for 0.5 h. The reaction mixture was concentrated to give 3-(7-amino-6-(aminomethyl)-1-oxoisoindolin-2-yl)piperidine-2,6-dione (70.0 mg, crude) as yellow oil.

MS (ESI) m/z 272.0 [M+H-17]+.

### Step 6. Procedure for preparation of 3-(7-amino-1-oxo-6-(((5-(spiro[3.3]heptan-2-yl)-1,3,4-oxadiazol-2-yl)amino)methyl)isoindolin-2-yl)piperidine-2,6-dione

To solution of 3-(7-amino-6-(aminomethyl)-1-oxoisoindolin-2-yl)piperidine-2,6-dione (70.0 mg, 242 umol, 1.00 eq), 2-bromo-5-(spiro[3.3]heptan-2-yl)-1,3,4-oxadiazole (59.0 mg, 242 umol, 1.00 eq) and N,N-diisopropylethylamine (125 mg, 971 umol, 169 uL, 4.00 eq) in dimethylsulfoxide (3.00 mL) stirred at 80° C. for 12 h. The reaction mixture was filtered to give a filtrate. The filtrate was purified by Prep-HPLC (column: Phenomenex luna C18 150*25 mm*10 um; mobile phase: [water(FA)-ACN]; B %: 32%-52%, 58 min) and Prep-HPLC (column: Phenomenex C18 150*30 mm*5 um; mobile phase: [water(NH4HCO3)-ACN]; B %: 30%-60%, 10 min) to afford 3-(7-amino-1-oxo-6-(((5-(spiro[3.3]heptan-2-yl)-1,3,4-oxadiazol-2-yl)amino)methyl)isoindolin-2-yl)piperidine-2,6-dione (5.38 mg, 11.9 umol, 4.92% yield) as a white solid.

1H NMR (400 MHz, DMSO-d6) δ=11.05-10.85 (m, 1H), 7.81 (t, J=6.0 Hz, 1H), 7.29 (d, J=7.5 Hz, 1H), 6.68 (d, J=7.5 Hz, 1H), 6.13 (s, 2H), 5.00 (dd, J=5.1, 13.3 Hz, 1H), 4.35-4.23 (m, 3H), 4.21-4.13 (m, 1H), 2.93-2.84 (m, 1H), 2.63-2.57 (m, 1H), 2.44 (br d, J=5.3 Hz, 1H), 2.39-2.29 (m, 3H), 2.23-2.15 (m, 2H), 2.05 (br t, J=7.4 Hz, 2H), 1.98 (br d, J=5.4 Hz, 1H), 1.94-1.87 (m, 2H), 1.82-1.73 (m, 2H).

MS (ESI) m/z 451.0 [M+H]+.

### Example 53. Synthesis of Compound 145

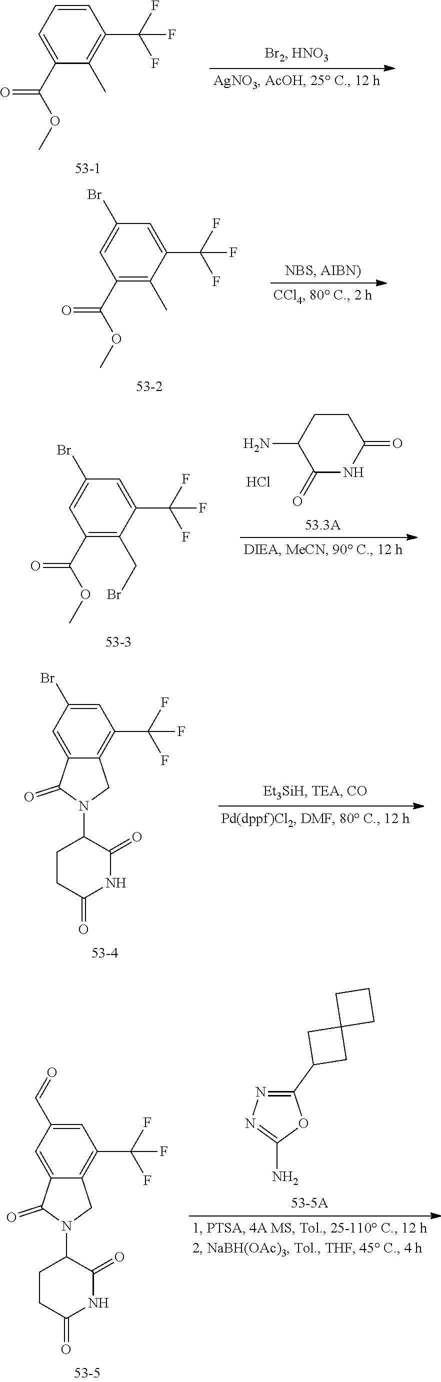

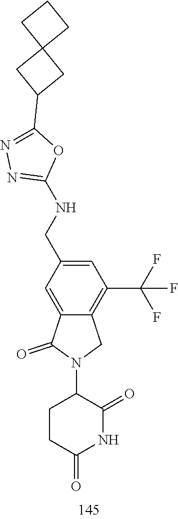 [SMILES: O=C1CCC(N2Cc3c(cc(CNc4nnc(C5CC6(CCC6)C5)o4)cc3C(F)(F)F)C2=O)C(=O)N1]

### Step 1. Procedure for compound 2—methyl 5-bromo-2-methyl-3-(trifluoromethyl)benzoate

To a solution of methyl 2-methyl-3-(trifluoromethyl)benzoate (5.00 g, 22.9 mmol, 1.00 eq) in acetic acid (20.0 mL) was added bromine (4.03 g, 25.2 mmol, 1.30 mL, 1.10 eq) and nitric acid (1.55 g, 24.1 mmol, 1.11 mL, 98% purity, 1.05 eq) at 25° C. Then silver nitrate (2.50 M, 9.63 mL, 1.05 eq) was added into the mixture over 30 min. And the mixture was stirred for 12 h at 25° C. The mixture was basified to 7 with 1 M sodium hydroxide. The mixture was diluted with water (200 mL) and extracted with ethyl acetate (100.0 mL*3). The combined organic layers were washed with brine (200 mL), dried over sodium sulfate and filtered. The filtrate was concentrated under reduced pressure and purified by column chromatography (SiO2, petroleum ether) to afford methyl 5-bromo-2-methyl-3-(trifluoromethyl)benzoate (5.10 g, crude) as yellow oil.

1H NMR (400 MHz, CDCl3) δ=8.07 (d, J=1.9 Hz, 1H), 7.90 (d, J=2.0 Hz, 1H), 3.95-3.94 (m, 3H), 2.60 (d, J=1.5 Hz, 3H)

### Step 2 Procedure for compound 3—methyl 5-bromo-2-(bromomethyl)-3-(trifluoromethyl)benzoate

To a solution of methyl 5-bromo-2-methyl-3-(trifluoromethyl)benzoate (3.00 g, 10.1 mmol, 1.00 eq) in tetrachloromethane (25.0 mL) was added N-bromosuccinimide (1.80 g, 10.1 mmol, 1.00 eq) and (E)-2,2′-(diazene-1,2-diyl)bis(2-methylpropanenitrile) (166 mg, 1.01 mmol, 0.10 eq) under nitrogen atmosphere. The mixture was stirred at 80° C. for 2 h. The mixture was concentrated under reduced pressure to give a residue. The residue was purified by column chromatography (SiO2, petroleum ether/ethyl acetate=50:1) to afford methyl 5-bromo-2-(bromomethyl)-3-(trifluoromethyl)benzoate (2.90 g, crude) as yellow oil.

1H NMR (400 MHz, CDCl3) δ=8.22-8.17 (m, 1H), 7.97-7.94 (m, 1H), 5.05 (s, 2H), 4.00 (s, 3H)

### Step 3. Procedure for compound 4—3-(6-bromo-1-oxo-4-(trifluoromethyl)isoindolin-2-yl)piperidine-2,6-dione

To a solution of methyl 5-bromo-2-(bromomethyl)-3-(trifluoromethyl)benzoate (2.76 g, 7.35 mmol, 1.10 eq) in acetonitrile (30.0 mL) was added N,N-diisopropylethylamine (2.59 g, 20.1 mmol, 3.49 mL, 3.00 eq) and 3-aminopiperidine-2,6-dione hydrochloride (1.10 g, 6.68 mmol, 1.00 eq, hydrochloric acid) at 25° C. And the mixture was stirred at 90° C. for 12 h. The reaction mixture was and concentrated under reduced pressure to give a residue. The residue was purified by column chromatography (SiO2, petroleum ether/ethyl acetate=3:1) to afford 3-(6-bromo-1-oxo-4-(trifluoromethyl)isoindolin-2-yl)piperidine-2,6-dione (309 mg, 774 umol, 11% yield, 98% purity) as a blue solid.

1H NMR (400 MHz, DMSO-d6) δ=11.03 (s, 1H), 8.28-8.16 (m, 2H), 5.20-5.10 (m, 1H), 4.71-4.57 (m, 1H), 4.55-4.43 (m, 1H), 2.98-2.83 (m, 1H), 2.63-2.58 (m, 1H), 2.42-2.24 (m, 1H), 2.06-1.99 (m, 1H)

### Step 4. Procedure for compound 5-2-(2,6-dioxopiperidin-3-yl)-3-oxo-7-(trifluoromethyl)isoindoline-5-carbaldehyde

To a solution of 3-(6-bromo-1-oxo-4-(trifluoromethyl)isoindolin-2-yl)piperidine-2,6-dione (309 mg, 790 umol, 1.00 eq) in N,N-dimethyl formamide (10.0 mL) was added [1,1′-bis(diphenylphosphino)ferrocene]dichloropalladium(II) (57.8 mg, 79.0 umol, 0.10 eq) and triethylsilane (276 mg, 2.37 mmol, 379 uL, 3.00 eq) and triethylamine (240 mg, 2.37 mmol, 330 uL, 3.00 eq). The mixture was degassed and purged with carbon monoxide (50 psi). The mixture was stirred at 80° C. for 12 h. The mixture was quenched with water (50.0 mL) and extracted with ethyl acetate (100 mL*3). The combined organic layer was washed with water (50.0 mL), dried over sodium sulfate, filtered and concentrated under reduced pressure to give a residue. The residue was purified by column chromatography (SiO2, petroleum ether/ethyl acetate=2:1) to afford 2-(2,6-dioxopiperidin-3-yl)-3-oxo-7-(trifluoromethyl)isoindoline-5-carbaldehyde (138 mg, 389 umol, 49% yield, 96% purity) as a yellow solid.

1H NMR (400 MHz, DMSO-d6) δ=11.09-10.95 (m, 1H), 10.27-10.10 (m, 1H), 8.54-8.44 (m, 1H), 8.09-7.92 (m, 1H), 5.24-5.10 (m, 1H), 4.84-4.49 (m, 2H), 2.98-2.89 (m, 1H), 2.64-2.56 (m, 1H), 2.53 (br s, 1H), 2.10-2.00 (m, 1H).

### Step 5. Procedure for 3-cyclopropylprop-2-yn-1-yl (1-(4-(2,6-dioxopiperidin-3-yl)-3,5-difluorophenyl)azetidin-3-yl)carbamate

To a solution of 2-(2,6-dioxopiperidin-3-yl)-3-oxo-7-(trifluoromethyl)isoindoline-5-carbaldehyde (138 mg, 406 umol, 1.00 eq) in toluene (16.0 mL) was added 5-(spiro[3.3]heptan-2-yl)-1,3,4-oxadiazol-2-amine (87.2 mg, 487 umol, 1.20 eq), p-toluene sulfonic acid (6.98 mg, 40.6 umol, 0.10 eq) and 4 A MS (138 mg) at 25° C. The mixture was stirred at 110° C. for 12 h. The mixture was cooled to 25° C. And then sodium triacetoxyhydroborate (429 mg, 2.03 mmol, 5.00 eq) in tetrahydrofuran (5.00 mL) was added into the mixture. The mixture was stirred at 45° C. for 4 h. The residue was purified by prep-HPLC (column: Phenomenex luna C18 150*25 mm*10 um; mobile phase: [water(formic acid)-acetonitrile]; B %: 34%-64%, 9 min) and lyophilized to afford 3-(1-oxo-6-(((5-(spiro[3.3]heptan-2-yl)-1,3,4-oxadiazol-2-yl)amino)methyl)-4-(trifluoromethyl)isoindolin-2-yl)piperidine-2,6-dione as a white solid.

1H NMR (400 MHz, DMSO-d6) δ=11.12-10.85 (m, 1H), 8.14 (t, J=6.1 Hz, 1H), 7.99 (br d, J=12.5 Hz, 2H), 5.15 (dd, J=5.2, 13.2 Hz, 1H), 4.68-4.60 (m, 1H), 4.54 (br d, J=6.3 Hz, 2H), 4.52-4.46 (m, 1H), 3.42-3.36 (m, 1H), 2.97-2.84 (m, 1H), 2.64-2.57 (m, 1H), 2.44 (br s, 2H), 2.35-2.30 (m, 2H), 2.22-2.14 (m, 2H), 2.06-2.02 (m, 2H), 1.93-1.85 (m, 2H), 1.81-1.71 (m, 2H).

MS (ESI) m/z 504.2 [M+H]+.

### Example 54. Compound binding to CRBN by HTRF assay

Compound activity was monitored in a Homogenous Time-Resolved Fluorescence (HTRF) assay using 1-[5-({2-[2-(2-{[2-(2,6-dioxopiperidin-3-yl)-1,3-dioxo-2,3-dihydro-1H-isoindol-4-yl]oxy}acetamido)ethoxy]ethyl}carbamoyl)pentyl]-3,3-dimethyl-2-[(1E,3E)-5-[(2E)-1,3,3-trimethyl-5-sulfo-2,3-dihydro-1H-indol-2-ylidene]penta-1,3-dien-1-yl]-3H-indol-1-ium-5-sulfonate as a fluorescent probe. Biochemical assays were conducted in Greiner white 384 well HiBase plates (Cat. No 784075-25) in 10 μL total volume. A one pot detection solution of CRBN-DDB1 (2.5 nM), Anti-His Terbium Cryptate Gold (1×, PerkinElmer Cat. #: 61HI2TLB), and Cy5-Thalidomide (100 nM, Tenova Cat.: T52461) was prepared in 20 mM HEPES, 20 mM NaCl, 0.2 mM TCEP, 0.2 mM EDTA, and 0.005% Tween20 was dispensed to each assay plate. Compounds were stored in dry, ambient temperatures at 10 mM. A 10-point, 1:3 dilution series was prepared from 10 mM stock concentrations in Echo-compatible LDV plates. 10 nL of each compound dilution series was dispensed into assays wells using an Echo 650 (Labcyte inc. USA). 10 nL of 10 mM Lenalidomide was transferred into the active-control wells for the assay and 10 nL of DMSO was transferred into the neutral-control wells. The assay was then allowed to incubate for 30 min at ambient temperature after transferring compound. Plate measurements were taken on a Pherastar FSX (BMG Labtech, Germany) using the HTRF Red filter (Ex. 337 nm, em1: 620 nm, em2: 665 nm) (Flashes: 50, Integration time: 60-400 us, Z-height: 10 mm, Ratio-multiplier: 10,000). The HTRF signal was then subsequently normalized to the neutral and active controls. Analysis and IC50 values were derived using KNIME analytics (KNIME Zurich) transformation and fitting within Collaborative Drug Discovery (Collaborative Drug Discovery USA). Ki was derived from the geometric mean of the IC50 values using the Cheng-Prustoff transformation.

### Example 55. HiBiT assay

HEK293 clonal lines with CRISPR KI HiBiT tag on CDK2 proteins and stably expressing LgBiT protein were obtained from Promega (Madison, WI). Cells were plated at 5000 cells per well using Multiflo (BioTek) in 384-well white solid bottom plates (Corning, 3570BC) in 25 ul volume in DMEM media (DMEM, high glucose, HEPES, no phenol red (ThermoFisher Scientific, 21063029)) containing 10% FBS (Corning, 35-075-CV), 1% Penicillin/Streptomycin (ThermoFisher Scientific, 15140-122). Cells were incubated for 16 hours at 37° C., 5% CO₂. 75 nL of a compound at 30 μM were dosed into the plate using an Echo® 650 liquid handler (Labcyte). Cells were incubated at 37° C., 5% CO₂ for 24 hours. 5 ul of HiBiT Lysis Reagent (Promega, N3030) is added using Combi Multidrop (ThermoFisher Scientific), plates are incubated for 15′ at 37° C., 5% CO₂ and then signal was read on a Pherastar FSX using “LUM plus” optic module.

Analysis was performed in Scinamic (Scinamic, Cambridge, MA). Luminescence response (R) was calculated by the formula: response=100*(S−N)/(P−N) where S is the signal of the well, N and P the mean negative and positive control values respectively of the same plate. The luminescence response was then fitted in Scinamic using a 3-parameter agonist logistic fit (hillslope=1, EC50>0, top/bottom unconstrained).

### Example 56. NanoBiT Assay

HEK293 clonal lines with CRISPR KI HiBiT tag on either CDK2 proteins and stably expressing LgBiT protein were obtained from Promega (Madison, WI). Cells were plated at 5000 cells per well using Multiflo (BioTek) in 384-well white solid bottom plates (Corning, 3570BC) in 25 ul volume in DMEM media (DMEM, high glucose, HEPES, no phenol red (ThermoFisher Scientific, 21063029)) containing 10% FBS (Corning, 35-075-CV), 1% Penicillin/Streptomycin ((ThermoFisher Scientific, 15140-122), and 1% Endurazine (Nano-Glo Endurazine Live Cell Substrate (Promega, N2571)). Cells were incubated for 16 hours at 37 C, 5% CO₂. 75 nL of a compound at 30 μM were dosed into the plate using an Echo® 650 liquid handler (Labcyte). Cells were incubated at 37° C., 5% CO₂ for 24 hours and then signal was read on a Pherastar FSX using “LUM plus” optic module.

Analysis was performed in Scinamic (Scinamic, Cambridge, MA). Luminescence response (R) was calculated by the formula: response=100*(S−N)/(P−N) where S is the signal of the well, N and P the mean negative and positive control values respectively of the same plate. The luminescence response was then fitted in Scinamic using a 3-parameter agonist logistic fit (hillslope=1, EC50>0, top/bottom unconstrained).

TABLE 2
HTRF, NanoBiT, and HiBiT Data

| | CDK2 | CDK2 | |
|---|---|---|---|
| Compound | HiBiT | NanoBiT | CRBN |
| No. | Dmax (%) | Dmax (%) | HTRF Ki |
| 1 | B | — | F |
| 2 | C | — | F |
| 3 | B | — | F |
| 4 | C | — | F |
| 6 | B | — | — |
| 12 | B | — | E |
| 13 | A | — | F |
| 14 | B | — | F |
| 15 | C | — | E |
| 16 | A | — | F |
| 17 | B | — | F |
| 18 | B | — | F |
| 19 | A | — | E |
| 20 | B | — | E |
| 21 | A | — | F |
| 22 | A | — | F |
| 23 | A | — | F |
| 24 | B | — | F |
| 25 | A | — | F |
| 26 | B | — | E |
| 27 | A | — | F |
| 28 | B | — | F |
| 29 | A | — | E |
| 30 | A | — | F |
| 31 | B | — | F |
| 32 | B | — | F |
| 33 | B | — | F |
| 34 | A | — | F |
| 35 | B | — | E |
| 36 | A | — | E |
| 37 | A | — | F |
| 38 | B | — | E |
| 39 | A | — | F |
| 40 | A | — | F |
| 41 | B | — | F |
| 42 | — | B | — |
| 43 | B | — | E |
| 44 | — | B | — |
| 45 | — | A | F |
| 46 | B | — | E |
| 47 | B | — | F |
| 49 | — | B | F |
| 50 | — | A | — |
| 51 | — | A | E |
| 52 | B | — | F |
| 53 | — | B | — |
| 54 | B | — | F |
| 55 | — | B | — |
| 56 | — | B | F |
| 57 | — | A | — |
| 58 | — | A | E |
| 60 | — | B | — |
| 61 | — | A | — |
| 62 | — | C | — |
| 63 | — | B | — |
| 64 | B | — | E |
| 65 | C | — | E |
| 66 | B | — | F |
| 67 | B | — | F |
| 68 | — | B | — |
| 69 | C | — | E |
| 70 | C | — | E |
| 71 | — | A | — |
| 72 | — | A | F |
| 73 | — | A | F |
| 74 | A | — | F |
| 75 | — | A | — |
| 76 | — | A | — |
| 77 | — | B | F |
| 78 | — | A | — |
| 79 | — | A | F |
| 80 | — | A | F |
| 81 | — | B | E |
| 82 | — | A | — |
| 83 | — | A | F |
| 84 | C | — | F |
| 85 | — | B | — |
| 86 | C | — | E |
| 87 | — | A | |
| 88 | B | — | F |
| 89 | C | — | F |
| 90 | B | — | F |
| 91 | — | A | F |
| 92 | — | A | — |
| 93 | C | — | E |
| 94 | B | — | F |
| 95 | B | — | F |
| 96 | — | B | — |
| 97 | — | A | — |
| 98 | — | B | — |
| 101 | — | B | F |
| 102 | — | B | E |
| 103 | — | B | E |
| 104 | — | C | E |
| 105 | — | C | E |
| 106 | — | C | F |
| 107 | — | C | F |
| 108 | — | B | F |
| 109 | — | B | F |
| 110 | — | B | F |
| 111 | — | B | E |
| 112 | — | B | F |
| 113 | — | B | F |
| 114 | — | B | F |
| 115 | — | C | F |
| 116 | — | B | F |
| 117 | — | D | F |
| 118 | — | C | F |
| 119 | — | B | F |
| 120 | — | B | F |
| 121 | — | B | F |
| 122 | — | C | F |
| 123 | — | B | F |
| 124 | — | B | F |
| 125 | — | B | F |
| 126 | — | B | F |
| 127 | — | C | E |
| 128 | — | B | F |
| 129 | — | B | F |
| 130 | — | B | F |
| 131 | — | B | F |
| 132 | — | B | F |
| 133 | — | B | F |
| 134 | — | B | E |
| 135 | — | C | F |
| 136 | — | C | F |
| 137 | — | C | E |
| 138 | — | C | E |
| 139 | — | C | F |
| 140 | — | C | F |
| 141 | — | B | F |
| 142 | — | C | F |
| 143 | — | B | E |
| 144 | — | B | F |
| 145 | — | B | F |

For CDK2 HiBiT: According to the code, A represents a Dₘₐₓ value of ≤10%, B represents a Dₘₐₓ value of >10% and ≤50%, C represents a Dₘₐₓ value of >50% and ≤80%, D represents Dₘₐₓ value of >80%.
For HTRF: According to the code E represents a Ki value of ≤0.1 μM, F represents a Ki value >0.1 μM and ≤1 μM, G represents an Ki value >1 μM.

### EQUIVALENTS

While specific embodiments have been discussed, the above specification is illustrative and not restrictive. Many variations of the embodiments will become apparent to those skilled in the art upon review of this specification. The full scope of what is disclosed should be determined by reference to the claims, along with their full scope of equivalents, and the specification, along with such variations.

What is claimed is:

1. A compound of Formula (I): 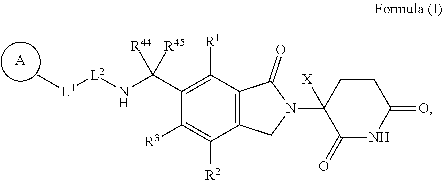
or a pharmaceutically acceptable salt thereof, wherein:
X is H or deuterium;
L¹ is selected from the group consisting of: 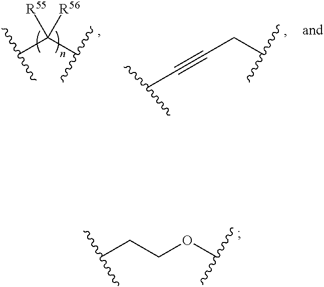
L² is selected from the group consisting of: 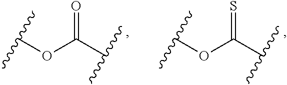 [SMILES: *OC(*)=O ; *OC(*)=S]
or a 5-6 membered heteroaryl;
each of R¹, R², R³ is independently selected from the group consisting of hydrogen, halogen, hydroxy, cyano, nitrogen, oxo, —N(R^{Za})(R^{Zb}), C₁₋₆ alkoxy, and C₁₋₆ alkyl, wherein C₁₋₆ alkyl is optionally substituted with one or more halogens;
each of R⁴⁴ and R⁴⁵ is independently H or C₁₋₆ alkyl;
or R⁴⁴ and R⁴⁵, together with the carbon to which they are attached, form a C₃₋₁₂ cycloalkyl ring;
each of R⁵⁵ and R⁵⁶ is independently selected from the group consisting of H, C₁₋₆ alkyl, aryl, and C₃₋₁₂ cycloalkyl, wherein C₁₋₆ alkyl is optionally substituted with one or more substituents selected from the group consisting of C₁₋₆ alkoxy, aryl, and C₃₋₁₂ cycloalkyl;
ring A is C₃₋₁₂ cycloalkyl or 3 to 10 membered heterocyclyl, wherein each of C₃₋₁₂ cycloalkyl and 3 to 10 membered heterocyclyl is optionally substituted with one or more occurrences of R⁴;
each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵;
each occurrence of R⁵ is independently halogen, C₁₋₆ alkyl, C₁₋₆ alkoxy, —SO₂—, or —C(O)OR^{D};
each of R^{A}, R^{B}, R^{C}, and R^{D} is independently C₁₋₆ alkyl;
R^{Z} is H or C₁₋₆ alkyl;
each of R^{Za} and R^{Zb} independently H or and C₁₋₆ alkyl; and
n is 0, 1, 2, 3, or 4.

2. The compound of claim 1, wherein L² is

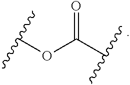 [SMILES: *OC(*)=O]

3. The compound of claim 1, wherein L² is

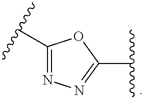 [SMILES: *c1nnc(*)o1]

4. The compound of claim 1, wherein the compound is a compound of Formula (I-C):

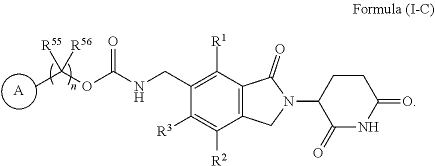

5. The compound of claim 1, wherein the compound is a compound of Formula (I-F):

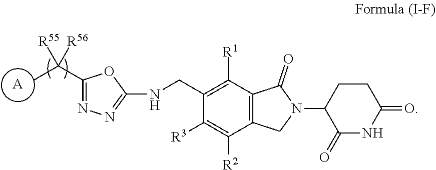

6. The compound of claim 1, wherein X is H.

7. The compound of claim 1, wherein R¹, R², and R³ are H.

8. The compound of claim 1, wherein R⁵⁵ and R⁵⁶ are H.

9. The compound of claim 1, wherein n is 3, 2, 1, or 0.

10. The compound of claim 1, wherein ring A is C₃₋₁₂ cycloalkyl optionally substituted with one or more occurrences of R⁴.

11. The compound of claim 1, wherein ring A is 3 to 10 membered heterocyclyl optionally substituted with one or more occurrences of R⁴.

12. The compound of claim 1, wherein ring A is selected from the group consisting of: 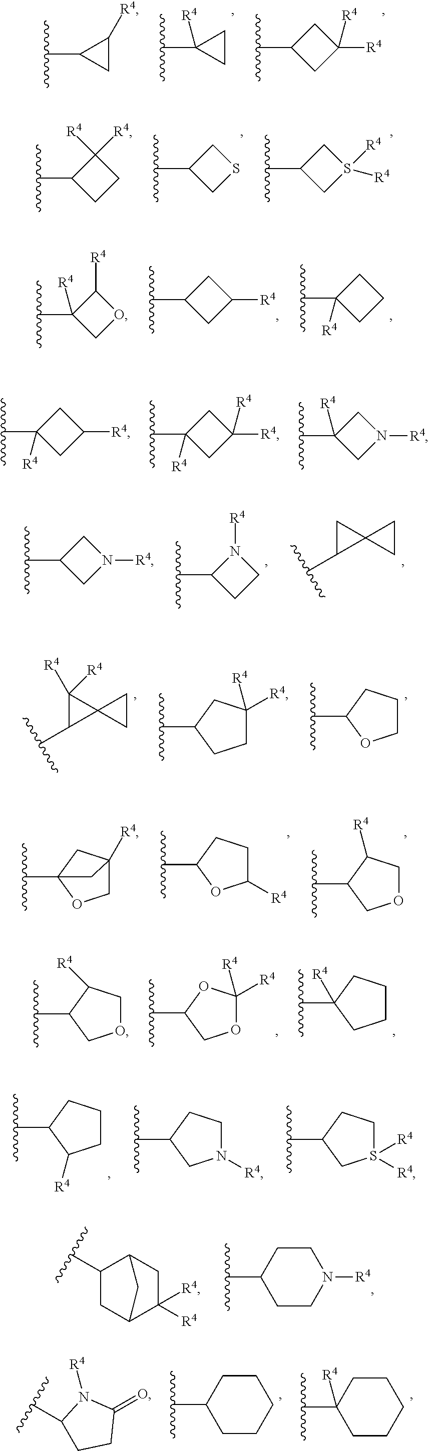 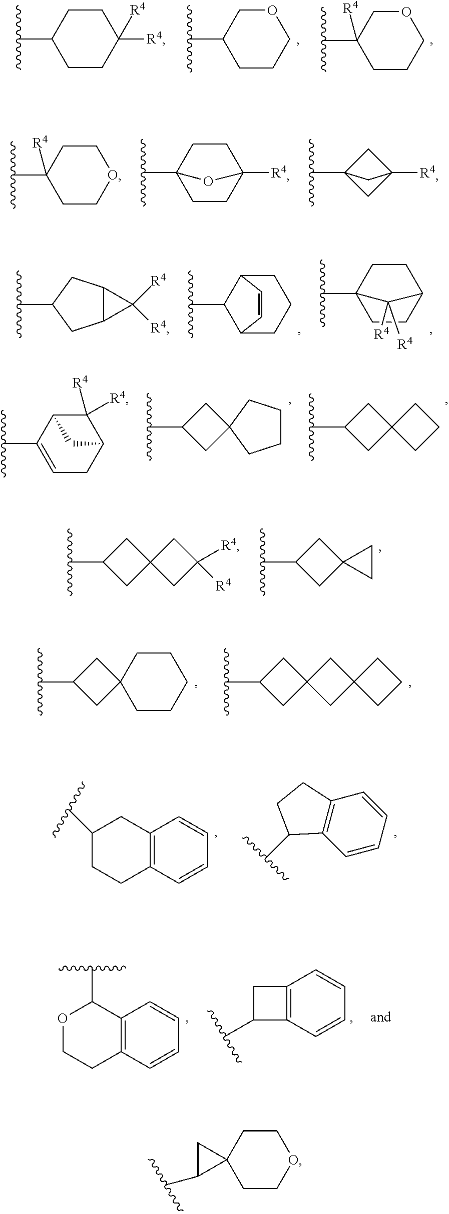
wherein each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; and R^{Z} is H or C₁₋₆ alkyl.

13. The compound of claim 1, wherein ring A is a spirocyclic C₃₋₁₂ cycloalkyl optionally substituted with one or more occurrences of R⁴.

14. The compound of claim 1, wherein ring A is selected from the group consisting of: 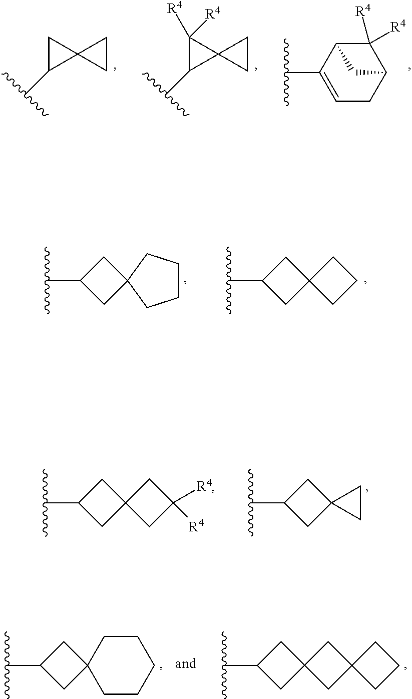
wherein each occurrence of R⁴ is independently halogen, cyano, hydroxyl, oxo, —S(O)₂R^{A}, —NR^{Z}C(O)OR^{B}, —C(O)R^{C}, —C(O)N(R^{C})₂, C₁₋₆ alkyl, C₁₋₆ alkoxy, C₃₋₁₀ cycloalkyl, 3 to 10 membered heterocyclyl, aryl, or heteroaryl, wherein each of C₁₋₆ alkyl, C₁₋₆ alkoxy, C₃₋₁₀ cycloalkyl, 3 to 10 membered heterocyclyl, aryl, and heteroaryl is optionally substituted with one or more occurrences of R⁵; each occurrence of R⁵ is independently halogen, C₁₋₆ alkyl, C₁₋₆ alkoxy, —SO₂—, or —C(O)OR^{D}; each of R^{A}, R^{B}, R^{C}, and R^{D} is independently C₁₋₆ alkyl; and R^{Z} is H or C₁₋₆ alkyl.

15. A compound selected from the group consisting of: 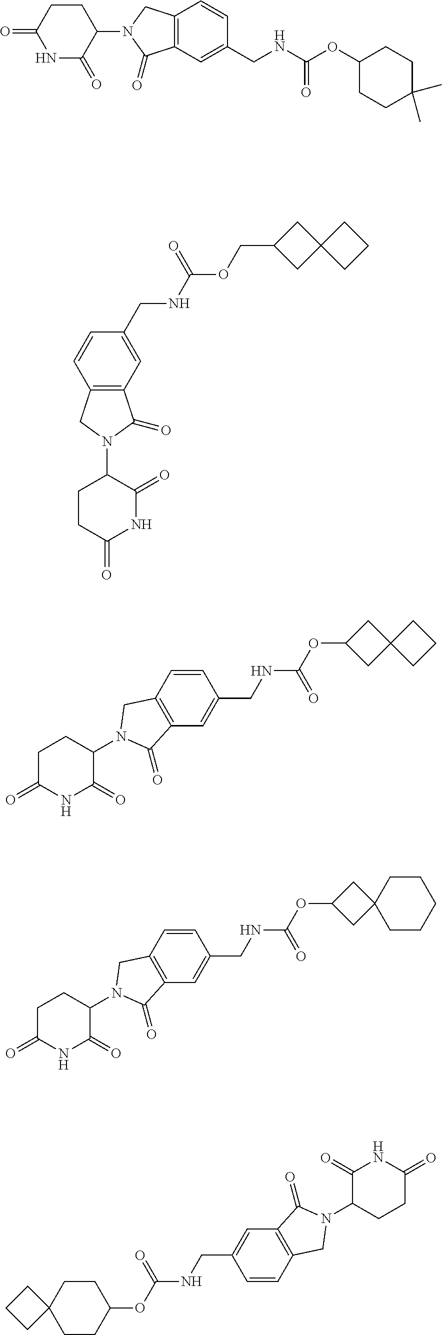 [SMILES: CC1(C)CCC(OC(=O)NCc2ccc3c(c2)C(=O)N(C2CCC(=O)NC2=O)C3)CC1 ; O=C1CCC(N2Cc3ccc(CNC(=O)OCC4CC5(CCC5)C4)cc3C2=O)C(=O)N1 ; O=C1CCC(N2Cc3ccc(CNC(=O)OC4CC5(CCC5)C4)cc3C2=O)C(=O)N1 ; O=C1CCC(N2Cc3ccc(CNC(=O)OC4CC5(CCCCC5)C4)cc3C2=O)C(=O)N1 ; O=C1CCC(N2Cc3ccc(CNC(=O)OC4CCC5(CCC5)CC4)cc3C2=O)C(=O)N1] 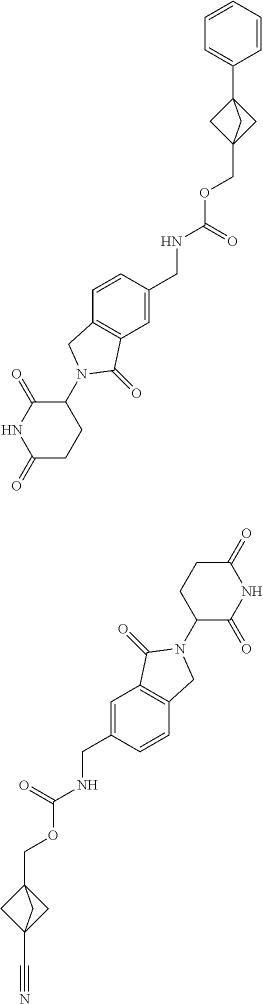 [SMILES: O=C1CCC(N2Cc3ccc(CNC(=O)OCC45CC(c6ccccc6)(C4)C5)cc3C2=O)C(=O)N1 ; N#CC12CC(COC(=O)NCc3ccc4c(c3)C(=O)N(C3CCC(=O)NC3=O)C4)(C1)C2] 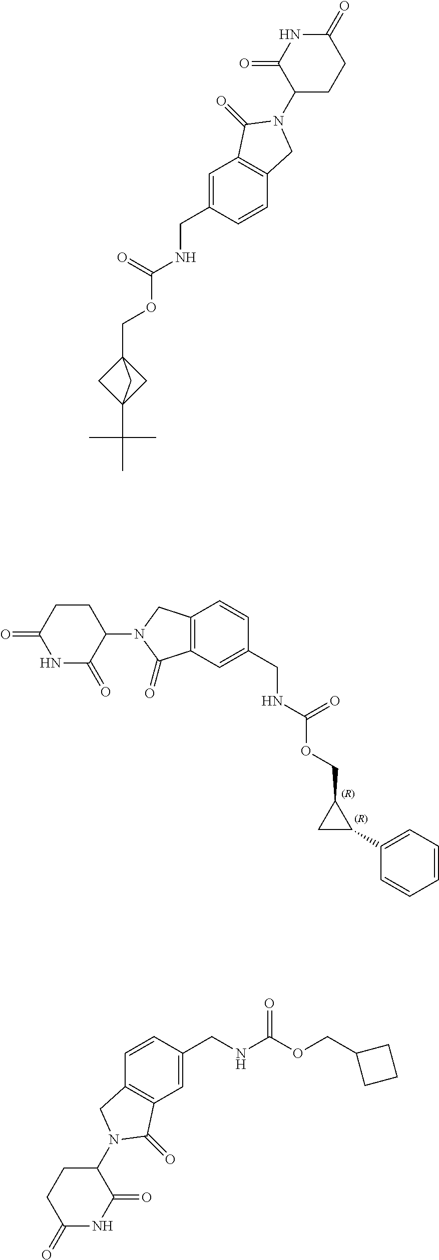 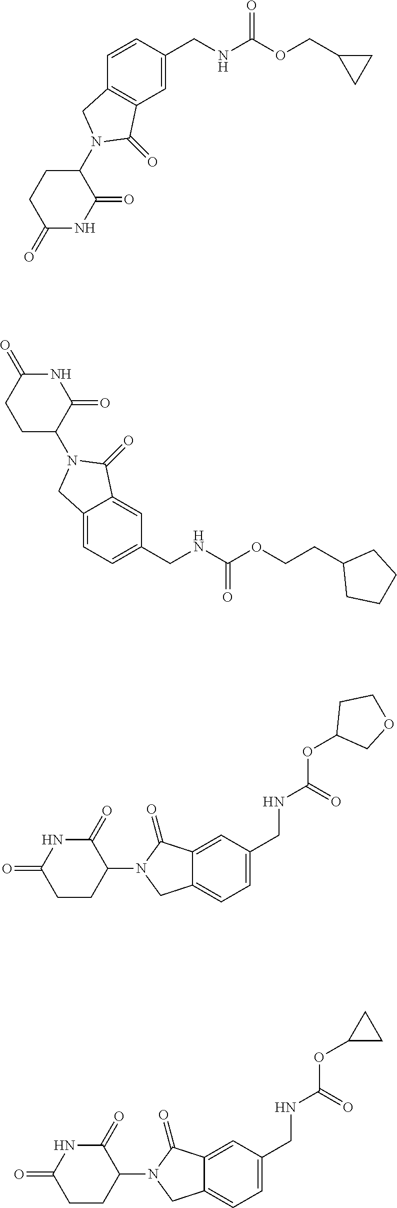 [SMILES: O=C1CCC(N2Cc3ccc(CNC(=O)OCC4CC4)cc3C2=O)C(=O)N1 ; O=C1CCC(N2Cc3ccc(CNC(=O)OCCC4CCCC4)cc3C2=O)C(=O)N1 ; O=C1CCC(N2Cc3ccc(CNC(=O)OC4CCOC4)cc3C2=O)C(=O)N1 ; O=C1CCC(N2Cc3ccc(CNC(=O)OC4CC4)cc3C2=O)C(=O)N1] 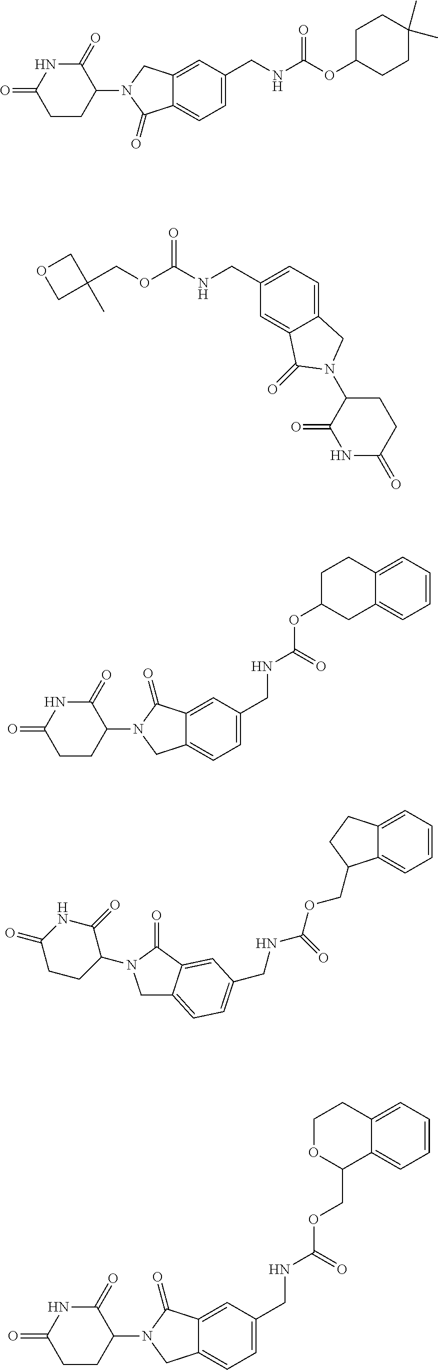 [SMILES: CC1(C)CCC(OC(=O)NCc2ccc3c(c2)CN(C2CCC(=O)NC2=O)C3=O)CC1 ; CC1(COC(=O)NCc2ccc3c(c2)C(=O)N(C2CCC(=O)NC2=O)C3)COC1 ; O=C1CCC(N2Cc3ccc(CNC(=O)OC4CCc5ccccc5C4)cc3C2=O)C(=O)N1 ; O=C1CCC(N2Cc3ccc(CNC(=O)OCC4CCc5ccccc54)cc3C2=O)C(=O)N1 ; O=C1CCC(N2Cc3ccc(CNC(=O)OCC4OCCc5ccccc54)cc3C2=O)C(=O)N1] 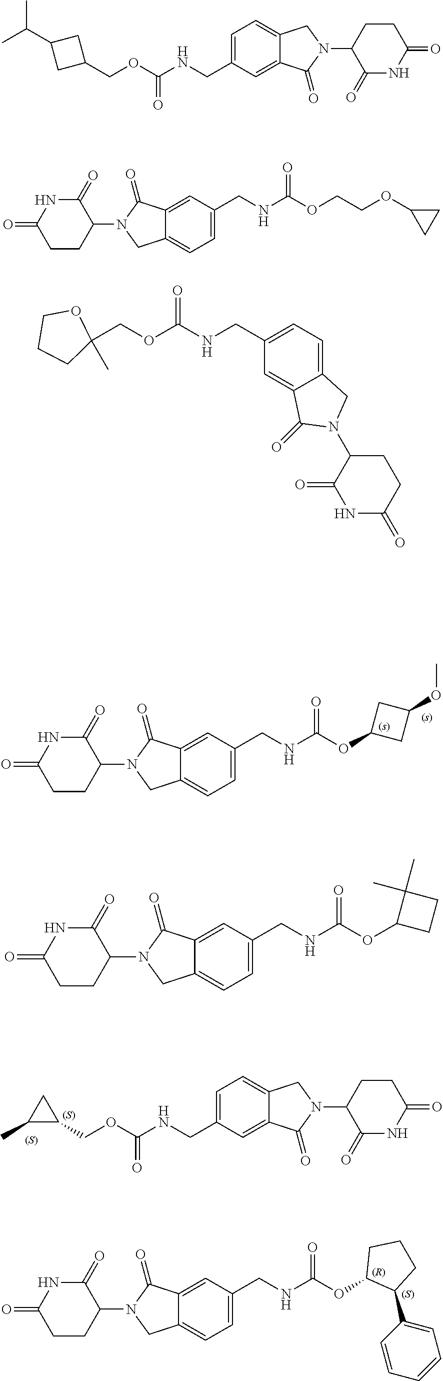 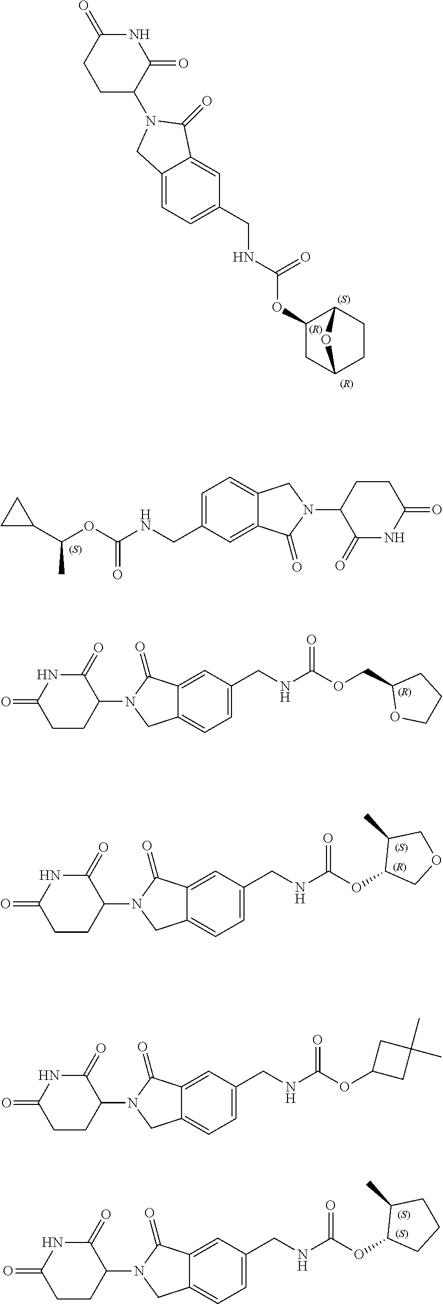 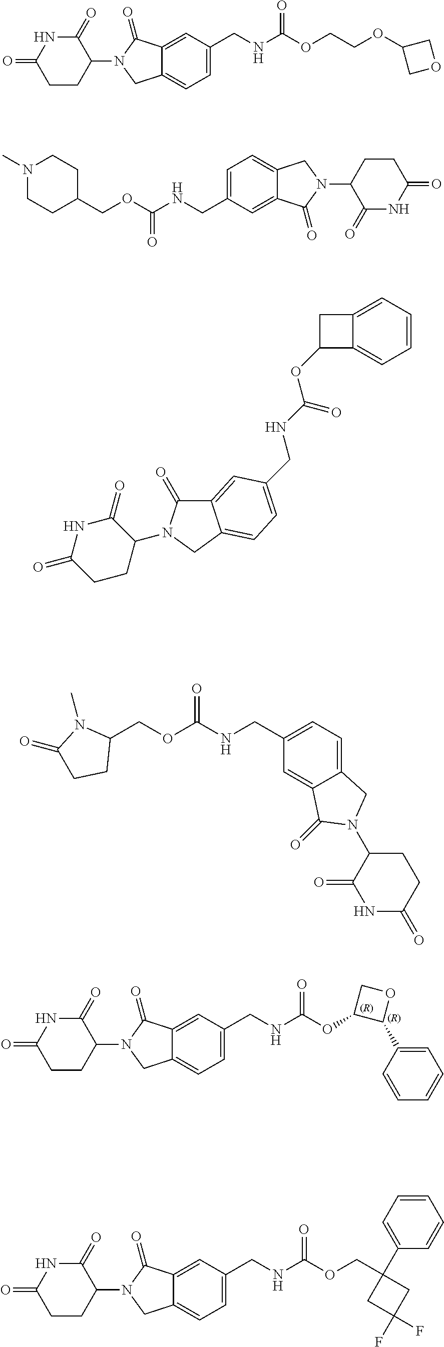 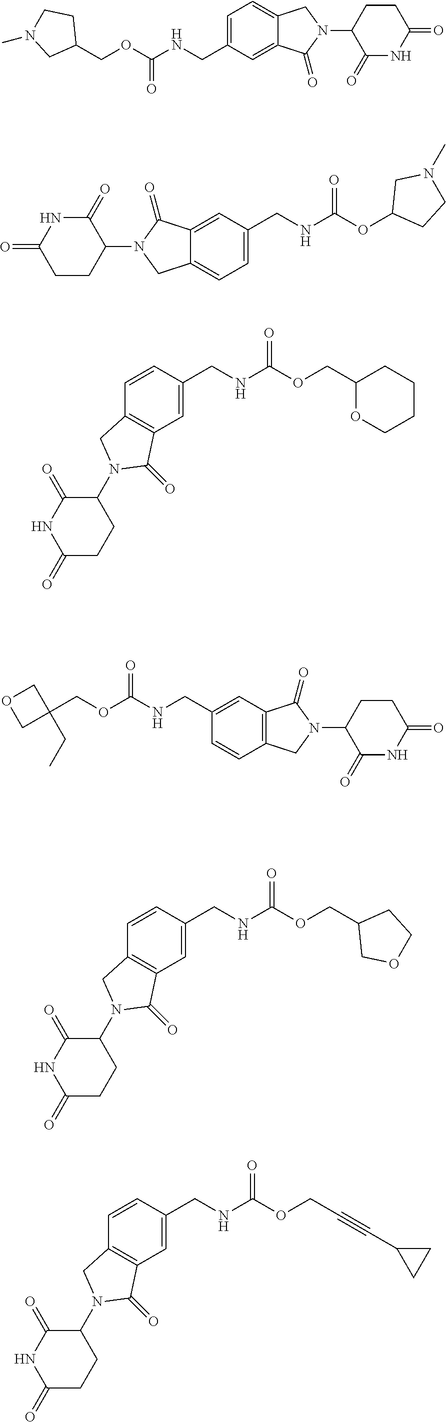 [SMILES: CN1CCC(COC(=O)NCc2ccc3c(c2)C(=O)N(C2CCC(=O)NC2=O)C3)C1 ; CN1CCC(OC(=O)NCc2ccc3c(c2)C(=O)N(C2CCC(=O)NC2=O)C3)C1 ; O=C1CCC(N2Cc3ccc(CNC(=O)OCC4CCCCO4)cc3C2=O)C(=O)N1 ; CCC1(COC(=O)NCc2ccc3c(c2)C(=O)N(C2CCC(=O)NC2=O)C3)COC1 ; O=C1CCC(N2Cc3ccc(CNC(=O)OCC4CCOC4)cc3C2=O)C(=O)N1 ; O=C1CCC(N2Cc3ccc(CNC(=O)OCC#CC4CC4)cc3C2=O)C(=O)N1] 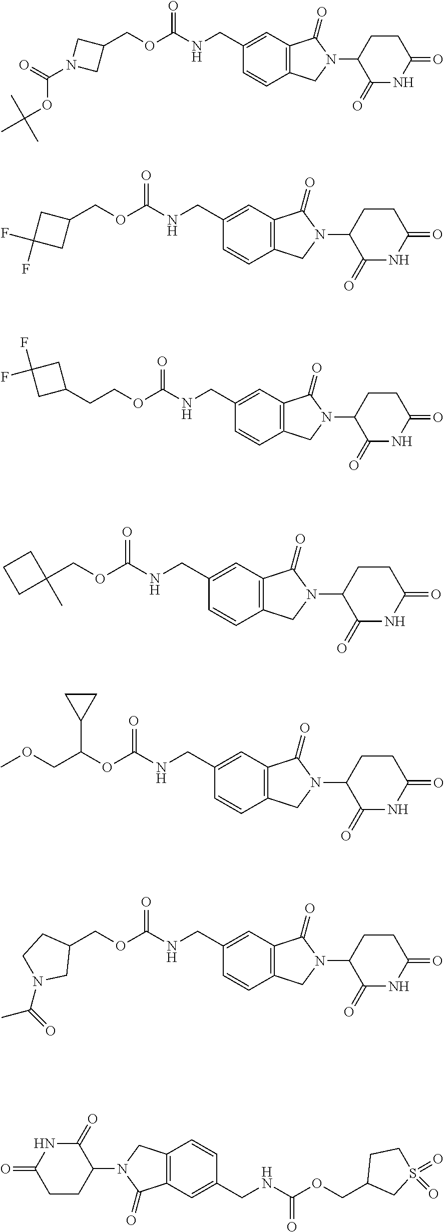 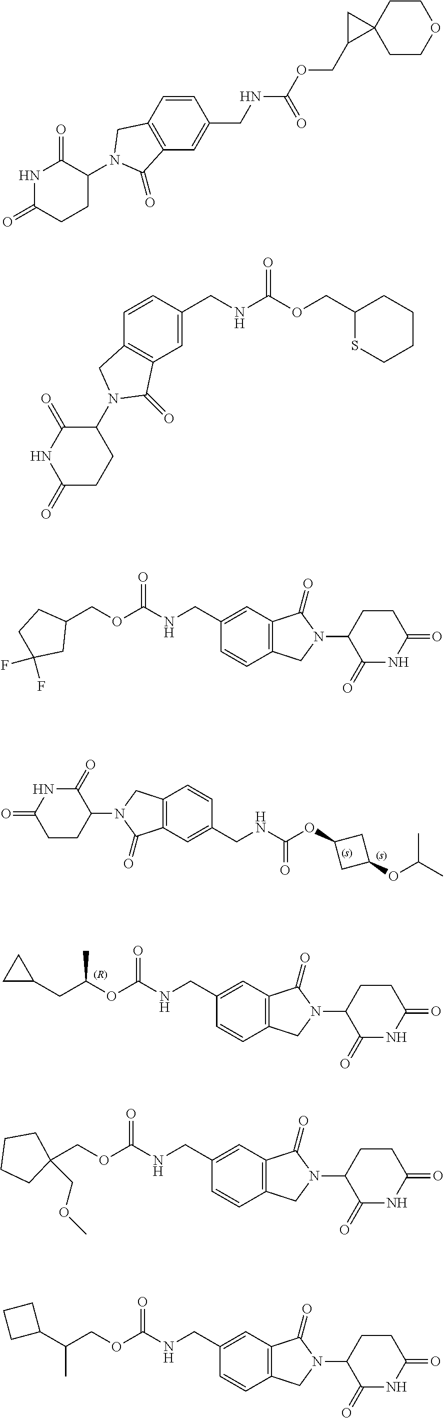 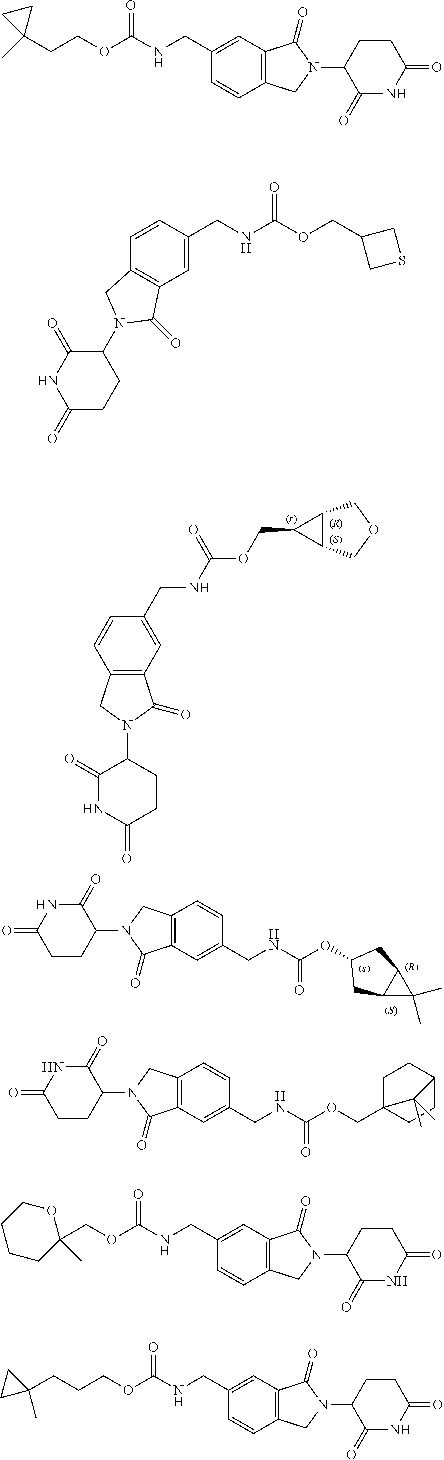 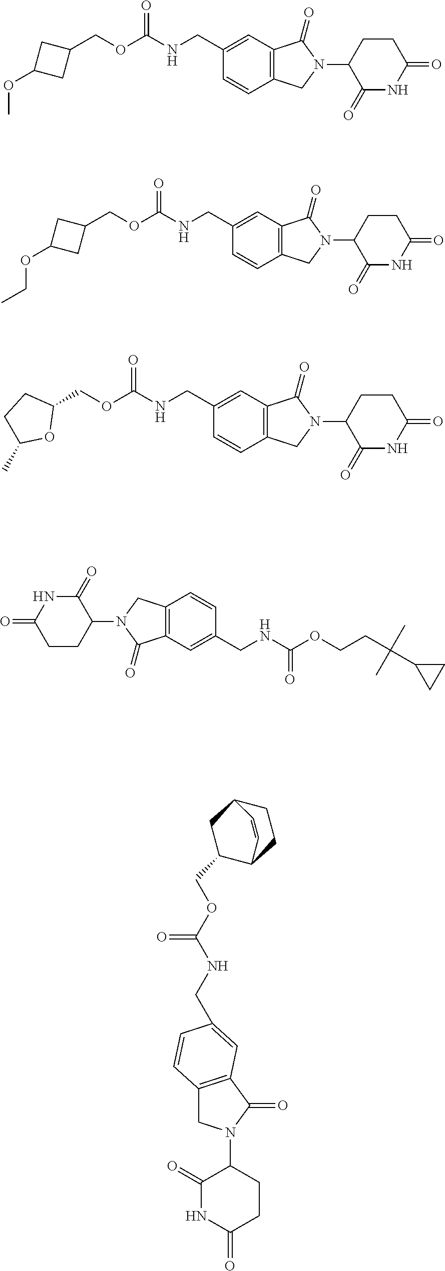 [SMILES: COC1CC(COC(=O)NCc2ccc3c(c2)C(=O)N(C2CCC(=O)NC2=O)C3)C1 ; CCOC1CC(COC(=O)NCc2ccc3c(c2)C(=O)N(C2CCC(=O)NC2=O)C3)C1 ; C[C@@H]1CC[C@H](COC(=O)NCc2ccc3c(c2)C(=O)N(C2CCC(=O)NC2=O)C3)O1 ; CC(C)(CCOC(=O)NCc1ccc2c(c1)C(=O)N(C1CCC(=O)NC1=O)C2)C1CC1 ; O=C1CCC(N2Cc3ccc(CNC(=O)OC[C@@H]4C[C@@H]5C=C[C@H]4CC5)cc3C2=O)C(=O)N1] 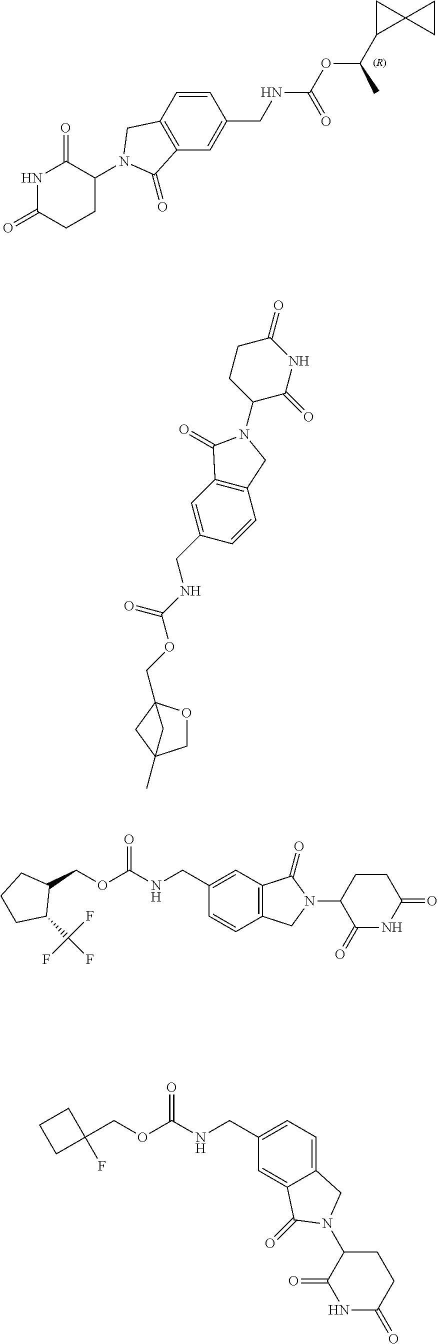 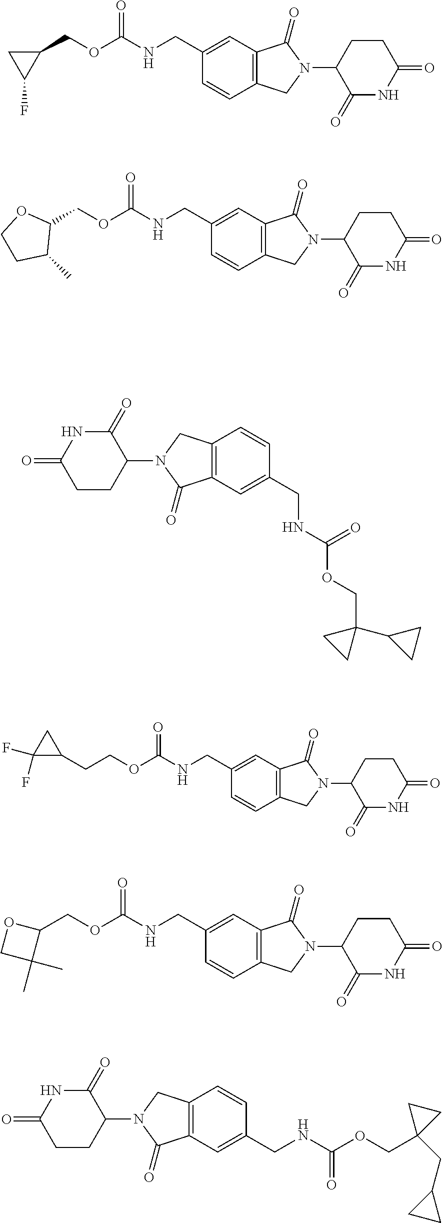 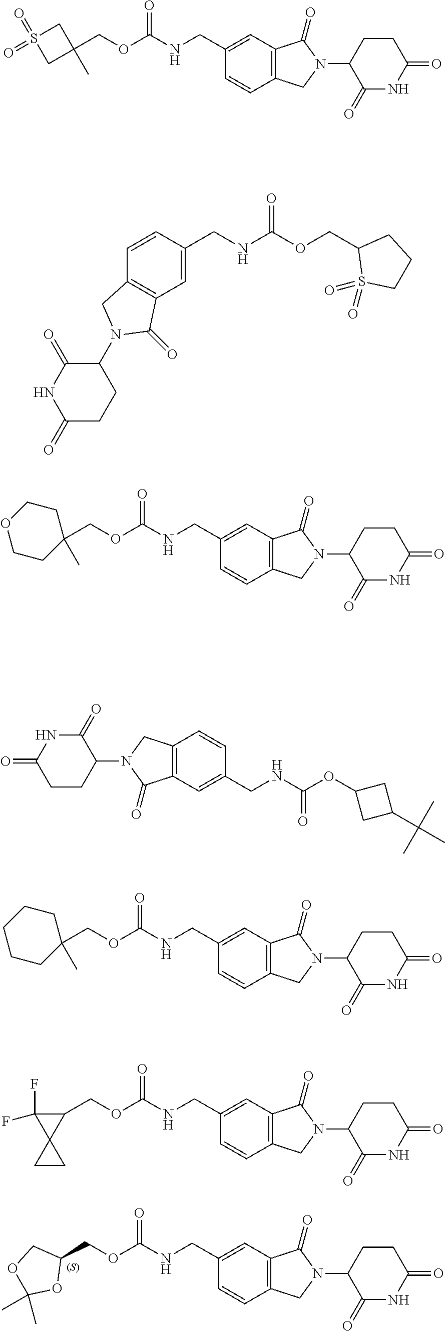 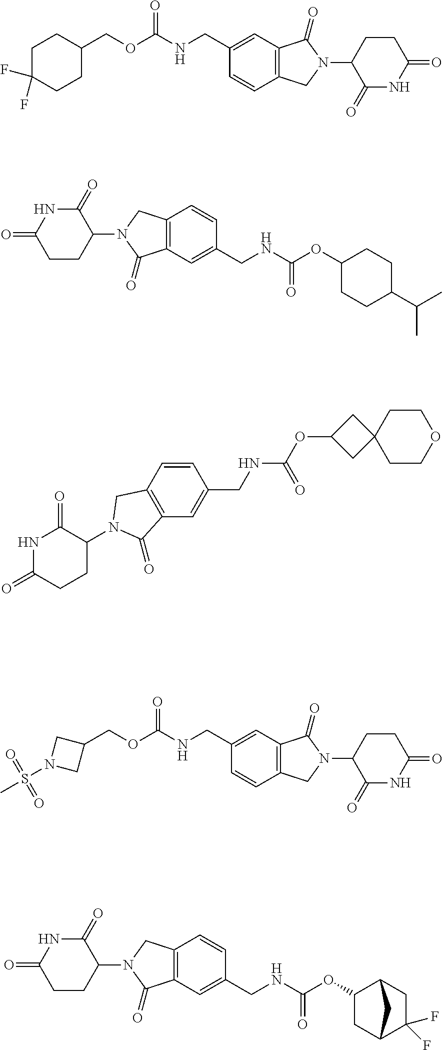 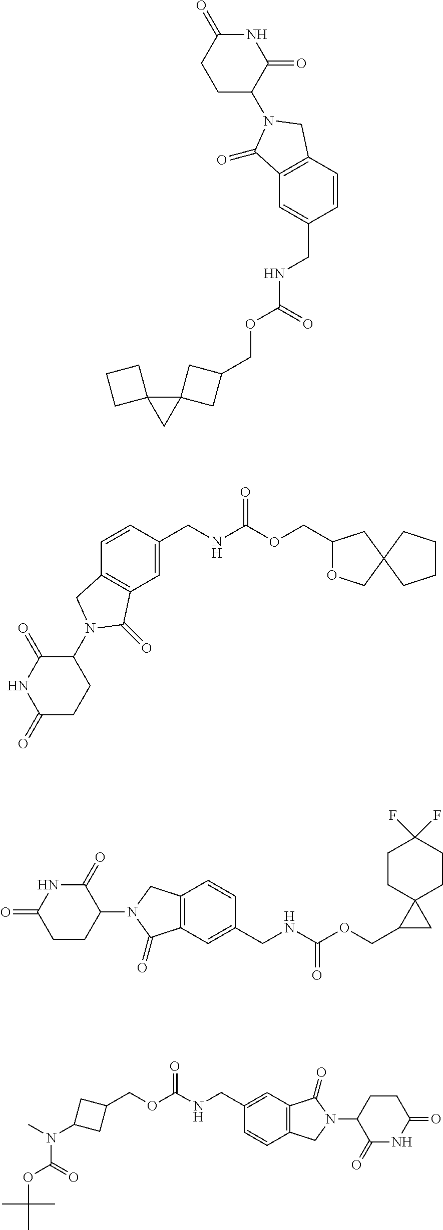 [SMILES: O=C1CCC(N2Cc3ccc(CNC(=O)OCC4CC5(C4)CC54CCC4)cc3C2=O)C(=O)N1 ; O=C1CCC(N2Cc3ccc(CNC(=O)OCC4CC5(CCCC5)CO4)cc3C2=O)C(=O)N1 ; O=C1CCC(N2Cc3ccc(CNC(=O)OCC4CC45CCC(F)(F)CC5)cc3C2=O)C(=O)N1 ; CN(C(=O)OC(C)(C)C)C1CC(COC(=O)NCc2ccc3c(c2)C(=O)N(C2CCC(=O)NC2=O)C3)C1] 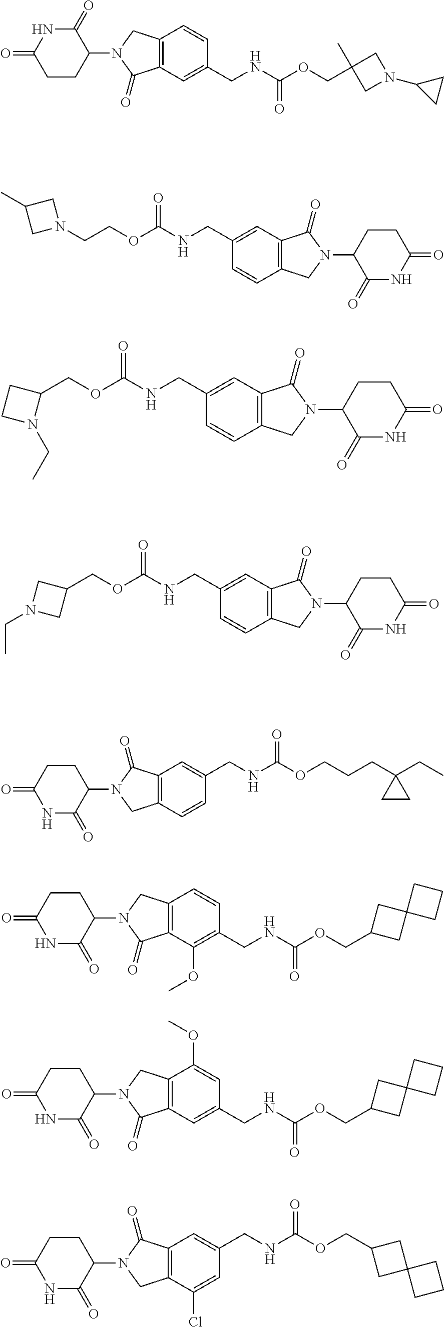 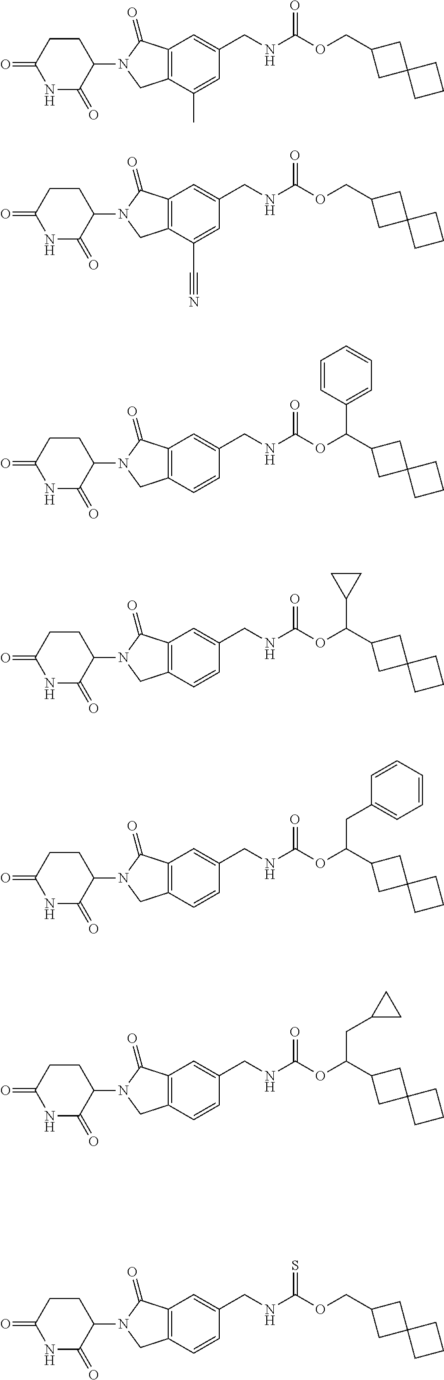 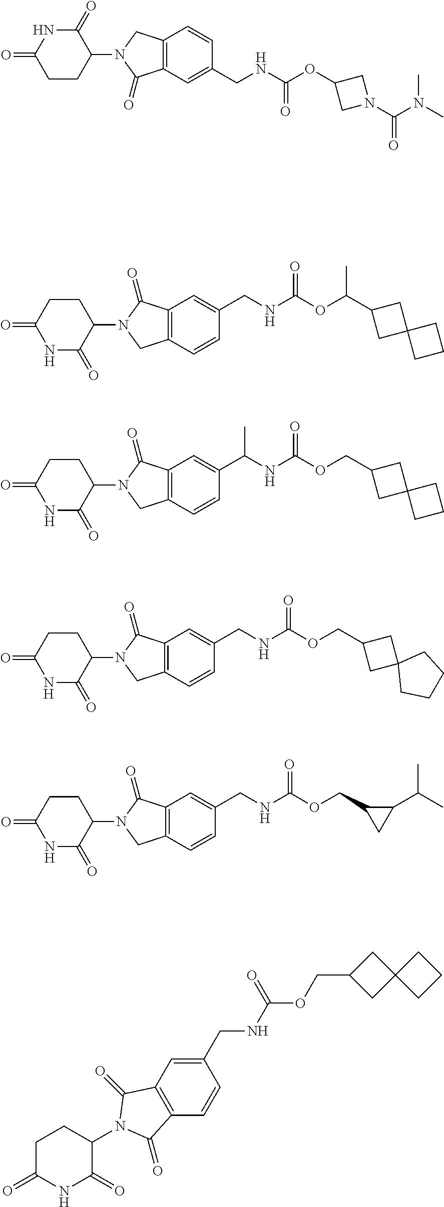 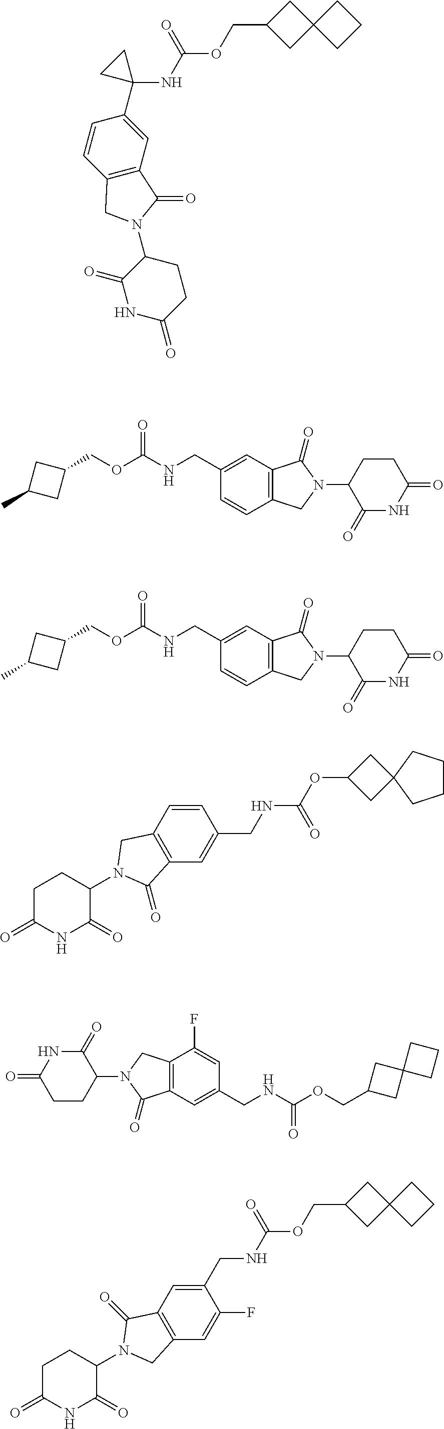 [SMILES: O=C1CCC(N2Cc3ccc(C4(NC(=O)OCC5CC6(CCC6)C5)CC4)cc3C2=O)C(=O)N1 ; C[C@H]1C[C@H](COC(=O)NCc2ccc3c(c2)C(=O)N(C2CCC(=O)NC2=O)C3)C1 ; C[C@H]1C[C@@H](COC(=O)NCc2ccc3c(c2)C(=O)N(C2CCC(=O)NC2=O)C3)C1 ; O=C1CCC(N2Cc3ccc(CNC(=O)OC4CC5(CCCC5)C4)cc3C2=O)C(=O)N1 ; O=C1CCC(N2Cc3c(F)cc(CNC(=O)OCC4CC5(CCC5)C4)cc3C2=O)C(=O)N1 ; O=C1CCC(N2Cc3cc(F)c(CNC(=O)OCC4CC5(CCC5)C4)cc3C2=O)C(=O)N1] 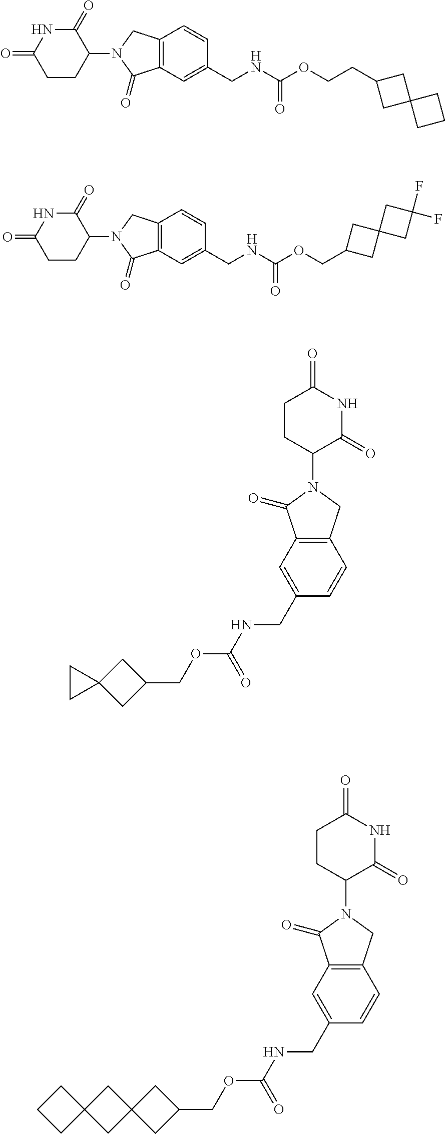 [SMILES: O=C1CCC(N2Cc3ccc(CNC(=O)OCCC4CC5(CCC5)C4)cc3C2=O)C(=O)N1 ; O=C1CCC(N2Cc3ccc(CNC(=O)OCC4CC5(C4)CC(F)(F)C5)cc3C2=O)C(=O)N1 ; O=C1CCC(N2Cc3ccc(CNC(=O)OCC4CC5(CC5)C4)cc3C2=O)C(=O)N1 ; O=C1CCC(N2Cc3ccc(CNC(=O)OCC4CC5(C4)CC4(CCC4)C5)cc3C2=O)C(=O)N1] 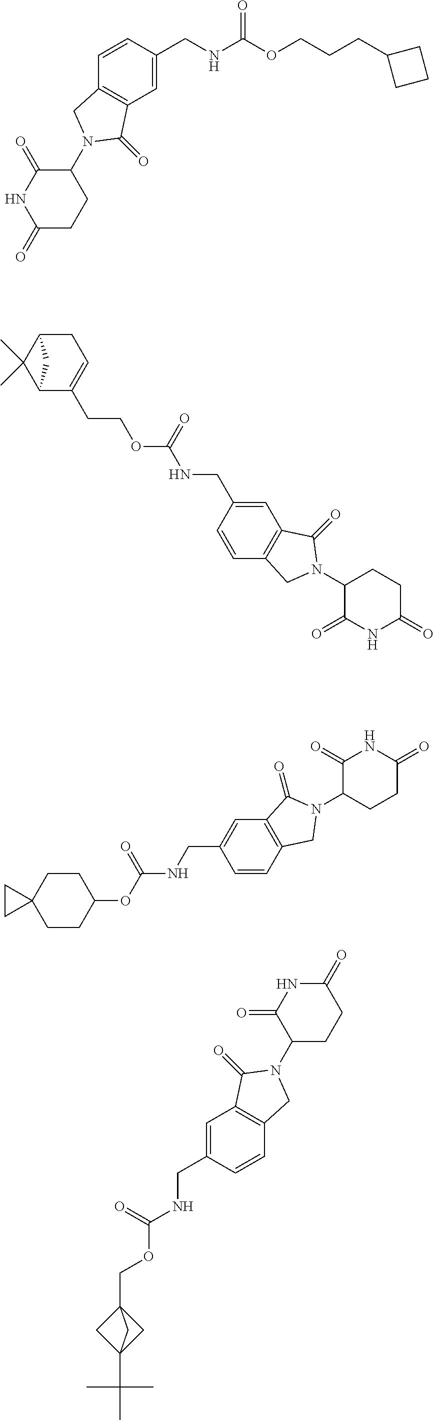 [SMILES: O=C1CCC(N2Cc3ccc(CNC(=O)OCCCC4CCC4)cc3C2=O)C(=O)N1 ; CC1(C)[C@H]2CC=C(CCOC(=O)NCc3ccc4c(c3)C(=O)N(C3CCC(=O)NC3=O)C4)[C@@H]1C2 ; O=C1CCC(N2Cc3ccc(CNC(=O)OC4CCC5(CC4)CC5)cc3C2=O)C(=O)N1 ; CC(C)(C)C12CC(COC(=O)NCc3ccc4c(c3)C(=O)N(C3CCC(=O)NC3=O)C4)(C1)C2] 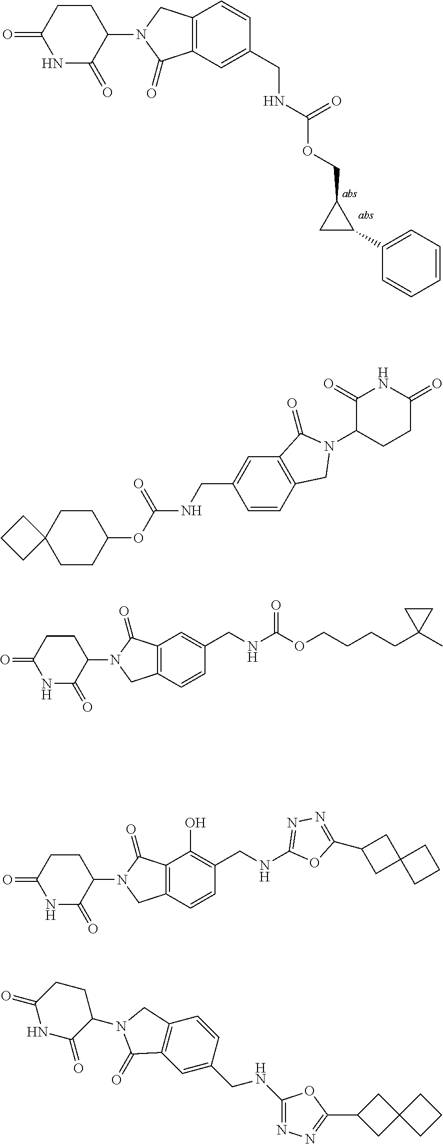 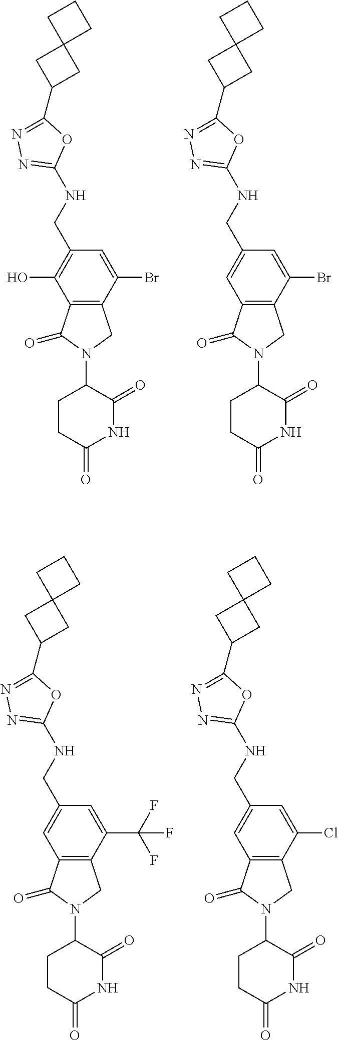 [SMILES: O=C1CCC(N2Cc3c(Br)cc(CNc4nnc(C5CC6(CCC6)C5)o4)c(O)c3C2=O)C(=O)N1 ; O=C1CCC(N2Cc3c(Br)cc(CNc4nnc(C5CC6(CCC6)C5)o4)cc3C2=O)C(=O)N1 ; O=C1CCC(N2Cc3c(cc(CNc4nnc(C5CC6(CCC6)C5)o4)cc3C(F)(F)F)C2=O)C(=O)N1 ; O=C1CCC(N2Cc3c(Cl)cc(CNc4nnc(C5CC6(CCC6)C5)o4)cc3C2=O)C(=O)N1] 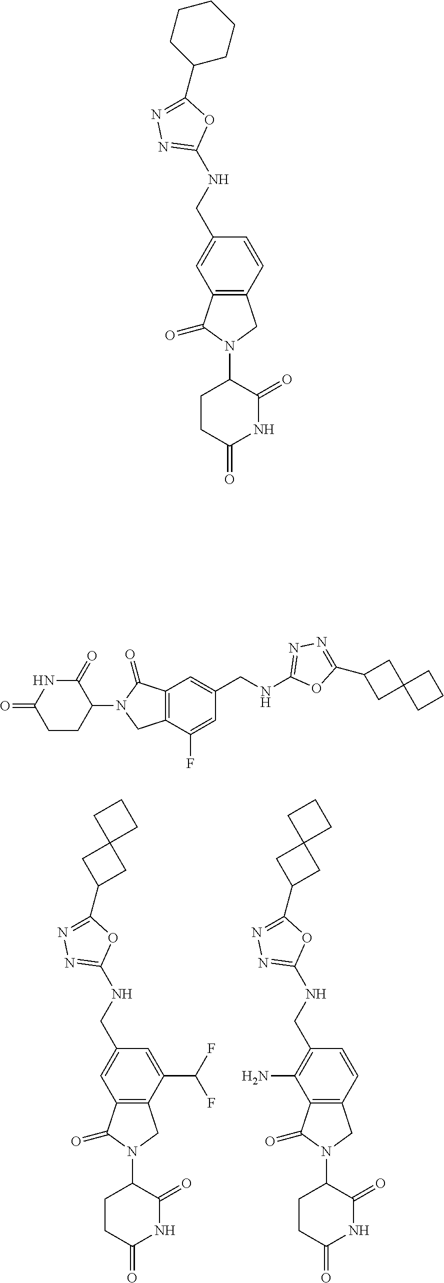 [SMILES: O=C1CCC(N2Cc3ccc(CNc4nnc(C5CCCCC5)o4)cc3C2=O)C(=O)N1 ; O=C1CCC(N2Cc3c(F)cc(CNc4nnc(C5CC6(CCC6)C5)o4)cc3C2=O)C(=O)N1 ; O=C1CCC(N2Cc3c(cc(CNc4nnc(C5CC6(CCC6)C5)o4)cc3C(F)F)C2=O)C(=O)N1 ; Nc1c(CNc2nnc(C3CC4(CCC4)C3)o2)ccc2c1C(=O)N(C1CCC(=O)NC1=O)C2] 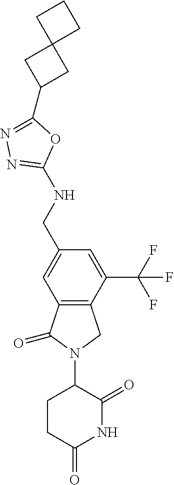 [SMILES: O=C1CCC(N2Cc3c(cc(CNc4nnc(C5CC6(CCC6)C5)o4)cc3C(F)(F)F)C2=O)C(=O)N1]
and pharmaceutically acceptable salts thereof.

16. A pharmaceutical composition comprising the compound of claim 1, or pharmaceutically acceptable salt thereof, and a pharmaceutically acceptable excipient.

17. A pharmaceutical composition comprising the compound of claim 15, or pharmaceutically acceptable salt thereof, and a pharmaceutically acceptable excipient.

18. A method of degrading CDK2 in a subject suffering from cancer, comprising administering to the subject an effective amount of the compound of claim 1.

19. A method of treating cancer in a subject in need thereof, comprising administering to the subject a therapeutically effective amount of the compound of claim 1.
